(19) United States
(12) Patent Application Publication
(10) Pub. No.: US 2022/0240762 A1
(43) Pub. Date: Aug. 4, 2022

(54) SPLIT OVERTUBE ASSEMBLY

(71) Applicants: The Regents of the University of Colorado, a Body Corporate, Denver, CO (US); Aspero Medical, Inc., Boulder, CO (US)

(72) Inventors: Mark E. Rentschler, Boulder, CO (US); Allison B. Lyle, Boulder, CO (US); Jeffrey P. Castleberry, Longmont, CO (US)

(21) Appl. No.: 17/721,157

(22) Filed: Apr. 14, 2022

Related U.S. Application Data
(63) Continuation-in-part of application No. 16/875,793, filed on May 15, 2020.
Application No. 16/875,793 is a continuation-in-part of application No. 16/805,303, filed on Feb. 28, 2020.
Application No. 16/805,303 is a continuation-in-part of application No. 16/249,550, filed on Jan. 16, 2019, now Pat. No. 11,089,944.
(60) Provisional application No. 63/175,151, filed on Apr. 15, 2021.
(60) Provisional application No. 62/849,592, filed on May 17, 2019.
(60) Provisional application No. 62/617,868, filed on Jan. 16, 2018.

(51) Int. Cl. A61B 1/00

(52) U.S. Cl. CPC A61B 1/00135; A61B 1/00082

(57) ABSTRACT

An overtube assembly for use with an elongate medical includes a flexible tubular body having a proximal end and a distal end, the flexible tubular body including a tube split extending longitudinally from the proximal end to the distal end. The flexible tubular body defines each of a primary lumen extending from the proximal end to the distal end and accessible through the tube split and a secondary lumen separate from the primary lumen.

### CROSS-REFERENCE TO RELATED APPLICATIONS

This non-provisional utility application is related to and claims priority under 35 U.S.C. § 119(e) from U.S. Provisional Application No. 63/175,151, filed Apr. 15, 2021, and titled “SPLIT OVERTUBE ASSEMBLY.”

This non-provisional utility application is also a continuation-in-part of U.S. patent application Ser. No. 16/875,793, filed May 15, 2020, and titled “SPLIT OVERTUBE ASSEMBLY,” which is related to and claims priority under 35 U.S.C. § 119(e) from U.S. Provisional Application No. 62/849,592, filed May 17, 2019, and titled “MEDICAL DEVICES INCLUDING TEXTURED SURFACES.”

U.S. patent application Ser. No. 16/875,793 is a continuation-in-part of U.S. patent application Ser. No. 16/805,303, filed Feb. 28, 2020, and titled “MEDICAL DEVICES INCLUDING TEXTURED INFLATABLE BALLOONS.”

U.S. patent application Ser. No. 16/805,303 is a continuation-in-part of U.S. application Ser. No. 16/249,550, filed Jan. 16, 2019, now U.S. Pat. No. 11,089,944, and titled “MEDICAL DEVICES INCLUDING TEXTURED INFLATABLE BALLOONS,” which is related to and claims priority under 35 U.S.C. § 119(e) from U.S. Patent Application No. 62/617,868, filed Jan. 16, 2018, titled “ENDOSCOPIC DEVICES AND METHODS OF USING SAME.”

The entire content of each of the foregoing applications is incorporated herein by reference for all purposes.

### GOVERNMENT SUPPORT STATEMENT

This invention was made with government support under Award Number 2013877 awarded by the National Science Foundation. The government has certain rights in the invention.

### TECHNICAL FIELD

Aspects of the present disclosure are directed to overtube assemblies for use in medical procedures and, in particular, to overtube assemblies including split overtubes.

### BACKGROUND

Endoscopy is a procedure wherein a highly trained physician pushes a long flexible endoscope through a physiological lumen of a patient, such as, but not limited to the colon or small bowel. Conventional endoscopes often struggle to complete procedures that involve irregular anatomy or small bowel examination. These factors can lead to missed diagnoses of early state conditions, such as colorectal cancer, which is the third deadliest cancer in America, but which has a 93% survival rate when detected in its initial stages.

To complete many of these examinations, double balloon enteroscopy (DBE) is often used. The double balloon system includes two balloons, one attached the front of the scope and one attached to a scope overtube. These balloons serve as anchoring points for the endoscope and provide extra support for the long flexible scope to be directed. When these anchoring balloons are inflated and deflated in succession, they aid in the advancement of the scope. When inflated, the balloons push against the wall of the colon, small bowel, or other physiological lumen, and grip the wall forming an anchor point, reducing movement while the scope pushes against the anchor point. DBE has been shown to be a very successful procedure for irregular anatomy patients and small bowel endoscopy.

Balloons commonly used in the art for DBE procedures are conventionally made of smooth latex-like materials. These materials have a low coefficient of friction, especially with the soft, mucous covered wall of the small bowel, colon, and other portions of the gastrointestinal (GI) tract. The low coefficient of friction can cause the balloon to slip prematurely, thus not allowing the scope to properly advance. Over-inflation of the balloons can increase friction with the wall of the small bowel or colon, but at the same time can also cause damage to the patient's GI tract.

Certain enteroscopy devices include the balloons in an overtube that is disposed over the enteroscope. Notably, due to their tubular shape, conventional overtubes require the enteroscope to be inserted through the overtube before insertion of the enteroscope into the patient. As a result, if a physician begins an enteroscopy procedure without an overtube and subsequently determines that an overtube is required, the enteroscope must be fully removed from the patient before attaching the overtube, effectively restarting the enteroscopy procedure.

There is thus a need in the art for novel devices that can be used to perform gastroenterology and other medical procedures. Such devices should increase the amount of successful completions of such procedures, and provide a more comfortable experience for the patient. By allowing for more colonoscopies to be completed fully, more cases of colorectal cancer would be found in early enough stages for successful treatment.

With these thoughts in mind among others, aspects of the devices and methods disclosed herein were conceived.

### SUMMARY

One aspect of the present disclosure includes an overtube assembly for use with an elongate medical device. The overtube assembly includes a flexible tubular body having a proximal end and a distal end, the flexible tubular body including a tube split extending longitudinally from the proximal end to the distal end. The flexible tubular body defines each of a primary lumen extending from the proximal end to the distal end and accessible through the tube split and a secondary lumen separate from the primary lumen.

Another aspect of the present disclosure includes another overtube assembly is provided. The overtube assembly includes a tubular body having a proximal end and a distal end with a tube split extending longitudinally from the proximal end to the distal end. The flexible tubular body defines each of (i) a primary lumen accessible through the tube split and extending from the proximal end to the distal end; (ii) a secondary lumen separate from the primary lumen; and (iii) a fluid supply lumen separate from each of the primary lumen and the secondary lumen. The overtube assembly further includes an inflatable balloon disposed on a distal portion of the tubular body an in communication with the fluid supply lumen such that inflation of the inflatable balloon is controllable by selectively providing or removing fluid via the fluid supply lumen.

In another aspect of the present disclosure, a method is provided that includes disposing an overtube assembly onto an elongate tool. The overtube assembly includes a flexible tubular body having a proximal end and a distal end. The flexible tubular body includes a tube split extending longitudinally from the proximal end to the distal end. The flexible tubular body also defines a primary lumen accessible through the tube split and a secondary lumen separate from the primary lumen. The method further includes disposing the overtube assembly onto the elongate tool includes inserting the elongate tool through the tube split; locating the overtube assembly within a patient; and, subsequent to locating the overtube assembly within the patient, inserting a secondary tool into the secondary lumen.

### BRIEF DESCRIPTION OF THE DRAWINGS

Example implementations of the present disclosure are illustrated in referenced figures of the drawings. It is intended that the implementations and corresponding figures disclosed herein are to be considered illustrative rather than limiting.

FIG. 1A is a side elevation view of a first medical device according to the present disclosure including a balloon in a deflated state.

FIG. 1B is a cross-sectional view of the medical device of FIG. 1A.

FIG. 1C is a side elevation view of the medical device of FIG. 1A in which the balloon is in an at least partially inflated state.

FIG. 1D is a cross-sectional view of the medical device of FIG. 1C.

FIG. 1E is a side elevation view of the medical device of FIG. 1A in the at least partially inflated state and further including a detail view illustrating protrusions disposed on the balloon.

FIGS. 2A-2AD are various views of example protrusions according to the present disclosure.

FIG. 3 is a side elevation view of an alternative balloon according to the present disclosure.

FIG. 4A is a schematic illustration of a textured portion of a balloon according to the present disclosure in a first state of strain.

FIG. 4B is a cross-sectional view of a protrusion of the balloon of FIG. 4A.

FIG. 5A is a schematic illustration of the textured portion of the balloon of FIG. 4A in a second state of strain.

FIG. 5B is a cross-sectional view of the protrusion of FIG. 4B when the balloon of FIG. 4A is in the second state of strain.

FIGS. 6A and 6B are more detailed illustrations of the cross-sectional views of FIGS. 4B and 5B.

FIG. 7 is a graph illustrating an example relationship between separation force and a strain applied to a balloon in accordance with the present disclosure.

FIG. 8 is a cross-sectional view of a first mold for manufacturing balloons in accordance with the present disclosure.

FIG. 9 is an isometric view of a second mold for manufacturing balloons in accordance with the present disclosure.

FIG. 10 is a schematic illustration of a medical device in the form of a catheter delivery tool in accordance with the present disclosure.

FIG. 11 is a schematic illustration of an example endoscopic medical device in accordance with the present disclosure and including a catheter-mounted balloon.

FIG. 12 is a schematic illustration of a second example endoscopic medical device in accordance with the present disclosure and including an endoscope-mounted balloon.

FIG. 13 is a schematic illustration of a third example endoscopic medical device in accordance with the present disclosure and including each of a catheter-mounted balloon and an endoscope-mounted balloon.

FIG. 14 is a schematic illustration of a fourth example endoscopic medical device in accordance with the present disclosure and including an overtube-mounted balloon.

FIG. 15 is a schematic illustration of a fifth example endoscopic medical device in accordance with the present disclosure and including each of a catheter-mounted balloon and an endoscope-mounted balloon.

FIG. 16 is a schematic illustration of a sixth example endoscopic medical device in accordance with the present disclosure and including each of a catheter-mounted balloon, an endoscope-mounted balloon, and an overtube-mounted balloon.

FIG. 17 is a graphical illustration of an example medical procedure performed using the medical device of FIG. 13.

FIG. 18 is a flowchart illustrating an example method of performing a procedure using a medical device according to the present disclosure.

FIG. 19 is a flowchart illustrating a method of modifying engagement between a balloon in accordance with the present disclosure and a physiological lumen.

FIGS. 20A and 20B are schematic illustrations of another example balloon in accordance with the present disclosure in each of an at least partially inflated state and a collapsed state, respectively.

FIGS. 21A-210 are schematic illustrations of yet another example balloon in accordance with the present disclosure in each of a collapsed state, a partially inflated state, and an inflated state, respectively.

FIGS. 22A and 22B are schematic illustrations of another example balloon in accordance with the present disclosure in each of a collapsed state and an at least partially inflated state, respectively, illustrating controlled collapse of the balloon.

FIGS. 23A-23C are schematic illustrations of still another example balloon in accordance with the present disclosure in each of an unstrained state, a collapsed state, and an inflated/strained state, respectively, illustrating an alternative approach to controlled collapse of the balloon.

FIG. 24 is a cross-sectional view of an example balloon having varying wall thickness to facilitate controlled collapse of the balloon.

FIGS. 25A-25D are isometric, plan, end, and partial cross-sectional views of an example balloon having textured portions including transverse protrusions.

FIGS. 26A-26D are isometric, plan, end, and partial cross-sectional views of another example balloon having textured portions including transverse protrusions.

FIGS. 27A-27D are isometric, plan, end, and partial cross-sectional views of an example balloon having texturing portions including radial protrusions.

FIGS. 28A and 28B are schematic illustrations of a first directional balloon in a collapsed state and an at least partially inflated state, respectively.

FIGS. 29A and 29B are schematic illustrations of a second directional balloon in a collapsed state and an at least partially inflated state, respectively.

FIGS. 30A and 30B are schematic illustrations of a balloon having non-uniform inflation in a collapsed state and an at least partially inflated state, respectively.

FIG. 31 is a cross-sectional view of a balloon including multiple and independently inflatable internal chambers.

FIG. 32 is a cross-sectional view of a balloon including an outer sheath/balloon and independently inflatable internal balloons disposed within the outer sheath/balloon.

FIGS. 33-35 illustrate various implementations of protrusion reinforcement on internal surfaces of balloons in accordance with the present disclosure.

FIGS. 36-38 illustrate various implementations of protrusion reinforcement on external surfaces of balloons in accordance with the present disclosure.

FIG. 39 is a schematic illustration of an overtube assembly according to the present disclosure including an integrated inflation/deflation assembly.

FIGS. 40A and 40B are schematic illustrations of an endoscope and split overtube according to the present disclosure in each of a decoupled and coupled arrangement, respectively.

FIG. 41 is a cross-section view of the split overtube of FIGS. 23A and 23B including an inner layer/coating.

FIG. 42 is a cross-section view of the split overtube of FIGS. 23A and 23B including inner texturing.

FIGS. 43-46 are cross-sectional views of alternative split overtubes.

FIG. 47 is an isometric view of a distal portion of a split overtube assembly in accordance with the present disclosure.

FIG. 48 is a plan view of the distal portion of the split overtube assembly of FIG. 47.

FIG. 49 is a side elevation view of the distal portion of the split overtube assembly of FIG. 47.

FIG. 50 a distal end view of the distal portion of the split overtube assembly of FIG. 47.

FIG. 51 is a cross-sectional side view of the distal portion of the split overtube assembly of FIG. 47.

FIG. 52 is a detailed view of a distal end of the split overtube assembly of FIG. 47.

FIGS. 53 and 54 are an isometric view and an end view of an inflatable balloon of the overtube assembly of FIG. 47.

FIGS. 55 and 56 are isometric views of the distal portion of the split overtube assembly illustrating the inflatable balloons in an unsealed and sealed state, respectively.

FIG. 57 is an isometric view of a distal portion of an overtube assembly according to the present disclosure.

FIG. 58 is a distal end view of the overtube assembly of FIG. 57.

FIG. 59 is an isometric view of another overtube assembly according to the present disclosure.

FIG. 60 is a detailed isometric view of a distal portion of the overtube assembly of FIG. 59.

FIG. 61 is a detailed view of a portion of the overtube assembly of FIG. 59 illustrating a closure mechanism.

FIG. 62 is a cross-sectional view of a split overtube assembly including a closure tool.

FIG. 63 is a flow chart describing an example method of manufacturing an overtube assembly, such as the overtube assembly of FIG. 47.

FIGS. 64A-64C illustrate insertion of an endoscope into a physiological lumen using an expandable overtube in accordance with the present disclosure.

FIG. 65 is a schematic illustration of an endoscope disposed within a physiological lumen, the endoscope including a textured endoscopic tool.

FIG. 66 is a schematic illustration of an endoscope disposed within a physiological lumen, the endoscope including a textured catheter.

FIG. 67 is a schematic illustration of a textured biliary/pancreatic stent according to the present disclosure.

FIGS. 68A-68C are schematic illustrations of a physiological lumen illustrating deployment of a tubular mesh stent according to the present disclosure.

FIG. 69 is a schematic illustration of a tapered stent according to the present disclosure.

FIG. 70 is an operational environment and, in particular, a cross-sectional view of a patient abdominal cavity including textured surgical tools in accordance with the present disclosure.

FIG. 71 is a side elevation view of a surgical tool of FIG. 64 in which the texturing is integrated with a shaft of the surgical tool.

FIG. 72 is a side elevation view of the surgical tool of FIG. 64 in which the texturing is provided by a sheath or wrap applied to the shaft of the surgical tool.

FIGS. 73A-73C are side elevation views of textured trocars according to the present disclosure.

FIGS. 74A and 74B are isometric views of a reinforced split overtube assembly alone and coupled to an elongate medical device, respectively.

FIG. 75 is an isometric view of a distal end of the split overtube assembly of FIG. 74B.

FIG. 76 is an isometric view of an intermediate section of the split overtube assembly of FIG. 74A.

FIGS. 77A and 77B are an isometric view of a distal end of a split overtube assembly including internal reinforcements and a corresponding cross-sectional view, respectively.

FIG. 78A is a cross-sectional view of a split overtube including embedded reinforcements in the form of embedded ribs.

FIG. 78B is a side elevation view of a split overtube including embedded reinforcements in the form of braided bands.

FIG. 78C is a side elevation view of a split overtube including embedded reinforcements in the form of coils.

FIG. 79 is an isometric view of a split overtube including various reinforcement structures.

FIG. 80A is an isometric view of a split overtube assembly and backbone-style reinforcing structure in a disassembled state.

FIG. 80B is an isometric view of the split overtube assembly and backbone-style reinforcing structure of FIG. 80A in an assembled state.

FIG. 81 is an isometric view of an alternative reinforcing structure for use with split overtube assemblies according to this disclosure.

FIG. 82A is an isometric view of a split overtube assembly and a wire-style reinforcing structure in a disassembled state.

FIG. 82B is an isometric view of the split overtube assembly and wire-style reinforcing structure of FIG. 82A in an assembled state.

FIG. 83 is an isometric view of a split overtube assembly including a magnetic closure.

FIGS. 84A and 84B are isometric views of a proximal end of a split overtube assembly including a split handle.

FIGS. 85A and 85B are isometric views of a proximal end of a split overtube assembly including a split handle showing a closure in an open and closed configuration, respectively.

FIGS. 86A and 86B are an isometric view of a distal end of a split overtube assembly including a secondary lumen disposed in a lobe and a corresponding cross-sectional view, respectively.

FIGS. 87A and 87B are isometric views of a distal end and a proximal end, respectively, of a split overtube assembly including a secondary lumen with a tool disposed therein.

FIG. 88A is an isometric view of a distal end of a split overtube assembly including a secondary lumen having an angled exit.

FIG. 88B is another isometric view of the distal end of a the split overtube assembly of FIG. 88A with a tool disposed in the secondary lumen.

FIGS. 89A and 89B are an isometric view of a distal end of a split overtube assembly including secondary lumens defined within a wall of a split overtube and a corresponding cross-sectional view, respectively.

FIGS. 90A and 90B are isometric views of a distal end and a proximal end, respectively, of a split overtube assembly including multiple secondary lumens with tools disposed therein.

FIG. 91 is an isometric view of a distal portion of a split overtube assembly including a secondary lumen having an exit located proximal a distal end of the split overtube assembly.

FIGS. 92A-92C are photographs illustrating insertion of an elongate medical tool into a split overtube assembly according to the present disclosure.

FIGS. 93A and 93B are an isometric view and a detailed isometric view of a split overtube including an insertion feature.

FIGS. 94 and 95 are cross-sectional views of split overtubes including insertion features formed by altering thickness and material, respectively.

FIG. 96 is a side elevation view of a split overtube including an insertion feature defined by selectively modifying reinforcement of the split overtube.

FIGS. 97-99 are side elevation views of split overtubes including insertion features defined by altering characteristics and configurations of reinforcing structures.

FIGS. 100A and 100B are a plan view and a cross-sectional view, respectively, of a split overtube defining an insertion feature by varying split dimensions of reinforcing structure.

FIGS. 101A-101C are isometric views illustrating assembly of a split overtube using a layering and thermoforming technique.

FIGS. 102 and 103 are an isometric view and an end view of a layered assembly for use in manufacturing split overtubes including secondary channels.

FIGS. 104A-104D are side elevation views of layered assemblies for manufacturing split overtubes including various configurations of reinforcing structures.

FIGS. 105A-105C are isometric views of a sheet-based manufacturing technique for split overtubes.

FIGS. 105D and 105E are plan views of layered sheets including braided band- and coil-based reinforcing structures, respectively.

FIGS. 106A and 106B are isometric views of a split overtube during manufacturing (e.g., disposed on a mandrel) and as assembled, respectively.

FIG. 107 is an isometric view of a split overtube manufactured using a mandrel-based technique and including secondary lumens.

FIG. 108 is an isometric view of a split overtube manufactured using a mandrel-based technique and including each of secondary lumens and an insertion feature.

FIG. 109 is an isometric view of a split overtube assembly including the split overtube of FIG. 108.

FIG. 110 is an isometric view of a distal end of a split overtube assembly including multiple secondary channels for providing enhanced functionality.

FIG. 111A is an isometric view of a distal end of the split overtube assembly of FIG. 110 coupled to an endoscope.

FIG. 111B is an isometric view of a distal end of the split overtube assembly of FIG. 110 coupled to a large diameter tool.

FIG. 111C is an isometric view of a distal end of the split overtube assembly of FIG. 110 coupled including an insertion sleeve for use with small diameter tools.

FIG. 112 is a cross-sectional view of a split overtube including auxiliary components disposed within and at a distal end of respective secondary lumens.

FIG. 113 is a cross-sectional view of a split overtube including a surface mounted auxiliary component including a communication line extending through a secondary lumen.

FIG. 114 is a distal end view of an elongate medical tool including a longitudinal guide.

FIG. 115 is a distal end view of a split overtube including a longitudinal rail configured to be received by the guide of the elongate medical tool of FIG. 114.

FIGS. 116A-116C are isometric views of the distal end of a split overtube assembly including the split overtube of FIG. 115 with the tool of FIG. 114 inserted therein and in various states of relative longitudinal displacement.

FIGS. 117A-117C are isometric views of a distal end of an assembly including the tool of FIG. 114 coupled to a tubular structure including a rail adapted to be received in the guide of the tool.

FIG. 117D is an isometric view of the distal end of the assembly of FIGS. 117A-C further including a supplemental tool extending through the tubular structure.

FIGS. 118A and 118B are isometric views of a distal end of an assembly including the tool of FIG. 114 coupled to a supplemental tool tubular including a rail adapted to be received in the guide of the tool.

FIGS. 119A and 119B are isometric views of a distal end of an assembly including a split overtube having an external guide with FIG. 119B illustrating a supplemental tool having a corresponding rail coupled to the split overtube.

FIG. 119C is an isometric view of the distal end of the assembly of FIG. 119A with a tubular structure having a rail corresponding to the groove of the split overtube coupled to the split overtube.

FIG. 119D is another isometric view of the distal end of the assembly of FIG. 119C with a supplemental tool extending through the tubular structure.

FIG. 120 is an isometric view of a distal end of an assembly including a split overtube having each of an internal and an external rail with the tool of FIG. 114 disposed within the split overtube.

FIG. 121A is an isometric view of a distal end of an assembly including a split overtube containing an elongate medical tool, the split overtube including a collapsible secondary lumen in a collapsed state.

FIG. 121B is a cross-sectional view of the split overtube of FIG. 121A with the secondary lumen in the collapsed state.

FIG. 122A is an isometric view of the distal end of the assembly of FIG. 121A with the secondary lumen in an expanded or open state and containing a supplemental tool.

FIG. 122B is a cross-sectional view of the split overtube of FIG. 121A with the secondary lumen in the expanded or open state.

FIG. 123 is a schematic illustration of an example working environment for implementations of split overtube assemblies according to this disclosure.

### DETAILED DESCRIPTION

The current disclosure relates in part to balloon designs that can be incorporated into medical devices, such as endoscopes. The current disclosure further relates to overtubes incorporating such balloons that may be coupled to medical devices, such as endoscopes. More particularly, the current disclosure relates to balloons having exterior surfaces that are at least partially textured. Texturing of the balloons is achieved by the inclusion of multiple pillar-like protrusions extending from the surface of the balloon. In at least one application of the current disclosure, a medical device including the balloon is disposed within a physiological lumen with the balloon in a substantially deflated state. The physiological lumen may be a portion of a patient's GI tract, but more generally may be any vessel, airway, duct, tract, stricture, sphincter, biliary stricture, or similar physiological structure. Once positioned within the physiological lumen, the balloon may be inflated such that the protrusions contact the lumen wall, thereby engaging the balloon and medical device with the lumen wall. The balloon may be subsequently deflated to facilitate disengagement of the protrusions from the wall of the lumen, thereby permitting movement of the medical device. Accordingly, the balloons (or similar structures) disclosed herein include textured/patterned surfaces that provide increased friction and adhesion with biological tissue as compared to conventional smooth balloons. As a result of such increased friction and adhesion, balloons in accordance with the present disclosure more reliably engage biological tissue as compared to conventional balloon designs.

As described below in further detail, the shape and distribution of the protrusions may vary in applications of the present disclosure to provide varying degrees of traction between the balloon and the biological tissue with which the balloon is in traction. In certain implementations, the protrusions may also be configured to deform in response to a strain applied to the balloon. Such deformation alters the adhesive and frictional properties of the protrusions. As a result, a physician may control the relative traction of the balloon to the biological tissue by selectively inflating or deflating the balloon. For example, a physician may apply a first strain to the balloon (e.g., by inflating the balloon to a first extent) resulting in a first degree of deformation of the protrusions and a corresponding first engagement level of the balloon (e.g., a first level of engagement based on the adhesive and frictional properties of the protrusions when in a first shape). Subsequently, the physician may apply a second strain (e.g., by modifying the degree to which the balloon is inflated) resulting in a second degree of deformation of the protrusions and a corresponding second engagement level of the balloon.

In certain implementations of the present disclosure, the foregoing balloons may be incorporated into an overtube assembly that may be coupled to an endoscope (or similar elongate medical device) to facilitate transit of the endoscope within a physiological lumen of a patient. In at least some implementations, the overtube assembly includes a split overtube that facilitates coupling of the overtube assembly without removing the endoscope from a patient.

Although discussed herein primarily in the context of endoscopic balloons for use in the GI tract, the present disclosure may be used in a variety of medical and non-medical applications. Accordingly, to the extent that any particular applications of the present disclosure are discussed herein, such applications should not be viewed as limiting the scope of the present disclosure. Nevertheless, example implementations of the present disclosure are discussed below to provide additional details regarding aspects of the present disclosure.

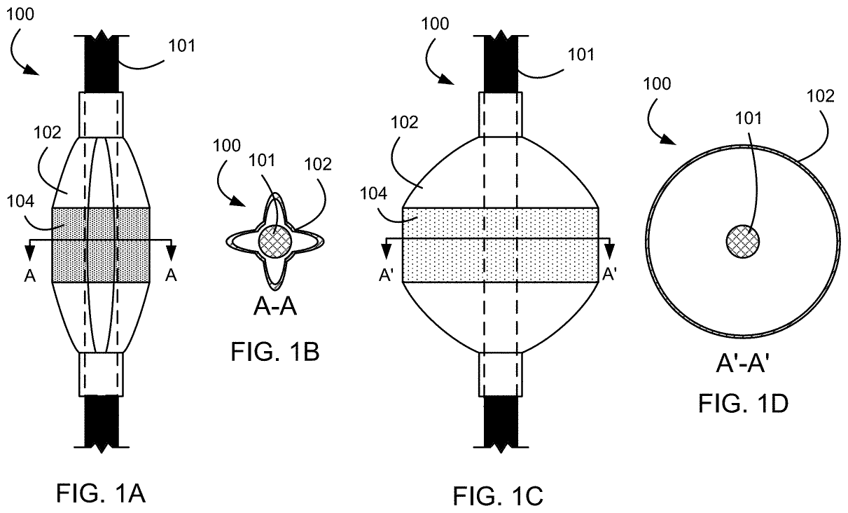

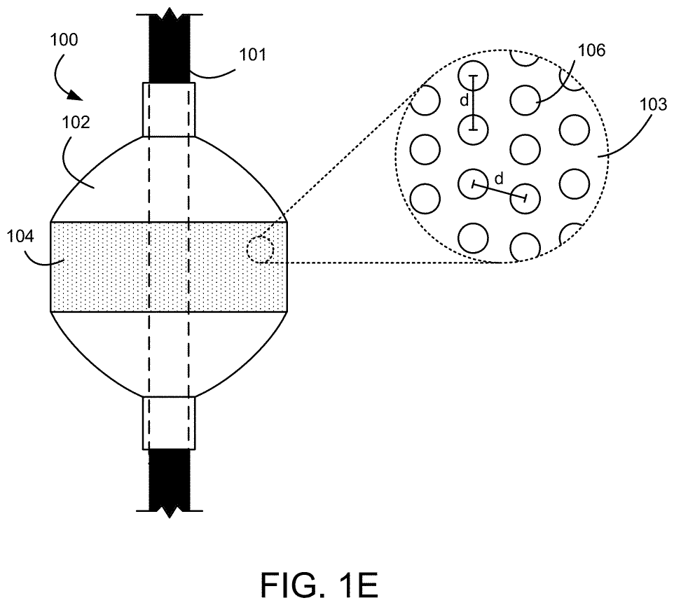

FIGS. 1A-1E are various views of an example medical device 100 including an inflatable balloon 102 in accordance with the present disclosure. More specifically, FIG. 1A is a side elevation view of the medical device 100 with the balloon 102 in deflated or collapsed state, FIG. 1B is a cross-sectional view along cross-section A-A of the balloon 102 of FIG. 1A, FIG. 1C is a side elevation view of the medical device 100 in an at least partially inflated state, FIG. 1D is a cross-sectional view along cross-section A′-A′ of the balloon 102 of FIG. 1C, and FIG. 1E is a side elevation view of the medical device 100 including an inlay illustrating a textured portion 104 of the balloon 102.

For purposes of the present disclosure, balloons disclosed herein are described as being in various states corresponding to various stages of inflation and deflation. An “unstrained state”, for example, refers to a state in which in which the corresponding balloon may be partially inflated but not yet subject to strain and, as a result, generally corresponds to the “as-molded” shape of the balloon. A “strained state” generally refers to a state in which a balloon is inflated beyond the extent necessary to achieve the unstrained state. A “collapsed state”, in contrast, generally refers to a state of the balloon in which at least a portion of the balloon constricts or is otherwise reduced as compared to the unstrained state. In certain implementations, balloons in accordance with the present disclosure may be biased into a collapsed state. Alternatively, balloons in accordance with the present disclosure may transition into the collapsed state in response to air (or other gas) being removed from the balloon or in response to the balloon being otherwise deflated from the unstrained state. Balloons herein may also be described as being “at least partially inflated”, which generally refers to a state of the balloon including the unstrained state and any degree of inflation beyond the unstrained state. Similarly, the “collapsed” state may generally refer to balloons that are in any degree of collapse up to but excluding the unstrained state.

During use, the medical device 100 may be inserted into and located within a physiological lumen of a patient. Such insertion may generally be performed while the balloon 102 is in the deflated state illustrated in FIG. 1A. Once properly located, air or a similar fluid medium may be provided to the balloon 102 to inflate the balloon, as shown in FIG. 1B. When such inflation is performed with the balloon 102 within the physical lumen, at least a part of the textured portion 104 may be made to abut an inner wall of the physiological lumen, thereby causing frictional and adhesive engagement between the textured portion 104 and the physiological lumen and mucosal lining.

Various arrangements for the balloon 102 on the medical device 100 are feasible. In the specific example of FIGS. 1A-1E, the balloon 102 has a cylindrical body capped by hemispherical ends. In another non-limiting example, the balloon 102 is disposed around an endoscope 101 or similar tubular body of the medical device 100 such that the balloon 102 forms a toroidal or spherical shape having a central lumen. In another non-limiting example, the balloon 102 is disposed around the endoscope 101 forming a cylindrical shape having hemispherical rounded ends, wherein the endoscope 101 runs along the major axis of the cylinder. In other implementations, the balloon 102 may be ellipsoid in shape or “pill” shaped. Regardless of the foregoing, balloons in accordance with the present disclosure may be substantially any shape as desired.

The balloon 102 may be made of at least one non-rigid material. For example, in one example implementation the balloon material may include one or more of low-density polyethylene (LDPE), latex, polyether block amide (e.g., PEBAX®), silicone, polyethylene terephthalate (PET/PETE), nylon, polyurethane, and any other thermoplastic elastomer, siloxane, or other similar non-rigid materials. In certain implementations, the balloon 102 may be formed from one material; however, in other implementations the balloon 102 may be formed from multiple materials. For example, the balloon 102 may include a body formed from a first material but may also include reinforcing or structural members formed from a second material.

Material selection for the balloon 102 may also be based, in part, on material hardness. Although material hardness may vary based on application, in at least one specific implementation, the balloon 102 may be formed from a material having a predetermined hardness of Shore 30A such as, but not limited to, Dow Corning Class VI Elastomer C6-530, which is a liquid silicone rubber elastomer.

In general, the balloon 102 has a first diameter or shape when in a collapsed or unstrained state and a second diameter when inflated into an unstrained state, the second diameter being larger than the first diameter. In certain implementations, the balloon 102 may be further inflatable beyond the unstrained state into a strained state. For example, in at least one implementation the balloon 102 can be strained up to about 1,000% relative to its uninflated state, although other maximum strain levels are possible. In other implementations, the balloon 102 does not have a set lower inflation limit. The balloon 102 may also be configured to be inflated to a first turgid state having a defined shape and then be further inflated up to a maximum strain while retaining the defined shape.

The balloon 102 may be structured such that, when deflated or due to biasing, the balloon 102 collapses into a particular shape. For example, as illustrated in FIGS. 1A and 1B, the balloon 102 may be configured to collapse into a star or similar shape. Such controlled collapse of the balloon 102 may be achieved in various ways including, without limitation, selectively reinforcing portions of the balloon 102 with additional material and including semi-rigid structural elements coupled to or embedded within the balloon 102. In other implementations, the balloon 102 may form a pill, ovoid, or similar elongated shape when deflated, including a shape that substantially corresponds to the inflated shape of the balloon 102.

As illustrated FIG. 1C, the balloon 102 includes at least one textured portion 104. In general, and as illustrated in the inlay of FIG. 1C, the textured portion 104 includes multiple protrusions, such as protrusion 106, extending from a surface 103 of the balloon 102. The protrusions 106 of the textured portion 104 may have any pattern. For example, and without limitation, the textured portion 104 may include evenly spaced protrusions arranged in a regular geometric pattern, such as a grid. The balloon 102 illustrated in FIG. 1C, for example, includes protrusions arranged in a triangular grid pattern. In other implementations, other grid patterns may be used including, without limitation, square, rectangular, hexagonal, and octagonal grid patterns or any other suitable grid pattern based on a tessellation of geometric shapes. In certain implementations, the textured portion 104 may include multiple areas of protrusions, with each area having a different protrusion density or protrusion pattern. In still other implementations, the protrusions may be arranged in a random or semi-random pattern across the textured portion 104. More generally, textured portions in accordance with implementations of the present disclosure may include any suitable arrangement of protrusions.

In certain implementations, the protrusions 106 may be evenly spaced such that the center-to-center dimension between adjacent protrusions is constant in a given state of the balloon 102 (e.g., the unstrained state). For example, in one implementation the center-to-center spacing between protrusions (as indicated in the inlay of FIG. 1E by dimension “d”) may be about 20 μm to about 1,000 μm in the unstrained state. In other implementations, the protrusions may be evenly spaced with a center-to-center spacing from and including about 50 μm to and including about 750 μm apart from one another. In yet another implementation, the protrusions may be evenly spaced with a center-to-center spacing from and including about 100 μm to and including about 600 μm apart from one another. In still other implementations, the center-to-center spacing between protrusions may be greater than 1000 μm.

The inset of FIG. 1E illustrates the protrusions 106 arranged in longitudinally extending rows with adjacent rows being offset but equally spaced. It should be appreciated, however, that in other implementations of the present disclosure, aspects of the arrangement of the protrusions 106 may vary. For example, in certain implementations, protrusions of adjacent longitudinal rows may be aligned with each other. Similarly, all rows may be spaced uniformly (e.g., all rows may be spaced 1000 μm apart). Alternatively, spacing between all rows may vary or may only be uniform for a subset of adjacent rows. As yet another example, rows of the protrusions may extend along varying lengths of the textured portion 104. Moreover, in at least certain implementations, the protrusions 106 may not be arranged in longitudinal rows. Rather, the protrusions may be arranged in any suitable pattern including, without limitation, circumferential rows, biased rows (e.g., rows extending both longitudinally and circumferentially), or in a random or pseudo-random pattern.

The protrusions 106 may be formed in various ways. For example and without limitation, the protrusions may be integrally formed with the balloon 102 (e.g., by simultaneously molding the balloon 102 and the protrusions), may be separately formed from and subsequently attached to the balloon 102 (e.g., by first extruding the balloon and then adhering the protrusions to the balloon 102), or may be formed directly onto the balloon 102 (e.g., by a co- or over-molding process in which the balloon 102 is first molded and then the protrusions are molded onto the balloon 102).

As illustrated, the textured portion 104 including the protrusion is disposed between the hemispherical end portions of the balloon 102; however, it should be appreciated that any portion of the balloon 102 may correspond to the textured portion 104. For example, in certain implementations, the textured portion may include either or both of the end portions of the balloon 102, an intermediate section disposed between the end portions, or any variations thereof. Moreover, balloons in accordance with the present disclosure may include multiple, separated textured portions. For example, in certain implementations, each of the end portions of the balloon may be textured while the intermediate portion of the balloon may be left untextured.

As previously discussed, balloons according to the present disclosure may be configured to inflate or deflate in a particular manner. For example, as illustrated in FIG. 1A, the balloon 102 is configured to collapse into a star- or clover-shape when deflated. More specifically, the balloon 102 is configured such that certain longitudinal sections of the balloon 102 are collapsed to a greater degree than others when air is removed from the balloon 102. Such selective collapse may be achieved, for example, by increasing the thickness of the balloon 102 in the longitudinal portions that are to remain protruding when the balloon 102 is deflated.

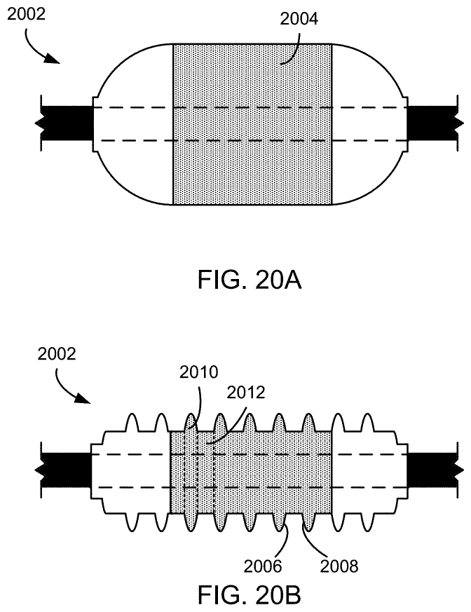

A similar design is illustrated in FIGS. 20A-20B. More specifically, FIG. 20A illustrates a balloon 2002 in an at least partially inflated state while FIG. 20B illustrates the balloon 2002 in a collapsed state. Similar to the balloon 102 of FIGS. 1A-1B, the balloon 2002 is configured to selectively collapse when deflated. More specifically, and as illustrated in FIG. 20B, the balloon 2002 is generally divided into alternating axial bands configured to have different diameters when collapsed. For example, a first band 2010 is configured to collapse to a lesser degree than a second band 2012. As previously noted, such selective collapse may be achieved by increasing the thickness of the first band 2010 or by otherwise reinforcing the first band 2010. In other implementations, the shape of at least some of the bands when in the deflated state may be dictated by a mandrel or similar body disposed within the balloon 2002 and about which the balloon 2002 collapses when deflated.

Varying the degree to which the balloon collapses, as illustrated in the examples of FIGS. 1A and 1B and FIGS. 20A and 20B, facilitates insertion and transportation of the balloon when in the deflated state. In particular, by reducing the proportion of protrusions that are outwardly/radially facing or otherwise disposed at a maximum diameter, the overall adhesion and friction provided by the balloon is reduced. As a result, the likelihood and amount of contact between the balloon and a wall of a physiological lumen is significantly reduced. Referring to FIGS. 20A-20B, for example, the balloon 2002 includes a textured portion 2004 having protrusions according to the present disclosure. When in the at least partially inflated state (as shown in FIG. 20A), each of the protrusions is directed substantially outwardly/radially and, as a result, is able to readily contact and engage the wall of the physiological lumen. However, when in the collapsed state (as shown in FIG. 20B), sections of the textured portion 2004 of the balloon 2002 (such as faces 2006 and 2008) and their respective protrusions are directed at least partially in a longitudinal direction and, as a result, are less likely to directly engage the wall of the physiological lumen. Similarly, sections of the textured portion 2004 (such as the second band 2012) may be recessed when the balloon 2002 is in the deflated state relative to other sections of the textured portion 2004 (such as the first band 2010). As a result, the recessed sections are less likely to contact and engage the wall of the physiological lumen.

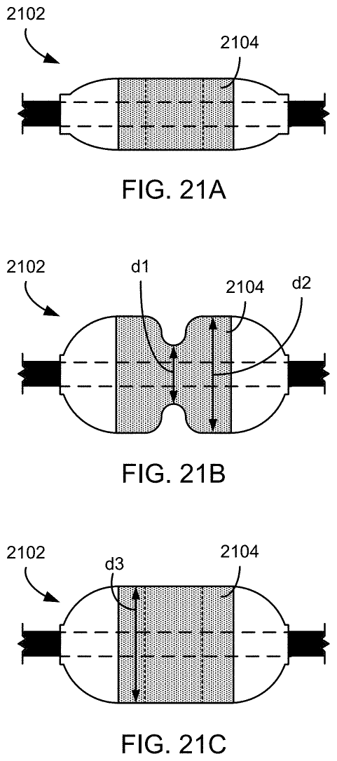

FIGS. 21A-210 illustrates another example balloon 2102 exhibiting non-uniform inflation/deflation. FIG. 21A illustrates the balloon 2102 in a collapsed or unstrained state and in which the balloon 2102 assumes a pill-shaped configuration. As shown in FIG. 21B, the balloon 2102 may be inflated to a first inflation level in which the balloon 2102 assumes an hourglass (or similar shape) in which at least a portion of the balloon 2102 expands to a diameter (d1) that is less than a diameter (d2) of other portions of the balloon 2102. At a second inflation level, the balloon 2102 may expand such that the diameter of the balloon is substantially uniform (d3).

In certain implementations, the controlled inflation of the balloon 2102 may be used to vary the adhesive and frictional force between the balloon 2102 and a wall of a physiological lumen within which the balloon 2102 is disposed. For example, the balloon 2102 includes a textured portion 2104 having protrusions according to the present disclosure. When in the partially inflated state (as illustrated in FIG. 21B), the diameter of the textured portion 2104 varies such that only a limited proportion of the protrusions are each of disposed at the maximum diameter of the balloon 2102 and oriented in an outward/radial direction. As a result, the adhesion and friction between the balloon 2102 and wall of the physiological lumen is reduced as compared to when the balloon 2102 is further inflated (as illustrated in FIG. 21C) such that substantially all of the textured portion 2104 is at the same diameter. Accordingly, a user of the balloon 2102 may inflate the balloon 2102 to the first inflation level to achieve a first degree of engagement and to the second inflation level to achieve a second, greater degree of engagement.

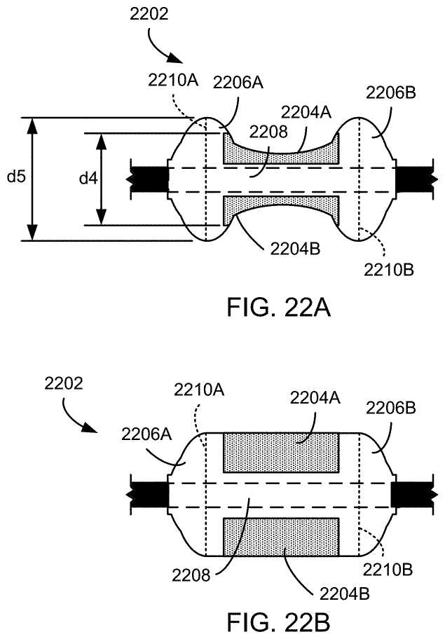

FIGS. 22A and 22B illustrate another example balloon 2202. FIG. 22A illustrates the balloon 2202 in a collapsed state while FIG. 22B illustrates the balloon 2202 in an at least partially inflated state. As shown, the balloon 2202 generally includes textured portions 2204A, 2204B disposed between two untextured ends 2206A, 2206B. The balloon 2202 also includes an untextured portion 2208 disposed between the textured portions 2204A, 2204B.

The textured portions 2204A, 2204B and the untextured ends 2206A, 2206B are structured such that, when in the collapsed state illustrated in FIG. 22A, the textured portions 2204A, 2204B have a maximum diameter (d4) that is less than a maximum diameter (d5) of the untextured ends 2206A, 2206B. In such an arrangement, the outermost surface of the balloon 2202 is provided by the untextured ends 2206A, 2206B while the textured portions 2204A, 2204B are disposed radially inward of the outermost surface. In other words, when in the collapsed state, the textured portions 2204A, 2204B may become concave. As a result, when in the collapsed state illustrated in FIG. 22A, contact between the balloon 2202 and an inner surface of a physiological lumen within which the balloon 2202 may be disposed is primarily between the inner surface of the physiological lumen and the untextured ends 2206A, 2206B.

As the balloon 2202 is inflated, the diameter of the textured portions 2204A, 2204B may expand to at least equal that of the untextured ends 2206A, 2206B, as illustrated in FIG. 22B. As a result, the textured portions 2204A, 2204B may come into contact with the inner surface of the physiological lumen, thereby increasing friction between the balloon 2202 and the inner surface of the physiological lumen.

In light of the arrangement illustrated in FIGS. 22A and 22B, the balloon 2202 may be inserted into and moved along the physiological lumen in the deflated/low-friction state illustrated in FIG. 22A. When the balloon 2202 is at an intended location, the balloon 2202 may then be inflated to expose the textured portions 2204A, 2204B and to cause the textured portions 2204A, 2204B to come into contact with the inner surface of the physiological lumen. Doing so increases friction between the balloon 2202 and the inner surface of the physiological lumen and may be used to anchor or otherwise reduce movement of the balloon 2202 within the physiological lumen.

As illustrated in FIGS. 22A and 22B, in at least some implementations of the present disclosure, an untextured portion 2208 may be disposed between textured portions of the balloon 2202. For example, one or more untextured portions 2208 may extend longitudinally between textured portions of the balloon 2202, such as the textured portions 2204A, 2204B. When in the collapsed state illustrated in FIG. 22A, the untextured portion 2208 may have a diameter similar to that of the untextured ends 2206A, 2206B, thereby providing another low-friction surface that contacts the inner surface of the physiological lumen during insertion and transportation. In such cases, when in the deflated configuration, the textured portions 2204A, 2204B may generally be concave about an axis extending perpendicular to a longitudinal axis of the balloon 2202. Alternatively, the untextured portion 2208 may deflate similar to the textured portions 2204A, 2204B. In such implementations, the untextured portion 2208 may similarly become concave when deflated, giving the balloon 2202 an “hourglass” or similar shape that tapers radially inward from the untextured ends 2206A, 2206B when in the deflated state.

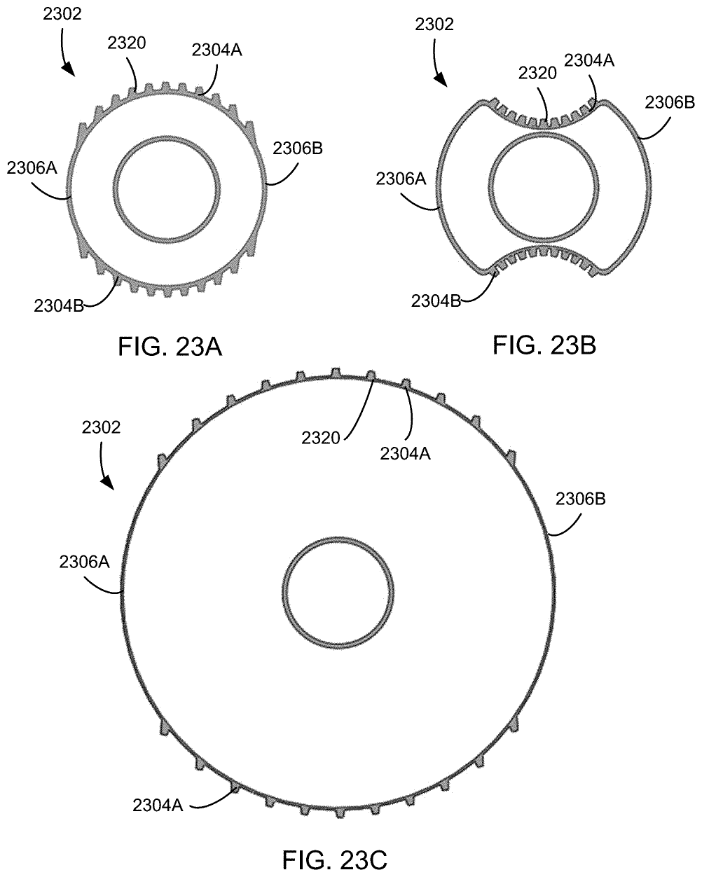

FIGS. 23A-23C are cross-sectional views of a third balloon 2302 including features to selectively collapse portions of the balloon 2302 when in the deflated state. More specifically, FIG. 23A illustrates the balloon 2302 in an unstrained state, FIG. 23B illustrates the balloon 2302 in a collapsed state, and FIG. 23C illustrates the balloon 2302 in a strained inflated state in which the balloon is inflated to a greater extent than as illustrated in FIG. 23A. As shown, the balloon 2302 generally includes textured portions 2304A, 2304B and untextured portions 2306A, 2306B extending circumferentially between the textured portions 2304A, 2304B. In at least certain implementations, the balloon 2302 may also include untextured proximal and distal ends, as included in other implementations of the present disclosure. As illustrated in each of FIGS. 23A-230, each of the textured portions 2304A, 2304B generally includes a plurality of protrusions, such as protrusions 2320.

In contrast to textured portions 2204A, 2204B of the balloon 2202 of FIGS. 22A and 22B, in which the textured portions 2204A, 2204B becomes concave about an axis perpendicular to a longitudinal axis of the balloon 2202, the balloon 2302 is configured such that the textured portions 2304A, 2304B become concave about an axis parallel to the longitudinal axis of the balloon 2302. As illustrated in FIG. 23B, when in the collapsed state, the concavity of the textured portions is such that the protrusions 2320 are disposed within a maximum radius defined by the untextured portions 2306A, 2306B. As a result, when in the deflated state, the balloon 2302 may be inserted into and/or transported through a physiological lumen with reduced interaction between the textured portions 2304A, 2304B and an inner surface of the physiological lumen. When in an intended position, the balloon 2302 may then be inflated such that the textured portions 2304A, 2304B expand from the concave configuration, thereby causing contact between the protrusions 2320 the inner surface of the physiological lumen. Doing so increases frictional engagement between the balloon 2302 and the inner surface, up to and including frictional engagement sufficient to anchor the balloon 2302 in place within the physiological lumen.

Controlled collapsing/concavity of balloons in accordance with the present disclosure may be achieved in various ways. For example, and without limitation, portions of the balloon intended to collapse or become concave (e.g., the textured portions 2204A, 2204B) may have a smaller wall thickness than other portions intended to substantially retain their shape (e.g., the untextured ends 2206A, 2206B). In other implementations, portions of the balloon intended to retain their shape may be selectively reinforced. For example, the balloon 2202 illustrated in each of FIGS. 22A and 22B includes internal ridges 2210A, 2210B disposed within the untextured ends 2206A, 2206B. During inflation and deflation, the internal ridges reinforce the untextured ends 2206A, 2206B such that the untextured ends 2206A, 2206B maintain a more consistent shape as compared to unreinforced portions of the balloon 2202, such as the textured portion 2204A, 2204B.

FIG. 24 illustrates an alternative structure for controlling collapse of an example balloon 2402 during deflation. The balloon 2402 includes a pair of textured portions 2404A, 2404B between which are disposed untextured portions 2406A, 2406B. As illustrated, each of the textured portions 2404A, 2404B has a first wall thickness (t1) and each of the untextured portions 2406A, 2406B has a second wall thickness (t2) that is greater than the wall thickness of the textured portions 2404A, 2404B. In one example implementation, the first wall thickness may be from and including about 100 μm to and including about 2000 μm while the second wall thickness may be from and including about 150 μm to and including about 3000 μm.

As a result, as the balloon 2402 collapses, the textured portions 2404A, 2404B will collapse and become concave prior to and to a greater extent than the untextured portions 2406A, 2406B. In certain implementations, the wall thickness of the untextured portions 2406A, 2406B may also be sufficient to prevent or substantially reduce collapse of the untextured portions 2406A, 2406B during deflation. As further illustrated in FIG. 24, controlled collapse of the balloon may also be facilitated by the use of notches 2410A-2410D or similar features that provide localized reduction of the wall thickness of the balloon 2402. For example, the notches 2410A-2410D of the balloon 2402 are formed at the transition between the textured portions 2404A, 2404B and the untextured portions 2406A, 2406B to facilitate collapse of the untextured portions 2406A, 2406B.

The specific ways in which balloons may be inflated/collapsed described above are provided merely as examples. More generally, balloons in accordance with the present disclosure may be configured to collapse and/or inflate in a non-uniform way. By doing so, different states of deflation/inflation may be used to disposed different proportions of the balloon protrusions at a maximum diameter of the balloon and/or to position different proportions of the protrusions in a substantially outwardly/radially extending direction.

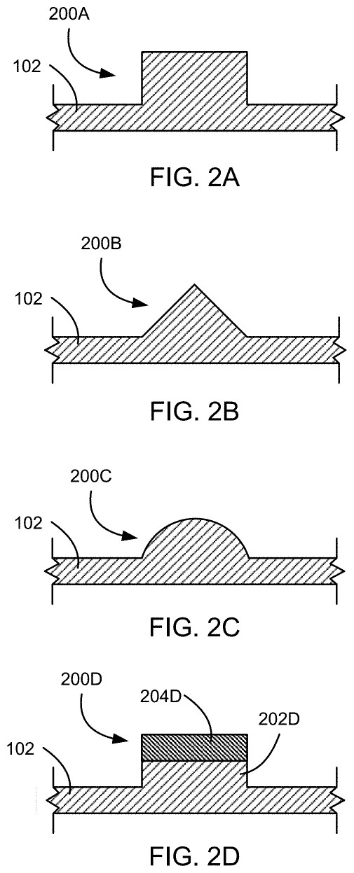

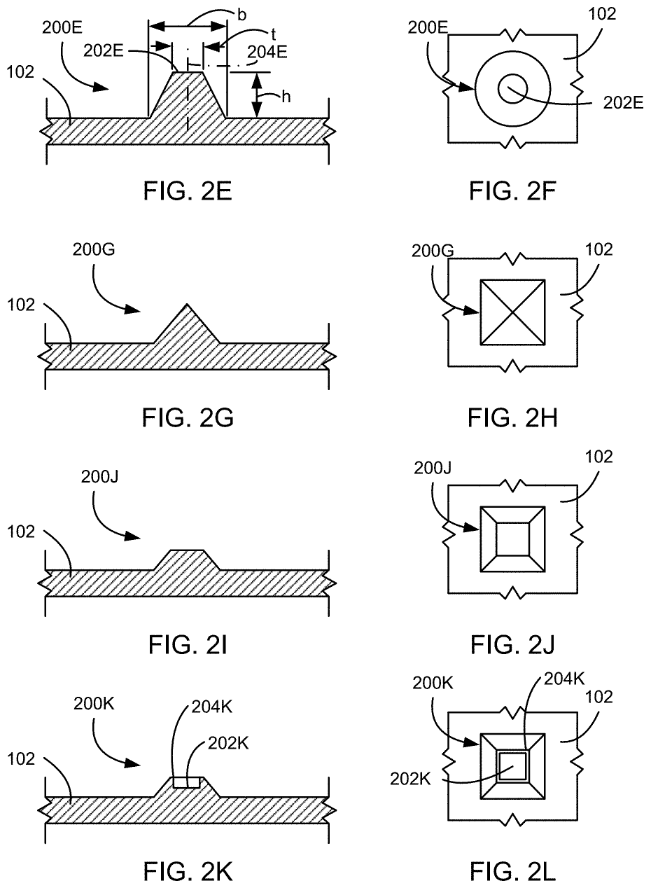

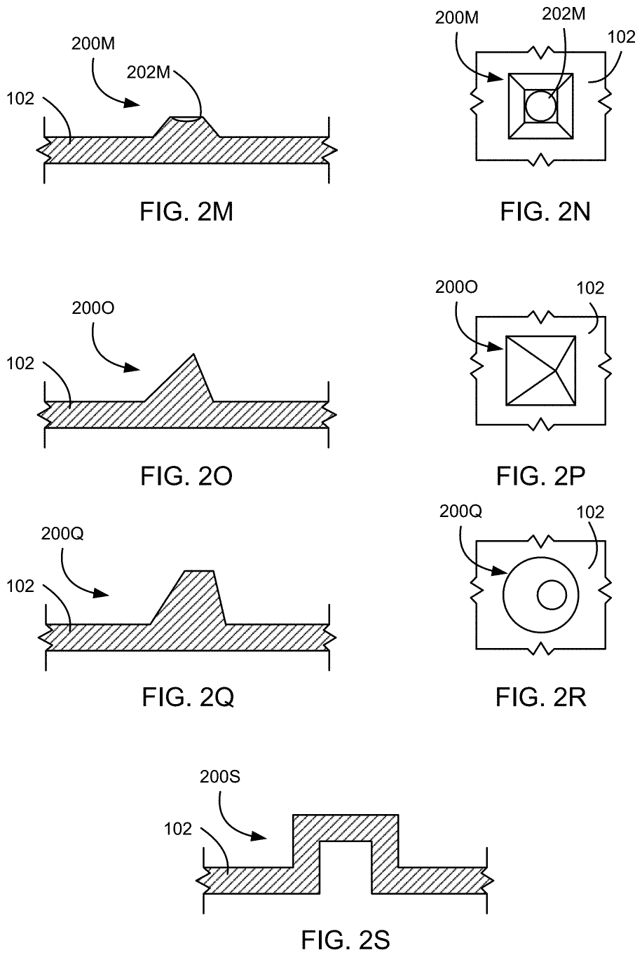

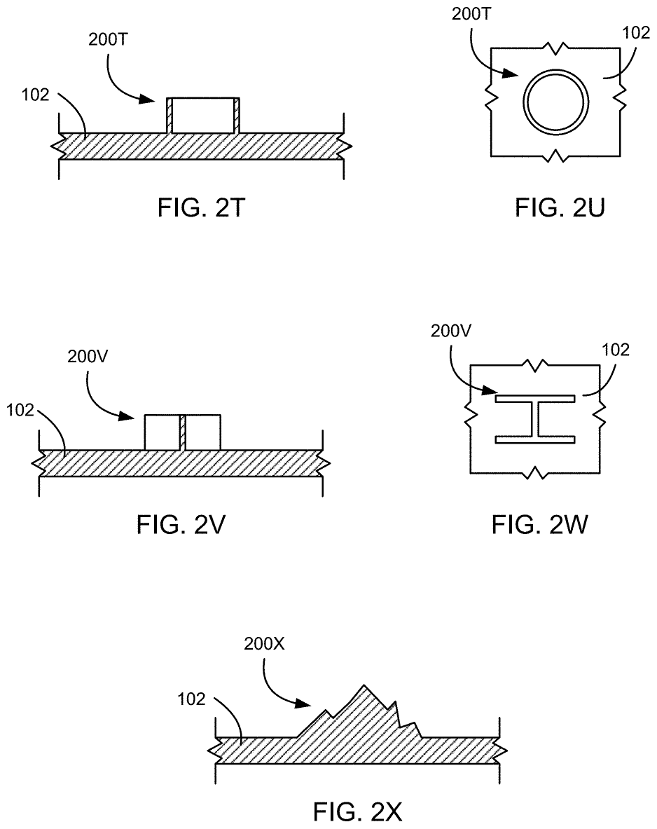

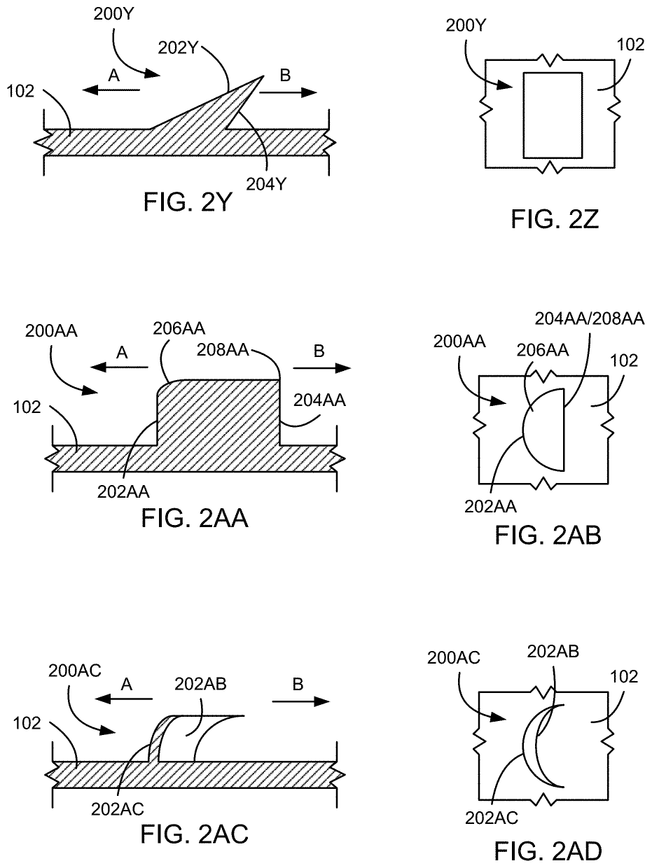

FIGS. 2A-2AD are various views of example protrusions in accordance with the present disclosure. These example protrusions are shown with the corresponding balloon in an unstrained state. Accordingly, inflation of the corresponding balloons into a strained state will generally alter the shapes of the example protrusions.

FIG. 2A illustrates a first protrusion 200A extending from the balloon 102 and having a cylindrical or rectangular shape, FIG. 2B illustrates a second protrusion 200B having a triangular or pyramidal shape, and FIG. 2C illustrates a third protrusion 200C having a rounded or hemispherical shape. FIG. 2D is a cross-sectional view of a fourth protrusion 200D composed of multiple materials.

The protrusion shapes illustrated in FIGS. 2A-2D are intended merely as examples and other protrusion shapes are possible. For example, and without limitation, other implementations of the current disclosure may include protrusions having any shape, including but not limited to rectangular, square, triangular, pentagonal, heptagonal, hexagonal, pyramidal, mushroom, or spherical shape. These protrusions are solid in one example, while in other embodiments the protrusions may be hollow. The ends of the protrusions distal to the surface of the balloon 102 may also be formed in various shapes. For example, and without limitation, the distal ends of the protrusions may be flat, rounded (including either of convex or concave), pointed, or mushroomed. The width/diameter of the protrusions may also vary. For example, the distal end of the protrusions may be larger in diameter than the proximal end, so as to resemble a mushroom. In other implementations, the proximal end of the protrusions may be larger in diameter than the distal end, such that the protrusions distally taper.

As noted above, FIG. 2D illustrates a protrusion 200D formed from multiple materials. More specifically, the protrusion 200D includes a first portion 202D proximal the balloon 102 and a second portion 204D distal the balloon 102. As illustrated, the first portion 202D is integrally formed with the balloon 102. The second portion 204D, on the other hand, forms a cap or tip of the protrusion 200D that may be coupled to or formed onto the first portion 202D after formation of the first portion 202D. In other implementations, each of the first and second portions 202D, 204D may be formed from different materials than the balloon 102.

The specific arrangement illustrated in FIG. 2D is intended merely as an example of a multi-material protrusion and other arrangements are possible. For example, and without limitation, multi-material protrusions may be formed by embedding or implanting structural elements of a first within protrusions formed of a second material or at least partially encompassing protrusions formed from a first material with a cap, sheath, or similar element formed from a second material. It should also be appreciated that while FIG. 2D illustrates a two-material protrusion 200D, any suitable number of materials may be used to form protrusions in accordance with the present disclosure.

FIGS. 2E-2AD illustrate additional example protrusions that may be implemented in embodiments of the present disclosure. FIGS. 2E and 2F, for example, are a cross-sectional view and a plan view, respectively, of a protrusion 200E extending from the balloon 102 and having a frustoconical shape. As illustrated in FIG. 2E, the shape of the protrusion 200E may be defined by a base diameter b, a height h, and a top diameter t of the protrusion 200E. Although any suitable dimension for b and h may be used, in at least certain implementations, b may be from and including about 50 μm to and including about 3000 μm, h may be from and including about 25 μm to and including about 3000 μm, and t may be from and including about 25 μm to and including about 2500 μm. Moreover, while the protrusion 200E of FIGS. 2E and 2F is illustrated as having a top 202E extending substantially perpendicular to an axis 204E of the protrusion 200E, in other implementations, the top 202E may instead be biased relative to the axis 204E. The performance characteristics of the protrusion 200E may be modified by altering various aspects of the protrusion 200E. For example, and without limitation, any of the base diameter, top diameter, or height of the protrusion 200E may be varied to modify the stiffness of the protrusion 200E.

FIGS. 2G-2N illustrate various implementations of pyramidal protrusions. Specifically, FIGS. 2G and 2H are a cross-sectional view and a plan view, respectively, of a protrusion 200G extending from the balloon 102 and having a pointed, square-based pyramid shape. FIGS. 21 and 2J are a cross-sectional view and a plan view, respectively, of a protrusion 200J extending from the balloon 102 and having a truncated, square-based pyramid shape. FIGS. 2K and 2L are a cross-sectional view and a plan view, respectively, of a protrusion 200K extending from the balloon 102 and having a truncated, square-based pyramid shape including a square recess 202K extending into the protrusion 200K from a top surface 204K of the protrusion 200K. Similarly, FIGS. 2M and 2N are a cross-sectional view and a plan view, respectively, of a protrusion 200M extending from the balloon 102 and having a truncated, square-based pyramid shape including a concave top surface 202M.

FIGS. 2O-2R illustrated example protrusions having an asymmetrical or “swept” configuration. More specifically, FIGS. 2O and 2P are a cross-sectional view and a plan view of another example protrusion 2000, the protrusion Q having a swept square-based pyramidal shape. Similarly, FIGS. 2Q and 2R are a cross-sectional view and a plan view of yet another example protrusion 200Q, the protrusion 200Q having a swept truncated conical shape. In certain implementations, such swept shapes may be the result of molding process limitations. For example, a mold for producing balloons in accordance with the present disclosure may be formed using electrical discharge machining (EDM). In such cases, a machining electrode is plunged into a mold half to form the protrusions. In applications in which the plunging path is linear and the mold half is curved, the resulting feature will inherently have a shadowed or swept shape. Nevertheless, in other implementations the swept shapes may be specifically controlled to provide improved traction, to otherwise bias the protrusions in a particular direction, to provide reinforcement in a specific direction, and the like.

FIG. 2S is a cross-sectional view of still another example protrusion 200S. The protrusion 200S is provided to illustrate that protrusions in accordance with the present disclosure may be hollow. While illustrated in FIG. 2S as being substantially rectangular or cylindrical in shape, it should be understood that any protrusion design discussed herein may be at least partially hollow and such hollow protrusions are not limited to any specific shape or dimensions.

FIGS. 2T and 2U are a cross-sectional view and a plan view of another example protrusion 200T. More specifically, the protrusion 200T has a tubular cylindrical shape and is intended to illustrate an implementation of a protrusion having a tubular or thin-walled construction. Although illustrated as having a cylindrical shape, it should be understood that thin-walled/tubular protrusions similar to that illustrated in FIGS. 2T and 2U are not limited to cylindrical shapes. Rather, thin-walled or tubular protrusions may have any suitable shape.

FIGS. 2V and 2W are a cross-sectional view and a plan view of still another example protrusion 200V. More specifically, the protrusion 200V has a barbell-type shape and is intended to illustrate an implementation of a protrusion formed from a series of interconnected ribs, walls, or similar structures extending from the surface of the balloon 102.

FIG. 2X is a cross-sectional view of a protrusion 200X having a jagged shape. Protrusion 200X is intended to illustrate that protrusions in accordance with the present disclosure are not limited to conventional shapes or surfaces. Rather, protrusions may be implemented having any suitable shape or surface, including random or pseudo-randomly generated shapes or surfaces.

FIGS. 2Y-2AD illustrate various protrusions having a directional design. For purposes of the present disclosure, directional protrusions refer to protrusions that are specifically shaped to provide reduced friction/adhesion or improved aero- or hydrodynamic behavior in a first direction and increased friction/adhesion or reduced aero- or hydrodynamic behavior in a second direction that is generally opposite the first direction. Among other things, such protrusions designs may be beneficial for facilitating translation or movement of a balloon within a lumen in a first direction while providing increased resistance to translation or movement of the balloon in a second opposite direction.

Referring first to FIGS. 2Y and 2Z, a cross-sectional and a plan view of a first directional protrusion 200Y is provided. The protrusion 200Y has a swept or saw tooth shape that provides variable resistance in opposite directions. More specifically, the shallower slope of a leading face 202Y of the protrusion provides reduced friction in a first direction a first direction (indicated by arrow A) as compared to a second, opposite direction (indicated by arrow B). In the specific implementation illustrated in FIG. 2Y, a trailing face of the protrusion 204Y is arranged such that the protrusion 200Y forms a barb or hook-like shape. However, it should be appreciated that variable directional performance may be achieved with a less aggressive design, such as the “swept” protrusions illustrated in FIGS. 2O-2R.

FIGS. 2AA and 2AB are a cross-sectional view and a plan view of a second direction protrusion 200AA having a semi-circular shape. More specifically, the protrusion 200AA includes a curved leading surface 202AA and a substantially flat tailing surface 204AA such that the protrusion 200AA provides reduced friction in a first direction (indicated by arrow A) as compared to a second direction (indicated by arrow B). Additional directional properties of the protrusion 200AA are provided by including a rounded or smoothed leading edge 206AA and a substantially sharper tailing edge 208AA. For example, in at least certain implementations, the tailing edge 208AA may have a radius from and including about 5 μm to and including about 500 μm, for example 75 μm, while the leading edge 206AA may have a radius having that is 1.1-2.0 times or greater than the radius of the tailing edge 208AA.

FIGS. 2AC and 2AD are a cross-sectional view and a plan view of a third direction protrusion 200AA having a scalloped crescent shape. More specifically, the protrusion 200AC includes a convex leading surface 202AC and a concave tailing surface 204AC such that the protrusion 200AC provides reduced friction in a first direction (indicated by arrow A) as compared to a second direction (indicated by arrow B). Similar to the protrusion 200Y illustrated in FIGS. 2Y and 2Z, the crescent shaped protrusion 200AC is also “swept” to further vary resistance between the indicated directions.

It should be understood that the protrusions illustrated in FIGS. 2A-2AD and elsewhere throughout this disclosure are intended merely as examples and should not be viewed as limiting the scope of the present disclosure. Implementations of the present disclosure may include protrusions combining features or characteristics of any of the protrusion designs discussed herein. For example, and without limitation, the concave tip illustrated in FIGS. 2M and 2N may be incorporated into protrusions having any suitable base shape. Similarly, “swept” protrusion designs, as illustrated in FIGS. 2O-2R may similarly include any suitable base shape.

While illustrated in FIGS. 2A-2AD as having substantially smooth exterior surfaces, in at least certain implementations, outer surfaces of protrusions in accordance with the present disclosure may instead be selectively roughened or textured to provide additional friction/adhesion. For example, and without limitation, such texturing may be applied to the protrusions by grit blasting or otherwise roughening the surfaces of the mold used to produce the protrusions. In such implementations, such additional texturing or roughening of the protrusions surfaces may be about 25 μm or less.

Although generally described above as being discrete structures, protrusions according to the present disclosure may also be in the form of elongate ridges, ribs, walls, or similar structures. Such structures may extend longitudinally, circumferentially, or a combination therefore. Moreover, in certain implementations, such elongate structures may be included in combination with one or more other protrusion shapes disclosed herein.

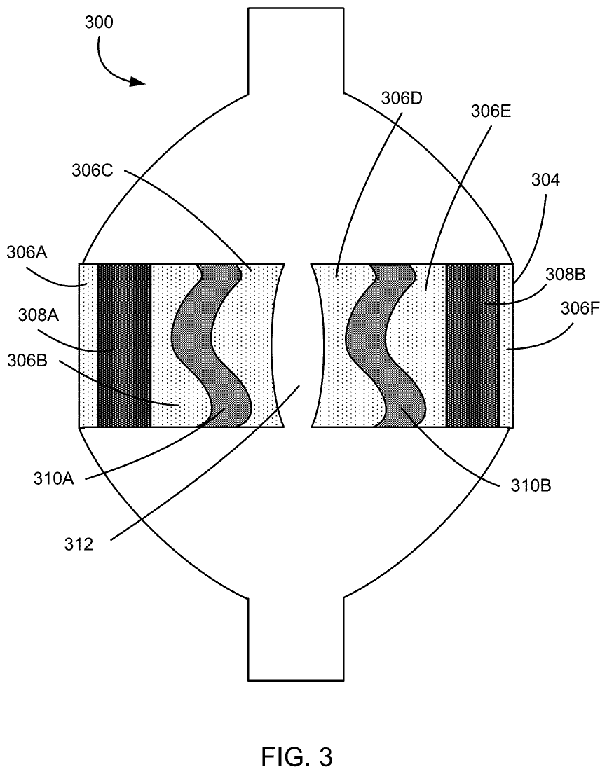

The example balloon 102 illustrated in FIGS. 1A-1E included a textured portion 104 having a substantially uniform distribution of protrusions extending therefrom. In contrast, FIG. 3 is a side elevation view of another example balloon 300 in accordance with the present disclosure in a minimally inflated state including a more complicated textured portion 304. More specifically, in contrast to the textured portion 104 of the balloon 102 illustrated in FIG. 1E, which included a substantially uniform pattern and distribution of substantially uniform protrusions, the textured portion 304 includes multiple areas 306A-312 of protrusions. More specifically, the textured portion 304 includes a first set of areas 306A-306F having a relatively low protrusion density; a second set of areas 308A, 308B having a relatively high protrusion density; a third set of areas 310A, 310B having an intermediate protrusion density; and a fourth area 312 that is substantially smooth. Although the areas are described as having different protrusion densities, it should be appreciated that each area may vary in other aspects including, without limitation, one or more of protrusion density, protrusion shape, protrusion rigidity, protrusion distribution pattern, protrusion material, and the like. Similarly, as illustrated in FIG. 3, each area of the textured portion 304 may vary in size and shape.

Referring back to the example medical device 100 of FIGS. 1A-1E, the height of the protrusions 106 may vary in different applications of the present disclosure. For example, and without limitation, in at least one implementation the protrusions 106 may be from and including about 5 μm to and including about 700 μm tall when the balloon 102 is in either an uninflated or inflated state. In another implementation, the protrusions may be from and including about 15 μm to and including about 200 μm tall. In yet other implementations, the protrusions may be from and including about 30 μm to and including about 110 μm tall. In at least one specific implementation, the protrusions are from and including about 300 μm to and including about 500 μm to enable the protrusions to penetrate mucosal layers of the physiological lumen. In contrast, in applications in which a mucosal layer may not be present (e.g., cardiac applications), the protrusions may be from and including about 50 μm to and including about 100 μm in height. Although implementations of the present disclosure are not limited to any specific protrusion heights, in at least certain implementations, the protrusions may have an overall height up to and including about 5000 μm or greater. Specific implementations of the present disclosure may also include protrusions having varying heights. Also, individual protrusions may have different portions extending to different heights (e.g., having a crenellated or other top having varying height).

As noted above, protrusion height for a given application may vary depending on the type of physiological lumen within which a balloon is being deployed and, more specifically, the thickness of any fluid layers that may be present. For example, and without limitation, the mucosal layer of the colon is generally around 800-900 μm thick while that of the ileum is generally around 400-500 μm thick. Accordingly, to adequately penetrate the respective mucosal layers, balloons intended for deployment in the colon may generally be provided with protrusions of greater length as compared to those of balloons intended for deployment in the ileum. Similar considerations may be made for fluidic layers (e.g., other forms of mucus, sinus fluid, perspiration, etc.) that may be present in other physiological lumens within which balloons according to the present disclosure may be deployed.

Similar to height, the cross-sectional width (e.g., the diameter in the case of protrusions having a circular or ovoid cross-section) of each protrusion may vary. For example, and without limitation, in one implementation the protrusions have a cross-sectional width from and including about 5 μm to and including about 1000 μm when the balloon 102 is in either the uninflated or inflated state. In another implementation the protrusions have a cross-sectional width from and including about 25 μm to and including about 300 μm. In yet other embodiments the protrusions have a cross-sectional width from and including about 70 μm to and including about 210 μm. In still another implementation the protrusions have a cross-sectional width from and including about 600 μm to and including about 1000 μm. In yet another implementation the protrusions have a cross-sectional width from and including about 300 μm to and including about 500 μm. In another implementation, the protrusions have a cross-sectional width from and including about 150 μm to and including about 250 μm. In at least one specific implementation, the protrusions have a cross-sectional width of about 400 μm. Implementations of the present disclosure may also include protrusions having varying diameters. Also, individual protrusions may have different portions having different diameters (e.g., a tapering shape). Although protrusion cross-sectional width for implementations of the present disclosure are not limited to any particular ranges or values, in at least certain implementations, the protrusions may have an overall cross-sectional width up to and including about 5000 μm or greater.

In certain implementations, the overall proportions of a protrusion may instead be defined according to an aspect ratio relating the height of the protrusion to the cross-sectional width/diameter of the protrusion. Although any suitable aspect ratio may be used, in one example implementation, the aspect ratio is less than about 5. In another example implementation, the aspect ratio may be from and including about 0.05 to and including about 10. In yet another example implementation the aspect ratio may be from and including about 0.1 to and including about 5.0. In another example implementation the aspect ratio may be from and including about 0.5 to and including about 1.0. In still another example implementation, the aspect ratio may be from and including about 1.0 to and including about 10.0. In another implementation, the aspect ratio may be from and including about 0.1 to and including about 1. In still another implementation, the aspect ratio may be from and including about 1 to and including about 2. In yet another example implementation, the aspect ratio may be about 0.5, about 1.0, or about 2.0. It should also be appreciated that the aspect ratio for protrusions within a given implementation of the present disclosure may vary such that a first set of protrusions of a balloon conforms to a first aspect ratio while a second set of protrusions for the same balloon conforms to a second aspect ratio. Moreover, the cross-sectional width/diameter of the protrusion for purposes of determining an aspect ratio may be any measure of cross-sectional width/diameter. For example, the cross-sectional width/diameter may be the maximum cross-sectional width/diameter of the protrusion, the minimum cross-sectional width/diameter of the protrusion, an average cross-sectional width/diameter of the protrusion, or the cross-sectional width/diameter of the protrusion at a particular location along the length of the protrusion.

The protrusions may also be configured to have a particular stiffness to avoid inadvertent bending or deformation while still allowing engagement of the protrusions with biological tissue. In at least certain implementations, the protrusions are formed such that they have a stiffness that is at least equal to the tissue with which the protrusions. For example, in certain implementations, the stiffness of the protrusions is from and including about 1.0 to and including 2.0 times that of the tissue with which it is to engage. The stiffness may also be expressed as a modulus of elasticity of the material from which the protrusions are formed. For example, in at least some implementations, the protrusions are formed from a material having a modulus of elasticity from and including about 50 kPa to and including about 105 kPa. In other implementations including stiffer protrusions, the protrusions may be formed of a material having a modulus of elasticity from and including about 0.8 MPa to and including about 2.0 MPa. It should be appreciated that the foregoing ranges are provided merely as examples and moduli of elasticity outside the ranges provided are within the scope of the present disclosure. For example, and without limitation, protrusions according to the present disclosure may have a modulus of elasticity from and including 10 kPa to and including 4.0 kPa depending on application.

In certain implementations, protrusions of balloons in accordance with the present disclosure may be configured to deform in response to a strain being applied to the balloon. Such deformation may then be used to dynamically control and adjust traction between the balloon and biological tissue.

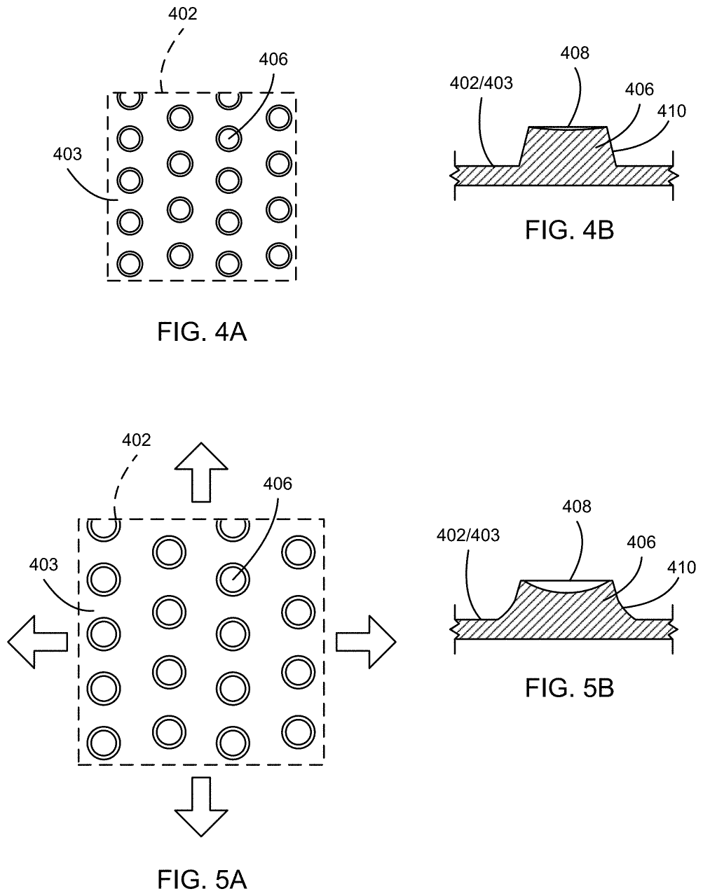

FIG. 4A illustrates a portion of a balloon 402 or similar structure in a first state of strain. In certain applications, the first state of strain may correspond to an unstrained state or, alternatively, may correspond to a state in which a first strain is applied to the balloon 402. As shown, the balloon 402 includes multiple protrusions, such as protrusion, 406 distributed across and extending from a surface 403 of the balloon 402. As illustrated in FIG. 4B, the protrusions 406 may, in certain implementations, have a frustoconical shape. FIG. 5A illustrates the portion of the balloon 402 in a second state of strain, in which a strain greater than that of the first state of strain is applied to the balloon 402. As shown in FIG. 5A, in at least some applications, the applied strain when in the second state of strain may be biaxial. Such strain may result, for example, from inflation of the balloon 402. As illustrated in FIG. 5A, the application of strain generally results in both the distance between adjacent protrusions increasing as well as a stretching/deformation of the protrusions. FIG. 5B is a cross-sectional view of the protrusion 406 when a biaxial strain is applied to the balloon 402. As illustrated, the frustoconical shape of the protrusion 406 deforms under the biaxial strain. In particular, each of a top surface 408 and side wall 410 of the protrusion 406 become increasingly concave in response to the application of biaxial strain.

The term “biaxial strain” is generally used herein to refer to a strain applied along two axes which, in certain implementations, may be perpendicular to each other. In certain cases, the biaxial strain may be approximately equal along each axis. For example, strain applied to the balloon may be equal in each of a longitudinal direction and a transverse direction. However, in other implementations, strain may be applied unequally along the axes, including strain resulting in non-uniform deformation of the protrusions (e.g., elongation of compression primarily along a single axis). Moreover, sufficient deformation of the protrusions may also be achieved by application of a uniaxial strain or a multiaxial strain other than a biaxial strain. Accordingly, while the examples described herein are primarily discussed with reference to a biaxial strain resulting in variations in frictional and adhesive engagement resulting from deformation of the protrusion, implementations of the present disclosure are more generally directed to variations in frictional and adhesive engagement from deformation of the protrusions in response to any applied strain.

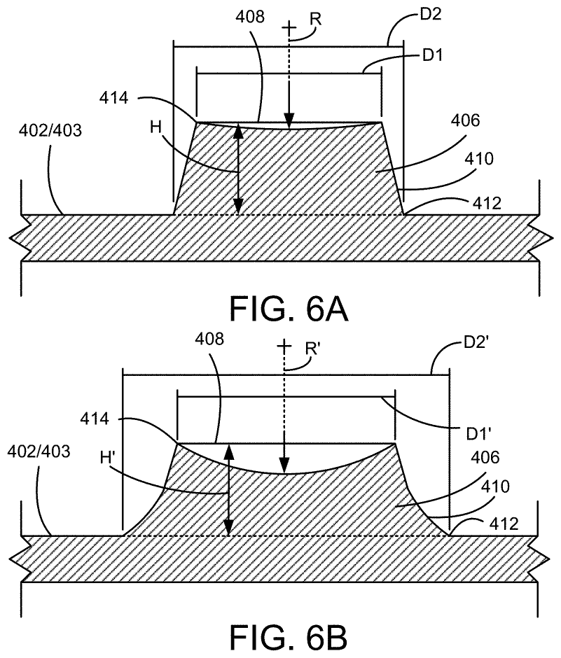

FIGS. 6A and 6B are cross-sectional views of the protrusion 406 illustrating further details of the protrusion in a strained and unstrained state, respectively. As illustrated in FIG. 6A, when in the unstrained state, the protrusion 406 has a top diameter (D1) corresponding to the top surface 408 of the protrusion and a base diameter (D2) corresponding to a base 412 of the protrusion 406. The top surface 408 of the protrusion 406 is shown as being disposed at a maximum height (H). The top surface 408 is also shown as being concave and having a concavity defined by a radius of curvature (R). The top surface 408 of the protrusion reaches a height (H) relative to the surface 403 of the balloon 402. It should be appreciated that while the top surface 408 of the protrusion is shown in FIG. 6A as being concave, in other implementations, the top surface 408 may be substantially flat. Also, while the top diameter D1 and base diameter D2 are illustrated in FIG. 6A as being different, in other implementations D1 and D2 may be equal such that the protrusion 406 is substantially cylindrical in shape.

As shown in FIG. 6B, the protrusion 406 may deform in response to a strain applied to the balloon 402. In particular, each of the top diameter (D1) and the base diameter (D2) may expand to a second base diameter (D1′) and a second base diameter (D2′), respectively. The radius of curvature (R) of the top surface 408 may also decrease to a second radius of curvature (R′), thereby causing the top surface 408 to become increasingly concave. In addition to the foregoing dimensional changes, the overall height of the protrusion 406 may change from the initial height (H) to a second height (H′).

As illustrated in FIGS. 6A and 6B, in at least some implementations of the present disclosure, each protrusion may include a lip or edge 414 at the transition between the side wall 410 and the top surface 408. In general, a relatively sharp lip or edge 414 may allow the protrusions to more readily engage the wall of the physiological lumen and may also facilitate penetration of mucosal or other layers that may be present on the wall. Accordingly, in at least some implementations, the edge 414 may have a radius of no more than about 3 μm.

The initial dimensions of the protrusion 406 may vary. For example, in certain implementations the unstrained upper diameter (D1) of the protrusion may be from and including about 100 μm to and including about 700 μm; the unstrained lower diameter (D2) of the protrusion may be from and including about 100 μm to and including about 750 μm; the unstrained height (H) of the protrusion may be from and including about 100 μm to and including about 700 μm; and the unstrained radius of curvature (R) of the top surface 408 of the protrusion may be from and including about 1 mm to and including about 2 mm. Similarly, in certain implementations, the strained upper diameter (D1′) of the protrusion may be from and including about 375 μm to and including about 750 μm; the strained lower diameter (D2′) of the protrusion may be from and including about 405 μm to and including about 825 μm; the strained height (H′) of the protrusion may be from and including about 200 μm to and including about 400 μm; and the strained radius of curvature (R′) of the top surface 408 of the protrusion may be from and including about 500 μm to and including about 750 μm. In one specific example, the D1 may be about 250 μm, D2 may be about 270 μm, H may be about 500 μm, and R may be about 1.5 mm. In the same example, the balloon 402 may be configured to be strained such that D1′ can be up to about 375 μm, D2′ can be up to about 400 μm; H′ may be decreased down to about 450 μm, and R′ may be decreased down to about 500 μm. In other implementations, deformation of the protrusion 406 in response to a strain applied to the balloon 402 may instead be based on a change in the surface area of the protrusion 406. For example, and without limitation, the balloon 402 may be configured such that the surface area of the protrusion 406 may increase up to about 25%.

During experimental testing, it was observed that separation force between a piece of material including protrusions similar to the protrusion 406 of FIGS. 6A and 6B and a flexible probe simulating biological tissue varied with the degree of biaxial stain applied to the material. More specifically, the probe was first made to contact the material sample, causing the probe to adhere to the material sample. The probe was then withdrawn from contact with the material sample. The force required to affect such separation was measured and observed to vary non-linearly with the degree of biaxial strain applied to the material sample.

As indicated in FIG. 6A, the protrusion 406 may be further characterized by the sharpness of the edge 414 at the transition between the side wall 410 and the top surface 408 of the protrusion 406. Although the edge 414 is not limited to specific degrees of sharpness, testing has indicated that particular sharpness ranges can be advantageous in fixing balloons in accordance with this disclosure within a physiological lumen, particular in the presence of mucus and other similar fluids that may be secreted or disposed along the inner surface of the physiological lumen. More specifically, sufficient sharpness of the edge 414 appears to facilitate penetration through layers of mucus (or similar fluids) to facilitate engagement between the balloon and inner wall of the lumen. Accordingly, in at least certain implementations, the edge 414 between the side wall 410 and the top surface 408 may have a radius from and including about 25 μm to and including about 500 μm, for example 75 μm. In other implementations, the radius is not greater than about 25 μm.

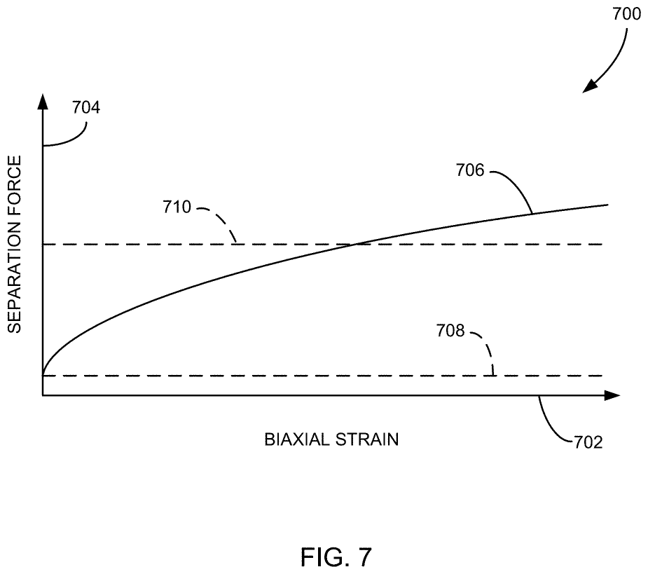

FIG. 7 is a graph 700 summarizing the experimental findings regarding the relationship between separation force and biaxial strain. More specifically, the graph 700 includes a first axis 702 corresponding to biaxial strain and a second axis 704 corresponding to the measured separation force when separating the probe and material sample. As indicated by line 706, the separation force varied in a non-linear fashion in response to changes in biaxial strain.

The graph 700 further indicates a base separation force line 708 corresponding to the separation force when the material sample is unstrained. The graph further includes a “flat” separation force line 710 corresponding to a second material sample substantially similar to the tested material sample but lacking any protrusions.

As illustrated in the graph 700, the separation force for the material having the protrusions may be varied to have a range of values by changing the biaxial strain applied to the material. For example, by applying no or relatively low biaxial strain, the material with protrusions may actually be made to have less separation force (i.e., be made to be less frictional and/or adhesive) than a flat sheet of the same material. However, as biaxial strain is increased friction and adhesion also increase such that, at a certain level of biaxial strain, the separation force of the material including protrusions may be made to exceed that of a flat sheet of the same material.

As shown in the graph 700, this may, in certain implementations, reduce the separation force when unstrained as compared to separation force of a flat material sheet. However, as strain is increased, the separation force may increase above that of the flat sheet. In other words, by selectively applying biaxial strain to the material sample, separation force may be varied, providing physicians with increased control and more reliable engagement for medical devices incorporating balloons in accordance with the present disclosure.

The specific example discussed in FIGS. 4A-7 generally includes protrusions having a flat or partially concave top surface that, when a strain is applied, causes the protrusions to become increasingly concave, thereby increasing their surface area. In other implementations of the present disclosure, the protrusions may instead include a rounded/convex or similar top surface such that when a strain is applied, the top surfaces of the protrusions at least partially flatten. Such flattening may result in a reduction of the surface area and, as a result, a change (generally a reduction) in the separation force between the protrusions and the physiological lumen. Accordingly, whereas in the previous examples a strain is applied to increase protrusion surface area to increase separation force, strain may also be used to decrease protrusion surface area and, as a result, decrease separation force. In either case, however, strain is used as the primary mechanism for altering the shape and the result separation force of the protrusions.

The separation force between the balloon and the physiological lumen may vary across different implementations of the present disclosure and across different states of inflation for any given implementation. However, in at least some implementations, the balloon may be configured to have a separation force less than about 5 N when the balloon is in its deflated state (e.g., as illustrated in FIGS. 1A-1B) to facilitate translation of the balloon along the physiological lumen with minimal adhesion and friction. In other implementations, the separation force when in the deflated state may be less than about 3 N. In a specific example, the separation force in the deflated state may be about 1 N. The balloon may also be configured to have a particular separation force in a minimally inflated state in which the balloon substantially engages the physiological lumen. For example, in at least some implementations, the separation force in the minimally inflated state may be from and including about 10 N to and including about 30 N. In other implementations, the separation force in the minimally inflated state may be from and including about 15 N to and including about 25 N. In one specific implementation, the separation force in the minimally inflated state may be about 20 N.

As previously discussed, in at least some implementations, a strain on the balloon may be applied or modified (e.g., by inflating or deflating the balloon) to modify the adhesive and frictional characteristics of the balloon and, as a result, the separation force between the balloon and physiological lumen. In one implementation, the separation force relative to a minimally inflated state may be reduced to 1% or lower by deflating the balloon and up to and including 200% by overinflating and straining the balloon. In another implementation, the deflated balloon may have a separation force of less than about 5% of the minimally inflated state and a maximum of about 150% by straining the balloon. In still another example implementation, the balloon may have a lower bound separation force of less than about 5% of the minimally inflated state and a maximum of about 125% by straining the balloon. Accordingly, in at least one specific example, the balloon may have a separation force of about 20 N in the inflated state, about 1 N in the deflated state, and about 25 N in a maximum strained state.

As previously noted, balloons in accordance with the present disclosure may be manufactured in various ways. For example, in at least one implementation, balloons including protrusions as discussed above may be manufactured through a casting process. FIG. 8 illustrates an example mold 800 for use in such a casting process. As illustrated the mold 800 includes an outer mold piece 802 within which an inner mold piece or core 804 is disposed. The combination of the outer mold piece 802 and the core 804 defines a cavity 806 providing the general shape of the balloon to be molded.

In addition to the outer mold piece 802 and the core 804, the mold 800 includes an insert 808 for forming protrusions on the balloon during casting. The insert 808 is separately formed to have the pattern and distribution of protrusion to be included on the final balloon. The insert 808 may be manufactured in various ways including, without limitation, machining, 3D printing, microlithography, or any other similar manufacturing process. Once formed, the insert 808 may be disposed within and coupled to the outer mold piece 802. In certain implementations, the insert 808 may be formed from a semi-rigid material such as, but not limited to, Kapton® or other polyimide material, silicone, latex, or rubber.

During the casting process, balloon material (such as but no limited to ECOFLEX® 50) is poured into the cavity and allowed to set. In certain implementations, a vacuum is also applied to the mold 800 to remove air from the mold cavity 806 and to facilitate the material poured into the cavity 806 to take on the shape of the mold cavity 806, including the protrusions defined by the mold insert 808.

In certain implementations, the overall thickness of the balloon may be modified by changing the thickness of the cavity 806. For example, the outer mold piece 802 may be configured to receive cores of varying sizes such that the thickness of the cavity 806 defined between the outer mold piece 802 and the core 804 may be modified by swapping cores into the mold 800.

Although illustrated in FIG. 8 as having a substantially uniform width, the cavity 806 defined between the outer mold piece 802 and the core 804 may also be non-uniform such that the cavity 806 is wider at certain locations within the mold 800. Accordingly, any balloon formed using the mold 800 will have corresponding variations in its thickness. By varying the thickness of the balloon, various characteristics may be imparted to the balloon. For example, the thickness of certain locations of the balloon may be increased to improve the overall durability and strength of the locations. In other cases, the thickness of the balloon may be varied such that reinforced regions of the balloon are formed that cause the balloon to collapse and/or expand in a particular way. Such reinforced regions may also cause the balloon to assume a particular shape in any of a deflated state, partially inflated state, or fully inflated state.

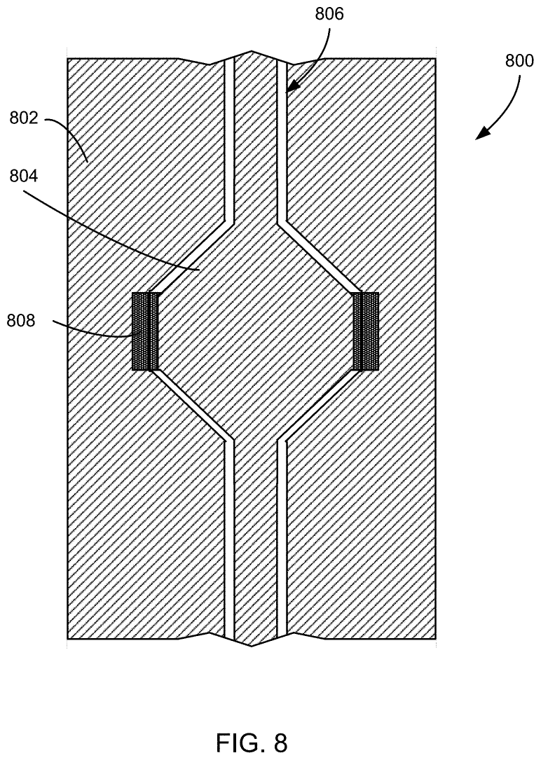

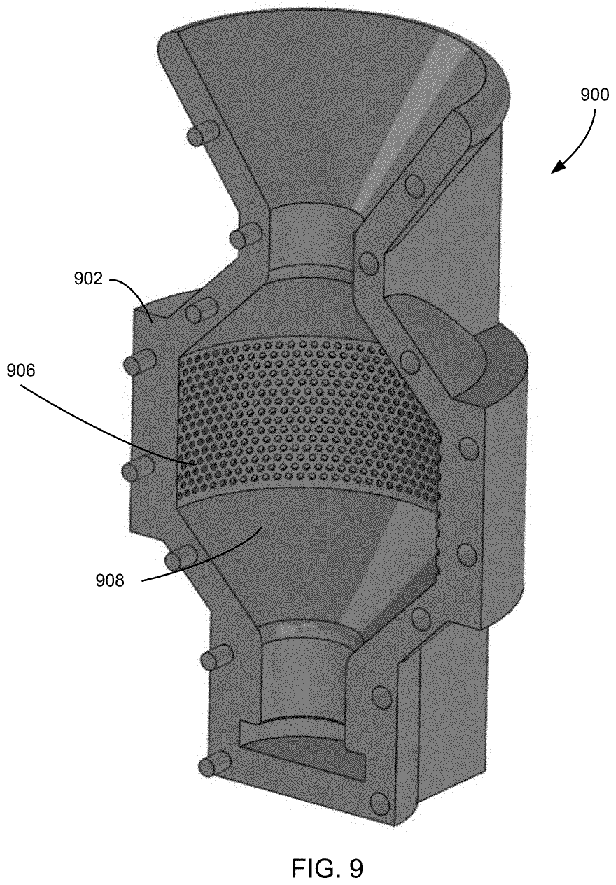

FIG. 9 is an isometric view of an alternative mold 900 for use in manufacturing balloons in accordance with the present disclosure. The mold 900 includes an outer mold piece 902 within which an inner mold piece or core (not shown) may be disposed. In contrast to the mold 800 of FIG. 8 in which a removable insert 808 is used to form the balloon protrusions, the outer mold piece 902 includes voids 906 formed directly into an inner surface 908 of the outer mold piece 902 that are used to form the protrusions during the casting process.

As discussed above, in at least some implementations, balloons in accordance with the present disclosure may be formed using a casting process. Such casting processes may include piece casting, slush casting, drip casting, or any other similar casting method suitable for manufacturing a hollow article. In a slush casting process, for example, an amount of material may be added to the mold and slushed to coat the internal surface of the mold prior to the material setting. Other fabrication methods may also be implemented including, without limitation, various types of molding (e.g., injection molding) and extrusion processes.

While previous fabrication methods included integrally forming the protrusions with the balloon, in other implementations the protrusions may instead be formed onto a previously formed balloon. For example, in at least one other fabrication method, a base balloon may first be formed. The protrusions may then be formed or coupled to the balloon using a subsequent process. In one example fabrication method, the base balloon is extruded and then the protrusions are then added to the base balloon using a spray method. In another example fabrication method, the base balloon is formed using a first casting or molding process and, once the base balloon is set, a second casting or molding process (e.g., an over-molding process) is applied to form the protrusions on the exterior surface of the base balloon.

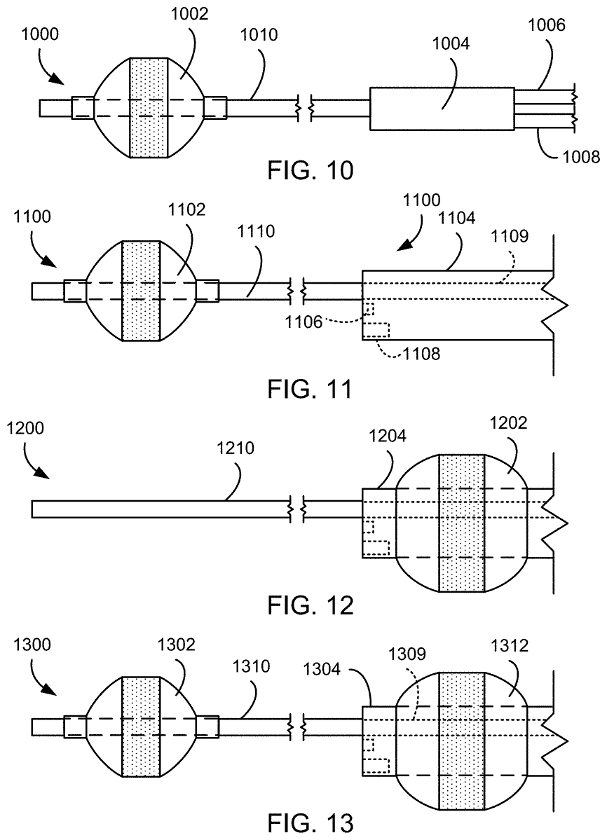

As previously discuss in the context of FIGS. 1A-1E, balloons in accordance with the present disclosure may be implemented for use in various medical devices. FIGS. 10-16 are schematic illustrations of various example medical devices and configurations of such medical devices including balloons of the present disclosure. It should be appreciated that the medical devices provided are merely example devices and are therefore non-limiting. More generally, balloons in accordance with the present disclosure may be used in conjunction with any medical device adapted to be inserted into a physiological lumen. In certain implementations, the medical device may include a lumen running its length. The device lumen may serve as a tool or catheter port such that tools and/or catheters can be threaded down the length of the medical device and out of a distal end of the device. Alternatively, the device may be threaded onto tools or catheters already disposed within the physiological lumen.

FIG. 10 is a schematic illustration of a first medical device 1000 in the form of a catheter delivery tool. As illustrated, the medical device 1000 includes a proximal hub 1004 from which each of a catheter tool channel 1006 and a balloon insufflation channel 1008. A distal portion 1010 of the catheter tool channel 1006 extends from the hub 1004 and includes a balloon 1002 that may be selectively inflated and deflated by providing air to or allowing air to escape from the balloon 1002 via the balloon insufflation channel 1008, respectively. Accordingly, the distal portion 1010 may be inserted into a physiological lumen of a patient with the balloon deflated. Once located at a point of interest within the physiological lumen, air may be provided to the balloon 1002 via the balloon insufflation channel 1008 to cause the balloon 1002 to expand and engage the wall of the physiological lumen. When so engaged, the catheter tool channel 1006 may be used to provide a clear and direct pathway to the location of interest.

The medical device 1000 is described above as being used in conjunction with or to guide a catheter or guide wire within the physiological lumen; however, in other implementations of the present disclosure, balloons in accordance with the present disclosure may be incorporated into catheters or guide wires. For example, and without limitation in at least one implementation of the present disclosure an inflatable balloon as described herein may be disposed along a guide wire or catheter (e.g., at or near the distal end of the guide wire or catheter). In such implementations, the guidewire or catheter may be inserted into a physiological lumen with the balloon in the deflated state. The balloon may be subsequently inflated to engage the physiological lumen and at least partially anchor the guide wire or catheter within the physiological lumen.

FIG. 11 is a schematic illustration of a second medical device 1100, which may be an endoscopic tool. The second medical device 1100 includes an endoscope body 1104 that may include, for example and without limitation, a light emitting diode (LED) 1106 and a camera 1108. The endoscope body 1104 may also define a catheter channel 1109 through which a catheter 1110 may be inserted. As illustrated in FIG. 11, the catheter 1110 may include a distal balloon 1102 that may be used to at least partially secure the catheter 1110 within a physiological lumen.

In one example application of the medical device 1100, the catheter 1110 may be used as a guide for the endoscope body 1104. More specifically, during a first process the catheter 1110 may be delivered to a point of interest along a physiological lumen with the balloon 1102 in an uninflated state. Once located, the balloon 1102 may be inflated to engage the balloon 1102 with the lumen and at least partially secure the catheter within the lumen. The endoscope body 1104 may then be placed onto the catheter 1110 such that the endoscope body 1104 may be moved along the catheter 1110, using the catheter as a guide.

FIG. 12 is a schematic illustration of a third medical device 1200. Similar to the medical device 1100 of FIG. 11, the medical device 1200 includes an endoscope body 1204 (or body of a similar tool) that may be configured to receive a catheter 1210. However, in contrast to the medical device 1100 of FIG. 11 in which the balloon 1102 was coupled to the catheter 1110, the medical device 1200 includes a balloon 1202 coupled to the endoscope body 1204 and which may be used to at least partially secure the endoscope body 1204 within a physiological lumen of a patient.

FIG. 13 is a schematic illustration of a fourth medical device 1300 that combines aspects of both the medical device 1100 of FIG. 11 and the medical device 1200 of FIG. 12. More specifically, the medical device 1300 includes an endoscope body 1304 that defines a catheter channel 1309 through which a catheter 1310 may be inserted. Like the medical device 1100 of FIG. 11, the catheter 1310 includes a distal balloon 1302 that may be used to at least partially secure the catheter 1310 within a physiological lumen. Also, like the medical device 1200 of FIG. 12, the endoscope body 1304 also includes a balloon 1312.

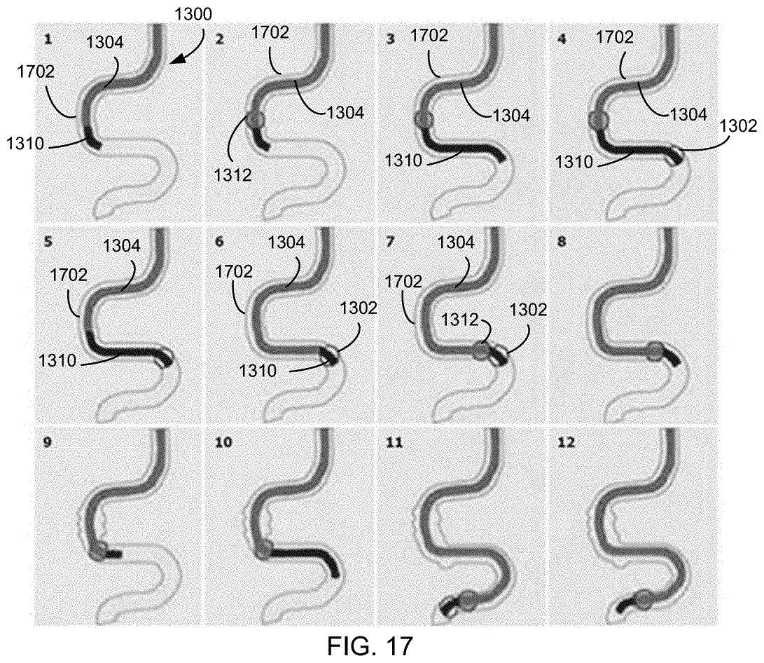

The two-balloon configuration of the medical device 1300 may be used to progress the medical device 1300 along the physiological lumen. For example, FIG. 17 provides a series of illustrations depicting progression of the medical device 1300 along a physiological lumen 1702 (indicated in Frame 1). As illustrated, the medical device 1300 may first be inserted into the physiological lumen in an uninflated/disengaged configuration (Frame 1). The endoscope balloon 1312 may then be inflated to engage the balloon 1312 with the lumen 1702 and to at least partially secure the endoscope body 1304 within the lumen 1702 (Frame 2). With the endoscope body 1304 secured, the catheter 1310 may then be extended from the endoscope body 1304 along the lumen (Frame 3) and the catheter balloon 1302 may be engaged with the lumen 1702 at a second location by inflating the catheter balloon 1302 at the second location (Frame 4). The balloon 1312 may then be deflated (Frame 5) and the endoscope body 1304 may be progressed along the lumen 1702 using the anchored catheter 1310 as a guide (Frame 6). When the endoscope body 1304 reaches the catheter balloon 1302, the endoscope body 1304 may again be secured within the lumen 1702 by inflating the balloon 1312 (Frame 7). As illustrated in Frames 8-12, this process may be repeated to progress the medical device 1300 along the physiological lumen 1702.

In certain implementations, the medical device may be a double balloon endoscope comprising a flexible overtube, as described in PCT Application Publication WO 2017/096350, wherein at least a portion of the outer surface of one or both of the first and second inflatable balloons includes a micro-patterned surface as described herein. In other embodiments, the endoscope does not include an overtube.

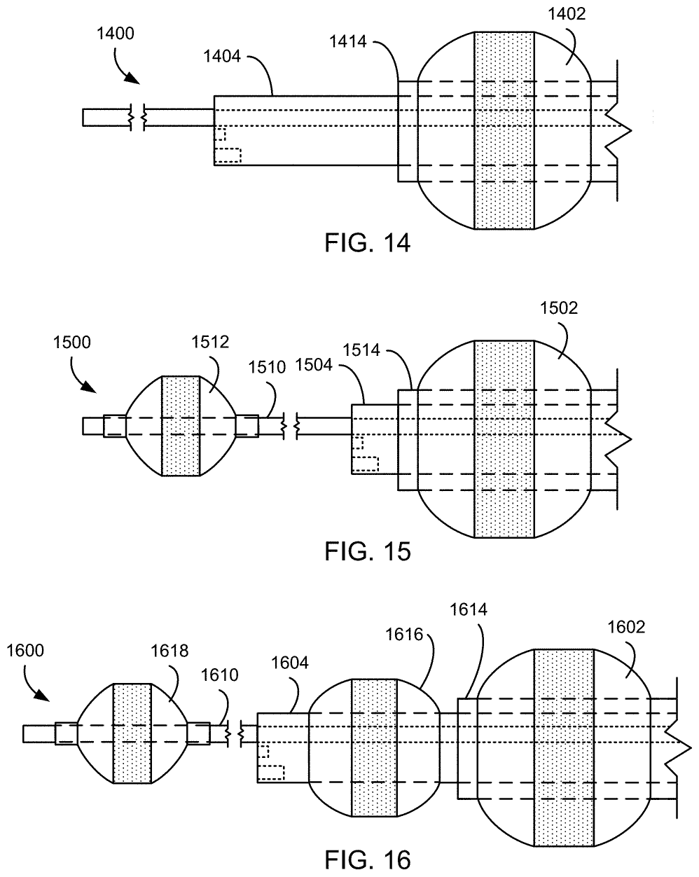

FIGS. 14-16 illustrate additional variations of the foregoing example medical devices. FIG. 14 is a schematic illustration of a medical device 1400 in which a balloon 1402 is coupled to an overtube 1414 through which an endoscope device 1404 may be inserted. FIG. 15 is a schematic illustration of a medical device 1500 similar to that of FIG. 14 in that it includes a balloon 1502 coupled to an overtube 1514 through which an endoscope body 1504 extends. In addition to the balloon 1502, the medical device 1500 includes a catheter balloon 1512 coupled to a distal end of a catheter 1510 extending through the endoscope body 1504. An example double balloon endoscope device similar to that of FIG. 15 and including a flexible overtube is described in detail in PCT Application Publication WO 2017/096350, which is incorporated herein by reference in its entirety. Finally, FIG. 16 is another schematic illustration of a medical device 1600 including three distinct balloons. Specifically, the medical device 1600 includes a first balloon 1602 coupled to an overtube 1614, a second balloon 1616 coupled to an endoscope body 1604 extending through the overtube 1614, and a third balloon 1618 coupled to a catheter 1610 extending from the endoscope body 1604.

In each of the medical tools, it is assumed that the described devices include suitable channels for delivering air or other fluid to the disclosed balloons to inflate the balloons and for removing air/fluid from the balloons to deflate the balloons. For example, each device may include a proximal manifold or coupling that may be connected to a pump or other fluid supply and that further includes a vent or return channel through which fluid may be removed from the balloons. In certain implementations, the medical device includes tubing that is in fluidic communication with one or more balloons of the device, the tubing allowing for controlled inflation and/or deflation of one or more of the balloons. In implementations in which the medical device includes multiple balloons, the tubing used to inflate one or more of the multiple balloons. Alternatively, different sets of tubing may be used to independently control inflation and deflation of respective subsets of the balloons of the medical device.

It should also be appreciated that in implementations of the present disclosure having multiple balloons, only one balloon need to have protrusions in accordance with the present disclosure. In other words, medical devices in accordance with the present disclosure may include one textured balloon as described herein, but may also include any number of non-textured balloons or balloons having designs other than those described herein. Moreover, while the example medical devices of FIGS. 10-17 illustrate balloons located near the distal end of components of the medical devices (e.g., catheters, endoscope bodies, overtubes), in other implementations, balloons may be disposed at any location along such components, including at multiple locations along a given component.

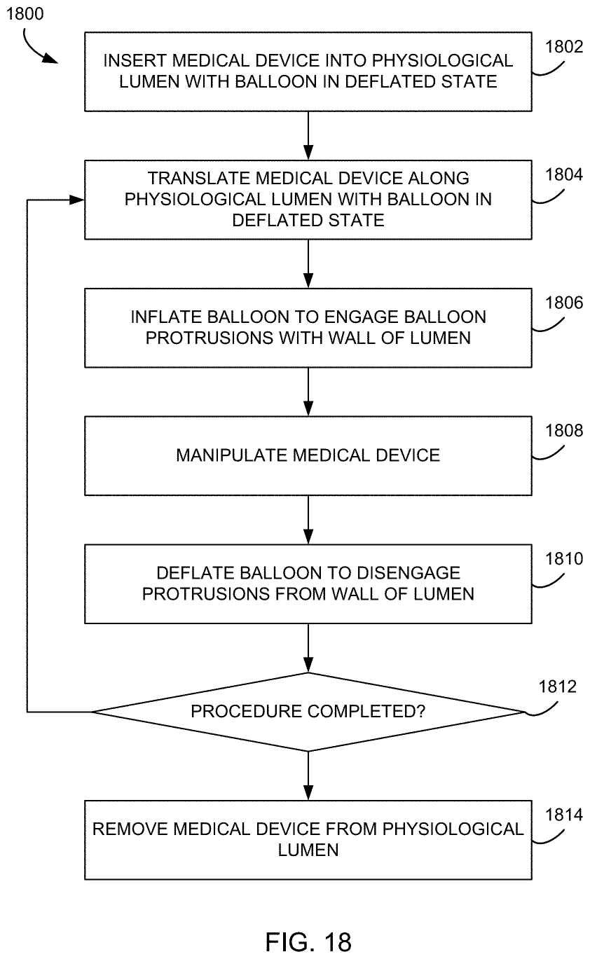

The current disclosure further provides methods of performing endoscopy or similar medical procedures within a body cavity. FIG. 18 is a flowchart illustrating an example method 1800 of such procedures which may be generally performed using medical devices in accordance with the present disclosure, including but not limited to the medical devices discussed in the context of FIGS. 1A-1E and 10-17.

At operation 1802, the medical device is introduced into a physiological lumen or body cavity at least with a balloon of the medical device in a deflated state. As previously discussed, in at least one application of the present disclosure, the physiological lumen may include (but is not limited to) a portion of a patient's GI tract. For example, in the context of a small bowel endoscopy, the physiological lumen may correspond to a portion of a patient's lower digestive system and the medical device may include distal components, such as a light and/or camera, adapted to facilitate examination of the physiological lumen.

Once inserted into the physiological lumen, at least a portion of the medical device is translated along the physiological lumen to an engagement location while the balloon is in the deflated state (operation 1804). For example, in certain implementations, the portion of the medical device may be a catheter including the balloon and translating the portion of the medical device may include extending the catheter and balloon along the physiological lumen while a second portion of the medical device (e.g., an endoscope body) remains at the initial insertion location. In another example implementation, translating the portion of the medical device may include moving an endoscope or similar portion of the medical device along a guide wire or catheter extending along the physiological lumen.

Following translation of the portion of the medical device, the balloon of the medical device is inflated such that protrusions of the balloon as described herein engage with the wall of the physiological lumen (operation 1806).

Once at least partially secured within the lumen, the medical device may be manipulated to perform various functions (operation 1808). In one example, the secured portion of the medical device may include a catheter and the medical device may be manipulated by translating an unsecured portion of the medical device along the physiological lumen using the secured catheter as a guide. In another implementation, the medical device may be manipulated to remove a foreign object or tissue from the physiological lumen. For example, manipulation of the medical device may include insertion and operation of one or more tools of the medical device configured to capture, excise, ablate, biopsy, or otherwise interact with tissue or objects within the physiological lumen. In one specific example, the balloon may be disposed distal a foreign object or tissue of interest within the lumen during operation 1804. The balloon may then be inflated in operation 1806 to obstruct the lumen. In one implementation, the balloon may then be moved proximally through the lumen to remove the foreign object. In another implementation, the balloon may instead be disposed within the lumen and moved distally to remove a foreign object distal the balloon. In another implementation, tools may be inserted through the medical device such that the tools may be used in a portion of the lumen proximal the inflated balloon. The foregoing examples may be useful for removing kidney stones from urinary ducts, removing gall stones from bile ducts, or clearing other foreign or undesirable matter present within the physiological lumen.

In another example medical procedure, a second balloon in accordance with the present disclosure may be disposed and inflated within the physiological lumen such that the protrusions of the second balloon partially engage the wall of the physiological lumen but otherwise remains at least partially movable within the physiological lumen. For example, the second balloon may be disposed on a guide wire or catheter that is then inserted through a medical device previously disposed within the physiological lumen (e.g., during operations 1804 and 1806). With the protrusions of the second balloon partially engaged, the second balloon may be translated along the physiological lumen to rub or scrape the wall of the physiological lumen.

Following manipulation of the medical device, the balloon is deflated to disengage the balloon from the physiological lumen (operation 1810) and an evaluation is conducted to determine when the medical procedure is complete (operation 1812). If so, the medical device is removed from the physiological lumen (operation 1814). Otherwise, the medical device may be repositioned within the physiological lumen for purposes of conducting any additional steps of the procedure (e.g., by repeating operations 1804-1812).

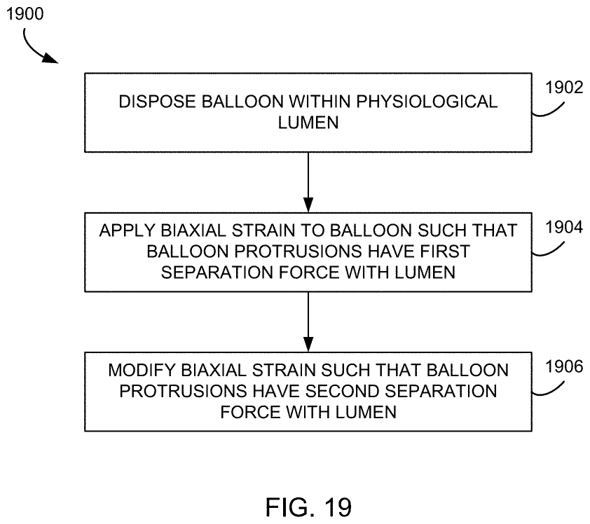

FIG. 19 is a second flowchart illustrating a method 1900 of modifying engagement between a balloon in accordance with the present disclosure and a physiological lumen. As previously discussed in the context of FIGS. 4A-7, the protrusions of balloons in accordance with the present disclosure may be configured to have adhesive and frictional properties that vary based on the biaxial strain applied to them. More specifically, applying strain to the balloon (e.g., by selectively inflating or deflating the balloon) causes deformation of the protrusions on the balloon's surface which in turn modifies adhesion and friction between the balloon and adjacent tissue. As previously discussed, by modifying the strain applied to the balloon, the adhesive and frictional properties may be dynamically manipulated by a physician to allow for improved control and flexibility during medical procedures.

With the foregoing in mind, the method 1900 begins with disposing a balloon having protrusions in accordance with the present disclosure within a physiological lumen (operation 1902). At operation 1904, a biaxial strain is applied to the balloon, such as by inflating the balloon, such that protrusions of the balloon interact with a wall of the physiological lumen and have a first separation force with the wall. At operation 1906 the biaxial strain is modified such that a second separation force different from the first separation force is achieved between the balloon and the wall of the physiological lumen.

With respect to the foregoing, modifying the biaxial strain in operation 1906 may include either of increasing or decreasing the biaxial strain on the balloon. Increasing the biaxial strain may include, for example, inflating the balloon beyond the extent to which the balloon was inflated during operation 1904. As discussed in the context of FIG. 7, increasing strain on the balloon in such a manner may generally result in an increase in the force required to separate the balloon from the wall of the physiological lumen (i.e., increase friction and/or adhesion). Decreasing the biaxial strain may include, for example, at least partially deflating the balloon to decrease the force required to separate the balloon from the wall of the physiological lumen (i.e., decrease friction and/or adhesion).

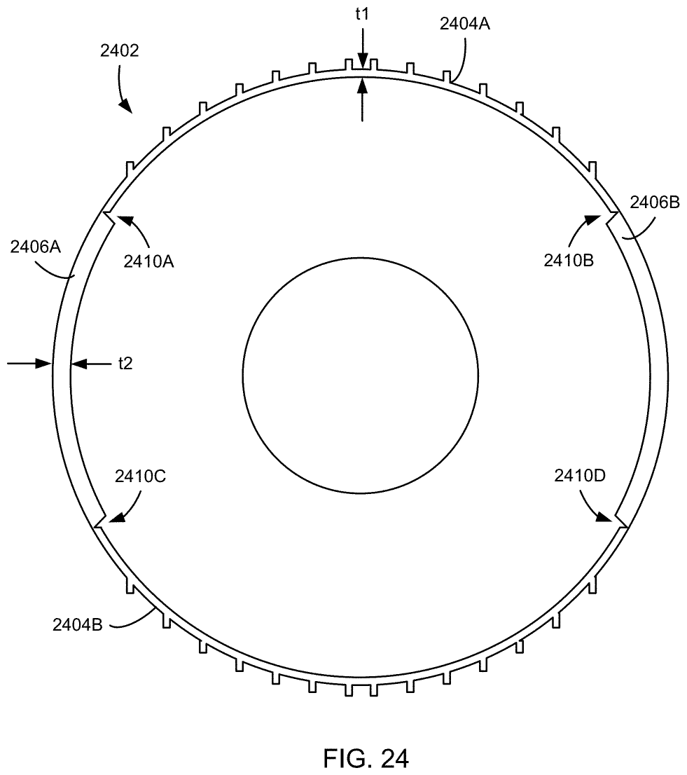

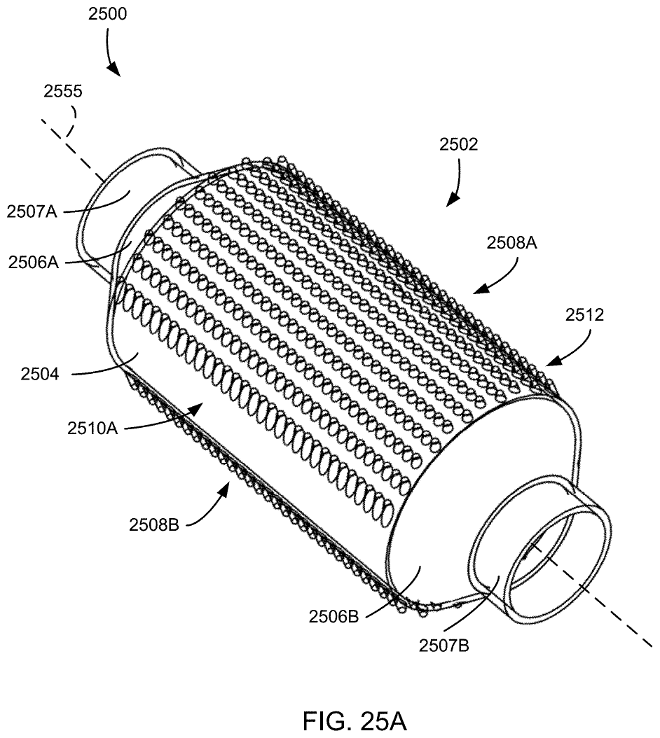

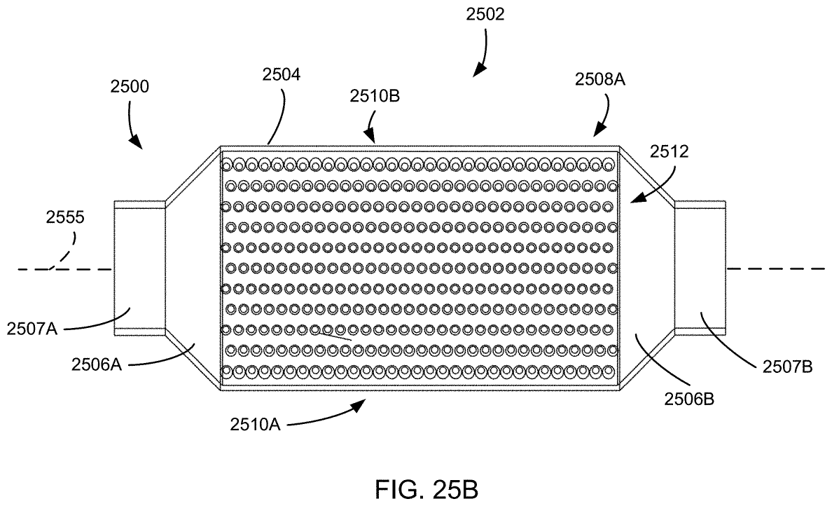

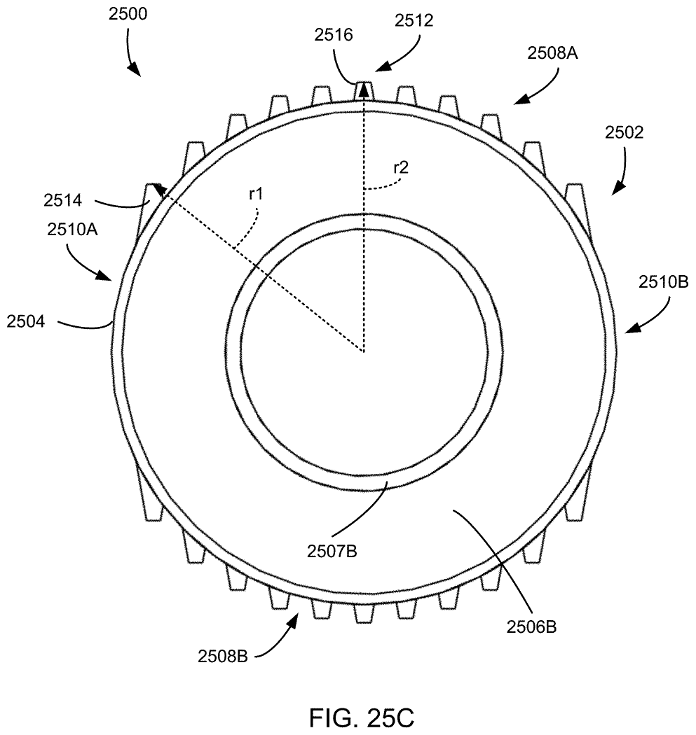

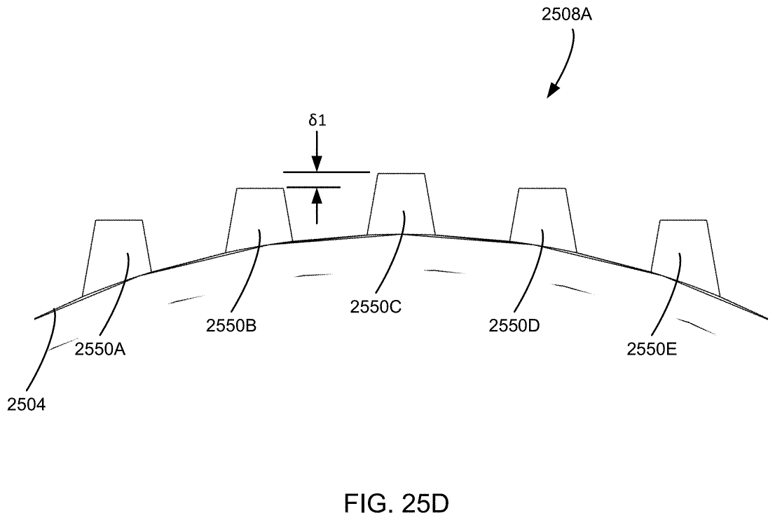

FIGS. 25A-25D illustrate one example implementation of a balloon 2500 in accordance with the present disclosure in an unstrained state. More specifically, FIG. 25A is an isometric view of the balloon 2500, FIG. 25B is a plan view of the balloon 2500, FIG. 25C is an end view of the balloon 2500, and FIG. 25D is a cross-sectional view of a textured surface of the balloon 2500.

Referring first to FIGS. 25A-25C, the balloon 2500 includes an elongate body 2502 extending along a longitudinal axis 2555. The elongate body 2502 generally includes a middle portion 2504 and tapering end portions 2506A, 2506B, each of which terminates in a respective annulus 2507A, 2507B. The middle portion 2504 of the balloon 2500 includes oppositely disposed textured portions 2508A, 2508B. Extending between the textured portions 2508A, 2508B are untextured portions 2510A, 2510B. In other implementations, the surface of the middle portion 2504 of the balloon 2500 may be divided into more than two textured portions and/or more than two untextured portions. Similarly, balloons in accordance with the present disclosure may include only one textured portion.

As best seen in FIG. 25B, the textured portions 2508A, 2508B of the balloon 2500 include uniformly distributed longitudinal rows of protrusions (e.g., protrusions rows 2512). As discussed below in further detail, the protrusions of the balloon 2500 have a truncated cone shape, although other protrusion shapes may be used in other implementations. Also, as visible in FIG. 25B, adjacent rows of protrusions of the balloon 2500 are offset relative to each other such that every other row is aligned. In other implementations other row configurations may be implemented. For example, all rows may be aligned or multiple offsets may be used between different pairs of rows.

In at least certain implementations, the frictional and adhesive properties of the protrusions within a given row may vary based on the longitudinal spacing between the protrusions. For example, if spacing between protrusions is relatively narrow (e.g., from around 25 μm to around 400 μm, or from around 5% to 50% of the width of the protrusions), traction in a collapsed or unstrained state is generally reduced as compared to implementations including wider spacing. Testing suggest that such variable traction is the result of narrowly spaced protrusions in a given row more closely approximating the drag and traction provided by a continuous structure (e.g., a rib) as opposed to a series of independent protrusions. For example, during certain tests, it was observed that when in a partially deflated state, traction for a given balloon having twenty rows of approximately forty protrusions each approximated the traction provided by twenty continuous ribs extending along the length of the balloon. However, as the spacing between the protrusions was increased (e.g., by inflating and expanding the balloon) traction was observed to increase significantly. Among other things, the increase in traction was attributable to substantially all of the leading edges of the 400 protrusions being exposed and able to fully engage and interact with the inner wall of the physiological lumen when in the expanded state as compared to when the protrusions were more closely spaced.

The protrusions are configured such that when in a partially inflated state, each protrusion of each respective textured portion 2508A, 2508B extends in a common transverse direction relative to the longitudinal axis. In other words, the protrusions of the textured portion 2508A extend parallel to each other in a first transverse direction while the protrusions of the textured portion 2508B extend parallel to each other in a second transverse direction that is opposite the first lateral direction. In other implementations, the textured portions 2508A, 2508B may not be oppositely disposed but nevertheless including protrusions that extend in respective transverse directions.

As shown in FIG. 25C, the textured portions 2508A, 2508B and the untextured portions 2510A, 2510B collectively extend around the circumference of the middle portion 2504 of the balloon 2500. In the particular example illustrated in FIG. 25C, each textured portion 2508A, 2508B extends around about a third of the surface of the middle portion 2504, while the remaining third of the surface is divided between the untextured portions 2510A, 2510B. It should be appreciated, however, that the distribution of the textured and untextured portions of the balloon 2500 may vary from that which is illustrated in FIGS. 25A-25D.

As previously noted, each of the tapering end portions 2506A, 2506B terminate in a respective annulus 2507A, 2507B. In general, each annulus 2507A, 2507B is sized and shaped to be fit onto an overtube, catheter, endoscope, or similar tool. Accordingly, the shape and dimensions of each annulus 2507A, 2507B may vary depending on the specific tool onto which the balloon 2500 is to be disposed. However, in at least certain implementations, each annulus 2507A, 2507B may be reinforced relative to other portions of the balloon 2500 that are intended to expand. For example, in certain implementation, the wall thickness of each annulus 2507A, 2507B may be from and including about 1.25 times to and including about 5 times thicker than the wall thickness of the rest of the balloon 2500. Among other things, thickening each annulus 2507A, 2507B facilitates improved retention of the balloon 2500 on an overtube or other tool, particularly when the balloon 2500 is subjected to inflation and deflation.

As illustrated in FIG. 25C, in at least certain implementations, the height of each protrusion may be defined such that each protrusion extends to a common radius. For example, protrusion 2514 has a height such that a center of the tip of the protrusion 2514 extends to a radius r1 while protrusion 2516 has a height such that a center of the tip of the protrusion 2516 extends to a radius r2 that is substantially the same as the radius r1 of protrusion 2514. An alternative interpretation of this approach to determining protrusion heights is that each protrusion extends from the surface of the balloon 2500 such that the midpoint of a top surface of each protrusion lies on a common circle.

Referring now to FIG. 25D, a partial cross-sectional view of the middle portion 2504 of the balloon 2500 is provided to illustrate further details of the protrusions of the textured portions 2508A, 2508B. In the particular illustrated design, each protrusion (e.g. protrusions 2550A-2550E) of the balloon 2500 has a truncated conical shape. While illustrated as having flat tops, in at least certain implementations, the top surface of each protrusion may instead be concave, as previously discussed herein.

FIG. 25D illustrates an alternative approach to selecting the height of each protrusion. More specifically, in at least certain implementations, the height of protrusions in each row may be selected such that there is a predetermined height difference between adjacent rows. For example, FIG. 25D includes a dimension 61 corresponding to the difference in height between adjacent rows. As illustrated, 61 may be maintained between successive pairs of adjacent rows such that the top surfaces of the protrusions in adjacent rows descend in a step-like manner. Alternatively, 61 may differ between adjacent rows. Although various values of 61 may be used in implementations of the present disclosure, in at least certain implementations 61 may be from and including about 5 μm to and including about 3 mm. The foregoing approach may be used as an alternative to the previously discussed approach in which each protrusion extends such that a midpoint of its tip is at a common radius or lies on a common circle.

Although the specific dimensions of the balloon 2500 may vary based on the particular application of the balloon 2500, in at least certain implementations, the balloon 2500 may have an overall length from and including about 10 mm to and including about 100 mm. In such implementations, the middle portion 2504 of the balloon may be from and including about 5 mm to and including about 90 mm and the end portions 2506A, 2506B may each be from and including about 2 mm to and including about 10 mm. The middle portion 2504 may also have a resting/partially inflated diameter from and including about 2 mm to and including about 50 mm, with the diameter corresponding to the surface of the middle portion 2504 from which the protrusions extend. The middle portion 2504 may also have a wall thickness from and including about 100 μm to and including about 3000 μm. Further in such implementations, each annulus 2507A, 2507B may have an outer diameter from and including 1 mm to and including 20 mm and a wall thickness from and including 100 μm to and including 5000 μm. The foregoing dimensions should be understood to be merely examples and designs in which the foregoing dimensions fall below or exceed the specified ranges should still be regarded as being within the scope of this disclosure.

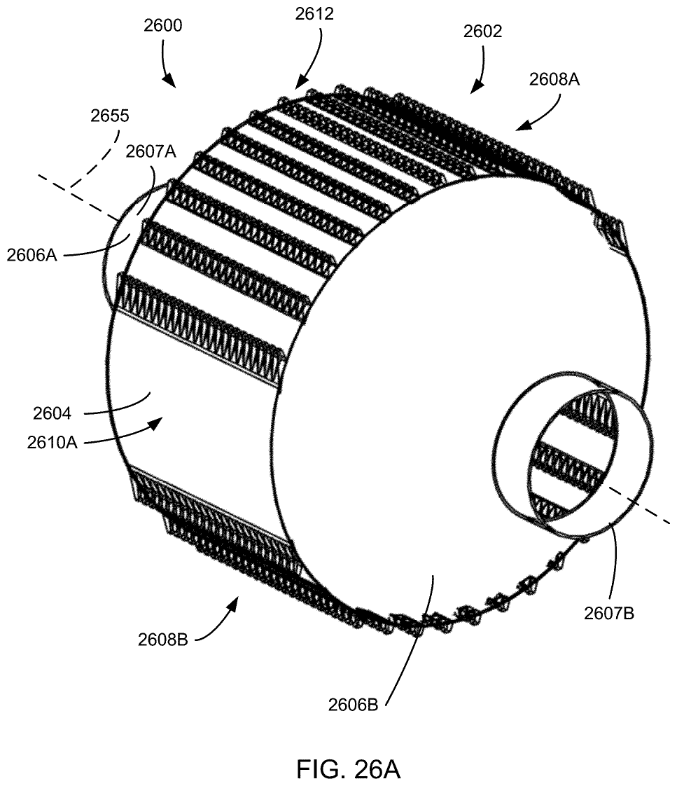

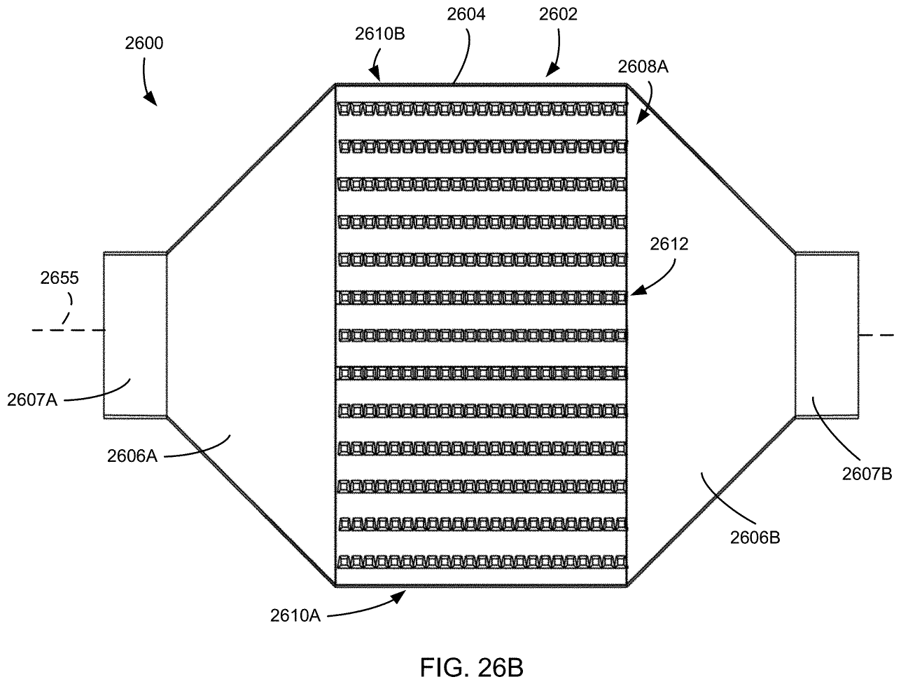

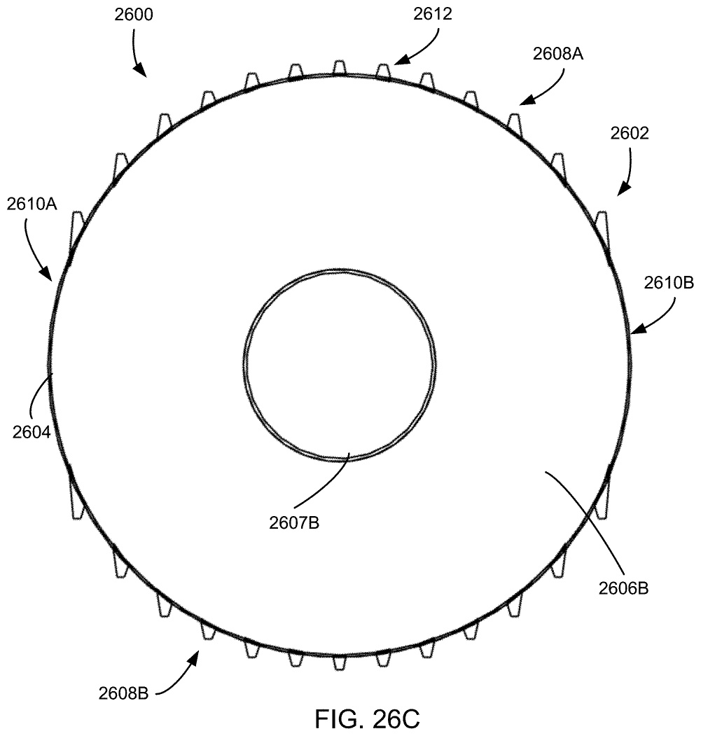

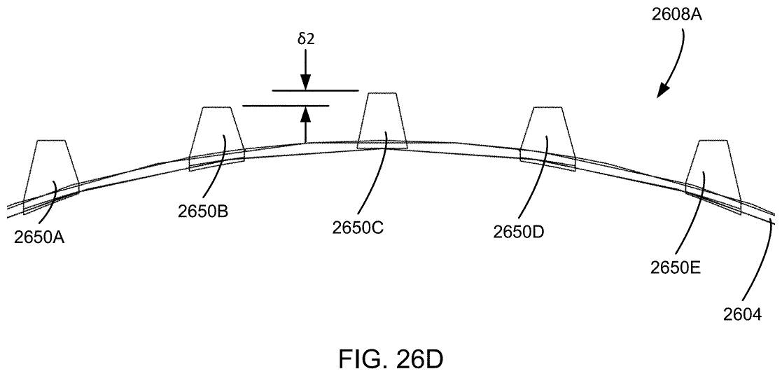

Referring next to FIGS. 26A-26D, a second balloon 2600 in an unstrained state is provided. Similar to the previously disclosed balloon 2500, the balloon 2600 includes an elongate body 2602 extending along a longitudinal axis 2655, the elongate body including a middle portion 2604 and tapering end portions 2606A, 2606B. Each of the end portions 2606A, 2606B similarly terminates in a respective annulus 2607A, 2607B for coupling the balloon 2600 to an overtube or similar tool. The middle portion 2604 of the balloon 2600 also includes oppositely disposed textured portions 2608A, 2608B and untextured portions 2610A, 2610B extending therebetween.

As best seen in FIG. 26B, the textured portions 2608A, 2608B of the balloon 2600 include uniformly distributed rows of protrusions 2612. In contrast to the truncated cone protrusions of the balloon 2500 discussed above, the protrusions of the balloon 2600 have a truncated pyramidal shape. Also, as shown in FIG. 26B, adjacent rows of protrusions of the balloon 2600 are aligned relative to each other, as compared to the offset configuration of the balloon 2500 and adjacent protrusions within a given row of the balloon 2600 are sized and shaped such that they contact each other. This in contrast to the rows of the balloon 2500 in which adjacent protrusions in a row were spaced apart.

Like those of the balloon 2500, the protrusions 2612 of the balloon 2600 are configured such that when in a partially inflated state, each protrusion of each respective textured portion 2608A, 2508B extends in a lateral direction relative to the longitudinal axis. In other words, the protrusions of the textured portion 2608A extend in a first lateral direction while the protrusions of the textured portion 2608B extend in a second lateral direction that is opposite the first lateral direction.

Referring now to FIG. 26D, a partial cross-sectional view of the middle portion 2604 of the balloon 2600 is provided to illustrate further details of the protrusions of the textured portions 2608A, 2608B (e.g., protrusions 2650A-2650E). As previously noted the protrusions 2650A-2650E have a truncated square-based pyramid shape having a flat top. Nevertheless, the top surface of each protrusion may instead be concave, as previously discussed herein. Like the protrusions of the balloon 2500, adjacent rows of the protrusions of the balloon 2600 may be configured such that the change in height (indicated as 62) between adjacent rows of protrusions may be from and including about 5 μm to and including about 3 mm. Alternatively, and as described above in the context of FIG. 25C, each protrusion may have a height such that a midpoint of a tip of each protrusion extends to a common radius/lies on a common circle.

Although the specific dimensions of the balloon 2600 may vary based on the particular application of the balloon 2600, in at least certain implementations, the balloon 2600 may have an overall length from and including about 10 mm to and including about 100 mm. In such implementations, the middle portion 2604 of the balloon may be from and including about 5 mm to and including about 90 mm and the end portions 2606A, 2606B may each be from and including about 2 mm to and including about 10 mm. The middle portion 2604 may also have a resting/partially inflated diameter from and including about 2 mm to and including about 50 mm, with the diameter corresponding to the surface of the middle portion 2604 from which the protrusions extend. The middle portion 2604 may also have a wall thickness from and including about 100 μm to and including about 3000 μm. Further in such implementations, each annulus 2607A, 2607B may have an outer diameter from and including 1 mm to and including 20 mm and a wall thickness from and including 100 μm to and including 5000 μm. The foregoing dimensions should be understood to be merely examples and designs in which the foregoing dimensions fall below or exceed the specified ranges should still be regarded as being within the scope of this disclosure.

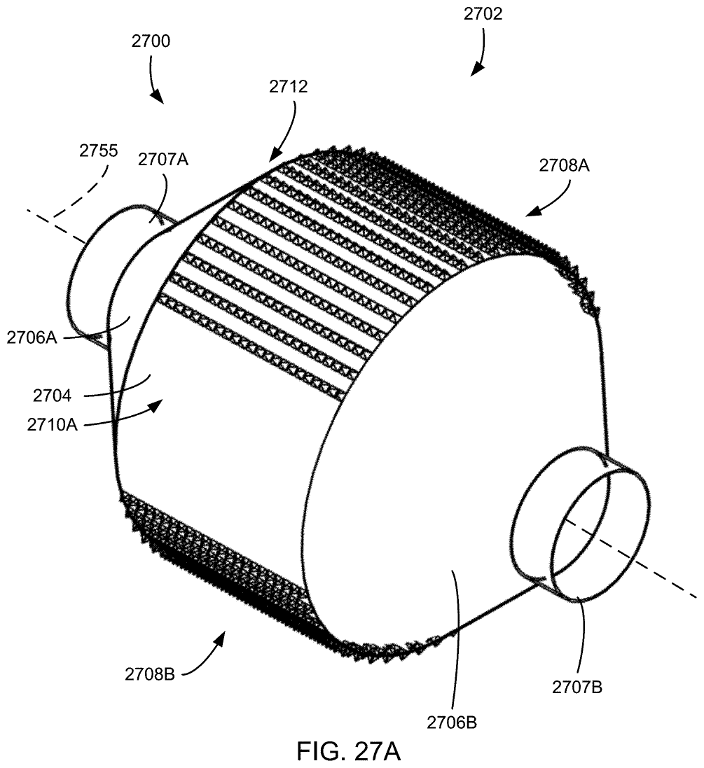

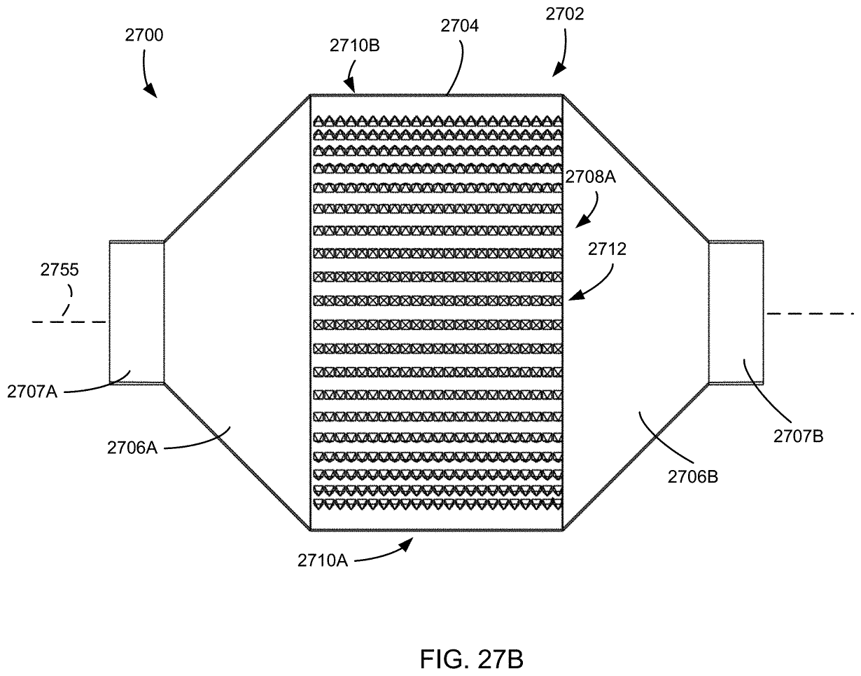

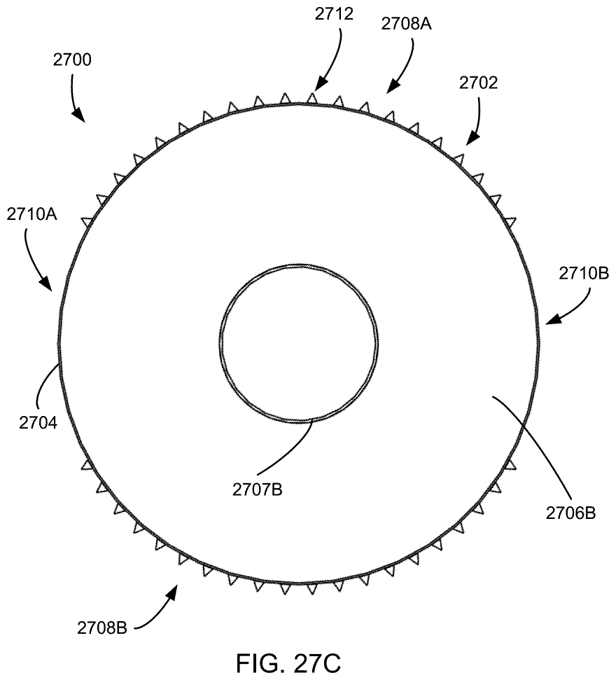

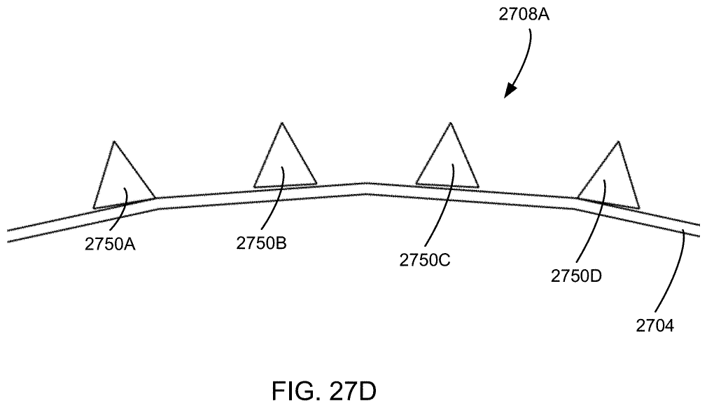

Referring next to FIGS. 27A-27D, a third balloon 2700 in an unstrained state is provided. Similar to the previously disclosed balloons, the balloon 2700 includes an elongate body 2702 extending along a longitudinal axis 2755, the elongate body including a middle portion 2704 and tapering end portions 2706A, 2706B. Each of the end portions 2706A, 2706B terminates in a respective annulus 2707A, 2707B for coupling the balloon 2700 to an overtube or similar tool. The middle portion 2704 of the balloon 2700 includes oppositely disposed textured portions 2708A, 2708B and untextured portions 2710A, 2710B extending therebetween.

The textured portions 2708A, 2708B of the balloon 2700 include uniformly distributed rows of protrusions 2712 and, more specifically, pyramidal protrusions. Similar to the rows of protrusions of the balloon 2600, the rows of protrusions 2712 of the balloon 2700 are aligned relative to each other and adjacent protrusions within a given row of the balloon 2700 are sized and shaped such that they contact each other. However, in contrast to the previous two example balloons 2500, 2600, the protrusions 2712 of the balloon 2700 are configured such that when in a partially inflated state, each protrusion of each respective textured portion 2708A, 2708B extends radially.

Referring now to FIG. 27D, a partial cross-sectional view of the middle portion 2704 of the balloon 2700 is provided to illustrate further details of the protrusions of the textured portions 2708A, 2708B. As previously noted the protrusions (e.g., protrusions 2750A-2750D) have a pyramidal shape; however, the pyramidal shaped protrusions may have any other suitable shape discussed herein, including shapes having concave top surfaces.

Although the specific dimensions of the balloon 2700 may vary based on the particular application of the balloon 2700, in at least certain implementations, the balloon 2700 may have an overall length from and including about 10 mm to and including about 100 mm. In such implementations, the middle portion 2704 of the balloon may be from and including about 5 mm to and including about 90 mm and the end portions 2706A, 2706B may each be from and including about 2 mm to and including about 10 mm. The middle portion 2704 may also have a resting/partially inflated diameter from and including about 2 mm to and including about 50 mm, with the diameter corresponding to the surface of the middle portion 2704 from which the protrusions extend. The middle portion 2704 may also have a wall thickness from and including about 100 μm to and including about 3000 μm. Further in such implementations, each annulus 2707A, 2707B may have an outer diameter from and including 1 mm to and including 20 mm and a wall thickness from and including 100 μm to and including 5000 μm. The foregoing dimensions should be understood to be merely examples and designs in which the foregoing dimensions fall below or exceed the specified ranges should still be regarded as being within the scope of this disclosure.

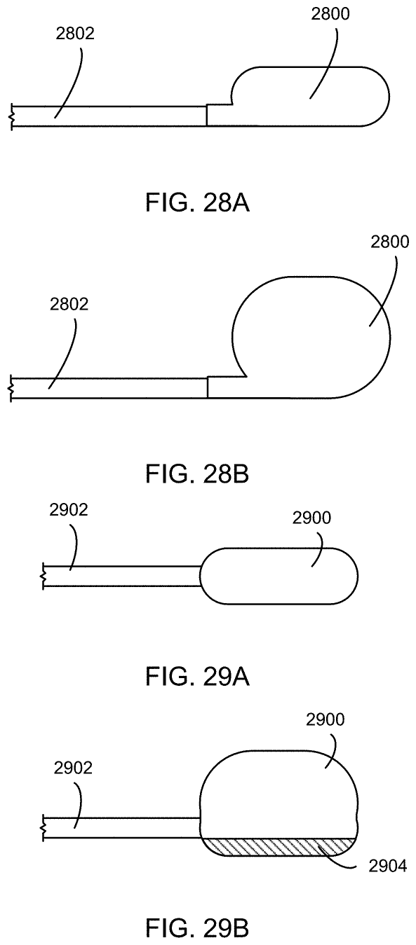

Previous implementations discussed herein generally include balloons that are mounted coaxially with an overtube or similar medical tool and expand in a substantially uniform, radial direction about the tube. Nevertheless, it should be appreciated that in at least certain implementations, such balloons may instead be configured to expand directionally. For example, 28A and 28B illustrates a first example balloon 2800 eccentrically mounted to an overtube 2802. Accordingly, as the balloon 2800 is inflated and expands (as illustrated in the transition from FIG. 28A to 28B), the balloon 2800 is biased to one side of the overtube 2802.

FIGS. 29A and 29B illustrate an alternative implementation in which a balloon 2900 is configured to expand directionally from an overtube 2902 or similar tool on which the balloon 2900 is mounted. Such directional expansion may be achieved, for example, by forming the balloon to have a localized region or side (indicated by hashed area 2904) having increased stiffness or rigidity as compared to other portions of the balloon 2900. Such reinforcement may be achieved, for example, by increasing the wall thickness of the balloon 2900 in the region having reduced expansion; using a stiffer material in the region having reduced expansion; including internal or external ribs, bands, or similar reinforcing structures in the area having reduced expansion; or using any other suitable technique for locally increasing stiffness.

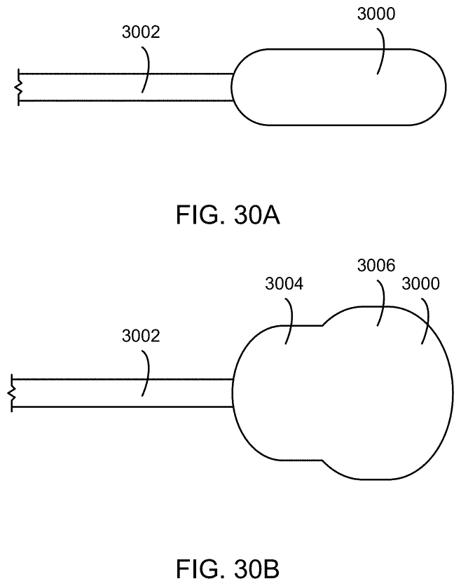

In addition to directional expansion, balloons in accordance with the present disclosure may have variable expansion along their length. For example, FIGS. 30A and 30B are schematic illustrations of a balloon 3000 disposed on an overtube 3002 or similar tool. As illustrated in the transition between FIGS. 30A and 30B, when inflated, a proximal portion of the balloon 3004 expands to a lesser extent than a distal portion of the balloon 3006. Similar to the balloon 2900 of FIGS. 29A and 29B, such variable expansion may be achieved by varying material, wall thickness, and reinforcement along the length of the balloon 3000.

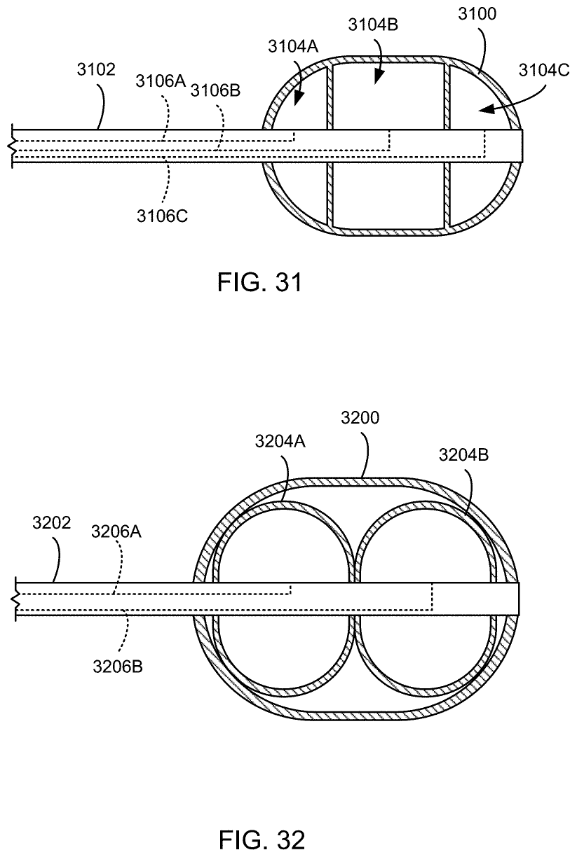

In addition to or as an alternative to selectively reinforcing sections of a balloon to provide variable expansion, balloons in accordance with the present disclosure may include distinct and selectively expandable compartments. For example, FIG. 31 illustrates an example balloon 3100 disposed on an overtube 3102 or similar tool and defining three distinct and isolated internal compartments 3104A-3104C. Each compartment 3104A-3104C is connected to an independently controlled air line 3106A-3106C such that air may be selectively supplied and removed from each of the compartments 3104A-3104C to selectively control their respective expansion and deflation.

FIG. 32 illustrates an alternative approach to providing a balloon having variably expandable regions. More specifically, FIG. 32 illustrates a sheath or outer balloon 3200 within which multiple and independently inflatable internal balloons 3204A, 3204B may be disposed. Each of the balloons 3200, 3204A, and 3204B may in turn be coupled to an overtube 3202 or similar tool. In such implementations, the outer balloon 3200 may include texturing or protrusions, as described herein, while the internal balloons may be substantially smooth. Similar to the compartmentalized balloon 3100 of FIG. 31, each internal balloon 3204A, 3204B may be in communication with a respective and independently controlled air line 3106A, 3106B to selectively control inflation and deflation of the internal balloons and, as a result, the overall shape of the outer balloon 3200.

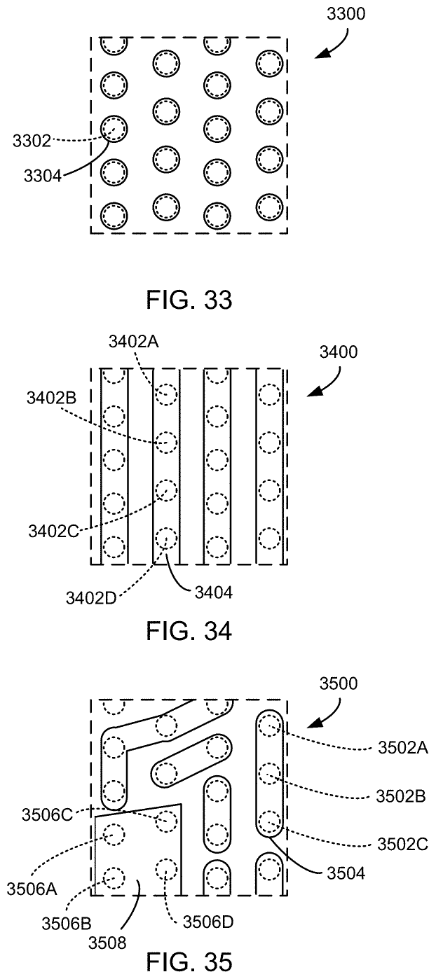

In certain implementations of the present disclosure, protrusions extending from the balloon may be reinforced to increase overall rigidity of the protrusions, thereby preventing or reduce bending or other deformation during transportation of the balloon within a physiological lumen or following anchoring of the balloon within the lumen. In certain implementations, such reinforcement of the protrusions may be provided on the internal surface of the balloon. For example, FIGS. 33-35 each illustrate non-limiting examples of internal reinforcement that may be applied to the protrusions. FIG. 33, for example, illustrates a portion 3300 of an example inner balloon surface in which each protrusion (e.g., protrusion 3302) is individually reinforced by a corresponding bump (e.g., bump 3304 corresponding to protrusion 3302) or similar localized thickening of the balloon wall opposite the protrusion. As another example, FIG. 34 illustrates a portion 3400 of another example inner balloon surface in which multiple protrusions (e.g., protrusions 3402A-3402D) are linked by a corresponding ridge, rib, or similar reinforcing structure (e.g., rib 3404) extending along the inner surface of the balloon. FIG. 35 illustrates another portion 3500 of an example inner balloon surface illustrating that such reinforcement may be non-uniform. For example, while protrusions 3502A-3502C are reinforced using a common and straight rib 3504, protrusions 3506A-3406D are reinforced by a patch 3508 of balloon material.

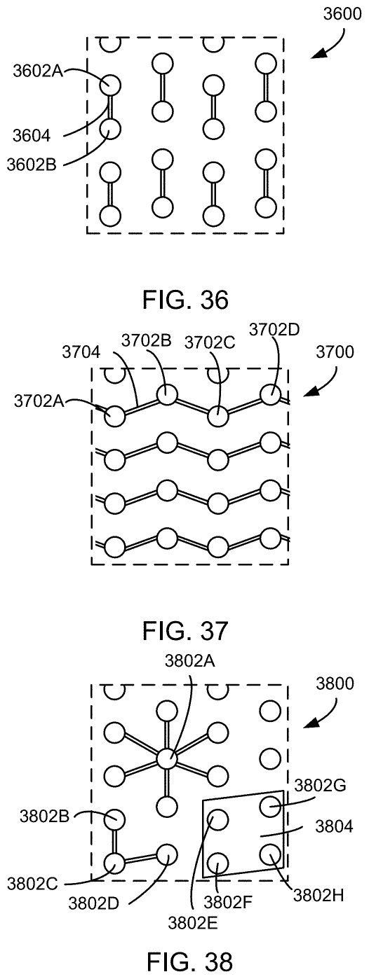

Reinforcement of the protrusions may also be achieved by linking or connecting protrusions on the exterior surface of the balloon. For example, FIG. 36 illustrates a portion 3600 of an external surface of a first example balloon in which adjacent protrusions (e.g., protrusions 3602A, 3602B) are linked or otherwise mutually reinforced by a rib 3604 extending therebetween. FIG. 37 illustrates a portion 3700 of a second example balloon in which protrusions (e.g., protrusions 3702A-3702D) are linked by continuous ribs (e.g., rib 3704). Finally, FIG. 38 illustrates a portion 3800 of a third example balloon having non-uniform protrusion reinforcement. For example, protrusion 3802A is coupled to and reinforced by each of its nearest neighboring protrusions, protrusions 3802-3802D are reinforced to form an “L” shaped pattern, and protrusions 3802E-3802H are reinforced by a patch 3804 or pad extending therebetween.

The foregoing examples of internal and external protrusion reinforcement are intended merely as non-limiting examples. More generally, reinforcement of protrusions in accordance with the present disclosure may be achieved by either or both of providing additional material on the inner surface of the balloon opposite the protrusions, providing additional material on the external surface of the balloon adjacent the protrusions, or forming a mechanical link between protrusions, such as by forming a rib or similar structure extending between protrusions.

The foregoing balloon designs are intended merely as examples and are not intended to limit the scope of the present disclosure. Rather, features of any balloon disclosed herein may be combined in any suitable manner. For example, any size, shape, and arrangement of protrusions may be implemented with any corresponding balloon shape or size. Similarly, other features, such as those related to controlled collapse, may be incorporated into any balloon design disclosure herein. Similarly, any specific dimensions or proportions provided in the context of specific balloon designs are intended merely as examples and should not be construed as limiting. More generally, any particular implementations of balloons discussed or illustrated herein should be regarded as one possible combination of features of balloons in accordance with the present disclosure.

Overtube Assemblies Including Balloon Inflation/Deflation Systems

An endoscopic overtube is a sleeve-like device designed to facilitate endoscopic procedures. During upper endoscopic procedures, for example, overtube may be used to protect, among other things, the hypopharynx from trauma during intubations, the airway from aspiration, and the esophagus during extraction of sharp foreign bodies. Similarly, during lower endoscopic procedures, such as enteroscopy and colonoscopy, overtubes may be used to protect various structures of the gastrointestinal tract while also preventing loop formation.

In endoscopic processes including endoscopic balloons, the balloon may be coupled to the overtube and the overtube may include passageways or ducts that extend along its length from the balloon to one or more proximal ports. For example, certain conventional balloon overtubes include a balloon and overtube with an inflation/deflation port and a fluid access port. Such conventional balloon overtubes are often operated using a separate and cumbersome inflation system coupled to the overtube by one or more small plastic tubes. The inflation system generally includes a pump and valves for providing air to and extracting air from the inflation/deflation port of the overtube via the plastic tubes. Such systems may be actuated by foot pedal or handheld button, either by the gastroenterologist user, or by a technician.

Among other issues, such conventional inflation systems are expensive to purchase and operate, time consuming to set up, and lack portability. Accordingly, such conventional systems generally preclude balloon endoscopy from being used in facilities that may lack the resources for a conventional system or in applications outside of an endoscopic center.

To address the foregoing issues, among others, an improved overtube assembly is provided. The improved overtube assembly includes an inflation/deflation system integrated with the overtube to provide a standalone or substantially standalone system.

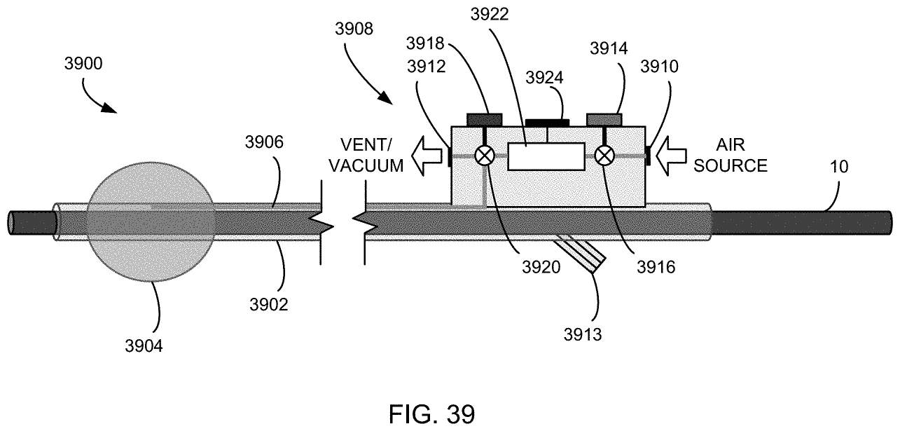

FIG. 39 is a schematic illustration of an example overtube assembly 3900 in accordance with the present disclosure. As illustrated, the overtube assembly 3900 is disposed on an endoscope 10. The overtube assembly 3900 includes an overtube 3902 coupled to a balloon 3904. A balloon line 3906 extends along or through the overtube 3902 from the balloon 3904 to an inflation/deflation assembly 3908. In certain implementations, the balloon line 3906 may be a lumen defined by the overtube 3902; however, in other implementations, the balloon line 3906 may be a separate lumen coupled to or embedded within the overtube 3902.

The balloon 3904 may be, but is not necessarily limited to, an endoscopic balloon including one or more textured portions according to any implementation discussed herein.

The inflation/deflation assembly 3908 includes various ports and controls to facilitate the inflation and deflation of the balloon 3904. For example, the inflation/deflation assembly 3908 includes each of an inflation port 3910 and a deflation port 3912. The inflation port 3910 is adapted to be coupled to a suitable source of pressurized air (not shown), which may include, without limitation, “house air” available within an endoscopy or operation room suite, a hand pump, a hand syringe, a foot-actuated floor pump, or a reservoir of compressed air. Similarly, the deflation port 3912 may be configured to be coupled to a vacuum to facilitate rapid deflation of the balloon 3904. Alternatively, the deflation port 3912 may vent to atmosphere. The overtube assembly 3900 may further include other ports, such as, but not limited to, a fluid in/out port 3913 to facilitate injection or removal of fluid from the physiological lumen within which the overtube assembly 3900 is disposed.

The inflation/deflation assembly 3908 further includes controls for selectively inflating and deflating the balloon 3904. In the specific implementation illustrated in FIG. 39, for example, the inflation/deflation assembly 3908 includes each of an inflation button 3914 for selectively opening an inflation valve 3916 and a deflation button 3918 for selectively opening a deflation valve 3920. When opened (e.g., by depressing the inflation button 3914), the inflation valve 3916 permits air flow from the air source through a regulator 3922 of the inflation/deflation assembly 3908 and to the balloon 3904 via the balloon line 3906. Similarly, when opened, the deflation valve 3920 permits air flow from the balloon 3904, through the balloon line 3906, and out of the deflation port 3912.

As noted, the inflation/deflation assembly 3908 may include a regulator 3922 disposed between the inflation port 3910 and the balloon line 3906. In certain implementations, the regulator 3922 may be fixed to provide a predetermined flow rate at a predetermined pressure; however, in at least some implementations the regulator 3922 may be adjustable (e.g., by an adjustment knob 3924 or similar control element coupled to the regulator 3922).

The various control elements included in the inflation/deflation assembly 3908 may be mechanical, electronic, or a combination of both. In implementations in which electronic components are included, the inflation/deflation assembly 3908 may generally include suitable circuitry, memory, and processing components to perform various functions such as, but not limited to, receiving inputs from the buttons 3914, 3918; actuating the valves 3916, 3920; and adjusting the regulator 3922. In certain implementations the inflation/deflation assembly 3908 may also be communicatively coupled to one or more remote computing devices that may be used to operator and/or collect data from the inflation/deflation assembly 3908. To the extent any electronic components are included in the inflation/deflation assembly 3908, the inflation/deflation assembly 3908 may further include an onboard power source (such as a battery) and/or may be electrically coupleable to an external power source, such as a wall socket or external battery.

In certain implementations, the inflation/deflation assembly 3908 may include an onboard pump between the inflation port 3910 and the regulator 3922 and the inflation port 3910 may simply be open to ambient air. In such implementations, the inflation/deflation assembly 3908 may further include one or more permanent or replaceable filter element disposed between the inflation port 3910 and the regulator 3922 to improve the quality of the air provided to the balloon 3904.

As shown in FIG. 39, the inflation/deflation assembly 3908 may be directly coupled to a proximal portion of the overtube 3902. In certain implementations, the inflation/deflation assembly 3908 may be specifically sized and shaped to be manipulated using one hand, thereby improving ease of use and freeing a user's second hand to perform other tasks. Accordingly, the size and shape of the inflation/deflation assembly 3908 may be chosen for any of right-, left-, or ambidextrous operation.

In at least certain implementations, the overtube assembly 3900, including the inflation/deflation assembly 3908, may be configured to be disposable in whole or in part. For example, in certain implementations, the overtube assembly 3900 may be disassembled in whole or in part, with certain of the components of the overtube assembly 3900 being recyclable or otherwise readily disposable.

It should be understood that the foregoing overtube assembly 3900 is merely an example and implementations of the present disclosure are limited to the specific implementation discussed above. Rather, overtube assemblies in accordance with the present disclosure more generally include an overtube to which flow and pressure regulating components are coupled and with which such flow and pressure regulating components are integrated into a unitary assembly.

Split Overtubes

Conventional overtubes, including balloon overtubes, are continuous tubular structures. As a result, such overtubes may only be installed on endoscopes (or similar tools) by inserting a distal end of the endoscope into a proximal end of the overtube and extending the endoscope through the overtube. This process necessarily requires that the endoscope be outside the patient and, as a result, must be performed at the outset of any endoscopic procedure. In certain instances, however, a physician may not know whether an overtube is required until mid-procedure. At such time in the procedure, it may be very difficult to fully intubate the patient due to irregular anatomy, or other complications. Physicians also sometime realize they cannot easily position the endoscope to successfully biopsy tissue. In these example cases, a physician would generally need to remove the endoscope from the patient, attach an overtube, re-intubate the patient, and deliver the endoscope to its prior location. This leads to increased procedure time and challenges of advancing the scope to the previous furthest point. Thus, there is a need to be able to attach an overtube mid-procedure and, more specifically, to attach an overtube to the endoscope and advance the overtube to the tip of the endoscope without losing any purchase with the endoscope, removing the endoscope from the patient, or otherwise backtracking in the procedure.

To address the foregoing issues, among others, a split or wraparound overtube is provided here. In general, the split overtube includes a longitudinally extending split that allows the overtube to be opened and placed onto an endoscope. To prevent separation of the split overtube and/or disengagement from the endoscope, the split overtube may include features to secure the overtube to the underlying endoscope. For example, in certain implementations, the overtube may have a high-friction inner surface adapted to frictionally engage the endoscope. Such high-friction properties may be a result of the material of the split overtube, a coating or adhesive applied to the inner surface, texturing of the inner surface, and the like. In certain implementations, friction between the overtube and the endoscope may be selectively modified by introducing a fluid into the annular space between the overtube and the endoscope, such that the fluid acts as a lubricant between the two components.

The overtube may also include features to prevent the overtube from splitting once coupled to the endoscope. For example, in certain implementations surfaces of the overtube that contact when closed about an endoscope may be textured or treated to frictionally engage each other. In certain implementations, the overtube may be configured to wrap about the endoscope such that portions of the overtube overlap. Like the previously mentioned contacting surfaces, the overlapping portions of the overtube may also include coatings, texturing, or structural features configured to engage each other and maintain the overtube in a closed configuration about the endoscope.

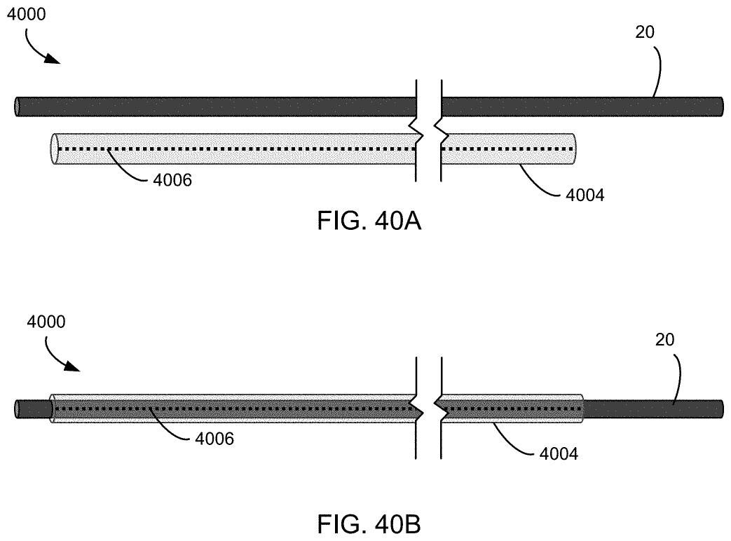

Referring first to FIGS. 40A and 40B, an endoscope and overtube assembly 4000 is illustrated in each of a separated and coupled configuration. More specifically, FIG. 40A illustrates the endoscope 20 adjacent the overtube 4004. The overtube 4004 includes a split 4006 extending along its length such that the overtube 4004 may be opened (e.g., into a “C”-shape) and an exposed/ex vivo portion of the endoscope 20 may be inserted laterally into the overtube 4004. Although illustrated in FIGS. 40A and 40B as being straight, the split 4006 more generally extends along the full length of the overtube 4004, but may extend both about and along the overtube 4004 in doing so. For example, instead of a straight split (such as illustrated), the split 4006 may be helical or include helically extending segments. FIG. 40B illustrates the endoscope and overtube assembly 4000 in an assembled configuration in which the endoscope 20 is disposed within the overtube 4004. Once disposed on the endoscope 20, the overtube 4004 may be advanced along the endoscope 20 (e.g., in vivo) to the tip of the endoscope 20.

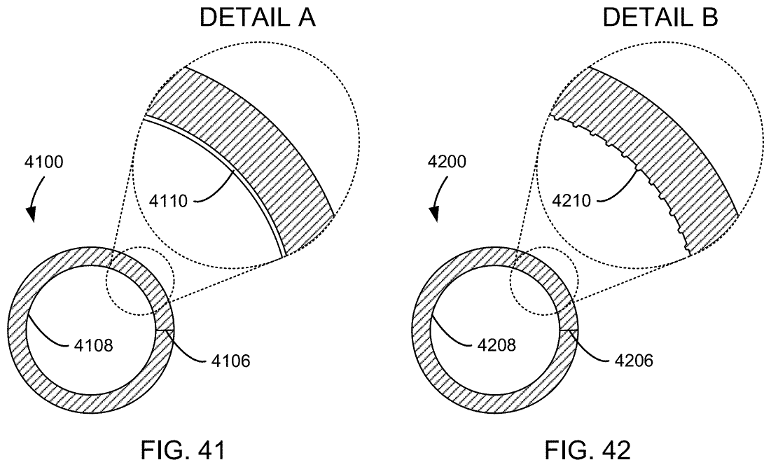

Although the overtube may be advanced along the endoscope 20, in certain implementations, the frictional engagement between the endoscope 20 and the overtube 4004 may be designed to provide at least some resistance to undesirable movement of the endoscope 20 relative to the overtube 4004 once the overtube 4004 is installed. FIGS. 41 and 42 provide two example approaches of modifying the engagement between the endoscope 20 and overtube 4004.

Referring first to FIG. 41, a cross-sectional view of a first example overtube 4100 is provided. As illustrated, the overtube 4100 includes a split 4106 that allows the overtube 4100 to be opened for insertion of the endoscope. As illustrated in Detail A, at least a portion of the inner surface 4108 of the overtube 4100 may include a coating or layer 4110 with predetermined frictional properties. Similarly, FIG. 42 is a cross-sectional view of a second example overtube 4200 is provided. As illustrated, the overtube 4200 also includes a split 4206 that allows the overtube 4200 to be opened for insertion of the endoscope. As illustrated in Detail B, at least a portion of the inner surface 4208 of the overtube 4200 may include texturing 4210 to modifying the frictional properties of the inner surface 4208. Although various textures may be used, in at least certain implementations, such texturing 4210 may be similar to the texturing described above in the context of endoscopic balloons. It should be appreciated that similar coating or texturing may also be applied to portions of the exterior surface of the overtubes 4100, 4200 to modify the frictional engagement between the overtubes 4100, 4200 and any physiological lumen within which they may be used.

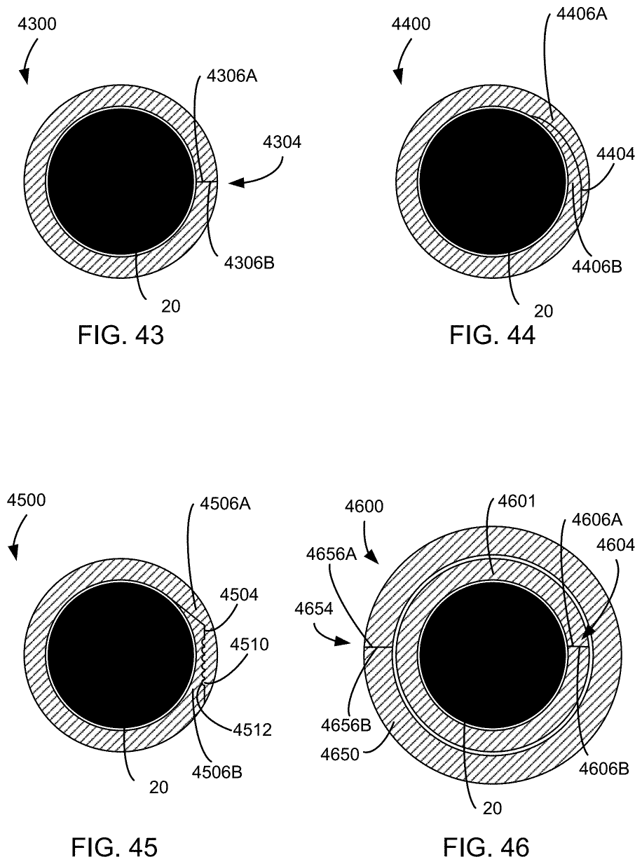

FIGS. 43-46 illustrate alternative configurations of split overtubes in accordance with the present disclosure and, in particular, different ways in which such overtubes may be retained on an endoscope.

Referring first to FIG. 43, a cross-sectional view of an overtube 4300 disposed on an endoscope 20 is provided. As illustrated, the overtube 4300 includes a lateral split 4304 including a first surface 4306A and a second surface 4306B. As illustrated, when disposed on the endoscope 20, the first surface 4306A and the second surface 4306B abut. In certain implementations, the overtube 4300 may be formed from a material having sufficient rigidity that the first surface 4306A and the second surface 4306B are in positive contact. Alternatively, or in addition, one or both of the first surface 4306A and the second surface 4306B may have a coating, layer, texture, adhesive, or similar treatment to increase frictional engagement between the first surface 4306A and the second surface 4306B.

FIG. 44 is a cross-sectional view of another overtube 4400 disposed on the endoscope 20. As illustrated, the overtube 4400 includes a split 4404 formed between overlapping portions of the overtube 4400. More specifically, when disposed about the endoscope 20 a first portion 4406A of the overtube 4400 is disposed inwardly of a second portion 4406B of the overtube 4400, forming an interface between the inward surface of the first portion 4406A and the outward surface of the second portion 4406B. In certain implementations, the overtube 4400 may be formed from a material having sufficient rigidity that the first portion 4406A of the overtube 4400 is maintained in positive contact with the second portion 4406B of the overtube 4400. Alternatively, or in addition, one or both of the inward surface of the first portion 4406A and the outer surface of the second portion 4406B may have a coating, layer, texture, or similar treatment to increase frictional engagement at the interface between the two portions 4406A, 4406B.

FIG. 45 is a cross-sectional view of another overtube 4500 disposed on the endoscope 20. As illustrated and similar to the overtube 4400 of FIG. 44, the overtube 4500 includes a split 4504 formed between overlapping portions of the overtube 4500. More specifically, when disposed about the endoscope 20 a first portion 4506A of the overtube 4500 is disposed inwardly of a second portion 4506B of the overtube 4500, forming an interface between the inward surface of the first portion 4506A and the outward surface of the second portion 4506B. In addition to the overlap at the interface, the first portion 4506A and the second portion 4506B may include mating or engaging structures. For example, as illustrated in FIG. 45, the first portion 4506A includes a series of longitudinal ridges 4510 shaped to be received by corresponding longitudinal grooves 4512 defined in the second portion 4506B.

As yet another example, FIG. 46 is a cross-sectional view of an overtube assembly 4600 disposed on the endoscope 20. As illustrated, the overtube assembly 4600 includes multiple overtubes and, more specifically an inner overtube 4601 and an outer overtube 4650. Each of the inner overtube 4601 and the outer overtube 4650 may be similar to any of the other split overtube designs discussed herein; however, for purposes of the current example, each of the inner overtube 4601 and the outer overtube 4650 are similar to the overtube 4300 of FIG. 43. More specifically, the inner overtube 4601 includes a lateral split 4604 including a first surface 4606A that abuts a second surface 4606B. Similarly, the outer overtube 4650 includes a lateral split 4654 including a first surface 4656A that abuts a second surface 4656B, the lateral split 4654 enabling insertion of the inner overtube 4601 with the endoscope 20 therein to be received within the outer overtube 4650. In certain implementations the inner overtube 4601 may be rotatable or otherwise movable within the outer overtube 4650.

It should be appreciated that in at least some implementations, the outer overtube 4650 extend along only a portion of the inner overtube 4601. In such implementations, multiple outer overtubes may also be distributed along the length of the inner overtube 4650. In still other implementations the outer overtubes 4650 may instead be substituted with split rings, straps, clips, or similar components adapted to extend around and maintain the inner overtube 4601 in a closed configuration.

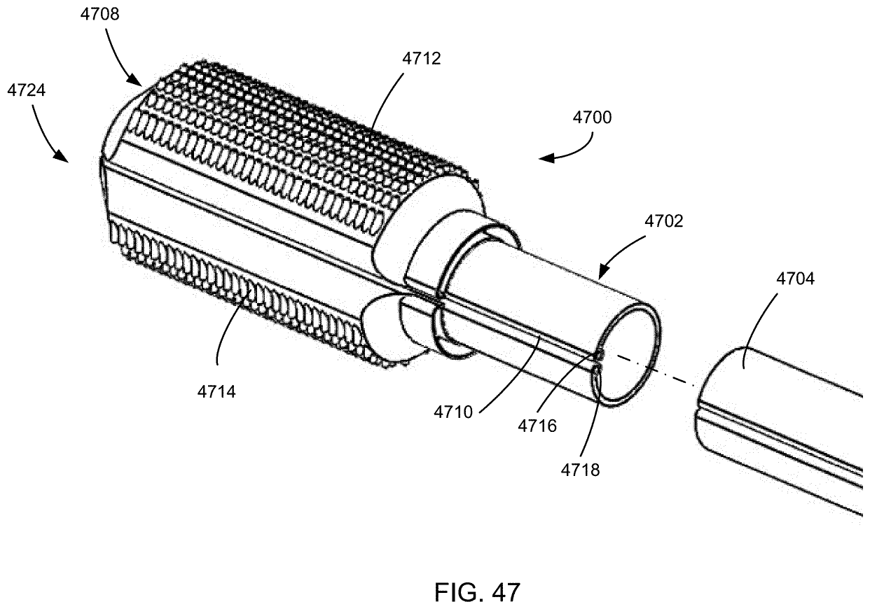

Further aspects of overtubes and overtube assemblies in accordance with the present disclosure are now provided with reference to FIGS. 47-63, which illustrate another example overtube assemblies and associated methods of manufacturing.

FIGS. 47-50 are an isometric view, a plan view, an elevation view, and a distal end view of the overtube assembly 4700. As previously discussed, the overtube assembly 4700 may be disposed on an elongate/tubular medical tool. For purposes of the following discussion, the tubular medical device is generally referred to as an endoscope, however, it should be understood that the overtube assembly 4700 may be configured to work with other medical devices having generally tubular shapes, including medical devices other than endoscopes.

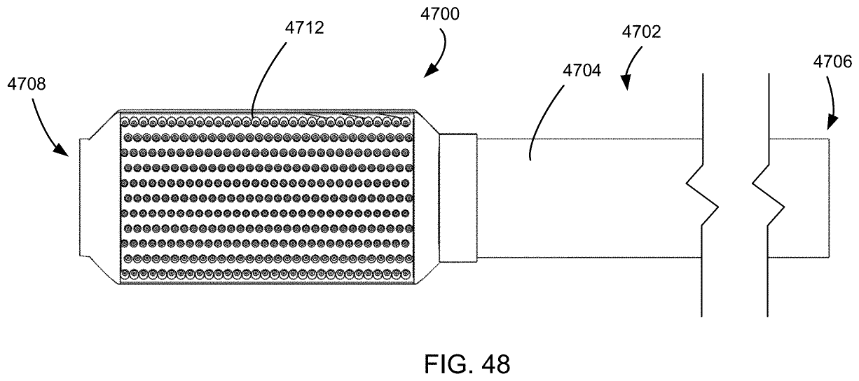

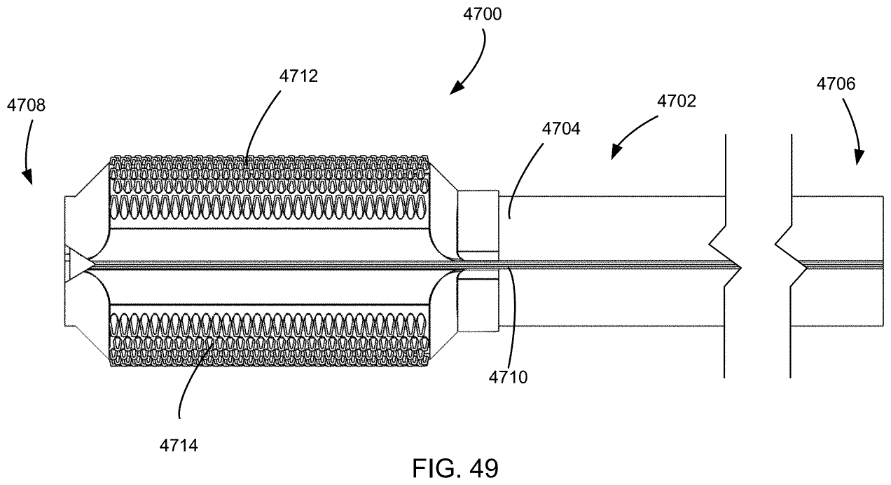

As illustrated in FIG. 47, the overtube assembly 4700 includes an overtube 4702 having a flexible tubular body 4704. The tubular body 4704 generally includes a proximal end 4706 (indicated in FIGS. 48 and 49) and a distal end 4708. The tubular body 4704 defines a split 4710 extending from the proximal end 4706 to the distal end 4708. As noted in the context of the foregoing example overtubes, the split 4710 permits the overtube assembly 4700 to receive an elongate medical device, such as an endoscope, by inserting the tool through the split 4710 as opposed to passing the tool through a lumen defined by the tubular body 4704. Notably, in at least some implementations, the split 4710 may include overlapping portions of the tubular body 4704 as previously discussed in the context of FIGS. 43-46.

The overtube assembly 4700 may further include one or more inflatable balloons, such as inflatable balloon 4712 and 4714, which are illustrated as being disposed on opposite sides of the tubular body 4704 on a distal portion 4724 of the tubular body 4704. Air may be provided to or removed from each of the inflatable balloons 4712, 4714 via respective air supply lumens 4716, 4718 defined by and extending through the tubular body 4704. Although not illustrated, in at least certain implementations, each of the air supply lumens 4716, 4718 may extend fully through the tubular body 4704 and may be capped by an insert or otherwise sealed at the distal end 4708 of the tubular body 4704. Also, while not illustrated, the proximal end of each air supply lumen 4716, 4718 may be coupled to one or more pumps or similar air supply devices that provide air to, remove air from, ventilate, etc. the inflatable balloons 4712, 4714. Although described herein as an “air supply lumen”, similar lumens may be implemented that deliver any suitable fluid to or remove fluid from the inflatable balloons 4712, 4714.

Although the overtube assembly 4700 includes inflatable balloons 4712, 4714, in other implementations, the inflatable balloons 4712, 4714 may be omitted or replaced with other fluid-controlled features. In implementations in which the balloons are removed and not replaced with another device, the air supply lumens 4716, 4718 may be omitted. The inflatable balloons of other implementations discussed herein may similarly be omitted.

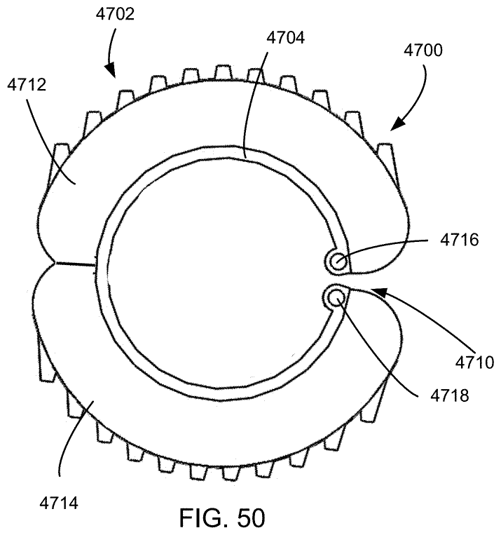

As most clearly shown in FIG. 50, in at least some implementations, the air supply lumens 4716, 4718 may be disposed on opposite sides of the split 4710 and may generally run parallel to the split 4710. In other implementations, the air supply lumens 4716, 4718 may be defined within the tubular body 4704 at a location other than adjacent the split 4710. Moreover, while the air supply lumens 4716, 4718 are shown as extending in a longitudinal direction, in other implementations, the air supply lumens 4716, 4718 may also extend in a circumferential direction as well. Also, while the split 4710 extends along the full length of the tubular body 4704, the air supply lumens 4716, 4718 may only extend along a portion of the tubular body 4704 sufficient to extend from the proximal end 4706 of the overtube 4702 to the inflatable balloons 4712, 4714.

Although illustrated in FIGS. 47-49 as being a single tubular structure, in at least certain implementations, the tubular body 4704 may be embedded with or otherwise include additional structural elements and features. For example, the tubular body 4704 may include reinforcement in the form of ribs, ridges, or other similar structural elements disposed along the length of the tubular body 4704. In certain implementations, such structural elements may be integrally formed with the tubular body 4704. In other implementations, such structural elements may instead be separate components that are embedded within, attached to, or otherwise coupled to the tubular body 4704. As another example, the tubular body 4704 may include one or more radiopaque markers to facilitate viewing of the overtube assembly 4700 using fluoroscopy. Similar to the reinforcing structures, in at least certain implementations such markers may be embedded within or attached to the tubular body 4704.

As noted above, in the specific implementation illustrate in FIGS. 47-49, the overtube assembly 4700 includes two inflatable balloons 4712, 4714 that are disposed near the distal end of the overtube 4702 and on opposite sides of the overtube 4702. As shown, the inflatable balloons 4712, 4714 include texturing in the form of frustoconical projections, similar to those of the balloon 2500 illustrated in FIGS. 25A-25D and discussed above. Although illustrated with frustoconical projections, it should be understood that the inflatable balloons 4712, 4714 may include any texturing disclosed herein on their exterior surfaces. It should also be appreciated that in at least some implementations, at least one of the inflatable balloons 4712, 4714 may be untextured.

This specific arrangement is provided merely as an example and other configurations are contemplated. For example, in certain implementations the overtube assembly 4700 may include any suitable number of inflatable balloons, including one. Also, the one or more inflatable balloons may be disposed at any location along the overtube 4702. To the extent the overtube assembly 4700 includes multiple inflatable balloons, such balloons may be disposed at different longitudinal locations along the overtube 4702. Similarly, while the inflatable balloons 4712, 4714 collectively extend around substantially the full circumference of the overtube assembly 4700, in other implementations, the inflatable balloons may instead be disposed only on one side of the overtube 4702 or otherwise extend around only a portion of the circumference of the overtube 4702.

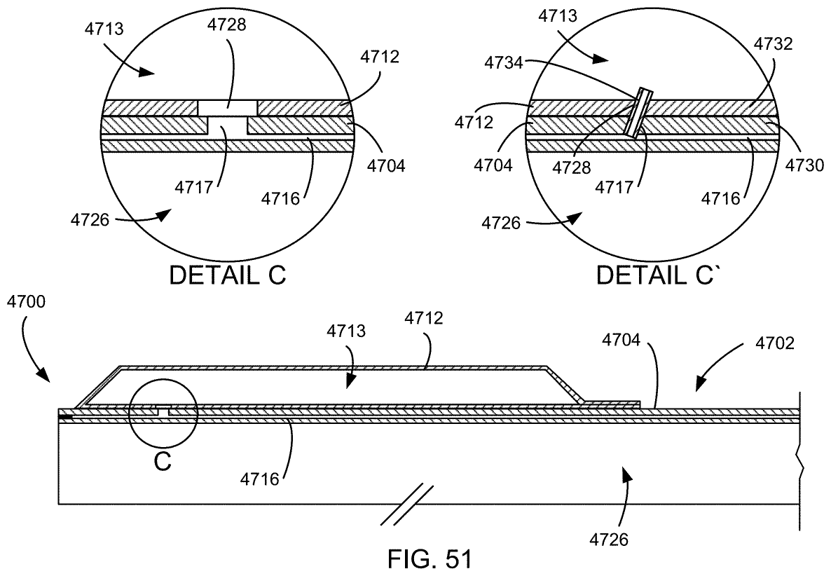

FIG. 51 is a partial longitudinal cross-section of the overtube assembly 4700. As illustrated, the tubular body 4704 of the overtube 4702 defines a tubular cavity 4726 within which the endoscope 20 or other medical tool is received via the split 4710 (shown in FIG. 49). FIG. 51 further illustrates the air supply lumen 4716, which is defined by and extends along the tubular body 4704. Each air supply lumen defined by the tubular body 4704 is in communication with an internal volume of one or more of the inflatable balloons 4712, 4714 (texturing of the balloons is omitted in FIG. 51 for clarity). In the specific example of the overtube assembly 4700, for instance, the air supply lumen 4716 is in communication with an internal volume 4713 of the inflatable balloon 4712. More specifically, the tubular body 4704 defines an overtube port 4717 in communication with the air supply lumen 4716. The inflatable balloon 4712 similarly defines a balloon port 4728 in communication with the internal volume 4713. During assembly and as illustrated in Detail C of FIG. 51, the inflatable balloon 4712 is coupled to the tubular body 4704 such that the overtube port 4717 and the balloon port 4728 are also in communication, thereby enabling air flow between the internal volume 4713 of the balloon 4712 and the air supply lumen 4716 during use of the overtube assembly 4700.

In certain implementations, each of the overtube port 4717 and the balloon port 4728 may be formed after initial extruding, molding, etc. of the tubular body 4704 and the balloon 4712. For example, following extrusion of the tubular body 4704, the overtube port 4717 may be formed by cutting, puncturing, etc. a wall 4730 of the tubular body 4704. Similarly, following forming of the balloon 4712, a wall 4732 of the balloon 4712 may be cut, punctured, etc. to form the balloon port 4728. Alternatively, in either case, either of the overtube port 4717 or the balloon port 4728 may be formed directly during the extrusion, molding, etc. process.

In certain implementations, a hollow conduit 4734 or similar reinforcing structure may also extend between the overtube port 4717 and the balloon port 4728 and provide an air channel between the internal volume 4713 of the inflatable balloon 4712 and the air supply lumen 4716. The hollow conduit 4734 may be inserted after formation of the overtube port 4717 and the balloon port 4728. In other implementations and as illustrated in Detail C′, the conduit 4734 may alternatively be used to puncture each of the wall 4730 of the tubular body 4704 and the wall 4732 of the balloon 4712 to form each of overtube port 4717 and the balloon port 4728.

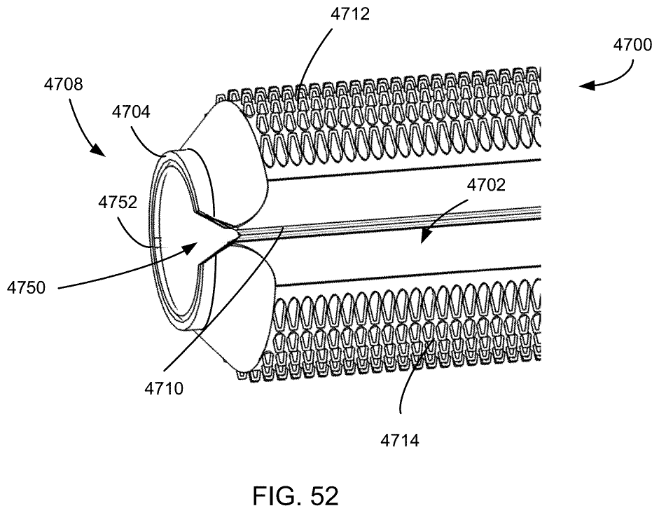

FIG. 52 is a detailed view of the distal end 4708 of the overtube assembly 4700. Among other things, FIG. 52 illustrates the inclusion of a notch 4750 formed in the distal end of the tubular body 4704, which may be included in implementations of the present disclosure. As illustrated, the notch 4750 generally extends proximally from a distal end 4752 of the tubular body 4704, tapering toward the split 4710, and ultimately being in communication with the split 4710

The notch 4750 is provided to facilitate placement of the overtube assembly 4700 onto an elongate medical device, such as an endoscope. More specifically, when disposing the overtube assembly 4700 onto the elongate medical device, the elongate medical device is first placed within the notch 4750. As the overtube 4702 is forced onto the tool, the notch 4750 provides a wedge-like action that opens the overtube 4702 along the split 4710, thereby facilitating placement of the overtube assembly 4700 onto the tool. Inclusion of the notch 4750 is particularly useful in implementations in which the overtube 4702 is particularly thick or stiff and, as a result, separation along the split 4710 may be difficult without the added leverage afforded by the notch 4750. Although the notch 4750 is shown as being triangular in FIG. 52, in other implementations, the notch 4750 may have other shapes. However, in general, the notch 4750 begins at the distal end 4752 of the overtube 4702 and tapers proximally.

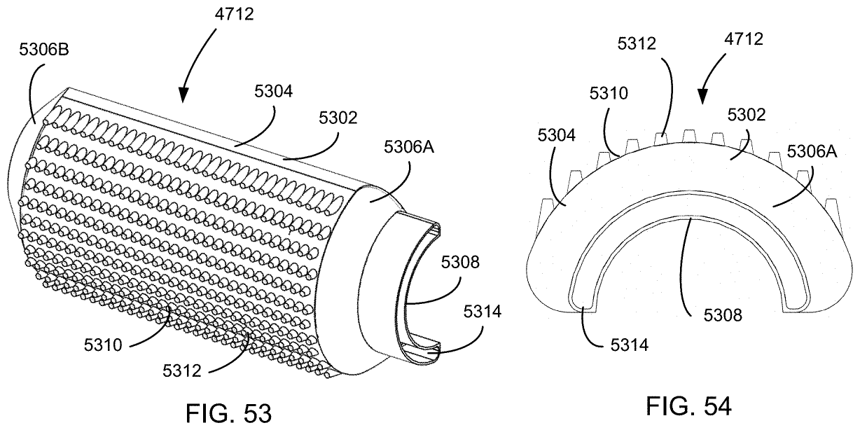

FIGS. 53 and 54 are an isometric view and an end view, respectively, of the inflatable balloon 4712 of the overtube assembly 4700. More specifically, FIGS. 53 and 54 illustrated the inflatable balloon 4712 in an unstrained state. Similar to the previously disclosed balloons, the balloon 4712 includes an elongate body 5302 including a middle portion 5304 and tapering end portions 5306A, 5306B. In contrast to the balloons previously disclosed herein, which had a substantially cylindrical shape through which an overtube or medical tool may extend, the inflatable balloon 4712 has a semi-annular shape intended to be disposed on the exterior of the overtube 4702 of the overtube assembly 4700. Accordingly, the inflatable balloon 4712 includes an inner concave surface 5308 shaped to receive the overtube 4702. In certain implementations, the balloon 4712 is formed to have the inner concave surface 5308 in others however, the balloon 4712 may have an oblong or “D”-shaped cross-section and the concave surface 5308 may be formed by indenting the inner surface of the balloon prior to application onto the overtube 4702.

The inflatable balloon 4712 may further include a textured outer convex surface 5310. As illustrated, the texturing 5312 on the outer convex surface 5310 includes longitudinally extending rows of frustoconical protrusions; however, texturing of the outer convex surface 5310 may generally conform to any texturing discussed herein.

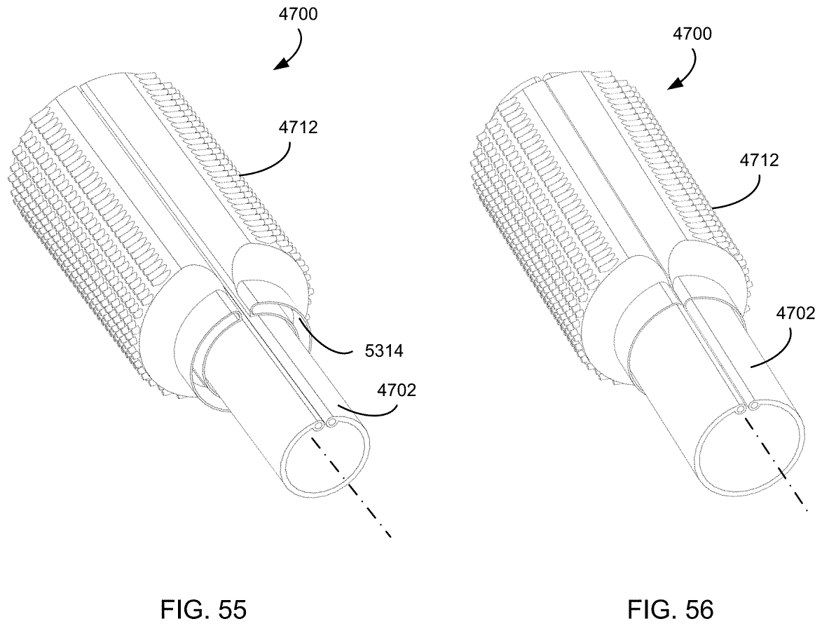

To facilitate assembly, the inflatable balloon 4712 may be formed with one or more open ends, such as open end 5314. During assembly, the open end 5314 permits access to the internal volume of the balloon 4712 to facilitate coupling of the balloon 4712 to the overtube 4702. For example, the balloon 4712 may be positioned onto the overtube 4702 and then each of the balloon 4712 and the overtube 4702 may be simultaneously pierced from within the balloon 4712 to form the overtube port 4717 and the balloon port 4728 previously discussed in the context of FIG. 51. Similarly, the open end 5314 of the balloon 4712 may be used to enable insertion of a conduit 4734, as illustrated in Detail C′ of FIG. 51. As illustrated in the transition between FIGS. 55 and 56 (each of which is an isometric view of the overtube assembly 4700, the open end 5314 is ultimately closed (e.g., using an adhesive, plastic welding, or similar process), thereby sealing the inflatable balloon 4712.

In certain implementations of the present disclosure, the tubular body of the overtube may include cutouts or similar voids to increase the flexibility of the overtube. In certain implementations, such voids may be evenly distributed along and about the length of the overtube to provide relatively uniform increased flexibility along the length of the tubular body. Alternatively, such voids may be disposed at specific locations (e.g., at particular longitudinal locations and/or on a particular side of the tubular body) to locally vary the flexibility of the tubular body. In certain implementations, localized thinning, scoring, grooves, etc. may similarly be used to vary the flexibility of the tubular body along its length.

In implementations in which voids or similar flexibility modifying features are disposed along the length of the tubular body, the tubular body may be wrapped, at least in part, in a low-friction sheath. For example, subsequent to coupling the tubular assembly to an endoscope or similar elongate tool, tape, a wrap, or similar layer formed of a low friction material (e.g., silicone) may be applied to the overtube of the overtube assembly to reduce interaction between the tubular body (and, in particular, any edges of the voids or flexibility modifying features) and the physiological lumen within which the tool is being used.

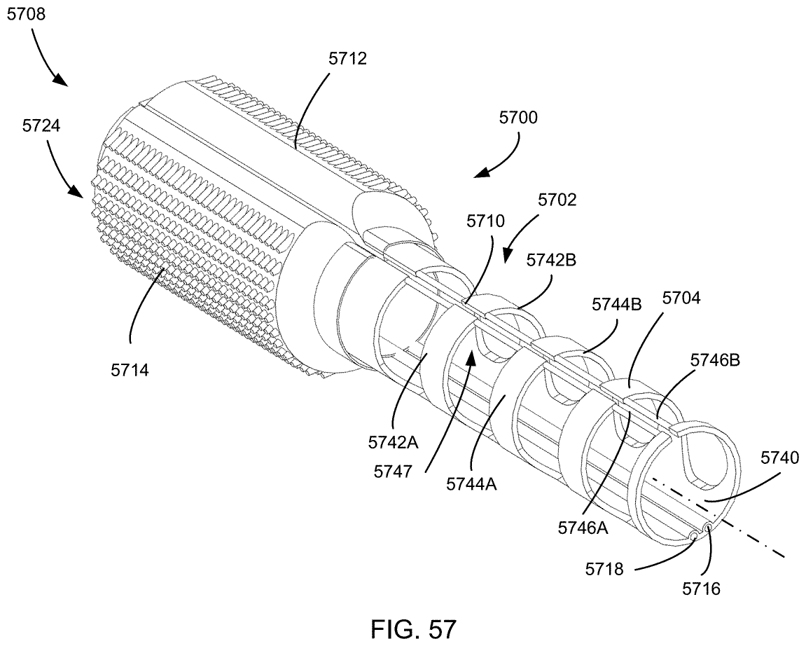

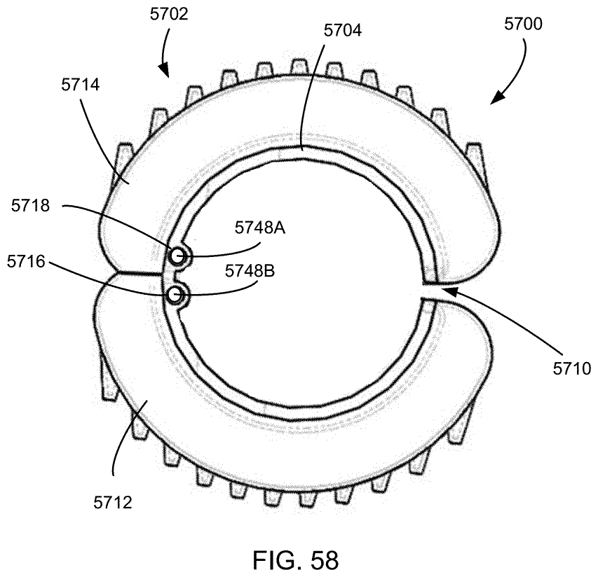

For example, FIGS. 57 and 58 are an isometric view and a distal end view, respectively, of an alternative overtube assembly 5700 in accordance with the present disclosure and which includes flexibility modifying features as discussed above. More specifically, FIG. 57 illustrates a distal portion of the overtube assembly 5700. The overtube assembly 5700 includes an overtube 5702 having a flexible tubular body 5704 that extends from a proximal end (not shown) of the overtube 5702 to a distal end 5708 of the overtube 5702. Similar to the tubular body 4704 of the overtube assembly 4700, the tubular body 5704 defines a split 4710 extending from its proximal end to the distal end 4708 to facilitate coupling of the overtube assembly 5700 to an endoscope or similar elongate tool. The overtube assembly 5700 further includes one or more inflatable balloons, such as inflatable balloon 5712 and 5714, which are illustrated as being disposed on opposite sides of the tubular body 5704 on a distal portion 5724 of the tubular body 5704.

As illustrated in FIG. 57, the tubular body 5704 of the overtube assembly 5700 includes a solid/continuous portion, referred to herein as a strip or backbone 5740, from which multiple ribs or bands (e.g., bands 5742A, 5742B and bands 5744A, 5744B) extend. As a result, voids or gaps (e.g., gap 5747 between band 5742A and 5744A) are formed between adjacent bands. As a result of the gaps, the overall flexibility of the tubular body 5704 is significantly increased as compared to the flexibility of a substantially continuous tubular body, such as the tubular body 4704 of the overtube assembly 4700 of FIG. 47.

In certain implementations, the tubular body 5704 may further include a pair of flexible rods 5746A, 5746B to which the bands are coupled and that extend along opposite sides of the split 5710. For example, each of bands 5742A and 5744A are coupled to rod 5746A while each of bands 5742B and 5744B are coupled to rod 5746B. Among other things, the rods 5746A, 5746B provide additional structural stability for the tubular body 5704.

While illustrated in FIG. 57 as being paired along the length of the tubular body 5704, implementations of the present disclosure may include bands that are offset relative to each other.

Air may be provided to or removed from each of the inflatable balloons 5712, 5714 via respective air supply lumens 5716, 5718 extending along the tubular body 5704. As shown in FIG. 57, the air supply lumens 5716, 5718 of the example overtube assembly 5700 extend inwardly from the backbone 5740, opposite the split 5710. In certain implementations, the air supply lumens 5716, 5718 may be integrally formed with the backbone 5740. Alternatively, the air supply lumens 5716, 5718 may be separately formed tubules that are coupled to the backbone 5740 using any suitable method. As yet another alternative, the air supply lumens 5716, 5718 may be defined by and extend through the rods 5746A, 5746B.

Other than their placement opposite the split 5710, the air supply lumens 5716, 5718 are structurally and functionally similar to those included in the overtube assembly 4700 discussed above. More specifically, during assembly, the air supply lumens 5716, 5718 are made to be in communication with internal volumes of the inflatable balloons 5712, 5714 (e.g., by using ports defined in the tubular body and balloons and/or suitable conduits extending between the internal volume of the balloons and the air supply lumens). A proximal end (not shown) of the air supply lumens 5716, 5718 is also configured to be coupled to a pump or other air supply device (not shown) to supply air to and/or remove air from the internal volumes of the inflatable balloons 5712, 5714 via the air supply lumens 5716, 5718. In certain implementations, the air supply lumens 5716, 5718 may extend along the full length of the tubular body 5704. In such implementations, the distal ends of the air supply lumens 5716, 5718 may also be capped, plugged, or otherwise sealed (e.g., using plugs 5748A, 5748B, shown in FIG. 58).

In alternative implementations of the backbone-style overtube, the rods 5746A, 5746B may be omitted and the tubular body 5704 may be configured similar to a comb-style binding spine. For example, the bands may extend from the backbone 5740, extend circumferentially about the tubular body 5704, and come into contact with either the internal or external surface of the backbone 5740. In such implementations, the bands may extend from only one side of the backbone 5740 or may extend from both sides of the backbone 5740 in an interdigitated manner. In at least some implementations, the bands may be configured to extend circumferentially past the backbone.

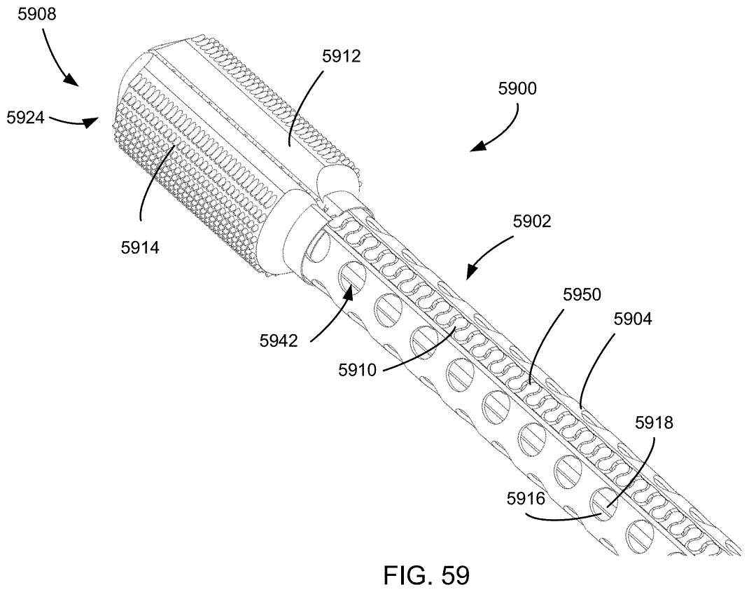

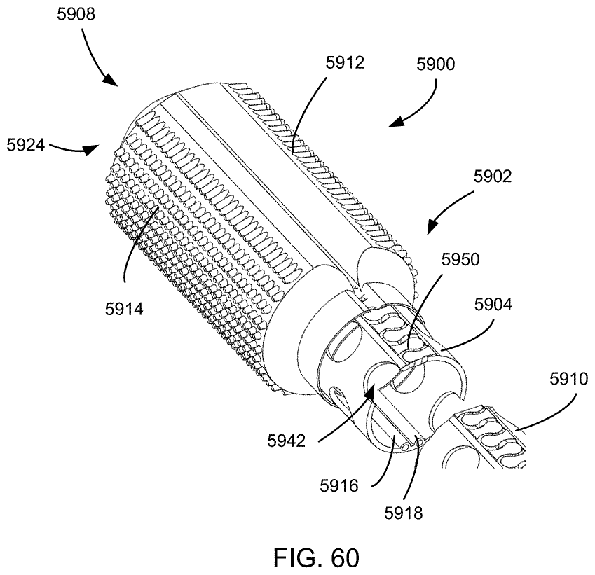

FIG. 59 is a partial isometric view of yet another overtube assembly 5900 in accordance with the present disclosure. FIG. 60 is a more detailed isometric view of a distal end of the overtube assembly 5900. The overtube assembly 5900 includes an overtube 5902 having a flexible tubular body 5904 that extends from a proximal end (not shown) of the overtube 5902 to a distal end 5908 of the overtube 5902. Similar to the tubular bodies of previously discussed implementations, the tubular body 5904 defines a split 5910 extending from its proximal end to the distal end 5908 to facilitate coupling of the overtube assembly 5900 to an endoscope or similar elongate tool. The split 5910 is shown in a closed configuration using a zipper-style closure 5950, which is discussed below in further detail. The overtube assembly 5900 further includes one or more inflatable balloons, such as inflatable balloon 5912 and 5914, which are illustrated as being disposed on opposite sides of the tubular body 5904 on a distal portion 5924 of the tubular body 5904.

Similar to the tubular body 5704 of the overtube assembly 5700, the tubular body 5904 includes features configured to modify the flexibility of the tubular body 5904 as compared to a substantially solid tubular body. In particular, the tubular body 5904 defines a plurality of voids or holes (e.g., void 5942) distributed along its length and around its circumference. Similar to the gaps between the bands of the tubular body 5704 illustrated in FIG. 57, the voids or holes of the tubular body 5904 similarly reduce the rigidity of the tubular body 5904.

Although illustrated in FIGS. 59 and 60 as being uniformly distributed along the tubular body 5904, such holes may instead be concentrated at particular locations to locally modify the flexibility of the tubular body 5704. Moreover, implementations of the present disclosure are not limited to holes or voids or any particular shape or size.

Air may be provided to or removed from each of the inflatable balloons 5912, 5914 via respective air supply lumens 5916, 5918. Similar to the air supply lumens 5716, 5718 of the overtube assembly 5700, the air supply lumens 5916, 5918 of the overtube assembly 5900 extend inwardly from a side of the tubular body 5904 opposite the split 5910, however, they may be disposed or otherwise routed in any suitable manner along the tubular body 5904 provided they enable air to be supplied/removed from the inflatable balloons 5912, 5914.

As noted above, the overtube assembly 5900 includes a closure mechanism and, in particular, a zipper-style closure 5950 to facilitate closing the split 5910. Although not necessary in all implementations of the present disclosure, closure mechanisms, such as the zipper-style closure 5950, can provide additional reinforcement and retention of the overtube assembly on the endoscope or other elongate tool in addition to any biasing of the tubular body into a closed shape resulting from its shape and material.

Mechanical closures in accordance with the present disclosure may include closures that are integrated into the tubular body and extend along at least a portion of the split. The zipper-style closure 5950, for example, is coupled to or otherwise integrated with the tubular body 5904 and extends along a substantial portion of the split 5910. Another example of an integrated closure is provided in FIG. 45. As discussed above, the overtube 4500 illustrated in FIG. 45 overlapping portions 4506A, 4506B that form an interface. The overlapping portions of the overtube further include corresponding ridges 4510 and grooves 4512 shaped to positively engage each other when the overtube 4500 is disposed on an endoscope or similar tool.

In other implementations, the tubular body of the overtube assembly may include interlocking tabs, snaps, clasps, or other similar closure mechanisms disposed along the length of the split.

Alternatively, closures may be separate components that are disposed along the tubular body and that provide retentive force onto the tubular body. For example, one or more of clips, bands, split rings, or similar elements may be disposed along the length of the tubular body after insertion of an elongate tool into the tubular body to provide additional retention of the tubular body onto the tool.

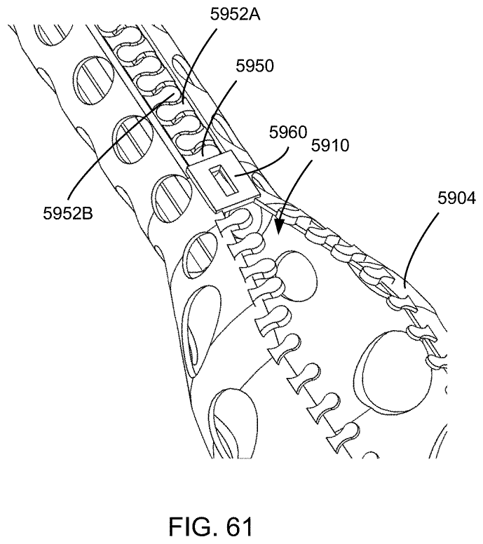

In certain implementations, the closures mechanisms may require additional tools or components to facilitate their use. For example, FIG. 61 illustrates a pull tab tool 5960 that may be used to open and close the zipper-style closure 5950 of the overtube assembly 5900. Similar to a conventional zipper, when the zipper-style closure 5950 is open/disengaged, distal ends of each half 5952A, 5952B of the zipper-style closure 5950 may be inserted into a proximal end of the pull tab tool 5960. The pull tab tool 5960 may then be translated proximally along the zipper-style closure 5950, engaging the interdigitating teeth of the closure halves 5952A, 5952B. In at least some implementations, the zipper-style closure 5950 may be configured such that the pull tab tool 5960 may be disengaged after closing the zipper-style closure 5950. For example, the pull tab tool 5960 may be disengaged by continuing to slide the pull tab tool 5960 beyond a proximal extent of the zipper-style closure 5950. It should also be noted that in alternative implementations, the zipper-style closure 5950 may be configured such that to close the zipper-style closure 5950, proximal ends of the halves 5952A, 5952B may be inserted into a distal end of the pull tab tool 5960 and the pull tab tool 5960 may be translated distally.

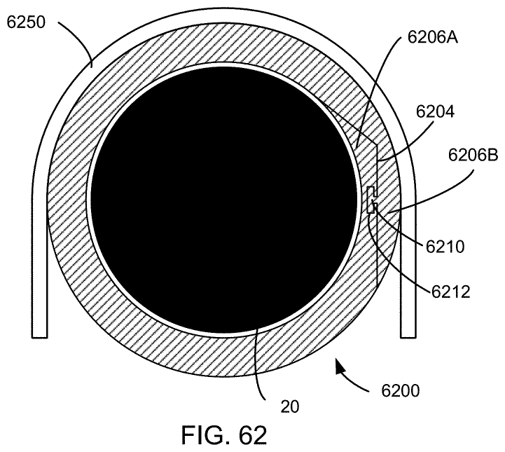

FIG. 62 is a cross-sectional view of another overtube 6200 and corresponding closure tool 6250. As illustrated, the overtube 6200 is disposed on an endoscope 20. As illustrated and similar to the overtubes 4400 of FIG. 44 and 4500 of FIG. 45, the overtube 6200 includes a split 6204 formed between overlapping portions of the overtube 6200. More specifically, when disposed about the endoscope 20 a first portion 6206A of the overtube 6200 is disposed inwardly of a second portion 6206B of the overtube 6200, forming an interface between the inward surface of the first portion 6206A and the outward surface of the second portion 6206B. In addition to the overlap at the interface, the first portion 6206A and the second portion 6206B may include mating or engaging structures. In particular, the first portion 6206A includes a T-shaped ridge 6210 shaped to be received by a corresponding T-shaped groove 6212 defined in the second portion 6206B.

In certain implementations, engagement of mating structures, such as those illustrated in FIGS. 45 and 62 may be facilitated by a tool that may be disposed on, applied to, or moved along the overtube. Such tools may be particularly beneficial in implementations in which closing the split by engaging the mating structures may be difficult to perform absent such a tool. For example, the tool 6250 illustrated in FIG. 62 is substantially rigid and shaped to be fit over and slid longitudinally along the length of the overtube. As the tool is slid along the overtube, it forces the ridge 6210 into the groove 6212, thereby closing the split 6204 of the overtube. More generally, however, the tool 6250 may be any device suitable to apply pressure onto the overtube 6200 to engage the mating structures of the overtube.

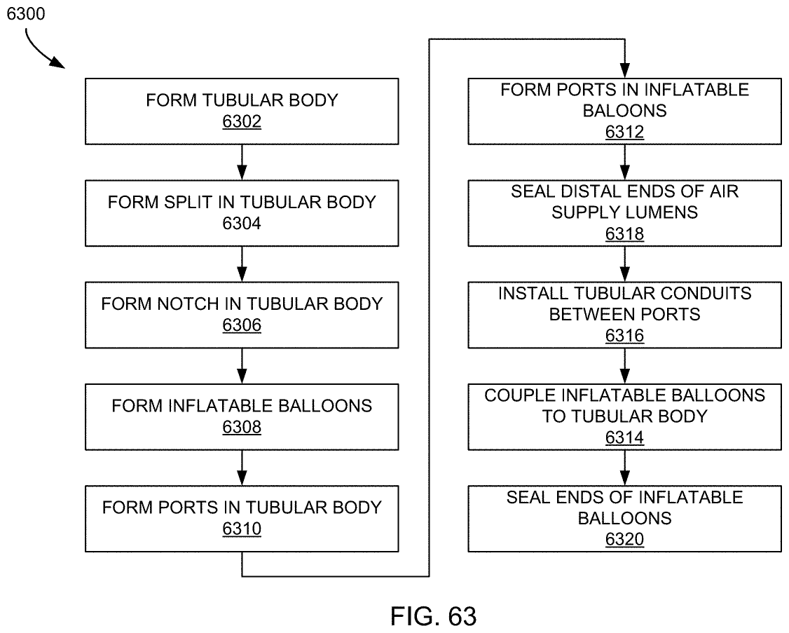

FIG. 63 is a method 6300 for manufacturing an overtube assembly, such as the overtube assembly 4700 of FIGS. 50-53. For explanatory purposes only, reference is made to the overtube assembly 4700 and its components. However, implementations of the method 6300 are not limited to the overtube assembly 4700 as illustrated in FIGS. 50-53.

In general, the method of manufacturing includes forming each of the tubular body 4704 of the overtube 4702 and each of the inflatable balloons 4712, 4714. Forming the tubular body 4704 generally includes forming the split 4710 extending along the tubular body 4704. The inflatable balloons 4712, 4714 are then coupled to the tubular body 4704 such that the internal volumes of the inflatable balloons 4712, 4714 are in communication with the air supply lumens 4716, 4718 of the overtube 4702. Accordingly, in certain implementations, manufacturing the overtube assembly 4700 may further include forming ports in the balloons 4712, 4714 and/or the tubular body 4704 and disposing the inflatable balloons 4712, 4714 onto the tubular body 4704 such that each of the ports of the tubular body 4704 are in communication with a respective port of an inflatable balloon 4712, 4714.

In light of the foregoing, operation 6302 includes forming the tubular body 4704. Although any suitable process may be used to form the tubular body 4704, in at least one implementation of the present disclosure, the tubular body 4704 is formed using an extrusion process. In such implementations, the tubular body 4704 may be formed using an extrusion machine having a die shaped to form each of the tubular cavity 4726 and the air supply lumens 4716, 4718 of the tubular body 4704.

In at least certain implementations, the tubular body 4704 may be formed from at least one of Nylon, PFA, PET, PTFE, FEP, HDPE, TPPE, silicone, PVC, other thermopolymers or any other suitable material. The material of the tubular body 4704 may also include additives to reduce surface friction of the tubular body 4704. For example, in one specific implementation, the tubular body may be formed from Hytrel Thermoplastic Polyester Elastomer with Everglide. In certain implementations, the tubular body 4704 may have a wall thickness from and including about 0.25 mm to and including about 1.0 mm. Although not limited to such implementations, thinner walled tubular bodies according to the present disclosure may generally be formed from a more rigid polymer than thicker-walled tubular bodies such that the thin-walled tubular bodies have sufficient rigidity to advance within the physiological lumen of the patient (e.g., the GI tract). In one specific implementation, the wall thickness of the tubular body 4704 may be about 0.75 mm. Although not limited to specific dimensions, in at least certain implementations, the air supply lumens 4716, 4718 may have a diameter of approximately 0.8 mm and a wall thickness of approximately 0.33 mm. In general, however, this air supply lumen diameter and wall may be made as small and thin as possible in order to minimize the size of the tubular body and, as a result, minimize the volume invaded within the physiological lumen. Similarly, other features of the tubular body may be formed to be as thin and small as possible as thinner and smaller features generally result in the tubular body being more flexible and better able to move through any turns of the physiological lumen within which it is deployed. Nevertheless, for certain materials (e.g., silastic polymers), minimum wall thickness and other dimensions may be limited by manufacturing. Also, if the lumen is intended to deliver/remove fluids other than air, the lumen diameter may need to be larger compared to air to account for the increased viscosity of the fluid.

Formation of the tubular body may include surface treating a portion of either the interior or exterior surface of the tubular body 4704 to provide increased friction. For example, and as discussed in the context of FIGS. 41 and 42, the internal surface of overtubes in accordance with the present disclosure may be coated or have integrally formed texturing at selective locations to increase friction with the medical tool disposed within the overtube. Similarly, and as discussed below in the context of FIGS. 59-66, the exterior surface of devices in accordance with the present disclosure, including the overtube 4702 of the overtube assembly 4700, may similarly have exterior surfaces adapted to increase friction with the interior wall of a physiological lumen. For example, such exterior surfaces may be coated or include integrally formed texturing similar to the interior surfaces previously noted.

In operation 6304, the split 4710 of the tubular body 4704 is formed. In at least certain implementations, formation of the split 4710 occurs during the extrusion process, e.g., by using an extrusion die where the wall of the tubular body 4704 is not continuous. Accordingly, the process of forming the tubular body 4704 (e.g., operation 6302) and forming the split 4710 along the tubular body 4704 (e.g., operation 6304) may occur simultaneously.

Alternatively, the wall 4730 of the tubular body 4704 may be extruded or otherwise formed to have a continuous circumference. In such cases, an additional cutting/splitting process may be required. In certain cases, splitting of the tubular body 4704 may be achieved using a knife or similar cutting tool disposed adjacent the extrusion machine such that the tubular body 4704 is split as it is extruded. Alternatively, a knife or similar cutting implement may be used to split the tubular body 4704 after the tubular body 4704 has been fully extruded. In at least certain implementations, the tubular body 4704 may be formed in operation 6302 with a seam or similar thin-walled portion to guide splitting. In such implementations, the seam may be designed such that splitting of the tubular body 4704 may be achieved by hand, e.g., by pulling apart the tubular body 4704 at the seam.

In operation 6306, a notch 4750 is formed in the distal end 4708 of the tubular body 4704. As previously discussed in the context of FIG. 52, a notch 4750 may be formed in the distal end 4708 of the tubular body 4704 to facilitate insertion of an endoscope 20 or similar elongate medical tube into the overtube 4702. More specifically, when disposing the overtube assembly 4700 on an endoscope 20, the endoscope 20 is first inserted into the distal extent of the notch 4750. Formation of the notch 4750 may include, among other things, trimming or otherwise cutting away the tubular body 4704 either by hand or using an automated machine.

Operations 6302-6306 generally correspond to manufacturing and forming of the tubular body 4704. As discussed above, other implementations of the present disclosure may include additional features and structures not included in the overtube assembly 4700. To the extent such features are not specifically included in the method 6300, formation of such features are nevertheless contemplated to be included in manufacturing methods according to the present disclosure. For example, and among other things, manufacturing methods according to the present disclosure may include operations directed to modifying the flexibility of the tubular body. For example, and referring to the overtube assembly 5700 of FIG. 57, manufacturing methods according to the present disclosure may include may include forming the bands (e.g., bands 5742A, 5742B and bands 5744A, 5744B) (and, as result the gaps/voids between the bands) and coupling the bands to the rods 5746A, 5746B. As another example and referring to the overtube assembly 5900 of FIG. 59, forming the tubular body may include forming the voids (e.g. void 5942). Manufacturing methods according to the present disclosure may also include the formation or inclusion of additional features to the tubular body. For example, and again referring to the overtube assembly 5900 of FIG. 59, manufacturing methods of the present disclosure may include adding a closure mechanism, such as the zipper-style closure 5950, to the tubular body.

In operation 6308, the balloons 4712, 4714 are formed. Non-limiting examples of balloon manufacturing methods are discussed above in the context of FIGS. 8 and 9. In general, however, forming the balloons 4712, 4714 generally includes molding or otherwise producing an initial shape of the balloons 4712, 4714. In certain implementations, the balloons 4712, 4714 may have integrally formed texturing, however, in other cases, texturing may be applied to the balloons 4712, 4714 after an initial molding process. To the extent the balloons 4712, 4714 are not produced having a shape that conforms to the overtube 4702, forming the balloons 4712, 4714 may further include manipulating or shaping the balloons 4712, 4714 to conform to the overtube 4702.

In operation 6310 ports are formed in the tubular body 4704. As described above, the overtube ports (e.g., overtube port 4717, illustrated in FIG. 51), are in communication with a respective one of the air supply lumens 4716, 4718. Forming each air overtube port generally includes forming a passage through the wall 4730 of the tubular body 4704 such that the passage extends from an exterior surface of the tubular body 4704 and terminates at one of the air supply lumens 4716, 4718. Accordingly, forming the overtube ports may include, among other things, cutting, puncturing, or similarly altering the tubular body 4704.

In operation 6312, balloon ports are formed in the inflatable balloons 4712, 4714. As previously discussed, each inflatable balloon generally includes a balloon port that enables air to be passed into or removed from an internal volume of the inflatable balloon, thereby inflating or deflating the balloon. Similar to the overtube ports, a balloon port for each inflatable balloon may be formed by cutting, puncturing or similarly altering the wall of the inflatable balloon.

In operation 6314 the inflatable balloons 4712, 4714 are coupled to tubular body 4704. Coupling of the inflatable balloons 4712, 4714 to the tubular body 4704 generally includes disposing the inflatable balloons 4712, 4714 onto the tubular body 4704 such each of the balloon ports of the inflatable balloons 4712, 4714 is in communication with one of the overtube ports of the tubular body 4704. The inflatable balloons 4712, 4714 may then be attached to the tubular body 4704, such as by using an adhesive, fusing the inflatable balloons 4712, 4714 to the tubular body 4704, or by any other suitable process.

In operation 6316, a tubular conduit 4734 is inserted through each pair of balloon ports and overtube ports to reinforce the pathway between the ports. In other implementations, the tubular conduit 4734 may be omitted.

In certain implementations, the inflatable balloons 4712, 4714 may be coupled to the tubular body 4704 prior to formation of either of the balloon ports or overtube ports. For example, in certain implementations, the balloons 4712, 4714 may be coupled to the tubular body 4704 and the balloon and overtube ports may then be formed in a substantially simultaneous manner by cutting, puncturing, etc. the tubular body 4704 and the balloons 4712, 4714 after coupling. In other implementations, the step of inserting the tubular conduit 4734 may also occur

In operation 6318 and if the air supply lumen extends along the full length of the overtube 4702, the distal end of the air supply lumens 4716, 4718 may be sealed. For example, caps or similar inserts may be disposed in the distal end of the air supply lumens. In other implementations, a filler or adhesive may be injected into the distal ends of the air supply lumens. Similarly, and as illustrated in FIGS. 55-56, the balloons 4712, 4714 may be sealed (operation 6320).

The forgoing example implementations are intended merely to illustrate various concepts of split overtubes in accordance with the present disclosure and should be regarded as non-limiting.

Expandable Overtubes

In certain use cases and with certain patients, only relatively small endoscopes may be advanced through a given physiological lumen. In other words, a gastroenterologist or similar physician or technician may be prevented from inserting larger diameter scopes and advancing such scopes as far as needed to perform a procedure. One specific example is with patients with altered anatomy resulting from bariatric or other similar procedures.

In other cases, a side-facing endoscope may ultimately be needed for the procedure, but advancing a larger, side-facing scope may be challenging due to the patient's anatomy, among other things. In such cases, the ability to use a forward facing endoscope to reach the desired location is valuable only if an overtube can then be placed so that the overtube may be used to guide a larger scope (e.g., a side facing scope) to the desired location.

To address the foregoing issues, among others, the current disclosure includes an expandable overtube. In a first configuration, such as may be used during insertion of first, smaller endoscope (or similar tool) the expandable overtube is compressed to a first, smaller diameter. Upon removal of the first endoscope, a second, larger endoscope (or similar tool) may be inserted into the overtube which expands to accommodate the larger tool. In certain implementations, for example, in the first configuration the overtube may have an inner diameter of approximately 10 mm but may be configured to expand to 15 mm or more in response to insertion of a larger tool. To facilitate the forgoing expansion and contraction, the overtube may include an embedded mesh that provides structural rigidity to the overtube in each of the compressed and expanded configurations.

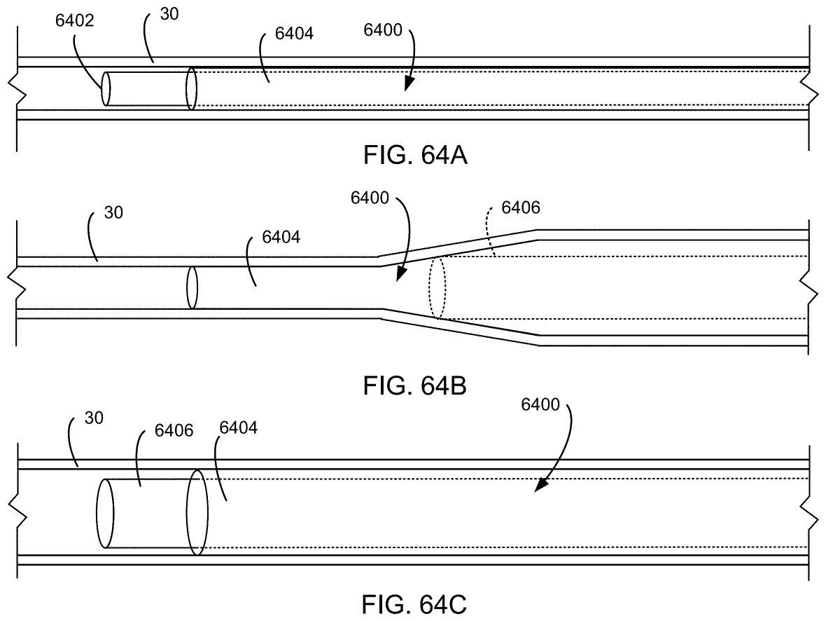

FIGS. 64A-64C illustrate an example procedure using an expandable overtube in accordance with the present disclosure. Referring first to FIG. 64A, a physiological lumen 30 is shown within which an endoscope assembly 6400 is disposed, the endoscope assembly 6400 including a first endoscope 6402 disposed within an expandable overtube 6404.

The first endoscope 6402 may have a first diameter for use in intubating the patient with the expandable overtube 6404. Once intubated, the first endoscope 6402 may be removed and a second endoscope or tool 6406 may be inserted into the overtube 6404, as illustrated in FIG. 64B. As the second endoscope or tool 6406 is advanced through the overtube 6404, an outward force is applied to the overtube 6404 causing it to expand. In certain implementations, such expansion may be facilitated, in part, by an embedded mesh within the overtube 6404 configured to retain its shape when expanded outwardly.

As shown in FIG. 64C, the second endoscope or tool 6406 may be advanced to extend beyond the now-expanded overtube 6404 to the original position of the first endoscope 6402 illustrated in FIG. 64A.

Any surface of the overtube 6404 may include texturing in accordance with the present disclosure. For example, and without limitation, the outer surface of the overtube 6404 may include texturing configured to facilitate frictional engagement of the overtube 6404 with the inner surface of the physiological lumen within which the overtube 6404 is disposed. Such frictional engagement may prevent slippage or shifting of the overtube 6404 during expansion of the overtube 6404 in response to insertion of the second, larger tool 6406 into the overtube 6404. In implementations in which the overtube 6404 is textured, such texturing may be applied to substantially the entire length of the overtube 6404 or may be applied to one or more segments of the overtube 6404. In certain implementations, the texturing may be configured to have a first engagement level when the overtube 6404 is in a first (e.g., the compressed) configuration, but to have a second engagement level when the overtube is in a second (e.g., the expanded) configuration, the second engagement level resulting from a difference in strain applied to the textured portions of the overtube 6404.

The forgoing example implementations are intended merely to illustrate various concepts and applications of an expandable overtube in accordance with the present disclosure and should be regarded as non-limiting.

Textured Endoscopic Tools

Endoscopic procedures may include a biopsy or similar removal of a portion of tissue. When a snare or a biopsy catheter is used, the location of the scope and the tissue of interest may be located such that holding the snare steady relative to the tissue and the scope may be extremely challenging, particularly because the snare/biopsy catheter is generally unsupported within the physiological lumen within which the biopsy is to be taken.

To address the foregoing issues, among others, textured endoscopic tools are provided herein. In one implementation, texturing is applied to a snare, biopsy forceps, or other endoscope gastroenterology tools. Such texturing may be used to frictionally engage or adhere the tool to an inner wall of a physiological lumen and to help steady the tool relative to the tissue being removed. In certain implementations, texturing is disposed on the snare, biopsy tool, etc., itself. Alternatively, or in addition to texturing of the tool itself, texturing may also be applied to a catheter through which the tool is delivered. In the latter case, the catheter adheres to the wall of the physiological lumen and is steadied by such adherence.

Texturing on the tool and/or catheter may also be used to pull tissue (e.g., a polyp or the wall of the physiological lumen) to facilitate tissue removal or to improve a physician's view of the physiological lumen. Notably, such tissue manipulation relies on relatively minimal engagement with the tissue, particularly when compared to conventional approaches in which a snare or similar tool is used to grasp the tissue.

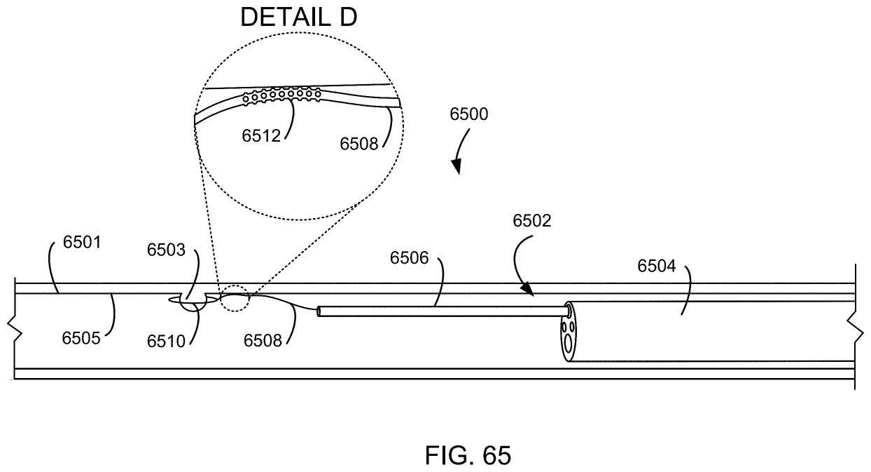

FIG. 65 is a schematic illustration of an operational environment 6500 including a physiological lumen 6501 in which an endoscopic tool 6502 is disposed. For purposes of the current example, the physiological lumen 6501 is assumed to include a polyp 6503 which is to be removed; however, it should be appreciated that implementations of the current disclosure are not limited to such applications.

As illustrated the endoscopic tool 6502 includes an endoscope body 6504 from which a catheter 6506 may be extended. The endoscopic tool 6502 further includes a snare 6508 disposed within and extending from the catheter 6506. As illustrated, the snare 6508 includes a loop 6510 which may be used to encircle and capture the polyp 6503 for subsequent removal. The snare 6508 of FIG. 65 is provided merely as a non-limiting example of an endoscopic tool. It should be understood that the present disclosure is equally applicable to other tools including, without limitation, biopsy forceps, brushes, rods, guidewires, or any other tool that may be delivered via the endoscopic tool 6502 for any purpose.

As illustrated in Detail D, at least a portion of the snare 6508 includes texturing 6512 configured to increase frictional engagement between the snare 6508 and an inner wall 6505 of the physiological lumen 6501. In the specific example illustrated, the texturing 6512 is in the form of a series of protrusions extending from the snare 6508 and disposed proximal to the loop 6510; however, it should be understood that any suitable texturing applied at any location along an endoscopic tool may be used instead.

During use, a physician or technician may extend the snare 6508 from the catheter 6506 and position the snare 6508 such that the texturing 6512 contacts the inner wall 6505 of the physiological lumen 6501. Such contact between the texturing 6512 and the inner wall 6505 adheres the snare 6508 to the inner wall 6505, thereby stabilizing the snare 6508. In certain implementations, the physician or technician may advance, retract, or otherwise manipulate the snare 6508 once adhered to the inner wall 6505 to manipulate the physiological lumen (e.g., to improve visibility of an area of interest or to move tissue to make biopsy or tissue removal easier).

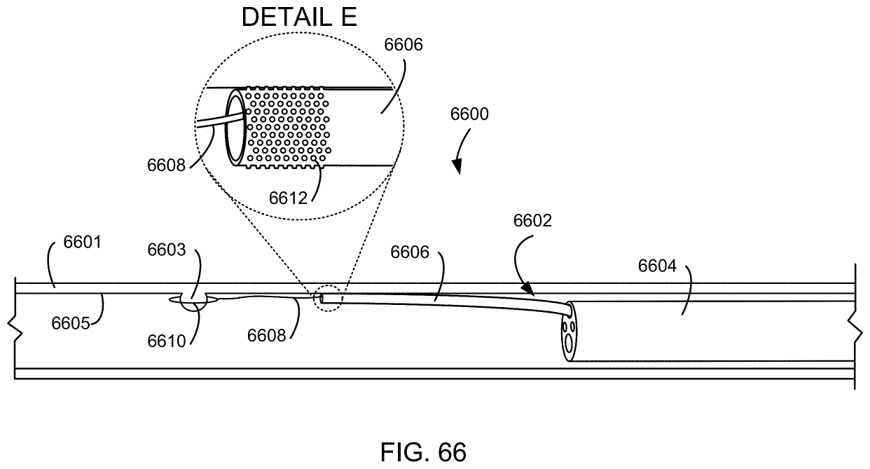

FIG. 66 is a schematic illustration of an operational environment 6600 including a physiological lumen 6601 in which an endoscopic tool 6602 is disposed. For purposes of the current example, the physiological lumen 6601 is assumed to include a polyp 6603 which is to be removed; however, it should be appreciated that implementations of the current disclosure are not limited to such applications.

As illustrated the endoscopic tool 6602 includes an endoscope body 6604 from which a catheter 6606 may be extended. The endoscopic tool 6602 further includes a snare 6608 disposed within and extending from the catheter 6606. As illustrated, the snare 6608 includes a loop 6610 which may be used to encircle and capture the polyp 6603 for subsequent removal. Similar to the previous discussion, the snare 6608 is provided merely as a non-limiting example of an endoscopic tool.

As illustrated in Detail E, at least a portion of the catheter 6606 includes texturing 6612 configured to increase frictional engagement between the catheter 6606 and an inner wall 6605 of the physiological lumen 6601. In the specific example illustrated, the texturing 6612 is in the form of a series of protrusions extending from a distal portion of the catheter 6606; however, it should be understood that any suitable texturing applied at any location along the catheter 6606 may be used instead.

During use, a physician or technician may extend the catheter 6606 from the endoscopic tool 6602 and position the catheter 6606 such that the texturing 6612 contacts the inner wall 6605 of the physiological lumen 6601. Such contact between the texturing 6612 and the inner wall 6605 adheres the catheter 6606 to the inner wall 6605, thereby stabilizing the catheter 6606. The snare 6608 may then be advanced, retracted, or otherwise manipulated relative to the catheter 6606 to perform a given procedure.

The foregoing implementations are intended merely as examples and, as a result, should be viewed as non-limiting. More generally, the present disclosure is directed to catheters and endoscopic tools including texturing adapted to adhere the catheter and/or tool to tissue. In certain implementations, the texturing may be in accordance with specific examples of texturing discussed herein; however, implementations of the present disclosure are not necessarily limited to such specific examples. Moreover, texturing may be applied to the tool/catheter using any suitable technique. For example, and without limitation, texturing may be integrally formed on the tool/catheter, may be applied as an outer layer or coating, or may be formed onto the tool/catheter (e.g., by overmolding or spray deposition).

Textured Stents

In yet another aspect of the present disclosure, textured stents are provided that improve anchoring of such stents, reducing potential for migration and additional interventions associated with repositioning or otherwise adjusting a stent.

In one specific implementation, a stent is provided for use in ducts, such as the biliary and pancreatic duct. In biliary and pancreatic duct applications, stents may be temporarily or permanently anchored to force open the duct to facilitate proper drainage into the gastrointestinal tract. For a variety of reasons, biliary and pancreatic ducts can become inflamed and be forced shut due to such inflammation. Accordingly, stents are commonly placed to allow the ducts to drain while the inflamed tissue is healed. However, as previously noted, stent migration can present a significant challenge.

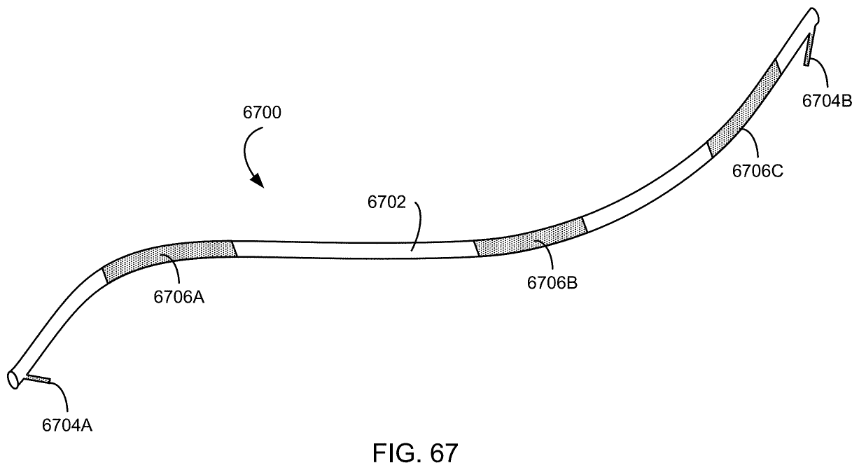

FIG. 67 is an example stent 6700 for use in duct-related applications with various features for improving anchoring relative to the duct. As shown in FIG. 67, the stent 6700 includes a tubular body 6702 which may optionally terminate in flared ends, hooks, barbs, or similar retention structures 6704A, 6704B. However, in certain implementations, the retention structures 6704A, 6704B may be omitted in favor of the other retention features discussed below.

As illustrated, the stent body 6702 may include texturing along its length. Such texturing may be applied along substantially the entire length of the body 6702 or along certain segments of the body 6702. For example, the stent 6700 illustrated in FIG. 67 includes three separate textured segments 6706A-6706C. Texturing is also applied to each of the end retention structures 6704A, 6704B. In use, the texturing on the stent 6700 improves anchoring by increasing friction/adhesion between the stent 6700 and a physiological lumen or structure within which the stent 6700 is inserted.

In certain implementations, the texturing may be integral to the stent body 6702. For example, the stent 6700 may be molded using silicone or other polymer materials with the texturing included on the surface as part of the molding process. In other implementations, the body 6702 may be initially formed without texturing and the texturing may be applied afterwards. For example, texturing may be applied by applying a layer or coating to the body 6702 including the texturing, overmolding the texturing onto the body 6702, or spraying the texturing onto the body 6702, among other manufacturing approaches.

The stent 6700 may be fabricated from various materials, each of which may have a durometer suitable for one or more specific applications. The stent 6700 may also be formed from multiple materials. For example, certain sections of the stent 6700 may be formed from relatively a low durometer material to facilitate bending of the stent 6700 while other sections may be formed from a relatively high durometer material to provide localized structural integrity. In another example implementation, the stent 6700 may include multiple layers with an interior layer of the stent 6700 having a higher durometer than exterior layers. In still another example implementation, the stent body 6702 may be formed from a first material having a first durometer while the textured portions or texturing applied to the body 6702 may have a second durometer.

The texturing of the stent 6700 may take various forms including, but not limited to, the various example texturing patterns discussed herein.

In another implementation of the present disclosure, a textured stent for implantation within a physiological lumen is provided. Such stents may be used, for example, within the gastrointestinal tract or vasculature of a patient.

Similar to the previously discussed stents, conventional gastrointestinal and vascular stents may migrate after being placed. Accordingly, placement and anchoring of such stents typically includes the use of sutures to hold the stents in place and/or mechanisms that apply outwardly radial loading to the stent such that it is maintained against the vascular or GI wall. In either case, placement of the stent and prevention of migration results in additional steps and procedures that may increase surgery time and/or raise the possibility of additional complications during implantation of the stent.

To address the foregoing issues, among others, the present disclosure includes a textured stent for implantation within a physiological lumen. The stents include an expandable body (e.g., an expandable mesh) that may be covered (entirely or in part) with a textured surface for increasing frictional engagement/adhesion between the stent and the inner wall of the physiological lumen.

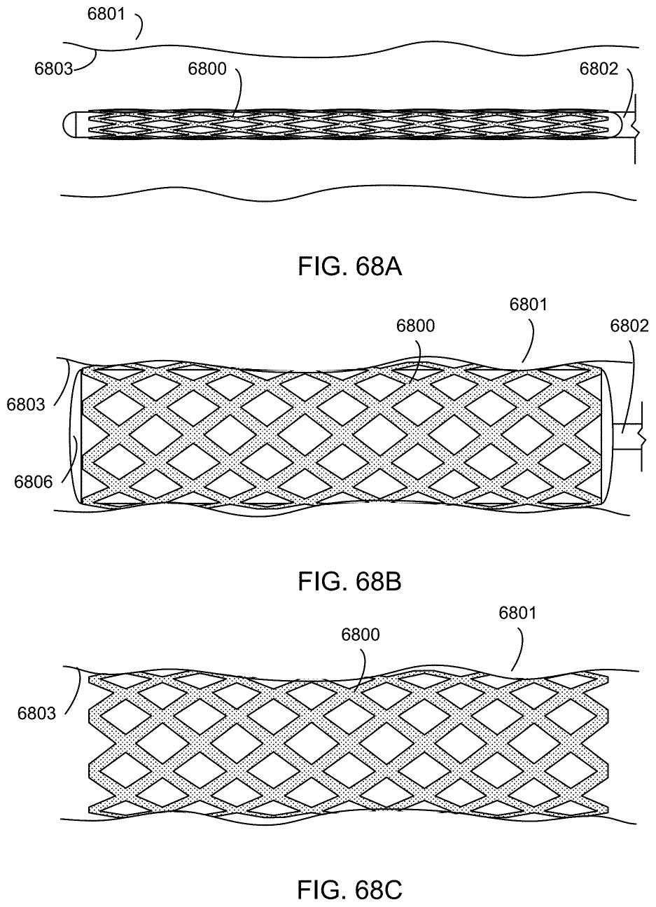

FIGS. 68A-68C illustrate an example process of implanting a textured stent 6800. Referring first to FIG. 68A, the textured stent 6800 may be disposed on a deployment tool 6802 in a first, compressed configuration. The deployment tool 6802 may then be advanced within the physiological lumen 6801 to position the stent 6800 at an implantation location.

When located, the stent 6800 may be deployed by expanding the stent 6800 such that its surface contacts an inner surface 6803 of the physiological lumen 6801. Although other deployment methods may be implanted, in the illustrated example, the deployment tool 6802 includes an expandable balloon 6806 that is inflated to expand the stent 6800 to contact the inner surface 6803 (as shown in FIG. 68B). When expanded, the textured surface of the stent 6800 abuts the inner surface 6803, with the texturing providing increased friction and adhesion as compared to conventional, smooth stents.

Following deployment of the stent 6800, the balloon 6806 may be deflated and removed from within the physiological lumen 6801, leaving the stent 6800 in place (as shown in FIG. 68C).

As previously noted, the texturing may be applied to some or the entire exterior surface of the stent 6800. For example, in certain implementations, texturing may be applied in one or more circumferential bands that extend about the stent 6800. In another implementation, texturing may be applied to discrete sections or blocks distributed about the exterior surface of the stent 6800.

Similar to the previous stent, the texturing may be integrally formed with the body of the stent 6800 or may be added in a subsequent process (e.g., by applying a layer or coating, overmolding, etc.).

As discussed in the context of the balloons, above, the texturing of the stent 6800 may be configured to have different frictional/adhesion properties in different configurations. For example, when in the compressed configuration illustrated in FIG. 68A, the texturing may have a relatively low friction coefficient to prevent or minimize adhesion to the physiological lumen during deliver of the stent 6800. However, in response to the strain applied during deployment of the stent 6800, the friction coefficient of the texturing may increase to facilitate anchoring of the stent 6800 within the physiological lumen.

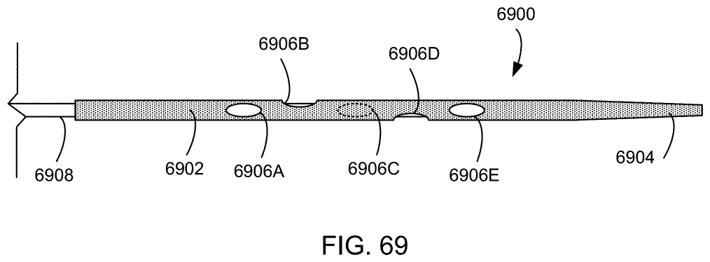

FIG. 69 is a schematic illustration of another stent 6900 according to the present disclosure. As illustrated, the stent 6900 includes a body 6902 having a tapered tip 6904. Such stents may be used to facilitate fluid in the bile duct. Similar to the previously discussed stents, the stent body 6902 may be at least partially textured such that when implanted, the texturing of the stent body 6902 frictionally engages/adheres to the wall of a physiological lumen or other tissue, thereby resisting migration of the stent 6900 following implantation. Although the diameter of the stent body 6902 may vary, in at least one implementation the stent body 6902 tapers from a first diameter of approximately 10 Fr down to a second diameter of approximately 8.5 Fr. In certain implementations, the tapered tip 6904 may be reduced to allow use of a pusher catheter 6908 (as described below) but may include a hole or lumen through which a guidewire may be passed.

In certain implementations, the body 6902 may define one or more ports or openings, along its length to permit fluid. For example, in the implementation at least one implementation, multiple ports 6906A-6906E may be distributed along the length of the body 6902 in a spiral/helical arrangement. In one specific implementation, the spacing of the ports 6906A-6906E may be approximately 1 cm.

Although stent 6900 may be advanced/implanted using various techniques, in at least one approach, a pusher catheter 6908 is inserted into the stent body 6902 and made to abut the inside of the tapered tip 6904. The stent 6900 may then be pushed from the proximal end using the pusher catheter 6908.

In certain stent applications, texturing of stents according to the present disclosure may include protrusions, ridges, or similar structures that extend outwardly from the exterior surface of the stent. In certain implementations, such protrusions extend in a substantially radial direction. However, in other implementations, at least a portion of the texturing may be swept or otherwise biased toward an end of the stent. By doing so, the texturing may provide additional resistance to movement in the direction of the bias while providing reduced resistance in the opposite direction. So, for example, a stent may include texturing that is backswept in a direction opposite a direction of advancement such that the friction provided by the texturing is reduced during insertion and advancement but increased in a direction opposite that of advancement following deployment (e.g., to counter potential movement caused by blood flow, peristalsis, etc.). Biased texturing and control of such biasing (e.g., by selectively expanding or compressing the stent to vary the angle of the texturing) may also facilitate removal of the stent as it allows physicians and technicians to dynamically modify the resistance/adhesion provided by the texturing.

In at least some implementations of stents according to the present disclosure, texturing of the stent may include applying texturing to a metallic or similar substrate. For example, texturing of a tubular or expandable metallic stent may be applied by coating the substrate, applying an adhesive layer including the texturing to the substrate, spraying texturing onto the substrate, overmolding texturing onto the substrate, or any other suitable method of applying the texturing to the substrate.

Laparoscopic and Similar Surgical Tools

As another example application, texturing in accordance with the present disclosure may be applied in the context of laparoscopic tools. For example, FIG. 70 illustrates an operational environment 7000 and, in particular a cross-sectional view of a patient abdomen 7002 including an abdominal wall 7004 and abdominal organs 7006.

The operational environment 7000 further includes a pair of surgical tool assemblies 7008A, 7008B, which in the particular example of FIG. 70, are manually operated laparoscopic tool assemblies. The surgical tool assembly 7008A includes a trocar/port assembly 7010A, which may extend through the abdominal wall 7004 to provide access to the internal abdominal cavity 7005, which, in the case of laparoscopic procedures, may be insufflated during surgery. The surgical tool assembly 7008A further includes a surgical tool 7012A including a tool shaft 7014A terminating in a tool end effector 7016A. The surgical tool assembly 7008B similarly includes a surgical tool 7012B including a tool shaft 7014B terminating in a tool end effector 7016B and further including a trocar/port assembly 7010B. For clarity and simplicity, the following discussion refers only to surgical tool assembly 7008A, however, the description of surgical tool assembly 7008A is generally applicable to surgical tool assembly 7008B.

As discussed below in further detail, at least a portion of the surgical tool 7012A may include a textured surface in accordance with the present disclosure. For example, one or both of the tool shaft 7014A and the tool end effector 7016A may be at least partially textured as described herein. Among other things, such texturing may facilitate manipulation and/or retention of tissue and organs of the abdomen. For example, and as illustrated in FIG. 70, during surgery, the tool shaft 7014A may be made to move aside or hold an internal organ. Texturing applied to the tool shaft 7014A may generally increase grip/adhesion between the tool shaft 7014A and the tissue/organ, thereby improving the degree of control over the tissue/organ and reducing the likelihood that the tissue/organ will slip from the tool shaft 7014A. As previously noted, texturing may also or alternatively be applied to the tool end effector 7016A to similarly increase adhesion and retention of the tool end effector 7016A.

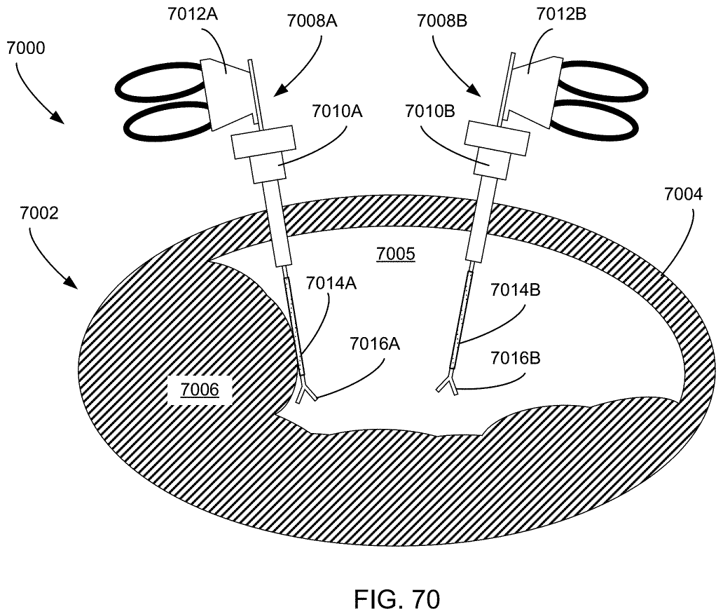

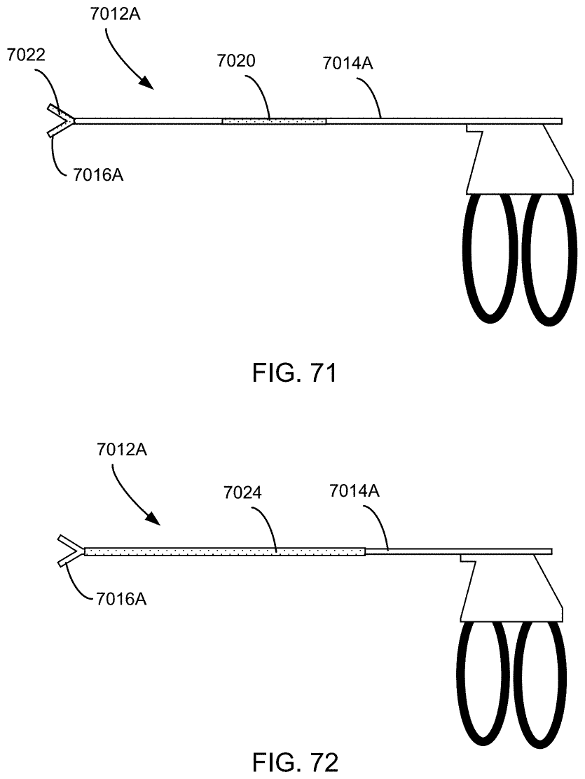

FIGS. 71 and 72 illustrate different implementations of the surgical tool 7012A and, in particular, different approaches to texturing the surgical tool 7012A. Referring first to FIG. 65, the surgical tool 7012A is shown as having a first textured portion 7020 disposed along the tool shaft 7014A and a second textured portion 7022 corresponding to the tool end effector 7016A.

The first textured portion 7020 may be formed in various ways. For example, and without limitation, in at least certain implementations, the textured portion 7020 may be integrally formed with the tool shaft 7014A. In other examples, the textured portion 7020 may be overmolded onto the tool shaft 7014A. In still other implementations, the textured portion 7020 may be a separate segment of the tool shaft 7014A that is inserted between and coupled to a proximal and/or distal segment of the tool shaft 7014A. In yet other implementations, the textured portion 7020 may be formed by applying a coating or similar treatment onto the tool shaft 7014A.

The second texture portion 7022 corresponding to the tool end effector 7016A may similarly be integrally formed with the tool end effector 7016A or formed onto the tool end effector 7016A, such as by overmolding or coating of the tool end effector 7016A. Although illustrated in FIG. 70 as being applied to the entire tool end effector 7016A, texturing may alternatively be applied to only a portion of the tool end effector 7016A. For example, and without limitation, in one application, texturing may only be applied to a proximal surface of the tool end effector 7016A. In another example implementation in which the tool end effector 7016A is a grasper-type tool including jaws, texturing may be applied only to the inner surface of the jaws.

FIG. 72 is an alternative implementation of the surgical tool 7012A in which a textured cover 7024 is disposed on the tool shaft 7014A. In certain implementations, the textured cover 7024 may be a sheath through which the tool shaft 7014A is inserted, the exterior surface of the sheath having texturing as described herein. The sheath may then be adhered to, shrunk onto, or otherwise retained on the tool shaft 7014A. In an alternative implementation, the textured cover 7024 may be in the form of a wrap, tape, etc. that is wrapped around the tool shaft 7014A. To retain the wrap/tape, an adhesive may be applied to the tool shaft 7014A or the wrap/tape prior to wrapping. Alternatively, the wrap/tape may have an adhesive backing.

Although illustrated in FIGS. 70-72 as manually-operated laparoscopic tools, implementations of the present disclosure may include actuated tools including robotically controlled tools. The various aspects of FIGS. 70-72 are also not limited to the grasper-type tools illustrated and application of the described texturing to other tools, including other laparoscopic tools and other non-laparoscopic tools, is contemplated.

Microtextured Trocars

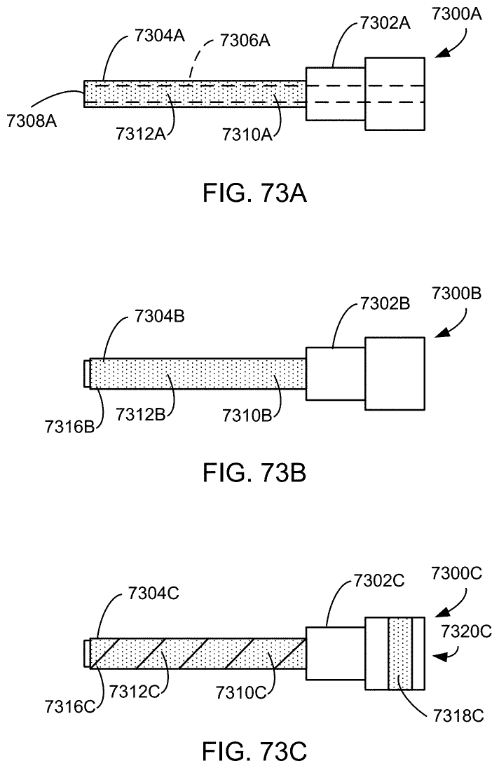

As previously discussed, microtexturing as disclosed herein may be applied to a range of medical devices and instruments. FIGS. 73A-73C illustrate additional examples of such microtextured medical instruments, and, more specifically, microtextured trocars.

FIG. 73A is a schematic illustration of a first trocar assembly 7300A according to the present disclosure. As shown, the trocar assembly 7300A includes a hub 7302A and a cannula 7304A extending distally from the hub 7302A. The hub 7302A and the cannula 7304A collectively define a lumen 7306A extending through the trocar assembly 7300A. The cannula 7304A terminates in a distal tip 7308A. In certain implementations, the distal tip 7308A may be blunt. In other implementations, the distal tip 7308A may be sharpened to facilitate insertion of the cannula 7304A into a patient. In still other implementations, the trocar assembly 7300A may further include a removable insert (not shown) disposed within the cannula such that, when assembled, a sharpened distal end of the insert extends distally out of cannula. In such implementations, the removable insert may be used to facilitate initial insertion of the trocar assembly 7300A into a patient, but may be removed from the cannula 7304A (e.g., by proximally retracting the insert) to permit access through the cannula 7304A. Following insertion of the cannula 7304A into a patient, the lumen 7306A may be used by medical personnel to access internal cavities of the patient with other tools, to enable venting of internal cavities, and to perform various other medical procedures.

In certain implementations of the present disclosure, texturing 7312A may be applied to an outer surface 7310A of the cannula 7304A. For example, texturing in the form of outwardly projecting protrusions may be disposed along some or all of the outer surface 7310A. Such protrusions may have various configurations, including, but not limited to, the various sizes, shapes, arrangements, etc. of protrusions and similar features disclosed herein.

As shown in FIG. 73A, the texturing 7312A may be integrally formed with the outer surface 7310A of the cannula 7304A. For example, in certain implementations, the texturing 7312A may be formed onto the cannula 7304A using a suitable process such as, but not limited to, overmolding, insertion molding, vapor deposition, and spraying. Stated differently, the outer surface 7310A of the cannula 7304A may provide a substrate onto which one or more coatings, layers, or similar treatment are applied to produce the texturing 7312A.

FIG. 73B is a schematic illustration of a second trocar assembly 7300B. Similar to the trocar assembly 7300A of FIG. 73A, the trocar assembly 7300B includes a hub 7302B and a cannula 7304B extending distally from the hub 7302B. The hub 7302B and the cannula 7304B collectively define a lumen (not indicated) extending through the trocar assembly 7300B.

In contrast to the integrally formed texturing 7312A of the trocar assembly 7300A, the trocar assembly 7300B includes texturing 7312B in the form of a sheath or sleeve 7316B through which the cannula 7304B may be inserted. For example, the sleeve 7316B may be formed of a biocompatible, flexible material and may include an outer surface 7310B including the texturing 7312B. Prior to insertion of the cannula 7304B, the sleeve 7316B may be stretched over the cannula 7304B (or the cannula 7304B may be pushed through the sleeve 7316B), thereby providing the texturing 7312B on the cannula 7304B.

FIG. 73C is a schematic illustration of a third trocar assembly 7300C. Similar to the trocar assembly 7300A of FIG. 73A, the trocar assembly 7300C includes a hub 7302C and a cannula 7304C extending distally from the hub 7302C. The hub 7302C and the cannula 7304C collectively define a lumen (not indicated) extending through the trocar assembly 7300C.

In contrast to the previous implementations, the trocar assembly 7300C includes texturing 7312C in the form of a wrap 7316C disposed onto the cannula 7304C. For example, the wrap 7316C may be in the form of a biocompatible strip having an outer surface 7310C onto which texturing 7312C is applied. Prior to insertion into a patient, the wrap 7316C may be wrapped about the cannula 7304C with the texturing 7312C facing outward, thereby applying the texturing 7312C to the cannula 7304C. In certain implementations, the wrap 7316C may be plain-backed and applying the wrap 7316C may include applying an adhesive to a back surface of the wrap 7316C. In other implementations, the wrap 7316C may be adhesive-backed, similar to tape. In still other implementations, the wrap 7316C may be retained on the cannula 7304C by friction. For example, the wrap 7316C may be formed of a high friction material or include texturing (e.g., texturing disclosed herein) on its back such that the wrap 7316C may be retained on the cannula 7304C by friction. Similarly, the wrap 7316C may be formed a flexible material such that the wrap 7316C may be wrapped about the cannula 7304C under tension. When tension is removed, the wrap 7316C may contract, thereby increasing retentive force of the wrap 7316C on the cannula 7304C.

In general, texturing of a cannula in trocar assemblies disclosed herein may be provided along substantially the entire cannula or only along select portions of the cannula. In general, the texturing provides increased retention and engagement of the cannula by a physiological wall (e.g., the abdominal wall) during use. For example, texturing of the cannula may reduce the likelihood of the cannula shifting inwardly or outward (e.g., medially) following insertion into a patient and, in particular, during use of the cannula to access a corresponding internal cavity of the patient.

Regardless of how texturing is applied to the cannula, the texturing may be formed from a variety of materials including, but not limited to, one or more of low-density polyethylene (LDPE), latex, polyether block amide (e.g., PEBAX®), silicone, polyethylene terephthalate (PET/PETE), nylon, polyurethane, and any other thermoplastic elastomer, siloxane, or other similar non-rigid materials.

In at least certain implementations, texturing may be applied to other portions of the trocar assembly other than the cannula. FIG. 73C, for example, further illustrates second texturing 7318C applied to a portion of the hub 7302C. Although the location of the second texturing 7318C may vary, in FIG. 73C the second texturing 7318C is shown as being applied to a proximal section 7320C of the hub 7302C that generally corresponds to a grip, e.g., for use during insertion or removal of the cannula 7304C or to stabilize the trocar assembly 7300C while accessing the internal cavity of the patient.

Reinforced Overtubes

As discussed herein, at least certain aspects of the present disclosure are directed to split overtubes and medical device assemblies including split overtubes. In at least certain implementations, the overtubes may be substantially homogenous along their length with respect to their construction and properties; however, as discussed below in further detail, in at least certain implementations, overtubes in accordance with the present disclosure may be reinforced along their length and, in particular, reinforced at discrete locations along their length.

Various approaches to reinforcing split overtubes are presented herein; however, in general, the reinforcement techniques discussed herein include disposing reinforcing features at discrete locations along the length of the split overtube. Such reinforcements may be in the form of ribs, rings, coils, or similar structures coupled to, disposed within, or otherwise integrated into the split overtube. Reinforcements many also include selectively altering properties of the overtube itself to create locally reinforced regions of the split overtube. For example, the wall thickness, material, or similar properties of the split overtube affecting strength, flexibility, etc. of the overtube may be modified within discrete regions of the split overtube to provide the reinforcing features.

Regardless of the particular type of reinforcement implemented, reinforcing the split overtube by including reinforcing features along its length can be used to achieve a variety of benefits as compared to conventional overtubes including, but not limited to, greater retention of the split overtube on medical tools (e.g., endoscopes), easier coupling of the split overtube to medical devices, increased structural integrity of the split overtube, and the like.

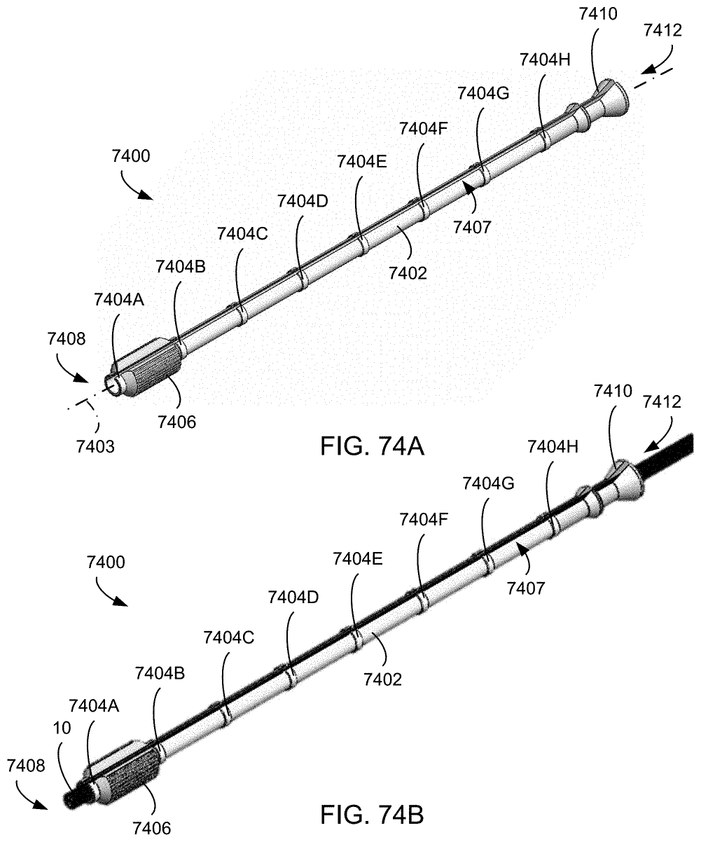

FIGS. 74A and 74B are isometric views a split overtube assembly 7400 including a reinforced overtube 7402 with a longitudinally extending split 7407 through which an elongate medical device may be inserted into the overtube 7402. More specifically, FIG. 74A illustrated the split overtube assembly 7400 alone while FIG. 74B illustrates the split overtube assembly 7400 coupled to a medical device, namely, an endoscope 10. As illustrated, the split overtube assembly 7400 generally includes a split overtube 7402 or similar elongate flexible body along which one or more reinforcing structures, such as reinforcing ribs 7404A-7404H, may be disposed. As shown, the split overtube assembly 7400 further includes an inflatable balloon 7406 disposed at a distal end 7408 and a handle 7410 disposed at a proximal end 7412; however, it should be appreciated that the inflatable balloon 7406 and the handle 7410 are included merely to illustrate one example implementation of a reinforced overtube assembly, namely, as an overtube for use in endoscopic procedures, such as colonoscopies.

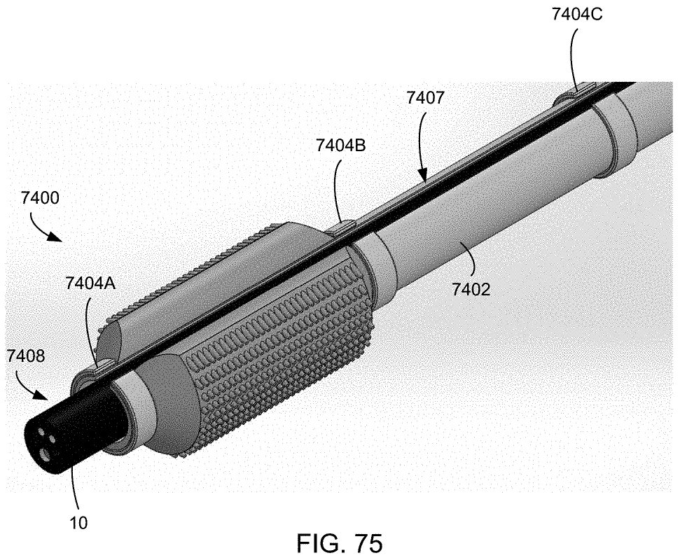

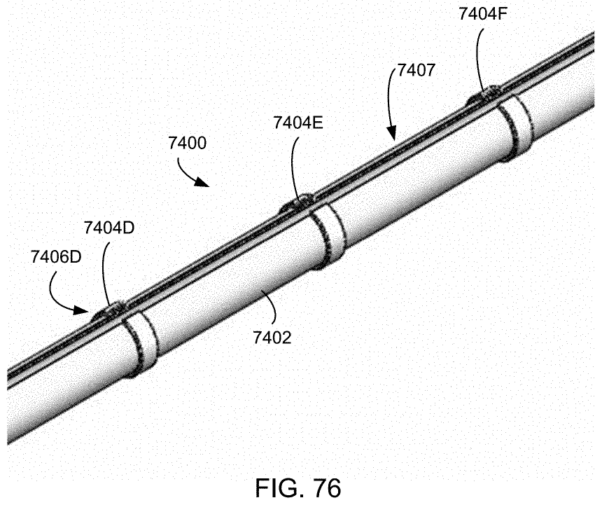

In at least certain implementations and as illustrated in FIGS. 74A and 74B, the reinforcing ribs 7404A-7404H are distributed along a length of the split overtube 7402 and extend circumferentially about a longitudinal axis 7403 of the split overtube 7402. Further details of ribs 7404A-7404C are visible in FIG. 75, which is a detailed view of the distal end 7408 of the overtube assembly 7400 as illustrated in FIG. 74B (i.e., coupled to an endoscope 10), and FIG. 76, which is a detail view of an intermediate section of the overtube assembly 7400.

As used herein, the term “longitudinal axis” in the context of split overtubes is used to refer to an axis through a center of the primary lumen and extending from a proximal end of the primary lumen to a distal end of the primary lumen. As a result, as the split overtube is bent, curved, or otherwise manipulated during use, the longitudinal axis of the split overtube also varies to follow the path of the primary lumen. Beyond the proximal and distal ends of the split overtube, the longitudinal axis extends normal to the opening of the split overtube at the proximal and distal end, respectively. Accordingly, while longitudinal axis 7403 is illustrated in FIG. 74A as being substantially straight, this is only a result of split overtube assembly 7400 and split overtube 7402 being illustrated in a substantially straight/unbent configuration. As split overtube 7402 is curved, bent, or otherwise manipulated during use, longitudinal axis 7403 will similarly vary.

As illustrated in FIG. 75A-76, each rib 7404A-7404H may define a rib split (e.g., rib split 7406D of rib 7404D, shown in FIG. 76) to permit insertion of the endoscope 10 (or other medical device) into the split overtube 7402. In certain implementations, an inner surface of the split overtube 7402 may be lubricated (e.g., by applying a lubricant or forming the split overtube 7402 with a lubricated or low-friction inner coating or layer) to further facilitate insertion of the endoscope 10 or other medical device therein. Lubrication or a lubricating layer/coating may also be applied to or disposed on an interior and/or on an exterior of the split overtube 7402 to facilitate use of the split overtube assembly 7400, such as to improve the ease with which the split overtube 7402 slides relative to the scope and/or the physiological lumen. In still other implementations, lubrication or a lubricating layer/coating may be applied along the edges of the split 7407 and/or on the edges of the ribs 7404A-7404H defining the rib splits to facilitate insertion of elongate medical devices into the split overtube 7402.

Reinforcement structures, such as the ribs 7404A-7404H of the overtube assembly 7400 may be integrally formed with the split overtube 7402 of the overtube assembly 7400 or may be separately formed and subsequently coupled to the split overtube 7402. Although the specific dimensions of ribs 7404A-7404H (and similar structures disclosed herein) ultimately depend on the size of split overtube 7402, in at least certain implementations, ribs 7404A-7404H may have a diameter from and including about 2 mm to and including 20 mm.

In at least certain implementations, ribs and similarly structures disclosed herein may be configured to be bistable in an open and closed configuration. For example, in the open configuration the ribs/tib-type structure may hold open the split overtube for placement on the scope. Once in place, the ribs may be pressed shut. As the ribs are pressed shut, the ribs may “snap” into a closed configuration to hold the scope within the split overtube. In the closed configuration, the ribs may completely surround the scope or may still leave a gap along the split of the overtube.

As illustrated in FIGS. 74A-76, reinforcement structures according to the present disclosure (such as ribs 7404A-7404H) may be coupled to or otherwise extend outwardly from the split overtube 7402. In such implementations, the reinforcement structures may be constructed to have leading or trailing surfaces/edges (relative to the longitudinal axis 7403) that are rounded, filleted, or that otherwise smoothly transition into an outer surface of the split overtube 7402 to minimize the engagement of the reinforcement structures with a wall of a physiological lumen within which the overtube assembly 7400 is disposed.

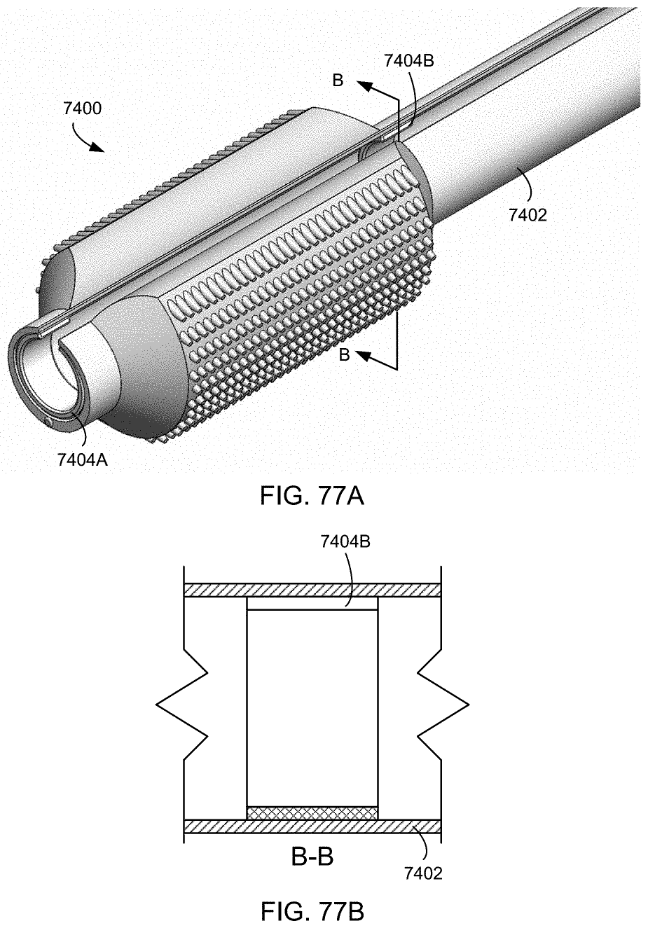

In other implementations, the reinforcement structures may instead be disposed on an interior surface of the split overtube. For example, FIG. 77A illustrates an alternative implementation of the overtube assembly 7400 in which reinforcement structures are disposed on or otherwise extend from an interior surface of the split overtube 7402. More specifically, and as illustrated in FIG. 77B (which is a cross-sectional view along section B-B) ribs 7404A and 7404B are illustrated as being coupled to an interior surface of the split overtube 7402.

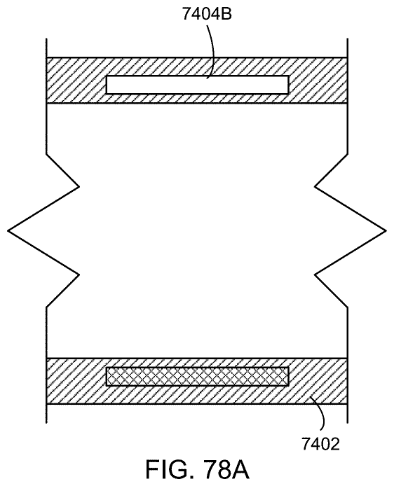

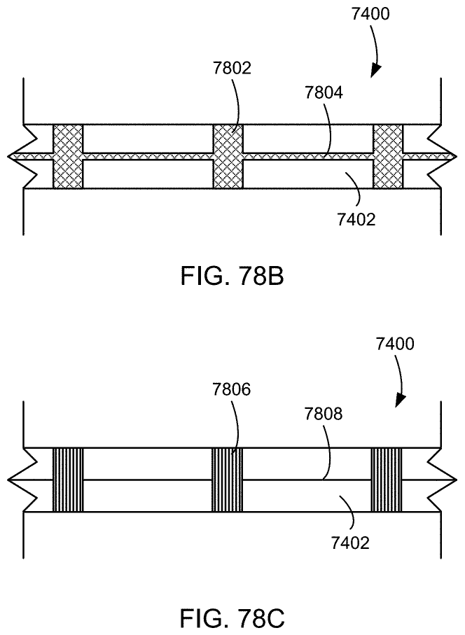

In still other implementations, the reinforcement structures may instead be embedded within the wall of the split overtube. For example, FIG. 78A is a partial cross-sectional view of the overtube assembly 7400 in which the rib 7404B is embedded within a wall of the split overtube 7402.

In at least certain implementations, ribs 7404A-7404H may be configured to expand during insertion of the endoscope 10 into the split overtube 7402. To facilitate such insertion, the ribs 7404A-7404H may be formed of a sufficiently flexible material that permits elastic deformation of the ribs (e.g., expansion) during insertion of the endoscope 10. For example, ribs according to the present disclosure may be formed from a range of materials including, but not limited to, one or more of polypropylene, polyethylene, nylon, polyurethane, and other similar polymers. Ribs according to the present disclosure may also be formed of metallic materials, such as Nitinol, or a combination of one or more polymers and/or metallic materials.

FIG. 78B is an elevation view of an alternative implementation of the split overtube assembly 7400 in which reinforcement structures are similarly embedded within the split overtube 7402 of the split overtube assembly 7400, but are formed from braided bands or similar reinforcement structures disposed along the length of the split overtube 7402. For example, the reinforcement structure may be in the form of circumferential braided bands (such as circumferential braided band 7802) that may be integrated into the split overtube 7402. In at least certain implementations, the circumferential bands may also be longitudinally coupled to each other, such as by a longitudinal band 7804, which may be integrally formed with the circumferential bands, or which may be a separate structure coupled to and/or extending adjacent the circumferential bands. However, in other implementations, the circumferential bands may be discrete structures distributed along the length of the split overtube 7402 and the longitudinal band 7804 may be omitted.

In certain implementations, the split overtube 7402 may be formed from a braided material. In such implementations, the split overtube 7402 may include a first layer of substantially homogeneous braided material. Braided bands may then be coupled to the first layer, either as discrete bands or as a second layer coupled to the first layer and along which the braided bands are disposed. In other implementations, the braided bands may be formed by altering characteristics of the braid along the length of the split overtube 7402. For example, the split overtube 7402 may be formed of a braided material that includes a first type of braid along the majority of its length; however, at discrete locations along the split overtube 7402, the braid may be altered to locally reinforce the split overtube 7402 at the discrete locations. Among other things, the density of the braid, the material of the braid, the dimensions of the braid wire, or other similar properties of the braid may be altered to form the reinforced portions of the split overtube 7402.

FIG. 78C is an elevation view of another alternative implementation of the split overtube assembly 7400 in which reinforcement structures are similarly embedded within the split overtube 7402 of the split overtube assembly 7400, but are coils (e.g., coil 7806, which may be formed, e.g., from a metallic wire or polymer strand) disposed along the length of the split overtube 7402. In at least certain implementations, the coils may be formed by wrapping wire about a body of the split overtube 7402 during formation of the body and then subsequently cutting the wrapped material when forming the split 7407. The resulting reinforcement structures would then appear as a series of split rings. Similar to the previously discussed circumferential bands, the coils may be longitudinally coupled to each other, such as by a longitudinal wire 7808, which may be formed of a similar material as the coils. However, in other implementations, the coils may be discrete structures distributed along the length of the split overtube 7402.

In other implementations, ribs according to the present disclosure may be formed from a relatively rigid material but may have a first configuration (e.g., an open configuration) to permit insertion of the endoscope 10 into the split overtube 7402. After insertion of the endoscope 10, the ribs may be transitioned into a second configuration (e.g., a closed configuration) to retain the endoscope 10.

FIG. 79 illustrates several alternative implementations of ribs according to the present disclosure disposed on an example split overtube 7902. Rib 7904A is a first example rib and, more specifically is a one-piece rib that may be formed of a material sufficiently flexible to permit insertion of an endoscope or similar tool into the split overtube 7902. More specifically, rib 7904A defines a rib split 7905A aligned with a split 7903 of the split overtube 7902. The rib 7904A is generally formed of a sufficiently flexible material such that the rib split 7905A may be expanded prior to or during insertion of an elongate medical device (e.g., an endoscope) into the split overtube 7902. Subsequently, the rib split 7905A may be reduced, e.g., by returning to its unstrained state, thereby retaining the elongate medical device within the split overtube 7902.

Rib 7904B is a second example rib in which closure of the rib 7904B is facilitated by magnets 7910A, 7910B. More specifically, the magnets 7910A, 7910B are disposed on opposite sides of rib split 7905B. To insert an elongate medical device into the split overtube 7902, sufficient force may be applied to separate the magnets 7910A, 7910B, (e.g., by pulling apart the split overtube 7902 or pressing the medical device along the split 7903 of the split overtube 7902), thereby opening the rib split 7905B and allowing insertion of the elongate medical device. Following insertion, the magnets 7910A, 7910B may be moved (e.g., by magnetic force and/or force applied by a user of the split overtube 7902) such that the magnets 7910A, 7910B become magnetically coupled and maintain the split overtube 7902 in a closed configuration. In certain implementations, the magnets 7910A, 7910B may be configured to be in contact when the split overtube 7902 is in the closed configuration. Alternatively, the magnets 7910A, 7910B may be configured to be magnetically coupled without being in physical contact when the split overtube 7902 is in a closed configuration.

Rib 7904C illustrates a third example rib in which the rib 7904C includes an interlocking feature 7912. The interlocking feature 7912 includes a first feature 7914 disposed on a first side of a rib split 7905C and a second feature 7916 disposed on a second side of the rib split 7905C such that, when the rib 7904C is in a closed configuration, the first feature 7914 positively engages or is otherwise retained by the second feature 7916. In the specific example illustrated in FIG. 79, the first feature 7914 and the second feature 7916 are mating hooked features. It should be appreciated that such mating hooked features are intended only as an example and that any suitable interlocking feature 7912 may be used instead. To insert an elongate medical device into the split overtube 7902, the interlocking feature 7912 of the rib 7904C may be disengaged, e.g., by pulling the features 7914, 7916 in opposite directions, sliding the features 7914, 7916 relative to each other and the like. Similarly, following insertion of an elongate medical device into the split overtube 7902, the rib 7904C may be transitioned into a closed configuration by reengaging the first feature 7914 and the second feature 7916.

Although the ribs illustrated in FIG. 79 are illustrated as unitary components, it should be appreciated that, in at least certain implementations, the ribs may be formed from multiple pieces that, when coupled together (e.g., by interlocking features, adhesives, magnets, etc.) form an annular structure. Accordingly, in such implementations, a rib may be formed from multiple rib sections that may be coupled to with each other about the split overtube 7902 following insertion of an elongate medical device therein.

It should also be appreciated that ribs in accordance with the present disclosure may be integrally formed with the split overtube 7902, may be permanently coupled to the split overtube 7902, or may be selectively coupleable to the split overtube 7902. For example, in certain implementations, an elongate medical device may be inserted into the split overtube 7902 and ribs may be subsequently snapped onto or otherwise coupled to the split overtube 7902 subsequent to insertion of the elongate medical device. Notably, in such implementations, it is not necessary that the rib split of the ribs be aligned with the split of the split overtube 7902 when the split overtube 7902 and the elongate medical device are fully assembled.

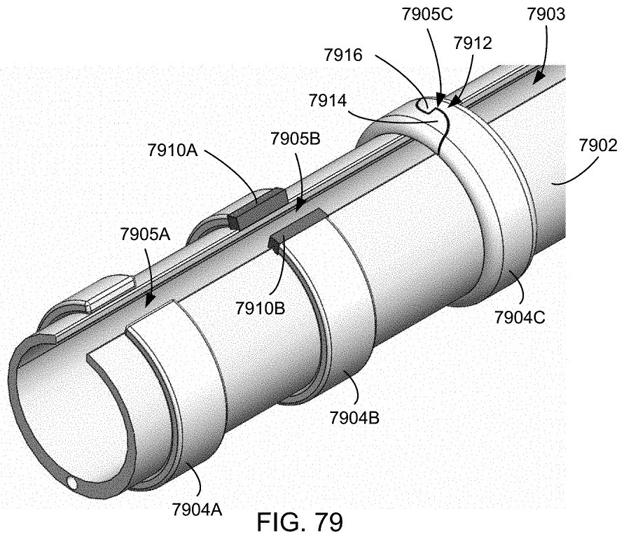

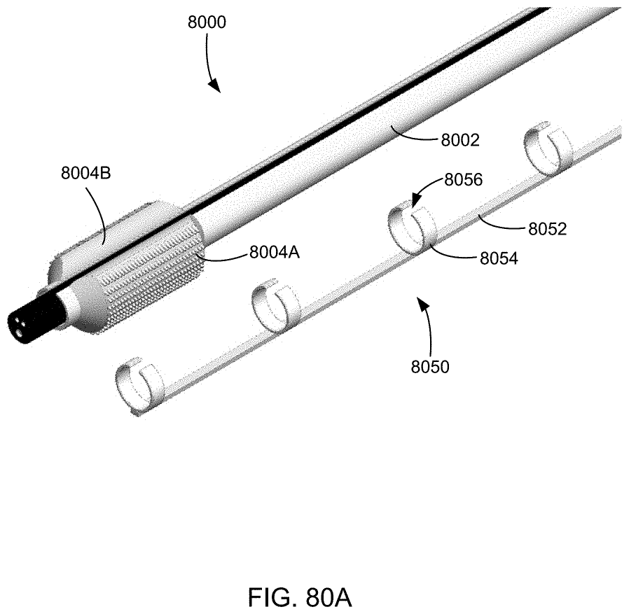

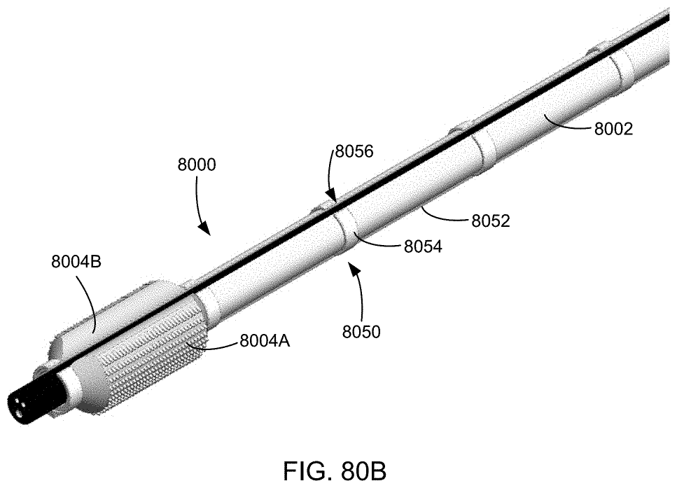

FIGS. 80A and 80B illustrate an alternative implementation of a split overtube assembly 8000 including reinforcing ribs in the form of a ring assembly. More specifically, FIG. 80A illustrates split overtube assembly 8000 in a partially disassembled state in which a ring assembly 8050 of split overtube assembly 8000 is decoupled from a split overtube 8002 of split overtube assembly 8000 while FIG. 80B illustrates split overtube assembly 8000 with ring assembly 8050 assembled onto split overtube 8002. As shown in FIG. 80A, ring assembly 8050 generally includes a backbone 8052. Ribs or split rings, such as split ring 8054, are placed along the length of and coupled to backbone 8052. To the extent the following discussion refers to split ring 8054 and unless otherwise noted, features of split ring 8054 discussed below should be assumed to apply equally to all split rings of ring assembly 8050.

As illustrated in FIG. 80A, split ring 8054 may couple to backbone 8052 at a location directly opposite an opening 8056 of split ring 8054 and on an outer circumference of split ring 8054. However, in other implementations, backbone 8052 may instead couple to backbone 8052 at another location about the inner or outer circumference of split ring 8054. Moreover, while illustrated as being substantially straight, backbone 8052 may instead be curved or have a non-straight shape (e.g., a corkscrew shape) such that the location at which backbone 8052 couples to the split rings varies along the length of backbone 8052. Implementations of this disclosure may also include multiple backbones extending along all or a portion of ring assembly 8050.

The split rings of ring assembly 8050 may be integrally formed with backbone 8052 or may be separately formed from backbone 8052 and subsequently coupled to backbone 8052 using any suitable method (e.g., ultrasonic welding, adhesive, magnetic coupling, mechanical coupling, etc). Ring assembly 8050 includes split rings evenly distributed along its length. However, in other implementation, the placement and distribution of split rings may vary. For example, increasing the spacing between split rings in a longitudinal segment of split overtube assembly 8000 can reduce rigidity within the segment. Similarly, decreasing the spacing between split rings in a longitudinal segment of split overtube assembly 8000 can increase rigidity within the segment. Similarly, varying characteristics of split rings of ring assembly 8050 along the length of ring assembly 8050 can also selectively modify rigidity and reinforcement along the length of split overtube assembly 8000. For example, ring assembly 8050 may split rings that are longitudinally wider, thicker, and/or made of a relatively rigid material in segments requiring greater reinforcement/rigidity and split rings that are longitudinally narrower, thinner, and/or formed of more flexible material in segments requiring less or otherwise reduced reinforcement/rigidity.

Spacing of the split rings may also be varied to accommodate other components of split overtube assemblies. For example, the split rings of ring assembly 8050 need to be adequately spaced to accommodate balloons 8004A, 8004B disposed at a distal end of split overtube assembly 8000.

Backbone 8052 is illustrated in FIGS. 80A and 80B as being substantially homogeneous over its entire length; however, by selectively modifying segments of backbone 8052, properties of backbone 8052 may be varied within those segments. For example, certain segments of backbone 8052 may be thicker than other segments such that the thicker segments are more rigid than the thinner segments. Similarly, certain segments of backbone 8052 may include stronger or less flexible materials than other segments such that the segments including the less flexible materials provide increased reinforcement. As yet another example, certain segments of backbone 8052 may include cutouts, scallops, slits, or other similar structural modifications to impact localized rigidity or flexibility. For example, segments of backbone 8052 may be include “kerf cutting” or similar modifications that create living hinges or similar localized areas of flexibility in select segments of backbone 8052.

Implementations of the present disclosure may include one or more ring assemblies distributed along the length of split overtube 8002. Also, while illustrated and discussed above as being included in the split overtube assembly 8000, in certain implementations, backbone 8052 may be configured to be cut away from or otherwise detached from the split rings after insertion of split overtube 8002 into the split rings. In such cases, backbone 8052 may primarily function as an assembly aid but not form part of the final split overtube assembly 8000.

Ribs and backbones of ring assemblies according to this disclosure may be formed from any suitable material, including any suitable metallic or plastic/polymer material. Similarly, ribs, backbones, and ring assemblies may be formed by any suitable method including, but not limited to, machining, molding.

As previously discussed in the context of FIG. 74, split rings and ribs disclosed herein may be configured to be bistable and, in particular, stable in each of an open configuration (e.g., to facilitate insertion of an overtube and/or scope into the split rings/ribs) and a closed configuration (e.g., to secure the overtube and/or scope once inserted).

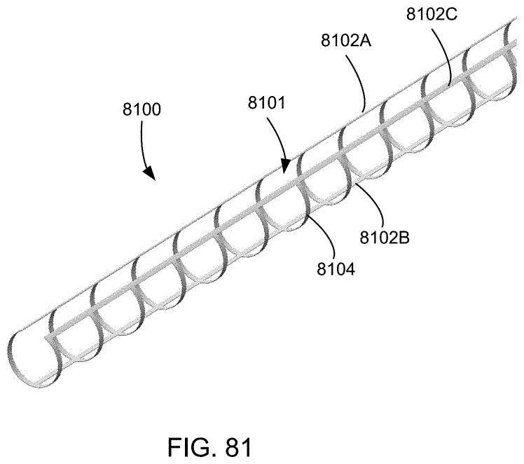

FIG. 81 illustrates an alternative implementation of a reinforcing structure 8100 similar to ring assembly 8050. Like ring assembly 8050, reinforcing structure 8100 is configured to be coupled to or otherwise assembled with a split overtube, such as split overtube 8002 (shown in FIGS. 80A and 80B).

As illustrated, reinforcing structure 8100 includes longitudinal members 8102A-C with longitudinal member 8102A and 8102C extending along opposite sides of a split 8101 and longitudinal member 8102B disposed opposite split 8101. When assembled to or integrated with a split overtube 8002, split 8101 may substantially align with the split of the split overtube 8002. Reinforcing structure 8100 further includes circumferential ribs (such as rib 8104) extending along its length and coupled together by longitudinal member 8102A-C.

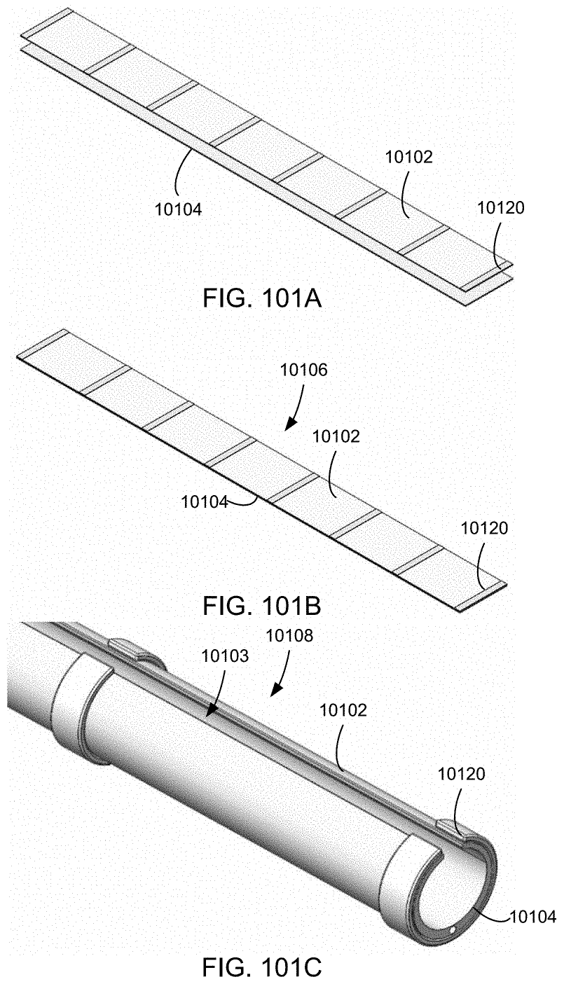

In certain implementations, reinforcing structure 8100 may be formed from a flat sheet of material and subsequently folded or curved to conform to the end shape of a split overtube assembly. For example, reinforcing structure 8100 may be laser or waterjet cut from a polymer or metal sheet and subsequently layered with other layers of the split overtube assembly, e.g., as described in the layer-based assembly process disclosed in the context of FIGS. 101A-109, below.

Like the split rings and backbone of ring assembly 8050, longitudinal members 8102A-C and ribs 8104 may be modified to impart different characteristics along the length of reinforcing structure 8100 and a split overtube assembly including reinforcing structure 8100. Among other things, the quantity, spacing, thickness, width, and material of either of the longitudinal members 8102A-C or ribs 8104 may be varied along the length or circumference of reinforcing structure 8100 to create segments of 8100 having relatively higher or lower rigidity. Moreover, while the members of reinforcing structure 8100 extend in either the longitudinal or circumferential direction, other implementations of this disclosure may include members that extend each of longitudinally and circumferentially. In still other implementations, reinforcing structure 8100 may instead be formed by cutting a uniform or non-uniform pattern (e.g., a pattern based on a basic geometric shape (e.g., a triangle), tessellation, etc.) into a sheet of material. The cut sheet may then be wrapped or otherwise bent to conform to the final shape of a split overtube assembly into which reinforcing structure 8100 is to be integrated.

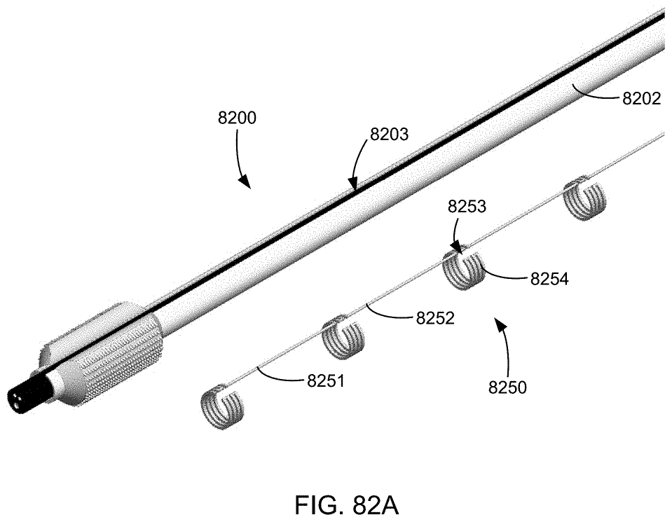

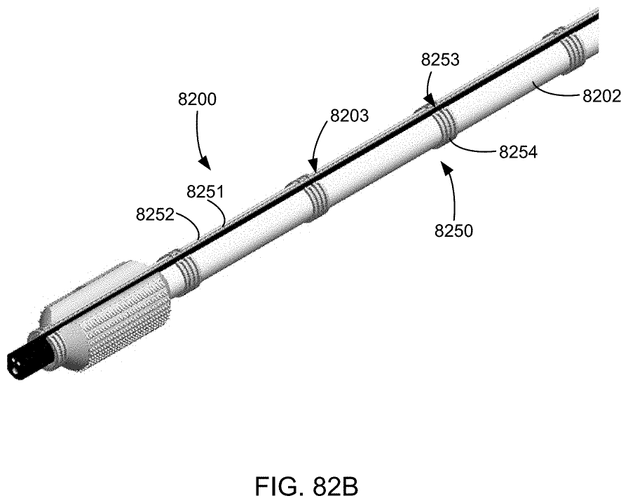

FIGS. 82A and 82B illustrate another alternative implementation of a split overtube assembly 8200 including a wire-based reinforcing structure. More specifically, FIG. 82A illustrates split overtube assembly 8200 in a partially disassembled state in which a wire assembly 8250 of split overtube assembly 8200 is decoupled from a split overtube 8202 of split overtube assembly 8200 while FIG. 82B illustrates split overtube assembly 8200 with wire assembly 8250 assembled onto split overtube 8202. As shown in FIG. 82A, wire assembly 8250 generally includes a wire 8251 that extends longitudinally (e.g., longitudinal segment 8252) and forms circumferential coils or wrappings (e.g., coil 8254).

In certain implementations, wire 8251 may be formed to have a shape similar to a cinch binding, wire binding spine, twin loop binding spine, binding comb, or similar binding structure typically used to bind papers, albeit with different spacing between coils. Notably, such binding structures may include a longitudinal slot or gap through which sheets of paper may be inserted. In the context of wire assembly 8250, each coil of wire 8251 may be formed to have a longitudinally extending gap (e.g., gap 8253) that may be aligned with a split 8203 of split overtube 8202 when wire assembly 8250 is assembled with split 8203 of split overtube 8202 to form split overtube assembly 8200. In other implementations, wire 8251 may be formed to extend about the full circumference of split overtube 8202, coupled to split overtube 8202, and subsequently cut along split 8203 to enable insertion of tools into split overtube assembly 8200.

As illustrated in FIGS. 82A and 82B, wire 8251 may be formed such that longitudinal segments 8252 of wire 8251 are aligned along the length of wire assembly 8250 and coils 8254 are substantially similar and evenly distributed along the length of wire assembly 8250. However, in other implementations, the configuration of wire 8251 may vary. For example, the circumferential location along which segments of wire 8251 between coils extend may vary along the length of wire assembly 8250. As another example, segments of wire 8251 between coils may also extend in both a longitudinal and circumferential direction such that the segments between coils form a spiral, corkscrew, or similar pattern along the length of wire 8251.

The configuration of coils may similarly vary from the illustrations of FIGS. 82A and 82B, particularly to provide localized areas of relatively more or less rigidity to split overtube assembly 8200. For example, decreasing the spacing between coils along a length of wire assembly 8250 increases the rigidity of the corresponding segment of split overtube assembly 8200 when assembled. Similarly, increasing the spacing between coils of a segment of wire assembly 8250 can decrease the rigidity of the corresponding segment of split overtube assembly 8200 when assembled. Additionally, or alternatively, one or more of the coil width (e.g., as measured in the longitudinal direction), coil density (e.g., winds of wire per unit length of the coil), wire material, wire diameter, and other similar aspects of the wire assembly 8250 may be varied along its length to selective impart different characteristics to the wire assembly 8250 and/or split overtube assembly 8200 when assembled.

Each of the foregoing reinforcing structures and other reinforcing structures disclosed herein may extend along the entire length or only along a partial length of a corresponding split overtube assembly. In certain implementations, multiple reinforcing structures may be applied along the length of a split overtube assembly. In such implementations, reinforcing structures may extend along substantially the full length of the split overtube assembly. Alternatively, segments of the split overtube assembly without any reinforcement may separate adjacent segments with reinforcing structures. In still other implementations, split overtube assemblies may include multiple reinforcing structures that at least partially overlap such that multiple reinforcing structures may support certain longitudinal segments of the split overtube assembly.

Split Overtube with Magnetic Closure

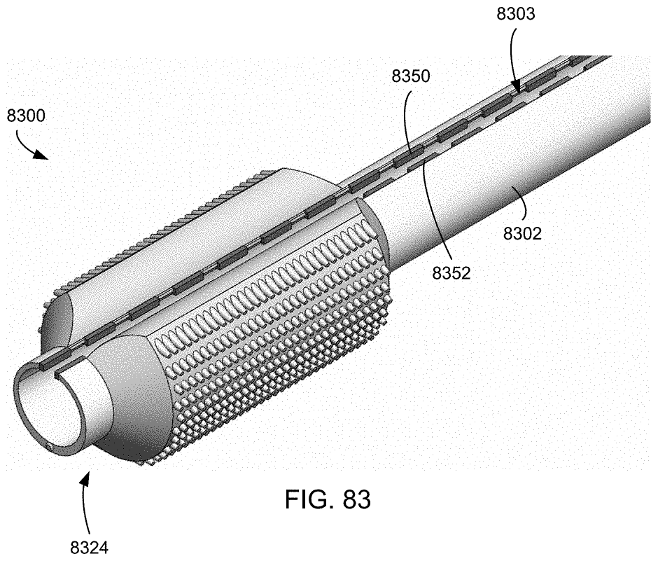

FIG. 83 is an isometric view of a split overtube assembly 8300 in accordance with the present disclosure and, more specifically, an isometric view of a distal portion 8324 of the split overtube assembly 8300. The split overtube assembly 8300 includes a split overtube 8302 defining a split 8303.

As previously discussed in the context of FIGS. 59-61, in at least certain implementations of the present disclosure, split overtubes in accordance with the present disclosure may include closure features, such as a zipper-style closure. FIG. 83 illustrates an alternative closure mechanism in the form of magnets distributed along the length of the split 8303. More specifically, a first set of magnets (e.g., including magnet 8350) is distributed along a first side of the split 8303 and a second set of magnets (e.g., including magnet 8352) are distributed along a second side of the split 8303.

In use, the sets of magnets may be pulled apart or otherwise separated to allow insertion of an elongate medical device into the split overtube 8302. Following insertion, the elongate medical device may be retained within the split overtube 8302 by permitting the each of the pairs of magnets to reengage. In certain implementations, reengagement of the pairs of magnets generally includes magnetic engagement but may include physical contact of the magnets.

Implementations of the present disclosure may include one or more pairs of magnets, which may be used alone or in combination with one of more other closure features discussed herein. In certain implementations and as illustrated in FIG. 83, the magnets may be directly coupled to the split overtube 8302. In other implementations, the magnets may instead be coupled to or otherwise integrated into reinforcing ribs, as discussed herein.

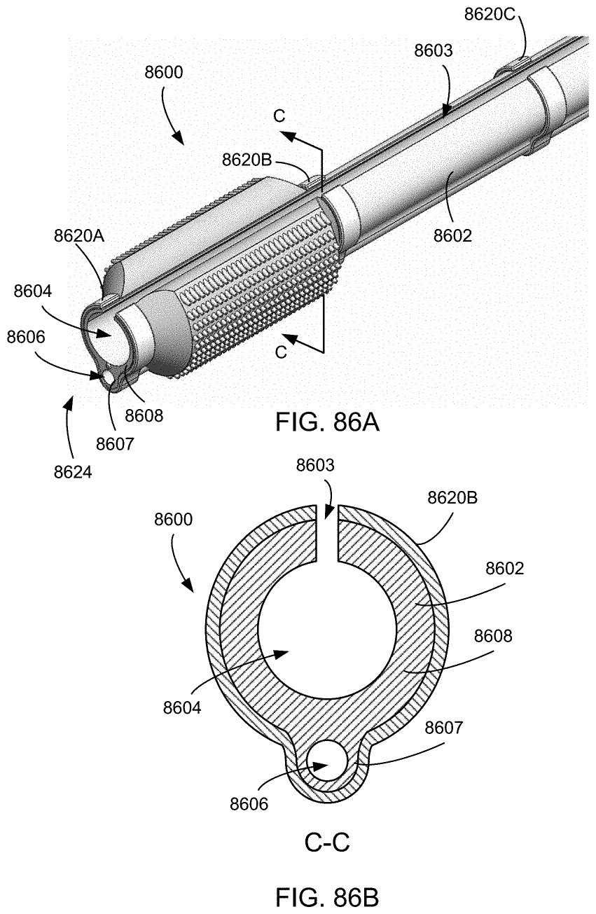

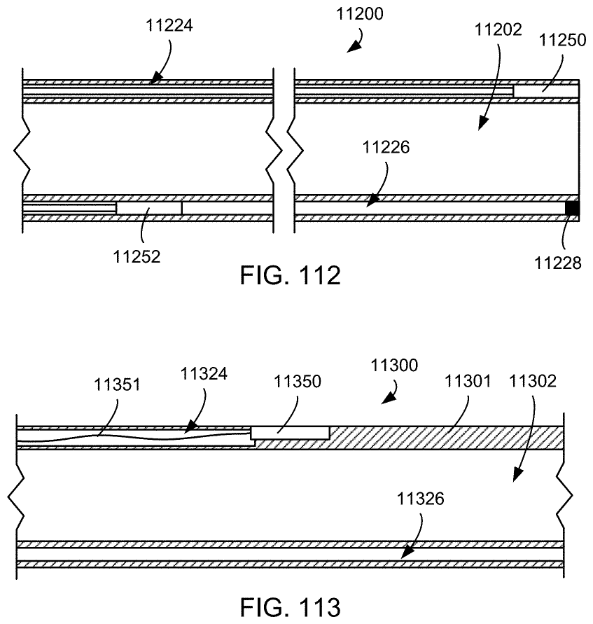

In still other implementations of the present disclosure, magnets may be disposed along the split interface within the split overtube 8302. For example, in certain implementations, magnets may be integrally formed (e.g., by overmolding the split overtube 8302 onto the magnets or disposing the magnets between layers of the split overtube 8302). In still other implementations, magnets may be disposed within the split overtube 8302 by forming lumens or pockets extending through the split overtube 8302 within which the magnets may be disposed. For example, lumens similar to the secondary or working lumens discussed below in the context of FIG. 86A-90B or the secondary lumens discussed below in the context of FIGS. 112 and 113 may be formed within the wall of the split overtube 8302 and extend along opposite sides of the split of the split overtube 8302. Magnets may then be disposed within the lumens to facilitate the closure functionality described above.

Split Overtube Assemblies Including Split Handles

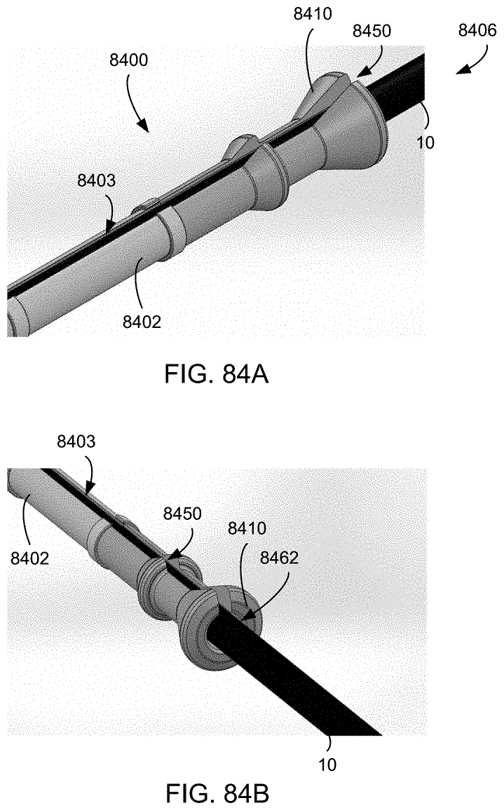

Split overtube assemblies may include proximal handles. For example, FIGS. 84A and 84B are isometric views of a split overtube assembly 8400 and, in particular, isometric views of a proximal portion 8406 of the split overtube assembly 8400, which includes a handle 8410. As illustrated, the handle 8410 is coupled to a proximal end of a split overtube 8402 of the split overtube assembly 8400. The handle 8410 defines a longitudinally extending handle split 8450 aligned with a split 8403 of the split overtube 8402. The handle 8410 further defines a primary lumen 8462 within which an elongate medical device, such as an endoscope 10, may be retained during use. In general, the primary lumen 8462 may be sized to permit longitudinal movement of the endoscope 10 relative to the handle 8410 during use. The handle 8410 may be formed of a more rigid material than the split overtube 8402. For example, and without limitation, the handle 8410 may be formed of one or more of HDPE, LDPE, ABS, polypropylene, polyethylene, nylon, polyurethane, PET, PTFE, FEP, TPPE, or similar polymers. In other implementations, the handle 8410 may be formed from metallic materials, such as stainless steel, or a combination of metallic and polymer materials.

Handles according to this disclosure may retain the endoscope 10 using various techniques. For example, in the implementation illustrated in FIGS. 84A and 84B, the handle split 8450 may have a width that is less than a width of the elongate medical device with which it is to be used. In such implementations, insertion of the endoscope 10 into the handle 8410 may generally rely on partially deforming the endoscope 10 to alter its width, thereby permitting insertion of the endoscope 10 through the handle split 8450. Once inserted, the endoscope 10 may return to its original shape and, as a result, be retained within the handle 8410. Accordingly, in at least certain implementations, the handle 8410 is formed of an at least partially deformable material that permits insertion of the endoscope 10 (or other elongate tool) through the split 8450, but that subsequently causes the handle 8410 to return to its original shape.

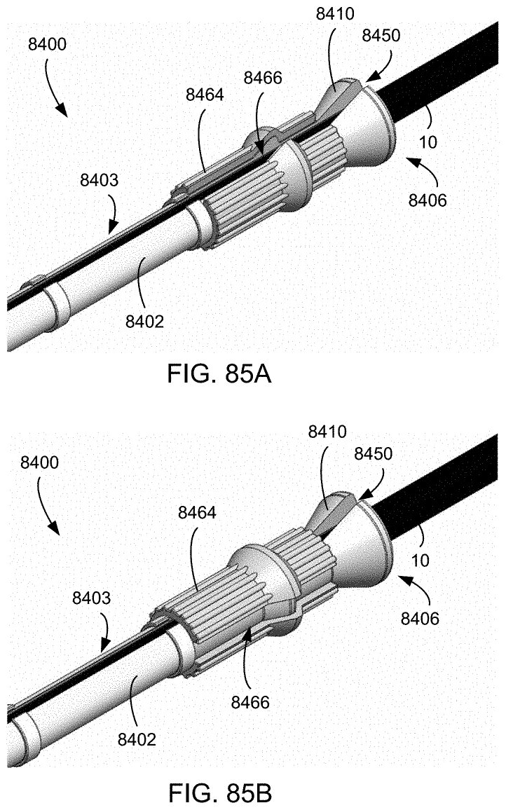

FIGS. 85A and 85B illustrate an alternative approach to retaining the endoscope 10 within the handle 8410. In general, the approach illustrated in FIGS. 85A and 85B relies on a closure mechanism that may be manipulated to selectively expose and cover the handle split 8450. In the specific implementation of FIGS. 85A and 85B, the closure mechanism is in the form of a rotatable closure 8464.

As shown in FIG. 85A, the rotatable closure 8464 defines a closure split 8466 such that, when the rotatable closure 8464 is in an open state, the closure split 8466 aligns with the handle split 8450, thereby permitting insertion and/or removal of the endoscope 10. Following insertion of the endoscope 10, the rotatable closure 8464 may be manipulated (e.g., rotated about a longitudinal axis of the handle 8410) such that the closure split 8466 and the handle split 8450 are no longer aligned, thereby retaining the endoscope 10 within the handle 8410. In such implementations, the handle split 8450 may have a width equal to or even greater than that of the endoscope 10, thereby precluding the need to deform the endoscope 10 for insertion.

The rotatable closure 8464 is one example of a closure according to the present disclosure. More generally, any suitable structure that may be manipulated to selectively cover/obstruct the handle split 8450 may be used. For example, in one alternative implementation, the closure may be a cover that may be selectively attached and detached from the handle 8410 to obstruct the handle split 8450. For example, any suitable cover may be selectively snapped onto or pulled off of the handle 8410 to obstruct the handle split 8450.

Similarly, while the closure illustrated in FIGS. 85A and 85B relies on rotation movement to transition the handle 8410 between an open and closed configuration, other forms of manipulation are also considered. For example, in other implementations transitioning the handle between an open and closed position may include manipulating by one or both of rotating and translating (e.g., longitudinally translating) a closure structure.

In certain implementations, the handle may include various features to control and/or restrict movement of the closure structure. For example, in certain implementations, the closure structure may be biased into a particular position, e.g., a closed position. In such implementations, biasing mechanisms may be incorporated into the handle to apply force on the closure structure in a closed direction, whatever that direction may be in the particular implementation. For example, and without limitation, the handle may include mechanical (e.g., springs or elastics), electric, magnetic, pneumatic, or other mechanisms adapted to bias the closure structure into one of an open and closed position. Similarly, the handle may include various mechanical stops configured to limit movement of the closure structure.

Closure structures may be retained on the handle using various approaches. For example, in certain implementations, the closure structure may be coupled to the handle by an interference fit. In other implementations, the closure structure may be coupled to the handle by one or more fasteners.

Split Overtubes Including Working Channels

Split overtubes according to the present disclosure generally define a primary lumen within which an elongate medical device or device, such as an endoscope, may be disposed. In certain implementations, such split overtubes may further define additional lumens for various purposes. For example, such additional lumens may be used to provide a channel through which additional tools may be introduced, through which fluids or other substances may be provided, or through which fluids may be removed, among other things.

FIG. 86A is an isometric view of an example split overtube assembly 8600 and, in particular, an isometric view of a distal portion 8624 of the split overtube assembly 8600. Similar to other assemblies disclosed herein, the split overtube assembly 8600 includes a split overtube 8602 defining a split 8603. FIG. 86B is a cross-sectional view of the overtube assembly 8600 taken along lines C-C.

The split overtube 8602 defines a primary lumen 8604 in communication with the split 8603 and for receiving an elongate medical device, such as an endoscope. The split overtube 8602 further defines a secondary or working lumen 8606 extending along the length of the split overtube 8602.

In the specific implementation illustrated in FIGS. 86A and 86B, the split overtube 8602 includes a lobe portion 8607 protruding from a substantially cylindrical primary body 8608 of the split overtube 8602. Although illustrated as being opposite the split 8603, in other implementations, the lobe portion 8607 may instead be located elsewhere on the circumference of the primary body 8608. Moreover, while only one lobe portion 8607 is illustrated, other implementations may include multiple lobe portions protruding from the primary body 8608 with each lobe portion defining a respective lumen extending along the length of the split overtube 8602.

As noted above, certain implementations of the present disclosure may include reinforcing structures, disposed along the length of the split overtube 8602. Accordingly, the split overtube assembly 8600 includes ribs, such as ribs 8620A-8620C, distributed along the length of the split overtube 8602. As illustrated, in implementations in which the split overtube 8602 includes a lobe portion, such as the lobe portion 8607, the ribs 8620A-8620C may be shaped to extend around the lobe portion.

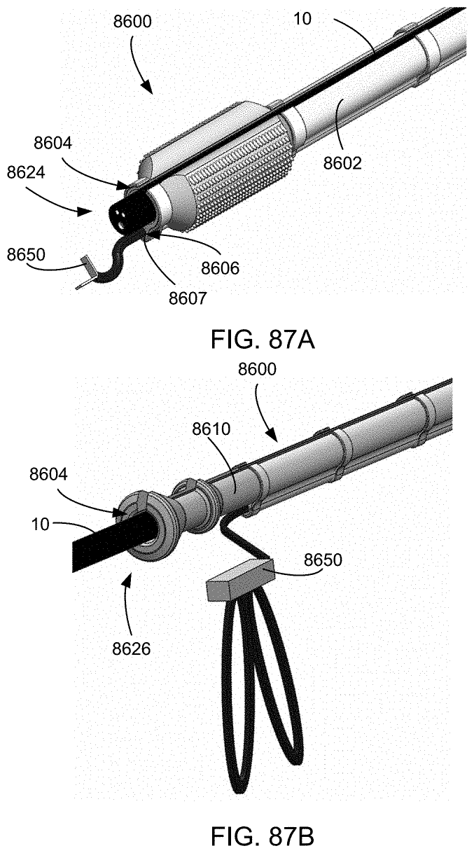

FIGS. 87A and 87B illustrate the split overtube assembly 8600 in use with each of an endoscope 10 and a tool 8650. More specifically, FIG. 87A is an isometric view of the distal portion 8624 of the split overtube assembly 8600 while FIG. 87B is an isometric view of a proximal portions 8626 of the split overtube assembly 8600. The tool 8650 is illustrated as a grasper-type tool and is disposed within the secondary lumen 8606; however, implementations of the present disclosure are not limited to use with any particular type of tool. Rather, any tool that is sized and shaped to be introduced through the secondary lumen 8606 may be used in conjunction with the overtube assemblies discussed herein.

Referring to FIG. 87B, the proximal portion 8626 of the split overtube assembly 8600 includes a handle 8610 through which the primary lumen 8604 extends and through which the endoscope 10 extends when coupled with the split overtube assembly 8600. As illustrated, the lobe portion 8607 of the split overtube 8602 terminates distal the handle 8610 such that the tool 8650 is disposed adjacent the handle 8610. Stated differently, the handle 8610 does not define any portion of the secondary lumen 8606. However, in other implementations, the handle 8610 may include a portion corresponding to the lobe portion 8607 such that the handle 8610 at least partially extends the secondary lumen 8606.

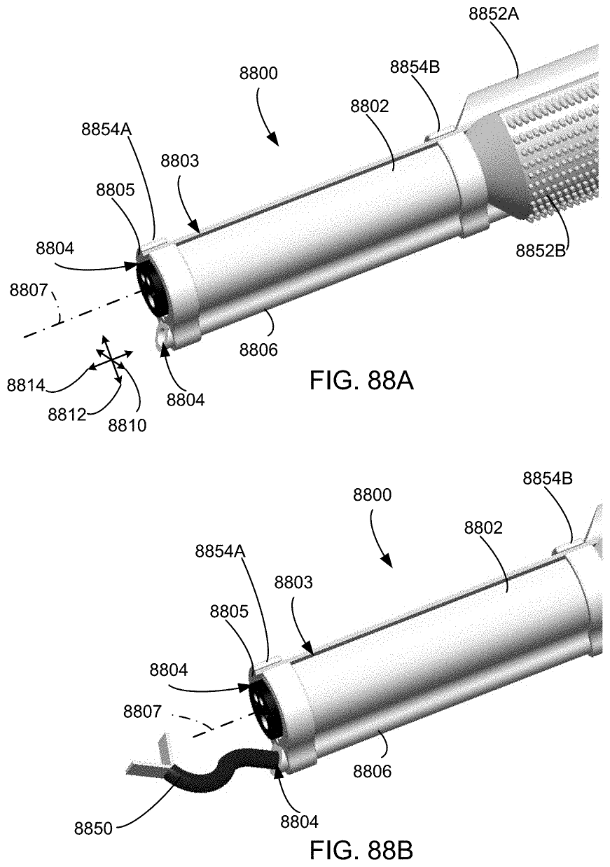

FIGS. 88A and 88B illustrate an alternative split overtube assembly 8800. Split overtube assembly 8800 includes a split overtube 8602 having a split 8803 and that defines a primary lumen 8804 in communication with split 8603. The split overtube 8802 further defines a secondary or working lumen 8806 extending along the length of the split overtube 8802 and substantially similar to secondary lumen 8606 discussed above in the context of FIGS. 86A-87B. FIG. 88A is an isometric view of a distal portion 8824 of split overtube assembly 8800 with an endoscope 10 inserted into primary lumen 8804 while FIG. 88B is an isometric view of distal portion 8824 of split overtube assembly 8800 further including a tool 8850 extended through secondary or working lumen 8806. Like previous implementations discussed herein, tool 8850 is illustrated as a grasper-type tool; however, implementations of the present disclosure are not limited to use with any particular type of tool. Rather, any tool that is sized and shaped to be introduced through secondary lumen 8806 may be used in conjunction with split overtube assembly 8800 and similar overtube assemblies.

As illustrated, for example, in FIG. 86A, secondary lumen 8606 of split overtube assembly 8600 may terminate at a distal end of split overtube 8602 such that a terminal end of secondary lumen 8606 extends substantially parallel to primary lumen 8604 (e.g., at zero degrees relative to a longitudinal axis of split overtube 8602). In contrast, and as illustrated in FIGS. 88A and 88B, secondary lumen 8806 may alternatively extend or otherwise terminate at a distal end 8805 of split overtube 8802 at a different angle relative to a longitudinal axis 8807 of primary lumen 8804. For example, secondary lumen 8806 of split overtube assembly 8800 is configured to terminate at an angle of approximately 30 degrees towards longitudinal axis 8807 (e.g., about axis 8810, which is substantially parallel to longitudinal axis 8807). Among other things, such angling of secondary lumen 8806 can provide direction and support of tool 8850 in a specific direction relevant to a particular application. Doing so can change the workspace of the tool and may allow for greater triangulation of the workspace relative to a camera or similar vision system that may be included in endoscope 10.

While illustrated as being angled at approximately 30 degrees towards longitudinal axis 8807, this disclosure contemplates that secondary lumen 8806 may be angled in any suitable direction and to any suitable degree for a given application. For example, secondary lumen 8806 may be angled toward or away from longitudinal axis 8807 (e.g., about axis 8810) at an angle other than 30 degrees. Secondary lumen 8806 may alternatively be angled such that it terminates/extends skewed relatively to longitudinal axis 8807 (e.g., about axis 8812, which is coplanar with and perpendicular to axis 8810). More generally, secondary lumen 8806 may terminate or extend at any angle from distal portion 8824 (e.g., any combination of rotation about axis 8810, axis 8812, or axis 8814 (which is perpendicular to each of axis 8810 and axis 8812)).

Split overtube assembly 8800 further illustrates that split overtube 8802 may extend distally beyond balloons 8852A, 8852B included in split overtube assembly 8800. Stated differently, balloons of split overtube assembly 8800 may be disposed proximal distal portion 8824 of split overtube 8802 such that split overtube 8802 protrudes distally beyond the balloons. Although the specific reasons for extending distal portion 8824 or split overtube 8802 beyond balloons 8852A, 8852B can vary, in at least certain implementations, doing so may permit articulate of distal portion 8824. For example, endoscope 10 may include an articulable end that can be curved in one or more directions. If endoscope 10 were to be coterminal with balloons 8852A, 8852B, balloons 8852A, 8852B may impede or preclude such articulation. In contrast, by extending 8802 beyond balloons 8852A 8852B, distal portion 8824 of split overtube 8802 may still protect and support endoscope 10 without substantially impeding its articulation.

In at least some implementations, reinforcing structures (e.g., split rings 8854A, 8854B) coupled to or integrated into split overtube 8802 may also extend or otherwise be disposed distally beyond balloons 8852A, 8852B to reinforce distal portion 8824 of split overtube 8802. However, in at least certain implementations, reinforcing structures may be omitted from distal portion 8824 of split overtube 8802 to facilitate articulation of endoscope 10. In still other implementations, primary lumen 8804 may have lower rigidity than other segments of split overtube 8802 to further facilitate articulation of endoscope 10. For example, distal portion 8824 may have a thinner wall or be formed from a less rigid material relative to proximal sections of split overtube 8802.

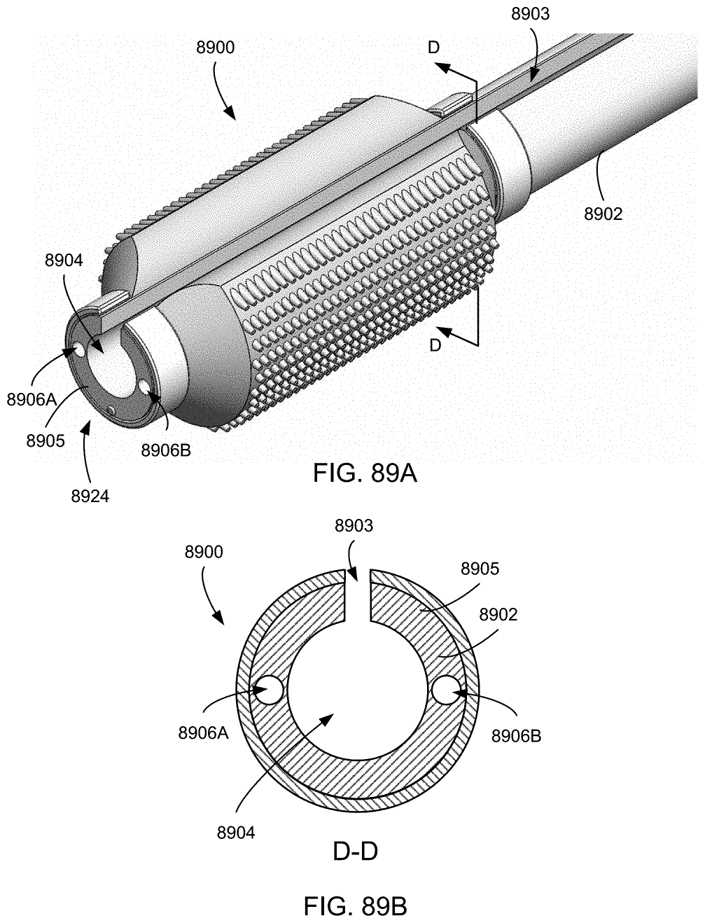

FIG. 89A is an isometric view of another example split overtube assembly 8900 and, in particular, an isometric view of a distal portion 8924 of the split overtube assembly 8900. The split overtube assembly 8900 includes a split overtube 8902 defining a split 8903. FIG. 89B is a cross-sectional view of the overtube assembly 8900 taken along lines D-D. The split overtube 8902 defines a primary lumen 8904 in communication with the split 8903 and for receiving an elongate medical device, such as an endoscope. The split overtube 8902 further defines a pair of secondary or working lumens 8906A, 8906B extending along the length of the split overtube 8902.

In contrast to the previously discussed example in which the secondary lumen 8606 was defined in a lobe portion 8607 protruding from a primary body 8608 of the split overtube 8602, the secondary lumens 8906A, 8906B are defined by a wall 8905 of the split overtube 8902 that further defines the primary lumen 8904. Although illustrated as being disposed on opposite sides of the primary lumen 8904, in other implementations, the secondary lumens 8906A, 8906B may be located elsewhere about the primary lumen 8904. Moreover, while two secondary lumens are illustrated, other implementations may include any suitable number of secondary lumens extending through the split overtube 8902.

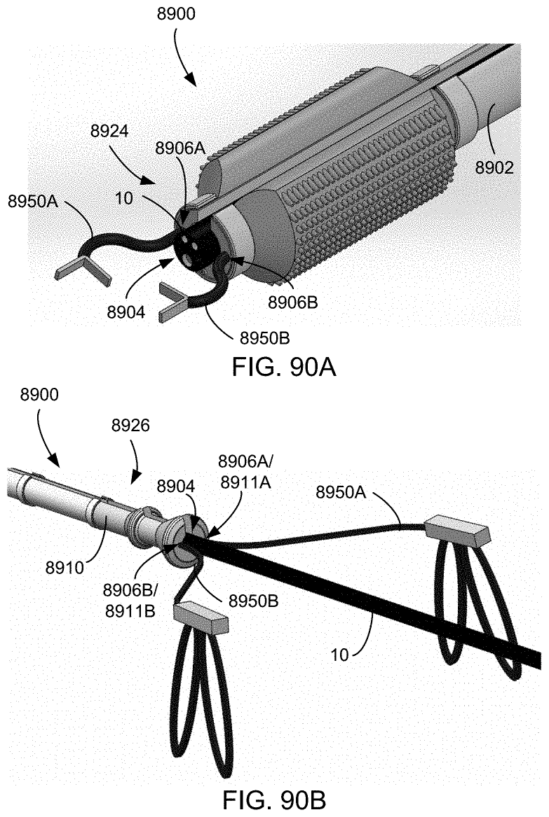

FIGS. 90A and 90B illustrate the split overtube assembly 8900 in use with each of an endoscope 10 and a pair of tools 8950A, 8950B. More specifically, FIG. 90A is an isometric view of the distal portion 8924 of the split overtube assembly 8900 while FIG. 90B is an isometric view of a proximal portion 8926 of the split overtube assembly 8900. The tools 8950A, 8950B are illustrated as grasper-type tools and are disposed within the secondary lumens 8906A, 8906B, respectively; however, implementations of the present disclosure are not limited to use with any particular type of tool. Rather, any the tool that is sized and shaped to be introduced through either of the secondary lumens 8906A, 8906B may be used.

Referring to FIG. 90B, the proximal portion 8926 of the split overtube assembly 8900 includes a handle 8910 through which the primary lumen 8904 extends and through which the endoscope 10 extends when coupled with the split overtube assembly 8900. As illustrated, each of the tools 8950A, 8950B extend through the handle 8910 and, more specifically, through extensions 8911A, 8911B of the secondary lumens 8906A, 8906B defined by the handle 8910. Nevertheless, it should be appreciated that in other implementations, the secondary lumens may instead terminate at a proximal end of the split overtube 8902 such that the tools 8950A, 8950B are disposed adjacent the handle 8910. In other implementations, the handle 8910 may alternatively define extensions in communication with the secondary lumens 8906A, 8906B but that open laterally at a location distal the proximal extent of the handle 8910.

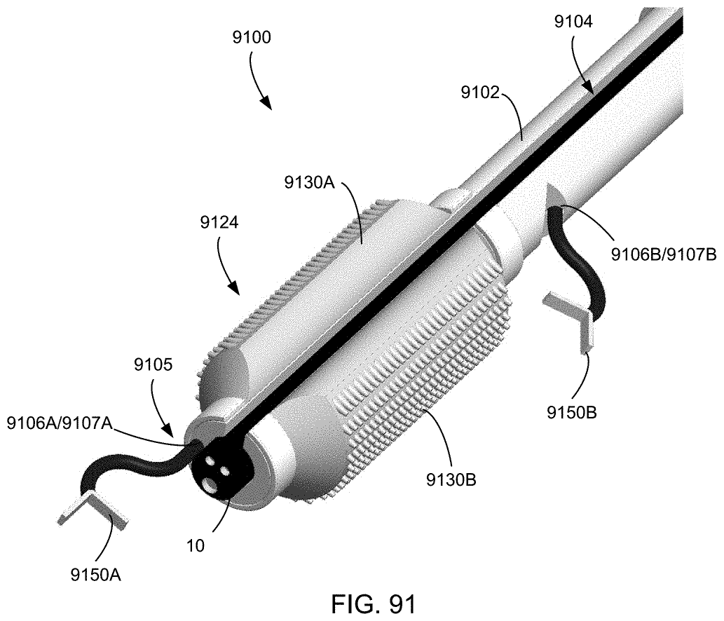

Secondary lumens of the previously discussed embodiments generally extended to and terminated at a distal end of the split overtube; however, in other implementations, however, secondary lumens may terminate at other locations along the length of the split overtube. FIG. 91 is an isometric view of a split overtube assembly 9100 illustrating an example of such embodiments. More specifically, FIG. 91 illustrates a distal portion 9124 of split overtube assembly 9100. Split overtube assembly 9100 includes a split overtube 9102 defining a primary lumen 9104 within which an elongate tool, such as an endoscope 10, may be inserted.

Split overtube 9102 further includes a pair of secondary lumens 9106A, 9106B that end in respective ports 9107A, 9107B. FIG. 91 illustrates each secondary lumen 9106A, 9106B with a respective tool 9150A, 9150B (e.g., gripper tools) extending from its respective port 9107A, 9107B.

As shown in FIG. 91, second lumen 9106A conforms to previously disclosed secondary lumens that extend along the length of 9102 such that respective port 9107A of secondary lumen 9106A opens at a distal end 9105 of split overtube 9102. In contrast, secondary lumen 9106B is illustrated as extending only partially along the length of split overtube 9102 such that port 9107B is located at proximal distal end 9105. In the specific illustrated example, port 9107B is located proximal balloons 9130A, 9130B. Accordingly, following insertion of split overtube assembly 9100 and anchoring of split overtube assembly 9100 by inflating proximal balloons 9130A, 9130B, secondary lumen 9106A may be used to access a first workspace distal balloons 9130A, 9310B while secondary lumen 9106B may be used to access a second workspace proximal balloons 9130A, 9130B.

The specific configuration illustrated in FIG. 91 is intended only as an example of split overtube assemblies with proximally located secondary lumen ports. More generally, implementations of this disclosure may include one or more secondary lumens with proximally located ports with or without one or more secondary lumens with distal ports. Similarly, while port 9107B of split overtube assembly 9100 is disposed proximal balloons 9130A, 9130B, in other implementations, split overtube 9102 may extend beyond balloons 9130A, 9130B such that port 9107B may be disposed between balloons 9130A, 9130B and a distal end of split overtube 9102. In still other implementations, balloons 9130A, 9130B may be omitted from split overtube 9102.

As discussed in the context of FIG. 88, distally located ports of secondary lumens may be perpendicular to a longitudinal axis of the primary lumen/split overtube or may be angled relative to the longitudinal axis of the primary lumen/split overtube. Proximally located ports of secondary lumens may similarly be perpendicular or angled relative to the longitudinal axis of the primary lumen/split overtube. For example, port 9107B is illustrated in FIG. 91 as being directed away from the longitudinal axis of split overtube 9102 by approximately 45 degrees; however other angles and directions of port 9107B are within the scope of this disclosure.

Secondary lumens included throughout this disclosure can be formed in a number of ways including, but not limited to, extrusions and lay-ups. In certain embodiments, the secondary lumens can be lined or coated with PTFE or other materials to reduce friction and facilitate insertion of tools. Secondary lumens may also be reinforced with coiled wire, braids, or other materials to prevent collapse or bucking when the split overtube is flexed, bent around corners, or similarly deformed. Also, such reinforcement may be used to keep the secondary lumens in an open state when no tool is present and to keep the secondary lumen in place so that tools can be advanced and rotated. Although secondary lumen size may vary, in at least some implementations, secondary lumens may have a maximum cross-sectional measurement from and including about 0.5 mm to and including about 15.0. Also, while generally illustrated as having a circular cross-section, secondary lumens may have any suitable cross-sectional shape.

Split Overtubes Including Insertion Areas

Conventionally, overtubes and overtube assemblies are coupled to elongate medical devices by inserting the medical devices through the overtube or otherwise sliding the overtube onto the medical device longitudinally. Notably, this conventional approach has the distinct disadvantage of requiring access to either a proximal or distal end of the medical device. In general, the proximal end of the medical device (e.g., an endoscope) includes hubs, ports, and various other structures and mechanisms such that it is not possible to dispose an overtube onto the medical device from the proximal end. Disposing the overtube onto the elongate medical device from the distal end is also disadvantageous to the extent that the elongate medical device cannot be disposed within the patient when coupling the elongate medical device and the overtube. Stated differently, in the event an overtube is required during the course of an operation, the overtube must be coupled to the elongate medical device at the outset of the operation or otherwise requires that the elongate medical device be fully removed from the patient, resulting in a longer operation with increased risks of various complications.

In contrast to the conventional approach described above, split overtubes according to the present disclosure are coupled to elongate medical devices by inserting the elongate medical device through a split defined in the overtube and extending along the length of the overtube. The split allows the overtube to be coupled to the elongate medical device laterally and, as a result, the overtube may be readily coupled to the elongate medical device without requiring removal of a distal portion of the elongate medical device from the patient. This technique permits the overtube to be implemented as- and when-needed. As another advantage, the split enables decoupling of the overtube and the elongate medical device such that the overtube may function as a sheath or guide that permits removal or swapping of the elongate medical devices.

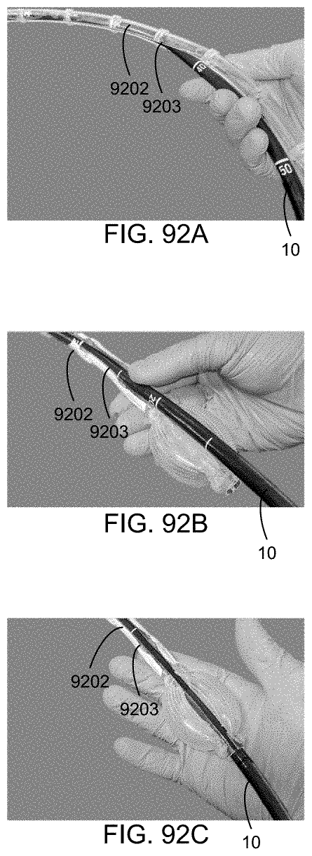

FIGS. 92A-92C are a series of photographs illustrating an example approach of coupling a split overtube 9202 according to the present disclosure to an elongate medical device 10, such as an endoscope. As illustrated, a physician (or other medical personnel) couples the split overtube 9202 to the elongate medical device 10 by laterally passing the medical device 10 through a split 9203 extending along the split overtube 9202.

In at least some implementations, this coupling process may include inserting a first portion of the elongate medical device 10 into the split overtube 9202 at an intermediate location of the split overtube 9202. Once the initial portion is inserted, the physician may work either proximally or distally from the initial insertion location, gradually inserting more of the medical device 10 into the split overtube 9202. After reaching a first extent of the split overtube 9202, the physician may work from the initial insertion location in the opposite direction until the split overtube 9202 is fully disposed about the medical device 10. In other implementations, the medical device 10 may be inserted at a first end of the split overtube 9202 and the split overtube 9202 may be gradually worked onto the medical device 10 in a direction from the initial insertion location to an end of the split overtube 9202 opposite the insertion location.

As shown in FIG. 92A, in at least certain implementations of the present disclosure, the split overtube 9202 may be configured for one-handed coupling to the medical device 10. In general, such coupling involves holding the split overtube 9202 in the hand such that the split 9203 is directed outwardly from the palm. The fingers may then be used to press the medical device 10 through the split 9203 and into the split overtube 9202, with the palm providing counterforce/resistance to the force applied by the fingers. In other implementations, the split overtube 9202 may be held with the fingers opposite the split 9203 such that the thumb may be used to press the medical device 10 through the split 9203.

Regardless of how the medical device 10 is inserted through the split 9203, the split overtube 9202 may include areas of reinforcement and/or weakening that facilitate insertion of the medical device 10 into the split overtube 9202. For example, in at least certain implementations, a portion of the split overtube 9202 opposite the split 9203 may be reinforced to provide additional leverage while pressing the medical device 10 through the split 9203.

Alternatively, or in addition to such reinforcement, portions of the split overtube 9202 adjacent the split 9203 may be weakened relative to other portions of the split overtube 9202 such that the weakened portions provide less resistance to insertion of the medical device 10. As described below in further detail, in at least certain implementations, such reinforcement and/or weakening may be used to form an insertion location of the split overtube 9202 where an initial portion of the medical device 10 is inserted into the split overtube 9202. With the initial portion of the medical device 10 inserted, the physician may work outwardly from the insertion location or otherwise along the split overtube 9202 from the insertion location to complete insertion of the medical device 10 into the split overtube 9202.

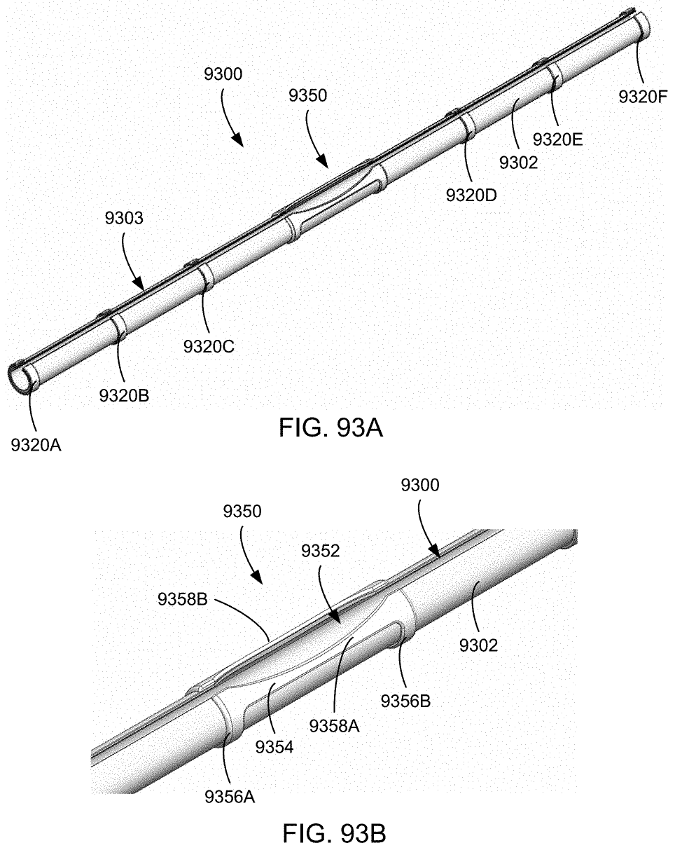

FIGS. 93A and 93B illustrate an example split overtube 9300 including selective reinforcement. More specifically, FIG. 93A is an isometric view of the split overtube 9300 while FIG. 93B is a detailed view of a reinforced portion of the split overtube 9300.

The split overtube 9300 includes a flexible body 9302 defining a longitudinal split 9303 and along which a series of optional reinforcing ribs 9320A-9320F are distributed. As illustrated in FIG. 93B, the split overtube 9300 further includes an insertion feature 9350 that generally forms an initial insertion section of the flexible tubular body 9302.

As illustrated, the insertion feature 9350 facilitates insertion of a medical device into the split overtube 9300 in at least two ways. First, the insertion feature 9350 includes a cutout 9352 or similar widening of the split 9303 in the area of the insertion feature 9350, which locally reduces resistance to insertion of the elongate medical device through the split 9303. Second, the insertion feature 9350 includes a reinforcement structure 9354 that strengthens/reinforces the flexible body 9302 in the area of the insertion feature 9350 to provide additional leverage when inserting the elongate medical device. In the specific example illustrated in FIG. 93B, the reinforcement structure 9354 is in the form of two ribs 9356A, 9356B (generally similar to reinforcing ribs 9320A-9320F) that are coupled together by webs 9358A, 9358B. By virtue of being coupled together, the ribs 9356A, 9356B provide increased reinforcement (relative to ribs 9320A-9320F) around the cutout 9352. As previously mentioned, such reinforcement provides additional leverage when inserting an elongate medical device into the split overtube 9300. Accordingly, the insertion feature 9350 provides each of reduced resistance and improved leverage for facilitating insertion of an elongate medical device into the split overtube 9300.

In the foregoing example, the insertion feature 9350 both lowered resistance to insertion of the elongate medical device into the split overtube while also providing additional leverage to facilitate such insertion. In other implementations, insertion features according to the present disclosure may provide only one of lowered resistance to insertion of the elongate medical device or additional leverage.

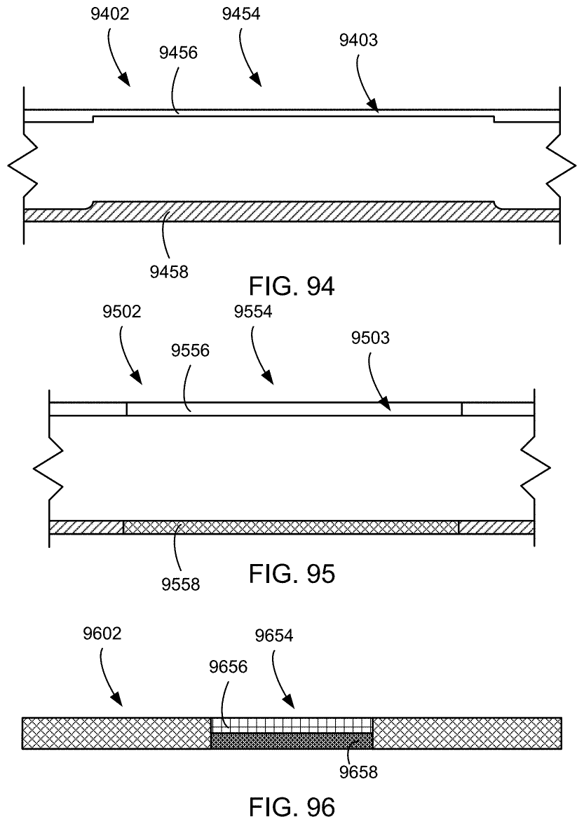

Insertion feature 9350 illustrated in FIGS. 93A and 93B is just one example of an insertion feature that may be used to facilitate insertion of an elongate medical device into a split overtube. In certain implementations, insertion features may be provided by locally altering characteristics of the flexible tubular body. As a first example, FIG. 94 is a cross-sectional view of a flexible tubular body 9402 defining a split 9403 in which an insertion feature 9454 is formed by altering the wall thickness of the flexible tubular body 9402. More specifically, the insertion feature 9454 includes a thin wall portion 9456 disposed adjacent the split 9403 having a wall thickness that is less than other portions of the flexible tubular body 9402 adjacent the split 9403. As a result, the thin wall portion 9456 provides less resistance to insertion of an elongate medical device through the split 9403. The insertion feature 9454 further includes a thick wall portion 9458 disposed opposite the split 9403. The thick wall portion 9458 reinforces the flexible tubular body 9402 opposite the thin wall portion 9456, thereby providing a leverage point for use during insertion of an elongate medical device through the split 9403.

FIG. 95 is a cross-sectional view of a second flexible tubular body 9502 defining a split 9503 in which an insertion feature 9554 is formed by altering the material of the flexible tubular body 9502. More specifically, the insertion feature 9554 includes a low resilience wall portion 9556 disposed adjacent the split 9503 formed of a material that is generally less resilient (e.g., more flexible) than other portions of the flexible tubular body 9502 adjacent the split 9503. As a result, the low resilience wall portion 9556 provides less resistance to insertion of an elongate medical device through the split 9503. The insertion feature 9554 further includes a high resilience wall portion 9558 disposed opposite the split 9503 and formed of a material that is general more resilient (e.g., less flexible) than other portions of the flexible tubular body 9502. As a result, the high resilience portion 9558 reinforces the flexible tubular body 9502, providing a leverage point for use during insertion of an elongate medical device through the split 9503.

FIG. 96 is an elevation view (e.g., a non-cross-sectional view) of another tubular body 9602 defining a split (obstructed in view) in which an insertion feature 9654 is formed by altering an embedded reinforcement (e.g., a braid, a weave, fibers, etc.) of the flexible tubular body 9602. More specifically, the insertion feature 9654 includes a low reinforcement wall portion 9656 disposed adjacent the split and within which no or relatively low reinforcement is embedded, the reinforcement being low relative to portions of the flexible tubular body 9602 not included in the insertion feature 9654. For example, the low reinforcement wall portion 9656 may have a relatively loose/low density braid or weave or may have a relatively low density of reinforcing fibers or nor reinforcing fibers embedded therein. As a result, the low reinforcement wall portion 9656 provides less resistance to insertion of an elongate medical device through the split. The insertion feature 9654 further includes a high reinforcement wall portion 9658 disposed opposite the split. In contrast to the low reinforcement wall portion 9656, the high reinforcement wall portion 9658 generally includes embedded reinforcement that provide greater reinforcement than that found in portions of the flexible tubular body 9602 not included in the insertion feature 9654. For example, the high reinforcement wall portion 9658 may have a high density or higher strength braid, weave, or fiber distribution as compared to other portions of the flexible tubular body 9602. Accordingly, the high reinforcement wall portion 9658 reinforces the flexible tubular body 9602, thereby providing a leverage point for use during insertion of an elongate medical device through the split.

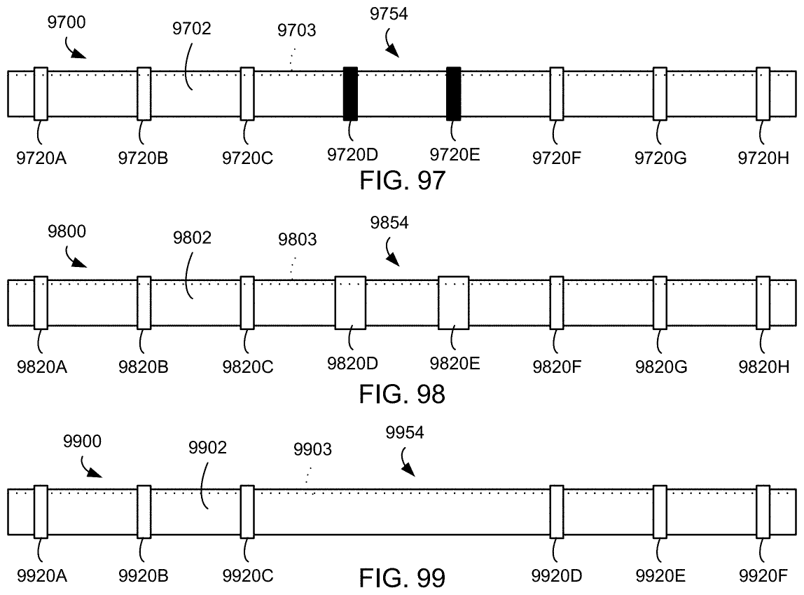

Insertion features according to the present disclosure may also be formed by modifying characteristics of reinforcing structures, such as ribs, that may be integrally formed with the flexible tubular body of the overtube. Examples of such implementations are illustrated in FIGS. 97-101BC and are discussed below in further detail.

Referring first to FIG. 97, an overtube 9700 is illustrated. The overtube 9700 includes a flexible tubular body 9702 defining a split 9703. The overtube 9700 further includes reinforcing structures distributed along its length. Although other reinforcing structures may be used, the reinforcing structures of the overtube 9700 include a series of ribs 9720A-9720H distributed along the flexible tubular body 9702. As discussed herein, the ribs 9720A-9720H generally include a rib split or similar opening that is aligned with the split 9703 to permit insertion of an elongate medical device into the flexible tubular body 9702.

In the example of FIG. 97, the ribs 9720A-9720H are illustrated as being formed of two different materials. More specifically, ribs 9720A-9720C and ribs 9720F-9720H are formed of a first material while ribs 9720D and 9720E are formed of a second material with the difference in material resulting in an insertion feature 9754 that facilitates insertion of an elongate medical device into the overtube 9700. In certain implementations, the first material may be less rigid than the second material such that the flexible tubular body 9702 is locally reinforce by ribs 9720D and 9720E at the insertion feature 9754. Doing so may facilitate additional leverage when inserting an elongate medical device into the flexible tubular body 9702 in the area of the insertion feature 9754. In other implementations, the first material may be more rigid than the second material such that the ribs 9720D and 9720E provides less resistance to insertion of the elongate medical device through the split 9703 in the area of the insertion feature 9754. In still other implementations, the ribs 9720D and 9720E may include a first portion disposed substantially opposite the split 9703 and formed of a more rigid material than the other ribs and second portions disposed adjacent the split 9703 and formed of a less rigid material than the other ribs. In such implementations, the ribs 9720D and 9720E would reduce resistance to insertion of the elongate medical device into the flexible tubular body 9702 while also providing a leverage point to facilitate insertion of the elongate medical device into the flexible tubular body 9702.

FIG. 98 is of another overtube 9800 that includes a flexible tubular body 9802 defining a split 9803. The overtube 9800 includes a series of ribs 9820A-9820H distributed along the flexible tubular body 9802. The ribs 9820A-9820H are illustrated as having variable dimensions. More specifically, ribs 9820A-9820C and ribs 9820F-9820H have a first width while ribs 9820D and 9820E have a second width, the ribs 9820D and 9820E defining an insertion feature 9854. In general, the increased width of the ribs 9820D and 9820E relative to the width of ribs 9820A-9820C and 9820F-H provides relatively greater reinforcement in the area of the insertion feature 9854, thereby providing increased leverage at the insertion feature 9854. In other implementations, ribs 9820D and 9820E may have a smaller width than ribs 9820A-9820C and 9820F-H, thereby providing less resistance to insertion of an elongate medical device at the insertion feature 9854. In still other implementations, the ribs 9820D and 9820E may include a first portion disposed substantially opposite the split 9803 and having a width greater than the other ribs and second portions disposed adjacent the split 9803 and having a width less than the other ribs. In such implementations, the ribs 9820D and 9820E would reduce resistance to insertion of the elongate medical device into the flexible tubular body 9802 while also providing a leverage point to facilitate insertion.

Although the example of FIG. 98 varies the width of the ribs 9820D and 9820E to define the insertion feature 9854, other implementations of the present disclosure may alter other dimensional characteristics of the ribs to provide similar effects. For example, and without limitation, in at least some implementations, variable rib thickness may instead be used to define the insertion feature.

FIG. 99 illustrates yet another overtube 9900 that includes a flexible tubular body 9902 defining a split 9903. The overtube 9900 includes a series of ribs 9920A-9920F distributed along the flexible tubular body 9902. The ribs 9920A-9920F are illustrated as having variable spacing. More specifically, the distance between ribs 9920A-C and between ribs 9920D-9920F is illustrated as a distance while the distance between ribs 9920C and 9920D is illustrated as having a second, greater distance. In general, the increased distance between ribs 9920C and 9920D relative to the distance between other pairs of adjacent ribs defines the insertion feature 9954 as the gap between ribs 9920C and 9920D generally provides less resistance to insertion of an elongate medical device through the split 9903. In other implementations, ribs 9920C and 9920D may be spaced more closely together relative to the spacing of other ribs of the overtube 9900, thereby providing additional reinforcement along the corresponding length of the flexible tubular body 9902 and a leverage point for use during insertion of an elongate medical device into the overtube 9900.

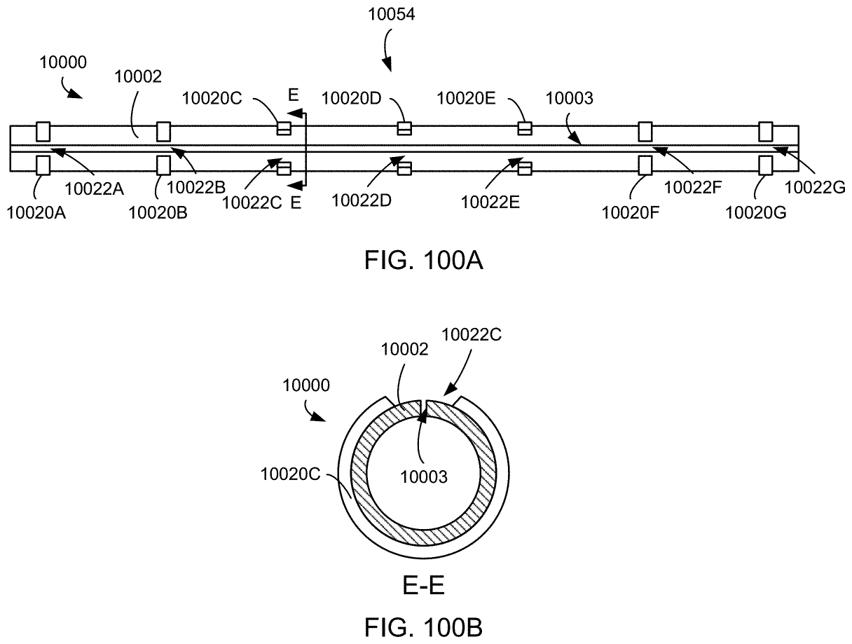

FIG. 100A illustrates another overtube 10000 that includes a flexible tubular body 10002 defining a split 10003. The overtube 10000 includes a series of ribs 10020A-10020G distributed along the flexible tubular body 10002. The ribs 10020A-10020G each define a respective rib split 10022A-10022G that is generally aligned with the split 10003 of the flexible tubular body 10002. In the implementation of FIG. 100A, resistance to insertion of an elongate medical device is controlled by varying the width of the rib splits. More specifically, rib splits 10022A, 10022B, 10022F, and 10022G are illustrated as having a first width while rib splits 10022C-E are illustrated as having a second width greater than the first width. As a result, ribs 10020C-E define an insertion feature 10054 in which resistance to insertion of an elongate medical device is reduced.

As further illustrated in FIG. 100B, which is a cross-sectional view taken along E-E, ribs 10020C-E further include guide features to facilitate insertion of an elongate medical device. More specifically, FIG. 100B includes rib 10020C and corresponding rib split 10022C. As shown, the portions of rib 10020C adjacent rib split 10022C may be sloped, chamfered, filleted, or otherwise formed to provide a gradual transition toward rib split 10022C. Such a transition helps to guide the elongate medical device during insertion while also providing a wedge-like interface that helps to expand rib 10020C while the elongate medical device is being inserted.

The foregoing discussion describes various techniques and approaches for providing controlled reinforcement of split overtubes. As discussed, such controlled reinforcement may be used to reduce resistance to an elongate medical device being inserted into the split overtube and/or to provide increased leverage. Accordingly, implementations of the present disclosure are not limited to the specific examples provided. Moreover, any of the examples disclosed herein may be combined with each other.

Sheet-Based Manufacturing of Split Overtubes

Split overtubes according to the present disclosure may be manufactured in various ways. In at least certain implementations, a sheet-based approach may be used in which layers of the split overtube are disposed on top of each other and subsequently formed into a tubular shape. More specifically, a strip is formed that defines a longitudinal axis and is subsequently formed into a split tube by curving the strip about the longitudinal axis. The strip may include reinforcements (e.g., ribs) such that, when formed into the split tube, the reinforcements similarly curve about the longitudinal axis.

FIGS. 101A-101C illustrate a first example manufacturing method for a split overtube. Referring to FIG. 101A, a reinforced strip 10102 including laterally extending reinforcing members (e.g., rib 10120) is aligned with and coupled to a substrate strip 10104, resulting in a layered strip 10106 (shown in FIG. 101B). Laterally extending reinforcing members may be integrally formed with the reinforced strip 10102 or may be coupled to the reinforced strip 10102. Coupling of the reinforced strip 10102 to the substrate strip 10104 may be achieved in various ways including, but not limited to, reflow, thermal bonding, thermal welding, adhesives, and the like. Subsequent to forming the layered strip 10106, the layered strip 10106 may be formed (e.g., thermoformed) into a tubular body 10108 having an open tubular shape and including a split 10103, as illustrated in FIG. 101C.

Forming the tubular body 10108 generally includes curving the layered strip 10106 about a longitudinal axis of the tubular body 10108. As illustrated in FIG. 101C, such forming may result in the reinforcing members (e.g., rib 10120) being disposed on an exterior of the flexible tubular body 10108. Alternatively, by curving the layered strip 10106 in an opposite direction, the reinforcing members may be disposed on an interior surface of the flexible tubular body 10108. In still other implementations, the layered strip 10106 may include a third strip (not shown) such that the reinforced strip 10102 is sandwiched between the substrate strip 10104 and the third strip. In such implementations, the reinforcing members would be embedded within the flexible tubular body 10108.

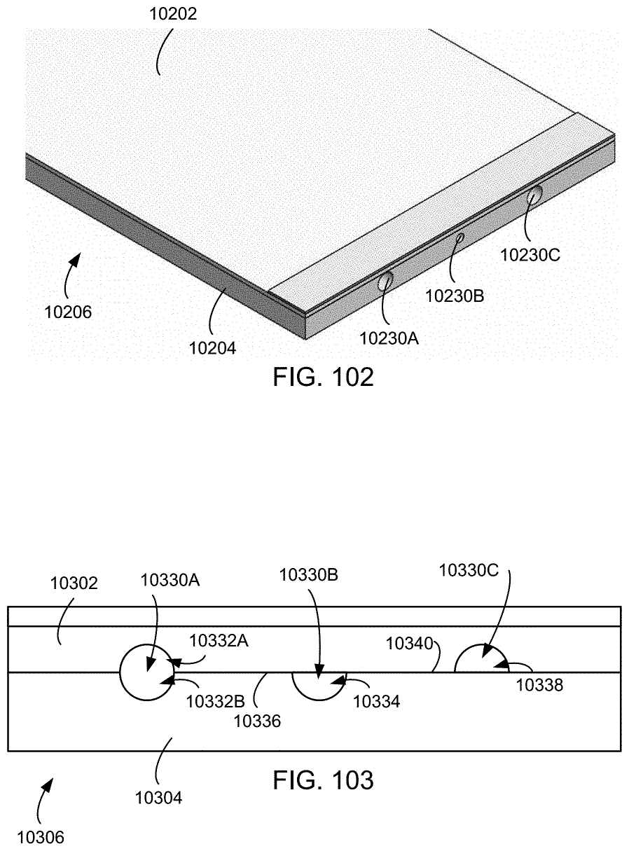

In certain implementations, longitudinal channels (e.g., working or fluid channels) may be defined within the layered strip. For example, FIG. 102 illustrates a layered strip 10206 including a reinforced strip 10202 coupled to a substrate strip 10204. As illustrated, the substrate strip 10204 defines three longitudinal channels 10230A-10230C extending through the substrate strip 10204. In at least certain implementations, the substrate strip 10204 may be formed by an extrusion or similar process to define the channels 10230A-10230C within the substrate strip 10204.

FIG. 103 illustrates an alternative layered strip 10306 including longitudinal channels 10330A-10330C. More specifically, the layered strip 10306 includes a reinforced strip 10302 coupled to a substrate strip 10304. As illustrated, channels extending through the layered strip 10306 may be formed by grooves or similar structures extending along adjacent layers of the layered strip 10306. For example, channel 10330A is defined by each of a first groove 10332A of the reinforced strip 10302 and a second groove 10332B of the substrate strip 10304. Channel 10330B, on the other hand, is defined by a groove 10334 of the substrate strip 10304 and a bottom surface 10336 of the reinforced strip 10302. Similarly, channel 10330C is defined by a groove 10338 of the reinforced strip 10302 and an interior surface 10340 of the substrate strip 10304.

The foregoing examples in which channels are defined by each of the reinforced strip 10302 and the substrate strip 10304 are provided merely as examples of how channels may be formed in split overtubes according to the present disclosure. More generally, implementations of the present disclosure may include channels defined by one or more layers of the layered strip. Also, while generally referred to herein as extending longitudinally, channels defined through the layered strip are not limited to extending in a purely longitudinal direction. Rather, the foregoing techniques may be used to form channels that extend one or both of circumferentially and longitudinally through the layered strip.

While air channels and secondary lumens of split overtubes according to the present disclosure may be formed by grooves or similar channels formed into layers of the split overtube, in other implementations, air channels and/or secondary lumens may alternatively be formed by disposing tubular structures between adjacent layers of the split overtube. For example, lengths of braided tube or similar tubular components may be disposed between adjacent layers of the split overtube such that when the layers are bonded and formed into the final split overtube shape, the tubular structures are embedded between layers of the split overtube and form passages through the split overtube.

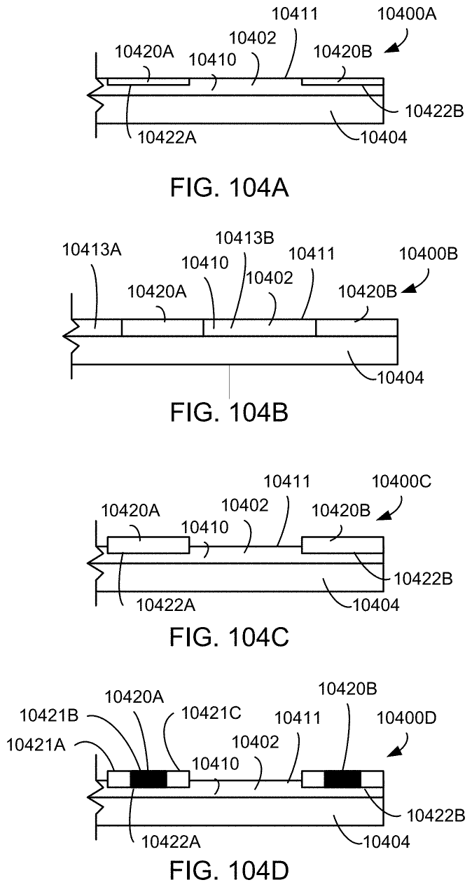

FIGS. 104A-104D illustrate various implementations of reinforced layers according to the present disclosure. Referring first to FIG. 104A, an elevation view of a layered strip 10400A is provided. The layered strip 10400A includes a reinforced strip 10402 coupled to a substrate layer 10404. As illustrated, the reinforced strip 10402 includes a base 10410 to which reinforcement structures, such as ribs 10420A, 10420B, are coupled. More specifically, the base 10410 defines recesses, e.g., recesses 10422A, 10422B, within which the ribs 10420A, 10420B are received such that the ribs 10420A, 10420B are flush with an outer surface 10411 of the base 10410.

FIG. 104B, an elevation view of a layered strip 10400B is provided. The layered strip 10400B includes a reinforced strip 10402 coupled to a substrate layer 10404. As illustrated, the reinforced strip 10402 includes reinforcement structures, such as ribs 10420A, 10420B that fully extend through the reinforced strip 10402. Stated differently, the reinforced strip 10402 is formed by the ribs 10420A, 10420B and base segments, such as base segments 10413A, 10413B, disposed between the ribs 10420A, 10420B. In certain implementations, the reinforced strip 10402 may be preformed by longitudinally coupling the ribs 10420A, 10420B and the base segments 10413A, 10413B. The resulting assembled layer may then be coupled to the substrate layer 10404 In other implementations, the ribs 10420A, 10420B and the base segments 10413A, 10413B may be individually disposed onto and coupled to the substrate layer 10404.

FIG. 104C is an elevation view of another layered strip 10400C. The layered strip 10400C includes a reinforced strip 10402 coupled to a substrate layer 10404. As illustrated, the reinforced strip 10402 includes a base 10410 to which reinforcement structures, such as ribs 10420A, 10420B, are coupled. Similar to the layered strip 10400A of FIG. 104A, the base 10410 defines recesses, e.g., recesses 10422A, 10422B, within which the ribs 10420A, 10420B are received. However, in contrast to the layered strip 10400A, the recesses 10422A, 10422B of the layered strip 10400C and ribs 10420A, 10420B are configured such that the ribs 10420A, 10420B protrude from an outer surface 10411 of the base 10410.

FIG. 104D is an elevation view of another layered strip 10400D. The layered strip 10400D includes a reinforced strip 10402 coupled to a substrate layer 10404. As illustrated, the reinforced strip 10402 includes a base 10410 to which reinforcement structures, such as ribs 10420A, 10420B, are coupled. Similar to the layered strip 10400C of FIG. 104C, the base 10410 defines recesses, e.g., recesses 10422A, 10422B, within which the ribs 10420A, 10420B are received such that the ribs 10420A, 10420B protrude from an outer surface 10411 of the base 10410. As illustrated, the ribs 10420A, 10420B are formed from multiple materials. For example, rib 10420A includes multiple segments 10421A-10421C with segments 10421A and 10421C formed from a first material and segment 10421B formed of a second, different material. In certain implementations, rib 10420A may be preformed by coupling segments 10421A-10421C together before being disposed in the recess 10422A. Alternatively, rib 10420A may be formed by separately disposing and coupling the segments 10421A-10421C into the recess 10422A.

The foregoing configurations of the reinforced layer are provided merely as non-limiting examples and this disclosure is not limited to the specific configurations illustrated. Moreover, any of the foregoing concepts may be combined together and be within the scope of this disclosure. For example, in certain implementations, a multi-segment reinforcement structure (such as the ribs illustrated in FIG. 104D) may be configured to be flush with an outer surface of the base (such as the ribs illustrates in FIG. 104A).

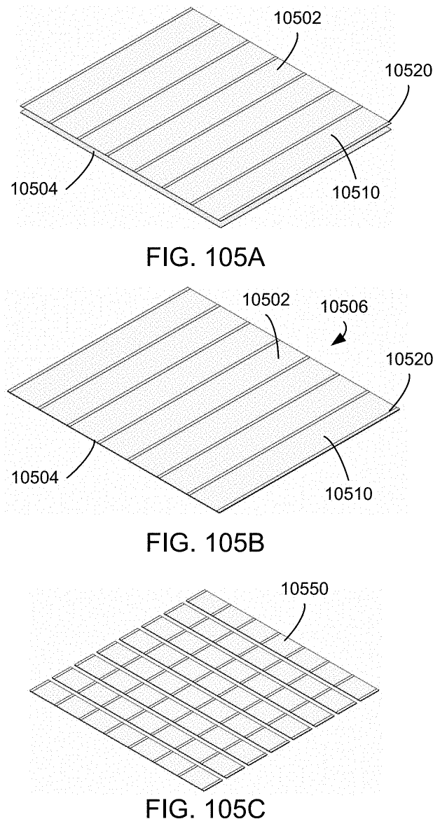

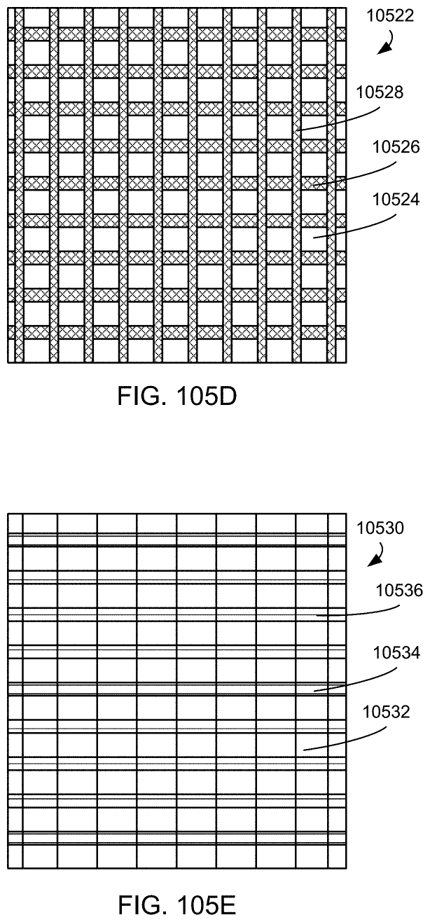

FIGS. 105A-105C illustrate an alternative manufacturing method for producing split overtubes according to the present disclosure. More specifically, the approach illustrated in FIGS. 105A-105C facilitates efficient production of multiple split overtubes by using a sheet-based construction technique.

Referring first to FIG. 105A, the manufacturing technique generally includes forming or otherwise obtaining each of a reinforced sheet 10502 and a substrate sheet 10504. Similar to the reinforced strips discussed above, the reinforced sheet 10502 may include multiple, laterally extending reinforcement structures (such as rib 10520). The reinforced sheet 10502 and the substrate sheet 10504 are coupled together to form a layered sheet 10506, as illustrated in FIG. 105B.

In at least certain implementations, the reinforced sheet 10502 may include a base 10510 into which the reinforcement structures are inserted or otherwise coupled. Accordingly, in certain implementations, forming the layered sheet 10506 may include first coupling the base 10510 to the substrate sheet 10504 and subsequently coupling the reinforcement structures to the base 10510. In still other implementations, the reinforced sheet 10502 may be formed from multiple segments and reinforcement structures. In such implementations, the layered sheet 10506 may be formed by sequentially disposing and coupling base segments and reinforcement structures to the substrate sheet 10504.

In certain implementations, various channels may be defined through the layered sheet 10506. As previously discussed in the context of FIGS. 102 and 103, such channels may be defined entirely within a particular layer of the layered sheet 10506 or may be collectively defined by more than one layer of the layered sheet 10506. Also, channels defined within the layered sheet 10506 may extend either or both of laterally and longitudinally through the layered sheet 10506.

Following assembly of the layered sheet 10506, the layered sheet 10506 may be cut into multiple strips, such as strip 10550 as illustrated in FIG. 105C. Similar to the layered strip 10106 illustrated in FIG. 101B, each strip may be subsequently curved into a tubular shape, e.g., using a thermoforming process.

FIG. 105D is a plan view of another layered sheet 10522 in accordance with the present disclosure. As previously discussed in the context of FIG. 78B, certain implementations of the present disclosure may include reinforcing structures in the form of circumferentially extending bands of braided or similarly reinforced materials. Such implementations may further include longitudinally extending bands or reinforcing structures that are integrated with, coupled to, or otherwise disposed adjacent the circumferentially extending bands to provide additional support. In accordance with such examples, a layered sheet 10522 may be formed using a substrate layer 10524 onto which a first layer 10526 including laterally extending bands of braided material and an optional second layer 10528 including longitudinally extending bands of braided material may be disposed. The substrate layer 10524 may then be fused or otherwise coupled to the first layer 10526 and the second layer 10528, thereby forming the layered sheet 10522, which may subsequently be cut into longitudinal strips. The strips may then be formed into tubular shapes that, as a result of the first layer 10526, include circumferential bands of reinforced material, as discussed above.

Notably, the braided material may be incorporated into the layered sheet 10522 in various ways. For example, as noted above, braided material may be disposed in separate layers, with each layer including braided material extending in different directions. In other implementations, the layered sheet 10522 may include alternating strips of a substrate material and a braided material. The alternating strips may then be coupled together (e.g., by fusing the strips together or by applying a second layer) to form a single layer including each of the substrate material and the laterally extending braided material. In other implementations, the layers including the laterally and longitudinally extending bands of braided material (e.g., the first layer 10526 and the second layer 10528, respectively) may be combined into a single layer. In still other implementations, each band of laterally extending material and longitudinally extending material may be separate and distinct as opposed to being formed with other similar bands into a single layer. The individual strips of material may then be laid onto a substrate sheet and coupled to the substrate sheet, e.g., by fusing the strips to the substrate or applying an additional layer such that the bands are sandwiched between the substrate and the additional layer.

FIG. 105E is a plan view of another layered sheet 10530 in accordance with the present disclosure. As previously discussed in the context of FIG. 78C, certain implementations of the present disclosure may include reinforcing structures in the form of wire or wire coils. In accordance with such examples, the layered sheet 10530 may be formed using a substrate layer 10532 onto which a first layer 10534 including laterally extending wires and an optional second layer 10536 including longitudinally extending wires may be disposed. The substrate layer 10532 may then be fused or otherwise coupled to the first layer 10534 and the second layer 10536, thereby forming the layered sheet 10530, which may subsequently be cut into longitudinal strips. Each strip may then be formed into a tubular shape, as noted above, that includes coils or rings of the wire material distributed along its length. The wire of the second layer 10536, if included, may couple to the wire of the first layer 10534 or may provide additional reinforcement of the layered sheet 10530.

Similar to the previously discussed embodiment, the wire may be incorporated into the layered sheet 10530 in various other ways. For example, in one implementation, the layers including the laterally and longitudinally extending wire (e.g., the first layer 10534 and the second layer 10536, respectively) may be combined into a single layer. In such implementations, the combined layer may be formed to include multiple laterally extending wires and multiple longitudinally extending wires. In alternative implementations, the wire material may be embedded into the substrate layer. In still other implementations, at least some of the laterally extending wire segment and the longitudinally extending wire segments may be formed from a contiguous wire. The wire material may be disposed onto the substrate layer and subsequently coupled to the substrate layer, e.g., by bonding or adhering the wire to the substrate layer or applying an additional layer such that the wire is sandwiched between the additional layer and the substrate layer.

It should be understood that any of the foregoing concepts regarding layered construction of split overtubes discussed herein may be combined in any suitable manner. For example, and without limitation, the layered construction techniques noted above may be used to produce wire- or braid-reinforced reinforced split overtubes that further include working or air channels.

Mandrel-Based Manufacturing of Split Overtubes

In certain implementations of the present disclosure, split overtubes may be manufactured using a mandrel-based technique. More specifically, split overtube may be formed by disposing multiple layers of material onto a mandrel (e.g., by pulling layers onto the mandrel or extruding layers onto the mandrel) and coupling the layers together (e.g., by a reflow operation). Subsequent to coupling the layers, the resulting multi-layer tubular structure may be removed from the mandrel and further processed, e.g., by forming a split along its length, to produce a split overtube.

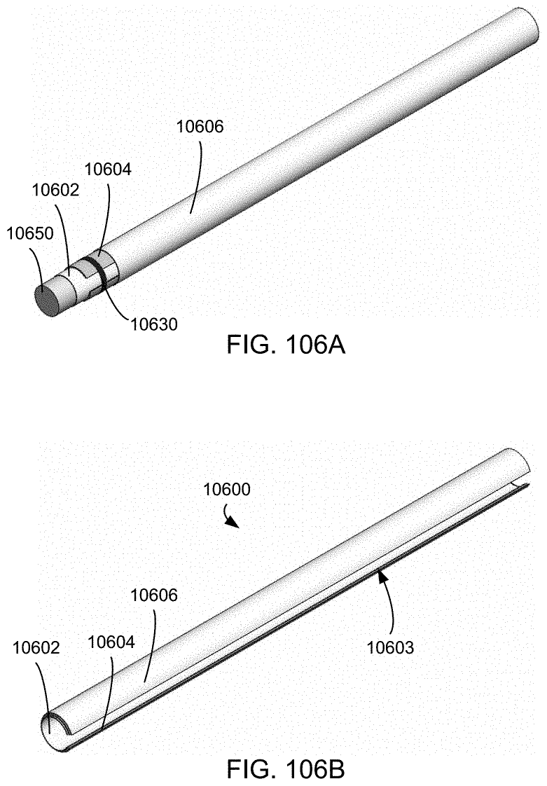

An example of mandrel-based construction of a split overtube 10600 is illustrated in FIGS. 106A and 106B, with the completed split overtube 10600 illustrated in FIG. 106B. Referring first to FIG. 106A, multiple layers of material are disposed onto a mandrel 10650, e.g., by pulling or extruding the layers onto the mandrel 10650. In the specific implementation illustrated in FIGS. 106A and 106B, such layers include a liner layer 10602, a reinforced layer 10604, and an outer layer 10606, each of which are illustrated in FIG. 106A in a staggered configuration for purposes of illustrating their arrangement.

In at least certain implementations, the liner layer 10602 may be formed of a material having a relatively low coefficient of friction, such as, but not limited to polytetrafluoroethylene (PTFE). In certain applications, the low coefficient of friction of the liner layer 10602 facilitates removal of the assembled layers from the mandrel 10650. The low coefficient of friction of the liner layer 10602 may also facilitate translation of an elongate medical device disposed within the split overtube 10600 and relative to the split overtube 10600 during use in medical procedures.

The reinforced layer 10604 generally provides structural integrity and resilience to the split overtube 10600. Accordingly, the reinforced layer 10604 may be formed of reinforced (e.g., braided) tubing material. Alternatively, and as illustrated in FIG. 106A, the reinforced layer 10604 may be in the form of a preformed sheet or split tube that is subsequently wrapped around or disposed around the mandrel 10650. In at least certain implementations, the reinforced layer 10604 may be formed from PEEK, FEP, ETFE, PFA, PVDF, or other similar materials.

Finally, the outer layer 10606 may be formed of a suitable medical polymer that exhibits characteristics suitable for the intended application. For example, in at least certain implementations, the outer layer 10606 may be formed of polyether block amide (e.g., PEBAX®), which generally has mechanical, chemical, and thermal properties suitable for a broad range of medical applications.

In general, the process of forming the split overtube 10600 includes disposing each of the liner layer 10602, the reinforced layer 10604 and the outer layer 10606 onto the mandrel 10650. Once disposed on the mandrel 10650, the layers 10602-10606 may be bonded together, e.g., by a reflow operation. Following bonding, the resulting assembled layers may be removed from the mandrel 10650. Following removal from the mandrel, further processing, such as cutting or otherwise forming a split 10603 along the length of the assembled layers may be performed to complete the split overtube 10600. In implementations in which a split is cut, additional operations may include sealing, bonding, forming a seam, etc. along the edges of the cut, e.g., by applying a suitable coating to the cut edges or reflowing the cut edges. Such processing of the cut edges may be particularly useful in implementations in which cutting the split includes cutting the reinforced layer 10604 and, in particular, reinforcement structure (e.g., a braid) that may be disposed within the reinforced layer 10604 in order to maintain the structural integrity of the reinforced layer 10604.

In certain implementations, discrete reinforcement of the split overtube 10600 may be provided by bands of braided material, coils of wire or similar elongate material, and the like distributed along the length of the split overtube. Examples of such implementations discussed above in the contexts of FIGS. 78B, 780, 105D, and 105E, Similar discrete reinforcements may be incorporated into split overtubes manufactured using mandrel-based techniques. For example, in certain implementations, discrete braids or coils may be incorporated into one or more layers (e.g., a layer that may be wrapped or a tubular layer) that are disposed onto the mandrel 10650 along with the other layers of the overtube (e.g., layers 10602-10606) and that may be bonded with the other layers by the reflow process noted above. In other implementations, reinforcing material may be in the form of preformed strips that are disposed onto the mandrel or inner layers of the overtube during manufacturing. Such layers may be maintained on the mandrel or inner layers by friction, by an adhesive (including an adhesive backing applied to the strips), or other suitable techniques. In still other implementations, the reinforcement may be applied directly onto the mandrel or an inner layer of the overtube. For example, in implementations in which the discrete reinforcements are provided by wire coils, the wire coil may be coiled about the mandrel or an inner layer of the overtube without being incorporated into a separate layer or strip.

The mandrel-based assembly approach permits integration and embedding of various components into the split overtube. For example, FIG. 106A includes a ring 10630 disposed on the mandrel between the reinforced layer 10604 and the outer layer 10606. In certain implementations, the ring 10630 may be formed from a radiopaque material and, as a result, may function as a radiopaque marker of the split overtube. In certain other examples, reinforcing structures, such as circumferentially extending ribs, may be disposed on the mandrel 10650 during assembly for incorporation into the final split overtube. Depending on the component and configuration of the split overtube, components may be disposed directly onto the mandrel 10650 such that they are disposed on an interior surface of the split overtube, disposed on the outer layer 10606 such that they form an exterior of the split overtube, or disposed between any layers of the split overtube such that they are integrated into the wall of the split overtube. As illustrated in FIG. 106A, embedded components, such as the ring 10630 may extend fully around the mandrel 10650. In such cases, the embedded component may be cut when forming the split of the split overtube.

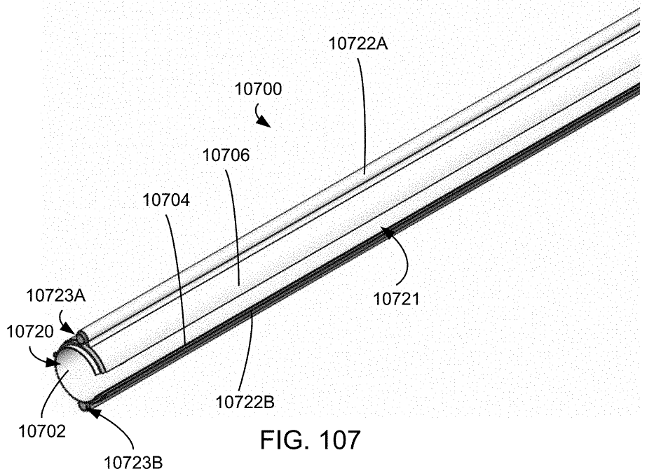

FIG. 107 illustrate another example of a split overtube 10700 that may be formed using a mandrel-based manufacturing technique. As illustrated, the split overtube 10700 includes an inner liner 10702, a reinforced layer 10704, and an outer layer 10706, similar to those discussed above. The inner liner 10702 and the reinforced layer 10704 extend about and define a primary lumen 10720 having a longitudinally extending split 10721. In addition to the primary lumen 10720, the split overtube 10700 further includes a pair of tubules 10722A and 10722B positioned adjacent the primary lumen 10720 and defining a pair of respective secondary lumens 10723A and 10723B. Among other things, the secondary lumens 10723A and 10723B may be used as working channels for tools, to provide or remove fluid, and the like.

Similar to the reinforced layer 10704, the tubules 10722A and 10722B may be reinforced structures. For example, in certain implementations, the tubules 10722A and 10722B may be PTFE tubes reinforced with an embedded braid or coil.

During assembly, the inner liner 10702 may first be disposed on the mandrel followed by the reinforced layer 10704. The tubules 10722A and 10722B may then be disposed adjacent the reinforced layer 10704. In certain implementations, the tubules 10722A and 10722B may be coupled to the reinforced layer 10704, e.g., using a bond or adhesive, or may be supported in their respective locations. Subsequently, the outer layer 10706 may be slid over top of the reinforced layer 10704 and the tubules 10722A and 10722B. A reflow or similar operation may then be conducted to bond the layers together and to retain the tubules 10722A and 10722B in their respective locations. Following reflow, the assembled layers may be removed from the mandrel and processed (e.g., cut) to produce the final split overtube 10700, as illustrated in FIG. 107.

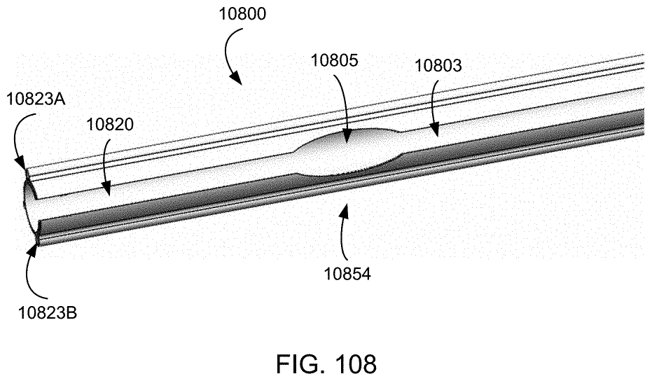

FIG. 108 illustrates another split overtube 10800 that may be formed using a mandrel-based manufacturing method. The split overtube 10800 is substantially similar to the split overtube 10700 illustrated in FIG. 107. Among other things, the split overtube 10800 includes a primary lumen 10820 and secondary lumens 10823A and 10823B adjacent the primary lumen 10820. As illustrated, the primary lumen 10820 is accessible by a split 10803 formed along the length of the split overtube 10800.

As previously discussed in the context of FIGS. 92A-100B, at least certain implementations of split overtubes according to the present disclosure may include features to facilitate insertion of elongate medical devices into the split overtubes. In general, such features include one or both of a local reduction of resistance to insertion of the elongate medical device and a local reinforcement of the split overtube to provide additional leverage during insertion of the elongate medical device.

As shown in FIG. 108, the split overtube 10800 includes an insertion feature 10854 in the form of a widened split portion 10805. Such widening of the split 10803 generally reduces resistance to insertion of an elongate medical device at the location of the widened split portion 10805. In certain implementations, the widened split portion 10805 may be formed when cutting the split 10803.

As previously discussed, various other techniques for forming the insertion feature 10854 may be used in implementations of the present disclosure and may be readily adapted to the mandrel-based manufacturing. For example, and among other things, the layers disposed on the mandrel may be configured to have varying characteristics (e.g., thicknesses, material compositions, etc.) to define the insertion feature. In other implementations, additional components (e.g., ribs, reinforcing plates, etc.) may be disposed onto the mandrel during manufacturing and embedded into the split overtube to define the insertion feature.

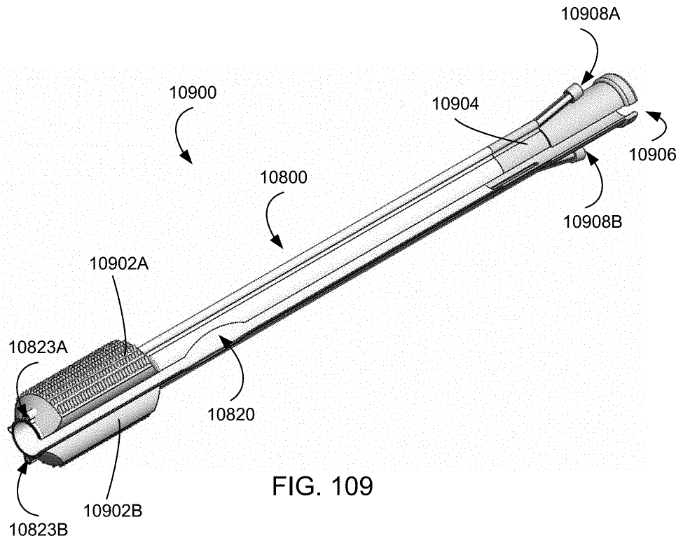

FIG. 109 illustrates the split overtube 10800 integrated into a split overtube assembly 10900, which includes the split overtube 10800, a pair of balloons 10902A, 10902B, and a handle 10904. More specifically, the pair of balloons 10902A, 10902B are disposed on a distal end of the split overtube 10800 while the handle 10904 is disposed on a proximal end of the split overtube 10800 to form the split overtube assembly 10900. Although other handle configurations are contemplated, in the illustrated implementation, the handle 10904 includes a primary handle lumen 10906 in communication with the primary lumen 10820 of the split overtube 10800. The handle 10904 further includes a pair of secondary handle lumens 10908A, 10908B in communication with the secondary lumens 10823A, 10823B of the split overtube 10800.

The foregoing description of a mandrel-based manufacturing method is provided merely as an example. For example, while the foregoing examples generally include three layers, implementations of the present disclosure may include any suitable number of layers. Similarly, any of the other split overtube features disclosed herein may be incorporated into split overtubes manufactured using a mandrel-based approach.

Split Overtube Including Electronic Components

Split overtube assemblies according to the present disclosure may include various electronic components to add functionality and expand the range of applications for which the split overtubes may be used. Among other things and in general, split overtube assemblies may be configured to include various sensors, actuators, output devices, communication media, and the like.

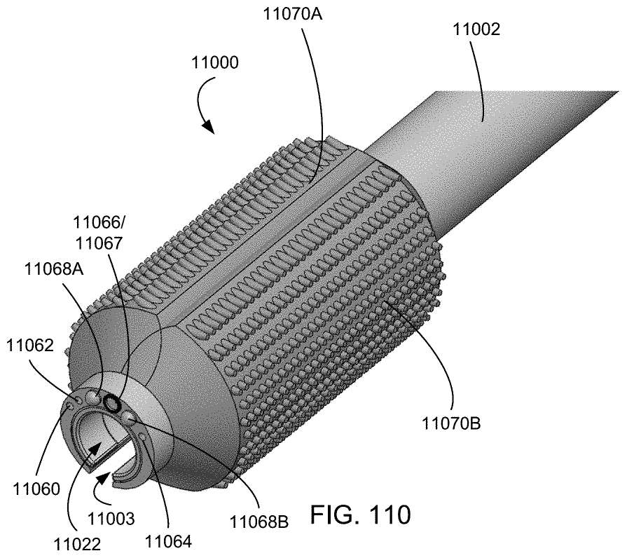

FIG. 110 is an isometric view of a distal end of a split overtube assembly 11000 according to the present disclosure. As illustrated, the split overtube assembly 11000 includes a flexible tubular body 11002 defining each of a primary lumen 11022 and a split 11003 in communication with the primary lumen 11022 and through which an elongate medical device may be inserted into the flexible tubular body 11002. The split overtube assembly 11000 further includes a pair of inflatable balloons 11070A, 11070B, which may be selectively inflated and deflated to anchor the split overtube assembly 11000 within a physiological lumen of a patient.

As previously discussed herein, the flexible tubular body 11002 may be further constructed to define additional lumens, generally referred to as “working” or “secondary” lumens, to provide additional features and functionality. In certain implementations, such secondary lumens may be used to deliver additional tools and devices to a working location at the distal end of the split overtube assembly 11000. In other implementations, secondary lumens may be used as passageways to facilitate fluid communication with a cavity within which the distal end of the split overtube assembly 11000 is disposed. Such fluid communication may be used for, among other things, irrigation (e.g., by providing a liquid into the cavity using a secondary channel), suction (e.g., removal of a fluid from the cavity), and insufflation (e.g., providing air or a gas into the cavity). In still other implementations, secondary lumens may be used to support, house, or otherwise enable the inclusion of various auxiliary components in the split overtube assembly 11000. Among other things and without limitation, such auxiliary components may include output devices (e.g., lights, laser sources, ultrasonic emitters), sensors (e.g., light sensors, pressure sensors, temperature sensors, electrical sensors, electrochemical sensors, etc.), communication media (e.g., wires, fiber optics), and other similar components.

Referring to FIG. 110, for example, the flexible tubular body 11002 defines a collection of six different secondary lumens, each providing a respective function. More specifically, the flexible tubular body 11002 includes each of a suction lumen 11060, an irrigation lumen 11062, and an insufflation lumen 11064, each of which is used to facilitate fluid communication between a proximal and distal end of the split overtube assembly 11000. For example, during use, any of the suction lumen 11060, the irrigation lumen 11062, and the insufflation lumen 11064 may be coupled to a corresponding pump and/or fluid source to provide or remove fluid from within the patient. The flexible tubular body further includes a camera lumen 11066 within which a camera 11067 or similar optical sensing device is disposed as well as a pair of illumination lumens 11068A, 11068B, which contain light-emitting diodes (LEDs) or similar illumination sources.

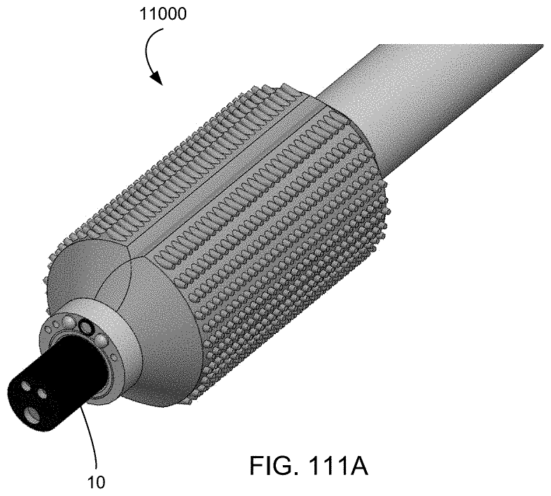

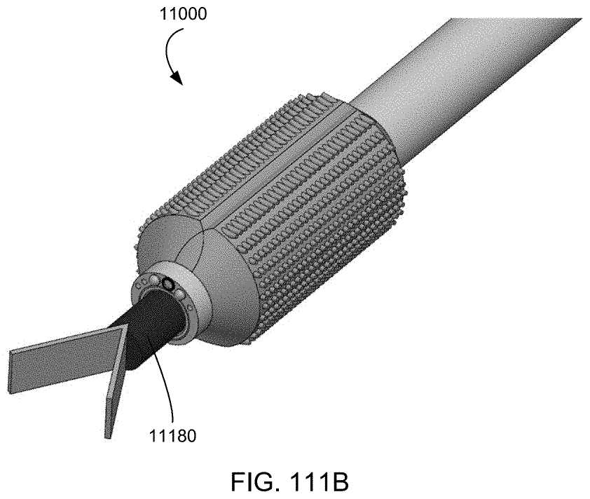

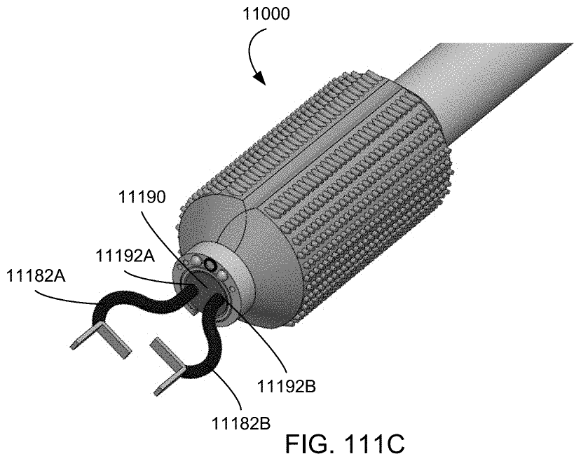

FIGS. 111A-111C illustrate the split overtube assembly 11000 in use with various elongate medical devices. In FIG. 111A, for example, the split overtube assembly 11000 is illustrated as being disposed on an endoscope 10, while FIGS. 111B and 111C illustrate the split overtube assembly 11000 disposed on a large grabber tool 11180 and a pair of small grabber tools 11182A, 11182B, respectively. Notably, the endoscope 10 and grabber tools are provided merely as example tools that may be used and implementations of the present disclosure are not limited to use with such tools and devices.

As previously noted, in at least certain implementations, the split overtube assembly 11000 may include a camera lumen 11066 within which a camera 11067 (each identified in FIG. 110) or similar optical device may be partially disposed. For example, the camera 11067 may be a fiber optic camera with a camera unit disposed proximal and external the flexible tubular body 11002. The camera unit may include a fiber optic extension and lens that may be disposed within the camera lumen 11066 to capture images of a region distal the split overtube assembly 11000.

When used with an endoscope, the camera 11067 may generally provide a second camera view. However, in certain implementations, the camera 11067 may be adapted to capture images using different wavelengths (e.g., IR or thermal) than the endoscope. Moreover, the split overtube design enables removal and replacement of the endoscope 10 with other tools (e.g., the grabber tools illustrated in FIGS. 111B and C), while the split overtube assembly remains disposed within the patient. In applications in which the subsequently inserted tools do not include camera-related functionality, such functionality may be provided by the camera 11067.

For example, in one use case, the endoscope 10 may be used to locate and position the endoscopist for a procedure. Subsequently, the split overtube assembly 11000 may be attached to the endoscope 10 and advanced to the distal end of the endoscope 10. Once positioned, balloons 11070A, 11070B may be inflated to anchor the split overtube assembly 11000 within the patient. The camera 11067 may then be activated and the endoscope 10 removed such that a view within the patient is maintained. The endoscope 10 may be subsequently replaced by other tools for use in completing the procedure and with the advantage of visual feedback provided by the camera 11067 of the split tube assembly 11000.

In certain applications, the primary lumen 11022 of the split overtube assembly 11000 may be sized to accommodate certain tools and devices. For example, as illustrated in each of FIGS. 111A and 111B, the primary lumen 11022 (identified in FIG. 110) is generally sized to receive each of the endoscope 10 and the large grabber tool 11180. In such implementations, smaller diameter tools and devices may nevertheless be delivered using the primary lumen 11022. For example, as illustrated in FIG. 111C, an insert sleeve 11190 may be disposed within the primary lumen 11022 to accommodate smaller diameter tools. More generally, the insert sleeve 11190 defines additional working/secondary lumens for use with the split overtube assembly 11000. As shown, the insert sleeve 11190 defines a first insert lumen 11192A and a second insert lumen 11192B shaped to receive the small grabber tools 11182A, 11182B, respectively. Accordingly, during use, the small grabber tools 11182A, 11182B may be inserted into the insert sleeve 11190, which may then be inserted into the split overtube assembly 11000 through the split 11003. Alternatively, the insert sleeve 11190 may be first disposed within the split overtube assembly 11000 and the small grabber tools 11182A, 11182B may be subsequently inserted through the first and second insert lumens 11192A, 11192B.

As discussed above, split overtube assemblies according to the present disclosure may include various components for providing additional functionality, such as, but not limited to, additional sensing, actuation, and communication functionality. Such components may generally make use of secondary lumens defined within the flexible tubular body of the split overtube, examples of which are discussed below in further detail.

FIG. 112 is a cross-sectional view of a split overtube 11200 defining each of a primary lumen 11202 and secondary lumens 11224, 11226. The split overtube 11200 further includes a first component 11250 disposed within secondary lumen 11224 and a second component 11252 disposed within secondary lumen 11226. More specifically, the first component 11250 is disposed at a distal end of secondary lumen 11224 while the second component 11252 is disposed at an intermediate location within secondary lumen 11226. As illustrated, a plug 11228 or similar structure may be disposed in a distal end of the secondary lumen 11226 to prevent fluid ingress into the secondary lumen 11226.

Although not limited to any specific type of component, in at least certain implementations, one or both of the first component 11250 and the second component 11252 may be sensor components. Examples of sensor components that may be used in implementations of the present disclosure include pressure sensors, temperature sensors, electromagnetic sensors, motion sensors (e.g., accelerometers), light sensors (including cameras), acoustic sensors, chemical sensors, electrochemical sensors, force sensors (e.g., strain gauges), or any other suitable sensor type. Alternatively, one or both of the first component 11250 and the second component 11252 may be output devices. Such output devices may include light devices (e.g., LEDs, lasers), vibration devices, sonic output devices (including ultrasonic emitters), electromagnetic emitters, and the like.

FIG. 113 is a cross-section of another split overtube 11300 including a flexible tubular body 11301 defining each of a primary lumen 11302 and secondary lumens 11324, 11326. The split overtube 11300 further includes a first component 11350 disposed on an outer surface of the flexible tubular body 11301. The first component 11350 is coupled to a communication line 11351 (e.g., a wire or fiber optic cable) that is routed through the secondary lumen 11324. Secondary lumen 11326 is shown as being unobstructed and, as a result, may be suitable for irrigation, suction, insufflation, or similar fluid communication functions. As illustrated, the secondary lumen 11324 extends only partially through the flexible tubular body 11301 of the split overtube 11300.

Sliding Coupling Structures for Overtubes and Elongate Tools

Implementations of the present disclosure may include specific structural features for coupling and guiding components of overtube assemblies, split overtubes, and elongate tools relative to each other. In general, the structural features are in the form of a longitudinally extending rail extending from a first component (e.g., an split overtube) and corresponding groove shaped to receive the rail defined by a second component (e.g., an endoscope). The first and second components can couple to each other by longitudinally sliding the rail into and along the groove. With the rail coupled to the groove, the components are fixed in the rotational and lateral directions but free to move relative to each other in the longitudinal direction.

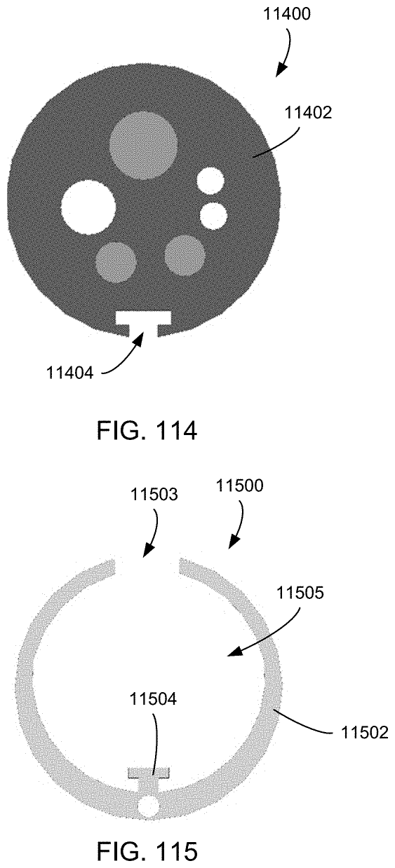

FIGS. 114-116C illustrate a first example of the foregoing concept implemented using an endoscope and a split overtube. FIG. 114 is a distal end view of an example endoscope 11400 according to the present disclosure. Endoscope 11400 includes a body 11402 that may contain various components (e.g., lights, cameras, sensors) and may define one or more lumens extending through elongate body 11402 (e.g., working lumens). Body 11402 further defines a groove 11404 that extends longitudinally along at least a portion of body 11402 and may extend along the full length of body 11402. Although other groove shapes may be used in implementations of this disclosure, groove 11404 is illustrated as having a T-shaped cross-section. Groove 11404 may extend longitudinally along only a portion of body 11402, along multiple portions of body 11402, or substantially along the full length of 11402.

FIG. 115 is a distal end view of an example split overtube 11500 configured to receive and be coupled to endoscope 11400. Split overtube 11500 includes a body 11502 with a longitudinally extending split 11503. During user, elongate tools, such as endoscope 11400 may be inserted through longitudinally extending split 11503 and retained within a primary lumen 11505 defined by body 11502. Although not illustrated, body 11502 may also define one or more secondary or working lumens. Split overtube 11500 may also include various reinforcing structures along its length as well as any other features of split overtubes discussed herein, such as inflatable balloons (which are included in FIGS. 16A-C). As shown in FIG. 115, body 11502 may include a rail 11504 that projects radially inward into primary lumen 11505. Rail 11504 is shown as having a T-shaped cross-section like that of groove 11404 such that rail 11504 may be received by groove 11404 and retained within groove 11404. Rail 11504 may extend longitudinally along only a portion of body 11502, along multiple portions of body 11502, or substantially along the full length of body 11502.

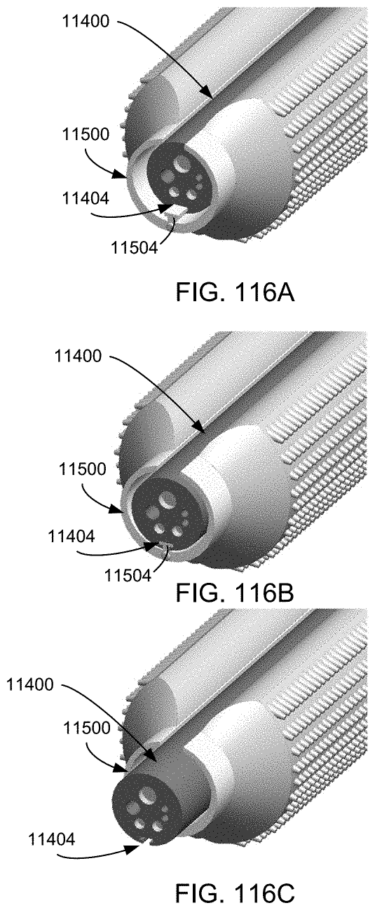

FIGS. 116A-C illustrate endoscope 11400 and split overtube 11500 coupled together, i.e., with rail 11504 of split overtube 11500 received within groove 11404 of endoscope 11400 and endoscope 11400 disposed within primary lumen 11505 (indicated in FIG. 115) of split overtube 11500. As illustrated by the transition between FIGS. 116A-C, when split overtube 11500 and endoscope 11400 are coupled by rail 11504 and groove 11404, split overtube 11500 and endoscope 11400 may be translated longitudinally relative to each other. Specifically, FIG. 116A illustrates a first configuration in which a distal end of endoscope 11400 is proximal a distal end of split overtube 11500. From the position illustrated in FIG. 116A, endoscope 11400 may be translated distally and/or split overtube 11500 may be translated proximally such that the distal end of split overtube 11500 is flush with the distal end of endoscope 11400. Further translation of endoscope 11400 and/or split overtube 11500 may then result in the distal end of endoscope 11400 extending distally beyond the distal end of split overtube 11500, as shown in FIG. 116C.

FIGS. 114-116C illustrate one example implementation in which a single rail of split overtube 11500 is received by a single groove of a tool, such as endoscope 11400. In other implementations, split overtube 11500 may include distributed about the inner circumference of body 11502 and endoscope 11400 may include multiple corresponding grooves. In other implementations, endoscope 11400 may include one or more rails configured to be received by corresponding grooves defined by body 11502 of split overtube 11500 and extending radially outward from primary lumen 11505. In still other implementations, split overtube 11500 may include a combination of one or more rails and one or more grooves configured to mate with one or more corresponding groove and one or more corresponding rails of endoscope 11400.

The specific shape of rails and grooves according to this disclosure may also vary. For example, while FIGS. 114-116C illustrate groove 11404 and rail 11504 as having T-shaped cross-sections, they may instead have semicircular, dovetail, square/rectangular, triangular, or any other regular or irregular cross-sectional shape provided groove 11404 is shaped to receive rail 11504.

FIGS. 114-116C also illustrate rail 11504 as being integrally formed with body 11502. For example, in certain implementations, split overtube 11500 may be formed by an extrusion process with the extruded shape including rail 11504. Alternatively, rail 11504 may be separately formed from and subsequently coupled to body 11502, e.g., by a welding process or adhesive. To permit movement and bending of split overtube 11500, rail 11504 can be formed from a flexible polymer or metal/metal alloy.

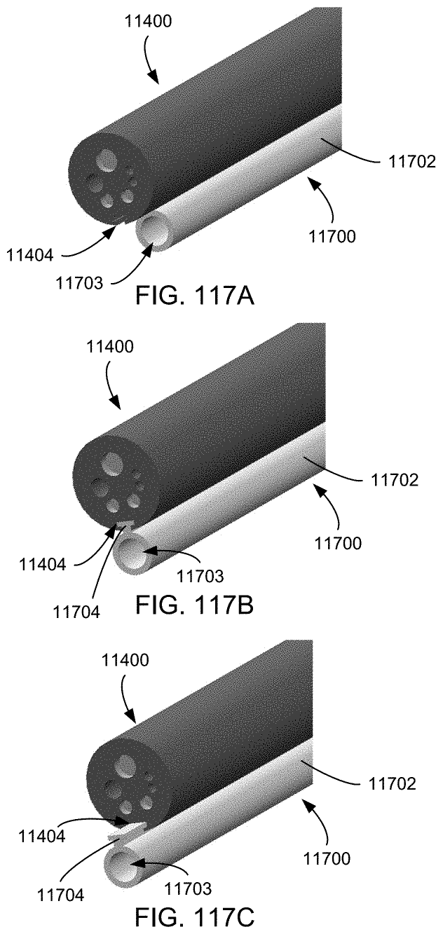

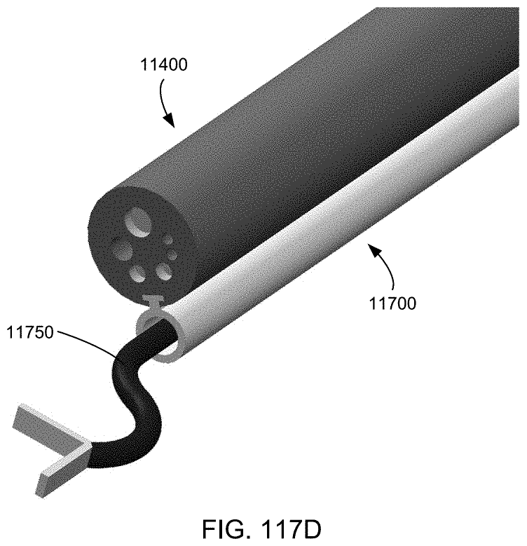

FIG. 117A-C illustrate an alternative implementation of the rail and groove concept. Specifically, FIGS. 117A-C illustrate an implementation in which a rail and groove coupling system is used to couple a tube 11700 to endoscope 11400. As most clearly seen in FIGS. 117B and 117C, tube 11700 includes a body 11702 defining a lumen 11703 that extends along a full length of tube 11700. Tube 11700 further includes a rail 11704 projecting from an exterior surface of body 11702. As shown in in FIGS. 117A-C, rail 11704 is shaped to be received within groove 11404 of endoscope 11400 such that endoscope 11400 and tube 11700 are rotationally and laterally fixed but permitted to longitudinally translate relative to each other. For example, FIG. 117A illustrates a first configuration in which a distal end of endoscope 11400 is distal a distal end of tube 11700. From the position illustrated in FIG. 117A, endoscope 11400 may be translated proximally and/or tube 11700 may be translated distally such that the distal end of tube 11700 is flush with the distal end of endoscope 11400. Further translation of endoscope 11400 and/or split overtube 11500 may then result in the distal end of tube 11700 extending distally beyond the distal end of endoscope 11400, as shown in FIG. 117C.

In certain implementations, tube 11700 may provide a working lumen to supplement the functionality of endoscope 11400. For example, FIG. 117D illustrates tube 11700 coupled to endoscope 11400 to provide a working lumen for a tool 11750 (e.g., a gripper tool). In other implementations, tube 11700 may be a suction or irrigation line.

Tube 11700 may have various shapes and sizes. For example, tube 11700 may have a diameter from about 0.5 mm to 15.0 mm. Also, while illustrates as having a circular cross-section, tube 11700 may have any suitable cross-sectional shape. Tube 11700 may be formed various materials (e.g., polymers or metallic materials) but may be at least partially flexible to permit bending of tube 11700 during use and, in particular, during bending and movement of any component coupled to tube 11700 by a rail and groove structure. Although flexible, tube 11700 may nevertheless include wire reinforcement or be reinforced with another material to prevent collapse of tube 11700 when bent.

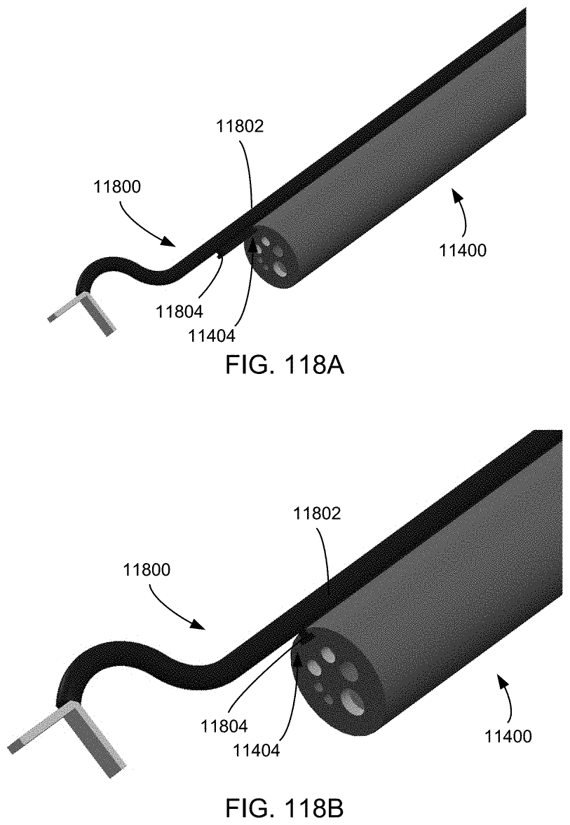

FIG. 118A-B illustrate yet another example implementation of the rail and groove concept in which a supplemental tool is directly coupled to a primary tool. More specifically, FIGS. 118A-B illustrate an endoscope 11400 coupled to a secondary tool 11800 (e.g., a gripper tool) using the rail and groove system. As shown, tool 11800 includes a body 11802 with a rail 11804 projecting from its exterior surface. Rail 11804 is shaped to be received within groove 11404 of endoscope 11400 such that endoscope 11400 and tool 11800 are rotationally and laterally fixed but permitted to longitudinally translate relative to each other. For example, FIG. 118A illustrates a first configuration in which a distal end of rail 11804 of tool 11800 is distal a distal end of groove 11404 of endoscope 11400 and FIG. 118B illustrates a second configuration in which the distal ends of rail 11804 and groove 11404 are substantially flush.

Although illustrated in FIGS. 118A-B as a gripper tool, tool 11800 may be any suitable tool, including tools that include balloons or elements for grasping and manipulating tissue. Also, while endoscope 11400 is shown as having a single groove 11404, endoscope 11400 may include multiple grooves to couple to and guide multiple tools, each of which may be inserted and operated independently. The specific materials of tool 11800 may vary, however, in at least certain implementations, tool 11800 may be generally formed from flexible polymers or metallic components that allow for bending and flexing during use. Although flexible, tools may be reinforced (e.g., by wire or similar reinforcing material) to prevent buckling when the endoscope (or other primary tool) is flexed and to allow for advancement of the tool when the scope is wrapped in a tortuous path. Tool 11800 may also vary in cross-sectional shape and size. For example, tool 11800 may have any suitable cross-sectional shape (e.g., circular or non-circular, constant or varying) and dimension suitable for its particular application. However, in at least certain implementations, tool 11800 may range from and including about 0.5 mm to and including about 15.0 mm in cross-sectional measurement.

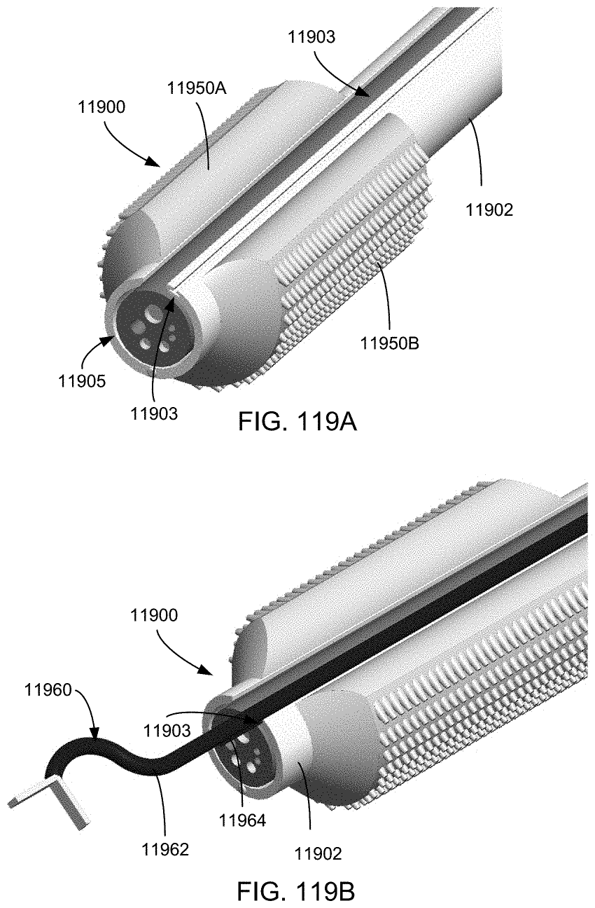

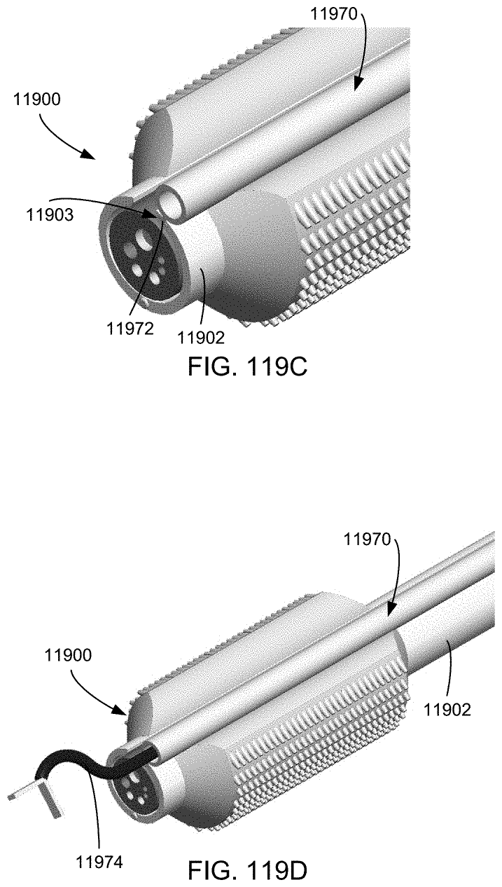

FIGS. 119A-D illustrate yet another implementation of the rail and groove concept in which a split overtube 11900 includes an external groove, each of which may be used to couple to and guide other components. Referring first to FIG. 119A, split overtube 11900 includes a body 11902 with a split 11903 and defining a primary lumen 11905. Body 11902 further defines an external groove 11904 extending adjacent split 11903. As illustrated, external groove 11904 has as T-shaped cross-section, but may have any other suitable shape and may be disposed at a different circumferential location on the exterior of body 11902. Also, to the extent split overtube 11900 is part of an assembly that includes additional components—such as balloons 11950A, 11950B—the additional components may be arranged to be clear of external groove 11904. For example, while balloon 11950A extends up to the edge of split 11903, balloon 11950B terminates away from the opposite edge of split 11903, such that external groove 11904 remains unobstructed.

External groove 11904 may facilitate guidance and delivery of various components with corresponding rails. For example, FIG. 119B illustrates split overtube 11900 coupled to and guiding a secondary tool 11960 where external groove 11904 of split overtube 11900 receives a corresponding rail (not shown, but see rail 11804 of tool 11800 shown in FIGS. 118A-B for a substantially similar structure) extending from a body 11962 of secondary tool 11960. As another example, FIG. 119C illustrates split overtube 11900 coupled to and guiding a tube 11970 where external groove 11904 of split overtube 11900 receives an external rail 11972 of tube 11970. In certain implementations, tube 11970 may then facilitate suction or irrigation to a workspace distal split overtube 11900. Alternatively, and as illustrated in FIG. 119D, tube 11970 may provide a working lumen through which a tool 11974 may be introduced.

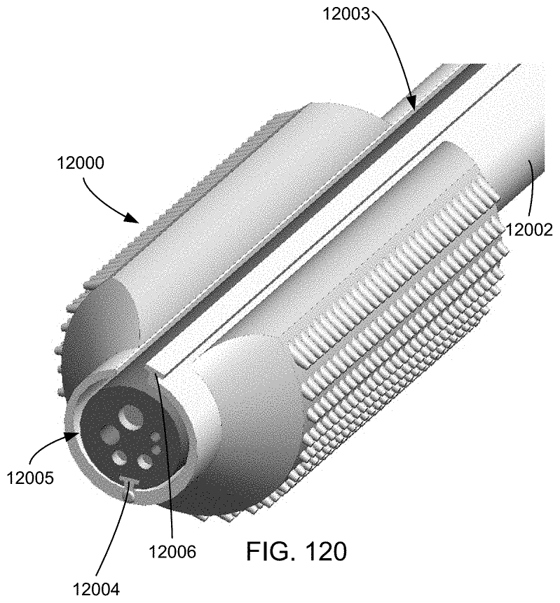

FIG. 120 illustrates yet another example implementation of the rail and groove concept in which a split overtube 12000 includes both internal and external rails. More specifically, split overtube 12000 includes a body 12002 defining a primary lumen 12005. Split overtube 12000 includes an internal rail 12004 that projects inwardly into primary lumen 12005 and which is T-shaped and shown received within groove 11404 of endoscope 11400. Body 12002 further includes an outwardly projecting rail 12006, which is illustrated as extending adjacent a longitudinal split 12003 formed along the length of body 12002. Like internal rail 12004, outwardly projecting rail 12006 has a T-shaped cross-section shaped to be received within a corresponding groove of a secondary component, such as a tool or tube.

Collapsible Secondary Lumens

Implementations of split overtubes in this disclosure generally include a body having a longitudinal split and an internal or primary lumen accessible through the split. Certain implementations may also include one or more secondary lumens in addition to the primary lumen. For example, FIGS. 86A-88B illustrate implementations in which a split overtube includes an external projection or lobe that defines a secondary lumen. FIGS. 89A-91 illustrate alternative implementations in which a secondary lumen is defined within a wall of the split overtube extending around the primary lumen. In either case, the secondary lumen may have different applications; however, in certain implementations, the secondary lumen may provide irrigation, suction, or a pathway for a supplemental tool. Split overtubes according to this disclosure may include multiple secondary lumens. Secondary lumens may extend along the full length of the split overtube or have openings that are proximal a distal end of the split overtube. Secondary lumens may also have openings that are substantially perpendicular to a longitudinal axis of the primary lumen or may be directed at an angle relative to the longitudinal axis of the primary lumen.

The examples of secondary lumens previously discussed in this disclosure are illustrated as having a circular, open cross-section; however, in other implementations, secondary lumens may be collapsible. For example, during insertion of a split overtube assembly including a collapsible secondary lumen, the collapsible secondary lumen may be maintained in a collapsed state to reduce the overall cross-sectional area of the split overtube assembly. Following insertion and locating of the split overtube assembly, the secondary lumen be expanded or opened, e.g., to permit insertion of supplemental tools, etc.

In certain implementations, opening/expanding the secondary lumen may include injecting air or fluid into the secondary lumen to increase the internal pressure of the secondary lumen and cause the secondary lumen to expand. In other implementations, an elongate tool may be inserted into the secondary lumen that expands or opens the secondary lumen as it is pushed along the length of the split overtube. In still other implementations, a tubular structure may be inserted into the secondary lumen to expand and reinforce the secondary lumen.

The collapsible secondary lumen may be biased into a particular state. For example, the secondary lumen may be biased into the closed state such that positive pressure must be maintained within the secondary lumen or a supporting structure must be inserted into the secondary lumen to maintain it in an open configuration. Alternatively, the secondary lumen may be bistable. For example, the secondary lumen may have be generally biased into the closed configuration; however once expanded to a certain extent (e.g., beyond a bistable point) the secondary lumen may “snap” into an open configuration and be subsequently biased into the open configuration until sufficiently collapsed (e.g., beyond the bistable point). To facilitate such functionality, bistable bands of polymer, metal, or similar materials or combinations of materials may be distributed along or embedded within a wall of the secondary lumen.

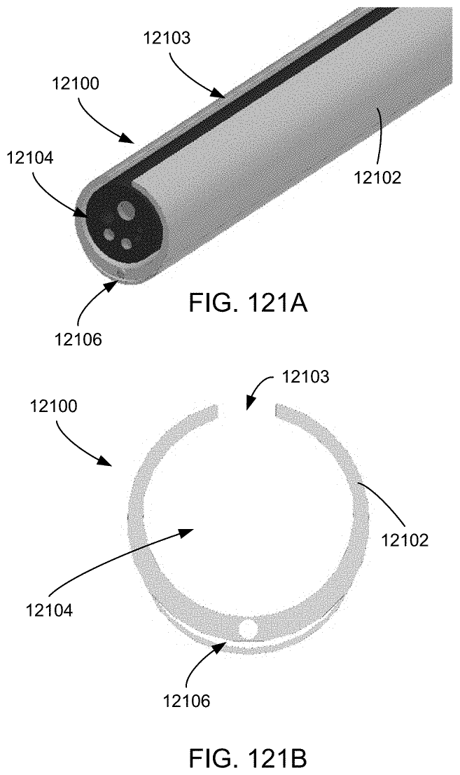

FIGS. 121A-B illustrate an example overtube assembly 12100 including a collapsible secondary lumen. Overtube assembly 12100 includes a tubular body 12102 including a longitudinal split 12103 and defining a primary lumen 12104. As shown in FIG. 121A and discussed throughout this disclosure, an elongate tool, such as an endoscope 10, can be inserted into the primary lumen 12104 through longitudinal split 12103.

Tubular body 12102 further includes a secondary lumen 12106 that may be used for various purposes including, but not limited to, injecting fluids, providing suction, or providing a working channel through which supplemental tools may be inserted. FIG. 121B is a cross-sectional view of tubular body 12102 illustrating secondary lumen 12106.

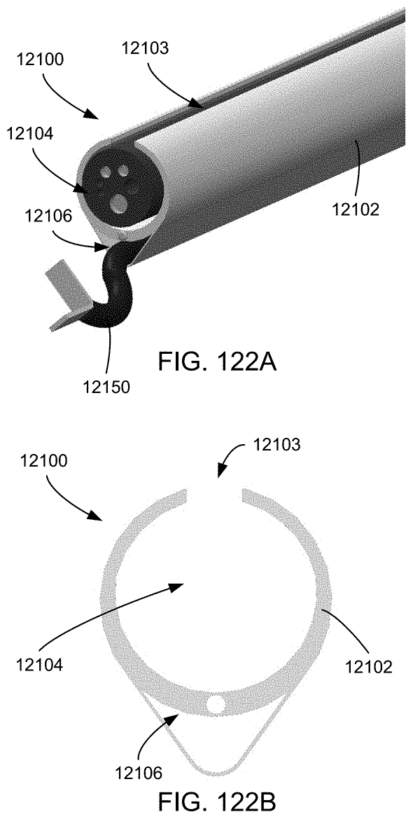

To facilitate insertion and manipulation of overtube assembly 12100, secondary lumen 12106 may be configured to be collapsible. FIG. 121B, for example, illustrates secondary lumen 12106 in the collapsed state. So, for example, secondary lumen 12106 may remain in a collapsed state as endoscope 10 and overtube assembly 12100 are traversed through a physiological lumen of a patient. Once located, secondary lumen 12106 may be expanded to facilitate fluid injection, suction, delivery of supplemental tools, etc. In certain implementations, secondary lumen 12106 may be subsequently collapsed to facilitate repositioning and overtube assembly 12100, including removal of overtube assembly 12100 from the patient. FIGS. 122A-B illustrate overtube assembly 12100 with secondary lumen 12106 in the expanded state with FIG. 122A further illustrating secondary lumen 12106 in use for enabling access to a workspace distal overtube assembly 12100 by a supplemental tool 12150, with FIG. 122B specifically illustrating secondary lumen 12106 in an open configuration.

As previously noted, secondary lumen 12106 may be transitioned between an open and closed configuration using various techniques. For example, in certain implementations, secondary lumen 12106 may be opened by injecting a fluid or expanding tool into secondary lumen 12106. In implementations in which secondary lumen 12106 is biased into the closed configuration, expanding secondary lumen 12106 for use may further include disposing a tubular or similar supporting body into secondary lumen 12106 to maintain secondary lumen 12106 in the open configuration while permitting access through secondary lumen 12106. In still other implementations, secondary lumen 12106 may be formed using bistable structures (e.g., bands, strips, laminated layers) such that secondary lumen 12106 is mechanically stable in each of the open and closed configurations and can be manipulated between both states by applying external or internal force to secondary lumen 12106. For example, secondary lumen 12106 may “snapped” into the open configuration by inserting a tool into secondary lumen 12106 that outwardly expands secondary lumen 12106 beyond a bistable point. Secondary lumen 12106 may then be subsequently collapsed by removing the tool and allowing external forces exerted on secondary lumen 12106 by the patient's body to collapse 121066// beyond the bistable point in the opposite direction.

The specific implementation of a collapsing secondary lumen illustrated in FIGS. 121A-122B, which includes a single collapsible secondary lumen disposed on an exterior surface of the split overtube assembly, is intended only as an example. In other implementations, split overtube assemblies may include multiple collapsible secondary lumens and/or a combination of collapsible and non-collapsible secondary lumens. Also, while secondary lumen 12106 is illustrated as expanding outwardly from tubular body 12102, in certain implementations, secondary lumen 12106 may instead expand inwardly toward primary lumen 12104. Similarly, secondary lumen 12106 may extend through and expand within a wall of tubular body 12102 defining primary lumen 12104. Although collapsible secondary lumens are not limited to any specific size or shape, in at least certain implementations, secondary lumen 12106 may accommodate tools or components having a cross-sectional measurement from and including about 0.5 mm to and including about 15 mm.

### Example Working Environment

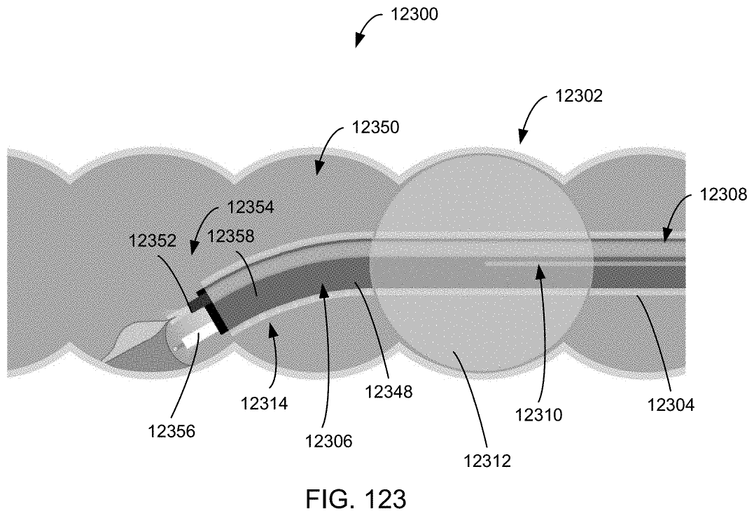

FIG. 123 illustrates an example working environment 12300 including a split overtube assembly 12302 according to the present disclosure. As shown, split overtube assembly 12302 is disposed within a digestive tract 12350 of a patient. Split overtube assembly 12302 includes a split overtube 12304 including a primary lumen 12306 within which a colonoscope 12348 is disposed. Split overtube 12304 further defines a secondary lumen 12308 and an air supply lumen 12310. As illustrated, secondary lumen 12308 is used to a gripper tool 12352 to a workspace 12354 distal split overtube assembly 12302. As shown, gripper tool 12352 is being used in conjunction with a cutting tool 12356 of colonoscope 12 to remove tissue from within digestive tract 12350. Air supply lumen 12310, on the other hand, is used to selective provide air to and remove air from a balloon 12312 of split overtube assembly 12302 that may be used to atraumatically anchor split overtube assembly 12302 within digestive tract 12350. In the specific illustrated example, split overtube 12304 includes a distal portion 12314 that extends distally beyond balloon 12312 and is sufficiently flexible such that distal portion 12314 bends and flexes in response to articulation of a distal portion 12358 of colonoscope 12348.

As used herein, each of the following terms has the meaning associated with it in this section.

As used herein, unless defined otherwise, all technical and scientific terms generally have the same meaning as commonly understood by one of ordinary skill in the art to which this disclosure belongs. Generally, the nomenclature used herein is those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “instructional material” includes a publication, a recording, a diagram, or any other medium of expression that may be used to communicate the usefulness of the compositions and/or methods of the present disclosure. The instructional material of the kit may, for example, be affixed to a container that contains the compositions of the present disclosure or be shipped together with a container that contains the compositions. Alternatively, the instructional material may be shipped separately from the container with the intention that the recipient uses the instructional material and the compositions cooperatively. For example, the instructional material is for use of a kit; and/or instructions for use of the compositions.

Throughout this disclosure, various aspects of the present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Every formulation or combination of components described or exemplified can be used to practice implementations of the current disclosure, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

Although the description herein contains many example implementations, these should not be construed as limiting the scope of the current disclosure but as merely providing illustrative examples.

All references throughout this disclosure (for example, patent documents including issued or granted patents or equivalents; patent application publications; and non-patent literature documents or other source material) are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. In general, the terms and phrases used herein have their art-recognized meaning, which can be found by reference to standard texts, journal references, and contexts known to those skilled in the art. Any preceding definitions are provided to clarify their specific use in the context of the present disclosure.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present disclosure.

The disclosures of each and every patent, patent application, and publication cited herein are hereby incorporated herein by reference in their entirety.

While this disclosure includes reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

### Illustrative Examples of the Disclosure Include

Aspect 1.1: A trocar assembly including a hub; a cannula having an outer surface, the cannula coupled to and extending proximally from the hub; and a textured layer disposed about the outer surface of the cannula, the textured layer including a plurality of outwardly projecting protrusions.

Aspect 1.2 The trocar assembly of claim Aspect 1.1, wherein the textured layer is integrally formed onto the cannula.

Aspect 1.3 The trocar assembly of claim Aspect 1.2, wherein the textured layer is integrally formed onto the cannula by at least one of overmolding, insertion molding, vapor deposition, and spraying the textured layer onto the cannula.

Aspect 1.4 The trocar assembly of claim Aspect 1.1, wherein the outer layer is a tubular sheath within which the cannula is inserted.

Aspect 1.5 The trocar assembly of claim Aspect 1.1, wherein the outer layer is wrapped about the cannula.

Aspect 1.6 The trocar assembly of claim Aspect 1.5, wherein the outer layer is an adhesive-backed tape.

Aspect 1.7 The trocar assembly of claim Aspect 1.5, wherein hub has a hub outer surface, the trocar assembly further including a hub textured layer disposed on at least a portion of the hub outer surface.

Aspect 1.8 The trocar assembly of claim Aspect 1.1, wherein the textured outer layer includes at least one of low-density polyethylene (LDPE), latex, polyether block amide (e.g., PEBAX®), silicone, polyethylene terephthalate (PET/PETE), nylon, or polyurethane.

Aspect 2.1. An overtube assembly for use with an elongate medical device, the overtube assembly including a flexible tubular body having a proximal end and distal end and defining a longitudinal axis therebetween, wherein: the flexible tubular body includes a split extending longitudinally from the proximal end to the distal end, and the flexible tubular body is disposable over a section of the elongate medical device by inserting the elongate medical device through the split; and a plurality of ribs distributed along the length of the flexible tubular body, each rib of the plurality of ribs extending circumferentially about the longitudinal axis and defining a rib split to permit insertion of the medical device into the flexible tubular body.

Aspect 2.2 The overtube assembly of claim Aspect 2.1, wherein a rib of the plurality of ribs is integrally formed with the flexible tubular body.

Aspect 2.3 The overtube assembly of claim Aspect 2.1, wherein a rib of the plurality of ribs is coupled to an exterior surface of the flexible tubular body.

Aspect 2.4 The overtube assembly of claim Aspect 2.1, wherein a rib of the plurality of ribs is coupled to an inner surface of the flexible tubular body.

Aspect 2.5 The overtube assembly of claim Aspect 2.1, wherein the flexible tubular body includes a wall and a rib of the plurality of ribs is disposed within the wall of the flexible tubular body.

Aspect 2.6 The overtube assembly of claim Aspect 2.1, wherein an inner surface of the flexible tubular body is coated with a lubricant.

Aspect 2.7 The overtube assembly of claim Aspect 2.1, wherein a rib of the plurality of ribs includes at least one of polypropylene, polyethylene, nylon, and polyurethane.

Aspect 2.8 The overtube assembly of claim Aspect 2.1, wherein a rib of the plurality of ribs includes a first rib portion disposed on a first side of the rib split of the rib and a second rib portion disposed on a second side of the rib split, wherein the rib is configured such that, during insertion of the elongate medical device into the flexible tubular body, the first rib portion and the second rib portion separate, thereby expanding the rib split.

Aspect 2.9 The overtube assembly of claim Aspect 2.8, wherein the first rib portion and the second rib portion are configured to positively engage each other, thereby closing the rib split.

Aspect 2.10 The overtube assembly of claim Aspect 2.9, wherein the first rib portion includes a first magnet and the second rib portion includes a second magnet such that closing the rib split includes contacting the first magnet with the second magnet.

Aspect 2.11 The overtube assembly of claim Aspect 2.9, wherein the first rib portion includes a first feature and the second rib portion includes a second feature such that closing the rib split includes interlocking the first feature and the second feature.

Aspect 2.12 The overtube assembly of claim Aspect 2.9, wherein the rib is formed of a non-rigid material and the first rib portion and the second rib portion are biased such that, during insertion, of the medical tool, the rib split expands to permit insertion of the elongate medical device and, following insertion of the medical tool, the rib split narrows to a width that is less than a width of the elongate medical device.

Aspect 2.13 The overtube assembly of claim Aspect 2.1, wherein a rib of the plurality of ribs includes a plurality of rib sections coupleable with each other to form an annular structure, wherein the rib is configured to be assembled about the flexible tubular body after insertion of the medical tool therein.

Aspect 2.14 The overtube assembly of claim Aspect 2.1, further including at least one inflatable balloon coupled to a distal portion of the flexible tubular body.

Aspect 3.1 An overtube assembly for use with an elongate medical device, the overtube assembly including a flexible tubular body having a proximal end and distal end and defining a longitudinal axis therebetween, the flexible tubular body including a tube split extending longitudinally from the proximal end to the distal end; and a handle assembly coupled to the proximal end of the flexible tubular body, the handle assembly including a handle body defining a handle split aligned with the tube split, wherein an elongate medical device is insertable into the flexible tubular body by inserting the elongate medical device through the tube split and the handle split.

Aspect 3.2 The overtube assembly of claim Aspect 3.1, wherein the handle split has a width that is less than a width of the elongate medical device, at least one of the handle body and the elongate medical device adapted to deform during insertion of the elongate medical device through the handle split to permit insertion of the elongate medical device through the handle split.

Aspect 3.3 The overtube assembly of claim Aspect 3.1, wherein, when the handle body defines an inner lumen adapted to permit longitudinal movement of the elongate medical device relative to the handle body following insertion of the elongate medical device into the handle body.

Aspect 3.4 The overtube assembly of claim Aspect 3.1, wherein the handle assembly further includes a closure adapted to selectively obstruct at least a portion of the handle split.

Aspect 3.5 The overtube assembly of claim Aspect 3.4, wherein the closure is a detachable cover that is selectively coupleable to the handle body.

Aspect 3.6 The overtube assembly of claim Aspect 3.4, wherein the closure is coupled to the handle body and moveable relative to the handle body between an open position and a closed position, in the open position, the handle split is unobstructed, thereby permitting insertion of the elongate medical device into the handle body, and in the closed position, the handle split is obstructed, thereby prohibiting removal of the elongate tool from the handle.

Aspect 3.7 The overtube assembly of claim Aspect 3.6, wherein transitioning the closure between the open position and the closed position includes rotating the closure about a longitudinal axis of the handle body.

Aspect 3.8 The overtube assembly of claim Aspect 3.6, wherein transitioning the closure between the open position and the closed position further includes longitudinally translating the closure.

Aspect 3.9 The overtube assembly of claim Aspect 3.6, wherein the closure is biased into the closed position.

Aspect 3.10 The overtube assembly of claim Aspect 3.6, wherein at least one of the closure and the handle body includes a stop feature configured to limit movement of the closure relative to the handle.

Aspect 3.11 The overtube assembly of claim Aspect 3.4, wherein the closure is coupled to the handle body by a frictional fit.

Aspect 3.12 The overtube assembly of claim Aspect 3.1, further including at least one inflatable balloon coupled to a distal portion of the flexible tubular body.

Aspect 4.1 An overtube assembly for use with an elongate medical device, the overtube assembly including a flexible tubular body having a proximal end and distal end and defining a longitudinal axis therebetween, the flexible tubular body including a tube split extending longitudinally from the proximal end to the distal end, the elongate medical device insertable into the flexible tubular body via the tube split; and an insertion feature disposed at an initial insertion section of the flexible tubular body, the insertion feature adapted to at least one of provide a leverage point and locally reduce resistance of the tube split at the initial insertion section thereby improving insertion of the elongate medical device at the initial insertion location relative to other locations along the flexible tubular body.

Aspect 4.2 The overtube assembly of claim Aspect 4.1, wherein the insertion feature locally reduces resistance of the tube split at the initial insertion section.

Aspect 4.3 The overtube assembly of claim Aspect 4.2, wherein the insertion feature includes a widening of the tube split at the initial insertion section.

Aspect 4.4 The overtube assembly of claim Aspect 4.2, wherein the insertion feature includes a thinning of a wall material of the flexible tubular body at the initial insertion section, the thinning being relative to other portions of the flexible tubular body outside of the initial insertion section.

Aspect 4.5 The overtube assembly of claim 4,2, wherein at least a portion of the flexible tubular body in the insertion section is formed from a first material and a substantial remainder of the flexible tubular body is formed from a second material, the first material being less stiff than the second material.

Aspect 4.6 The overtube assembly of claim Aspect 4.2, further including a plurality of ribs disposed along the flexible tubular body and extending circumferentially about the flexible tubular body, each of the plurality of ribs defining a rib split through which the elongate medical device may be inserted, the plurality of ribs including at least one first rib disposed in the initial insertion section and at least one second rib disposed outside of the initial insertion section, the at least one first rib configured to reduce resistance of the tube split at the initial insertion section relative to the at least one second rib.

Aspect 4.7 The overtube assembly of claim Aspect 4.6, wherein the at least one first rib is formed of a first material and the at least one second rib is formed of a second material, the first material being less stiff than the second material.

Aspect 4.8 The overtube assembly of claim Aspect 4.6, wherein the at least one first rib has a first width and the at least one second rib has a second width, the first width being less than the second width.

Aspect 4.9 The overtube assembly of claim Aspect 4.6, wherein the at least one first rib has a first thickness and the at least one second rib has a second thickness, the first thickness being less than the second thickness.

Aspect 4.10 The overtube assembly of claim Aspect 4.6, wherein the rib split of the at least one first rib has a first width and the rib split of the at least one second rib has a second width, the first width being greater than the second width.

Aspect 4.11 The overtube assembly of claim Aspect 4.6, wherein the at least one first rib includes two first adjacent ribs and the at least one second rib includes two second adjacent ribs, the first adjacent ribs being spaced further apart than the second adjacent ribs.

Aspect 4.12 The overtube assembly of claim Aspect 4.1, wherein the insertion feature provides a leverage point.

Aspect 4.13 The overtube assembly of claim Aspect 4.12, wherein the insertion feature includes a thickening of a wall material of the flexible tubular body at the initial insertion section, the thickening being relative to other portions of the flexible tubular body outside of the initial insertion section.

Aspect 4.14 The overtube assembly of claim Aspect 4.12, wherein at least a portion of the flexible tubular body in the insertion section is formed from a first material and a substantial remainder of the flexible tubular body is formed from a second material, the first material being more stiff than the second material.

Aspect 4.15 The overtube assembly of claim Aspect 4.12, further including a plurality of ribs disposed along the flexible tubular body and extending circumferentially about the flexible tubular body, each of the plurality of ribs defining a rib split through which the elongate medical device may be inserted, the plurality of ribs including at least one first rib disposed in the initial insertion section and at least one second rib disposed outside of the initial insertion section, the at least one first rib configured to at least partially provide the leverage point.

Aspect 4.16 The overtube assembly of claim Aspect 4.15, wherein the at least one first rib is formed of a first material and the at least one second rib is formed of a second material, the first material being more stiff than the second material.

Aspect 4.17 The overtube assembly of claim Aspect 4.15, wherein the at least one first rib has a first width and the at least one second rib has a second width, the first width being greater than the second width.

Aspect 4.18 The overtube assembly of claim Aspect 4.15, wherein the at least one first rib has a first thickness and the at least one second rib has a second thickness, the first thickness being greater than the second thickness.

Aspect 4.19 The overtube assembly of claim Aspect 4.15, wherein the at least one first rib includes two first adjacent ribs and the at least one second rib includes two second adjacent ribs, the first adjacent ribs being spaced closer together than the second adjacent ribs.

Aspect 4.20 The overtube assembly of claim Aspect 4.15, wherein the at least one first rib includes two adjacent ribs coupled to each other.

Aspect 4.21 The overtube assembly of claim Aspect 4.1, wherein the insertion feature is configured to each of provide the leverage point and locally reduce resistance of the tube split at the initial insertion section.

Aspect 4.22 The overtube assembly of claim Aspect 4.1, further including at least one inflatable balloon coupled to a distal portion of the flexible tubular body.

Aspect 5.1 An overtube assembly for use with an elongate medical device, the overtube assembly including a flexible tubular body having a proximal end and distal end and defining a longitudinal axis therebetween, the flexible tubular body including a tube split extending longitudinally from the proximal end to the distal end, the elongate medical device insertable into the flexible tubular body via the tube split, wherein the flexible tubular body defines each of a primary tool lumen accessible through the tube split and a working lumen separate from the primary tool lumen, the working lumen extending along the length of the flexible tubular body.

Aspect 5.2 The overtube assembly of claim Aspect 5.1, wherein the flexible tubular body includes a primary tubular portion defining the primary tool lumen and a lobe portion coupled to the primary tubular portion defining the working lumen.

Aspect 5.3 The overtube assembly of claim Aspect 5.1, further including a plurality of ribs disposed along the flexible tubular body and extending about the flexible tubular body, wherein each of the plurality of ribs defines a rib split through which the elongate medical device may be inserted into the primary tool lumen and is shaped to extend around each of the primary tool lumen and the working lumen.

Aspect 5.4 The overtube assembly of claim Aspect 5.1, wherein the flexible tubular body includes a wall defining the primary tool lumen, and the wall defines the working lumen.

Aspect 5.5 The overtube assembly of claim Aspect 5.1, further including a handle disposed on the proximal end of the flexible tubular body.

Aspect 5.6 The overtube assembly of claim Aspect 5.5, wherein the working lumen includes a proximal opening disposed distal at least a portion of the handle.

Aspect 5.7 The overtube assembly of claim Aspect 5.5, wherein the working lumen is at least partially defined by the handle.

Aspect 5.8 The overtube assembly of claim Aspect 5.7, wherein the working extends through a proximal end of the handle.

Aspect 5.9 The overtube assembly of claim Aspect 5.1, further including at least one inflatable balloon coupled to a distal portion of the flexible tubular body.

Aspect 6.1 An overtube assembly for use with an elongate medical device, the overtube assembly including a flexible tubular body having a proximal end and distal end and defining a longitudinal axis therebetween, the flexible tubular body including a tube split extending longitudinally from the proximal end to the distal end, the elongate medical device insertable into the flexible tubular body via the tube split; and a first plurality of magnets disposed on a first side of the tube split; and a second plurality of magnets disposed on a second side of the tube split opposite the first side of the tube split, the second plurality of magnets aligned with the first plurality of magnets.

Aspect 6.2 The overtube assembly of claim Aspect 6.1, further including at least one inflatable balloon coupled to a distal portion of the flexible tubular body.

Aspect 7.1. A method of manufacturing an overtube assembly, including forming a strip defining a longitudinal axis and including a strip reinforcement; and forming a split tube by curving the strip about the longitudinal axis.

Aspect 7.2 The method of claim Aspect 7.1, wherein the strip reinforcement is one of a plurality of strip reinforcements distributed along the length of the strip.

Aspect 7.3 The method of claim Aspect 7.1, wherein the strip is a first strip, the method further including, prior to forming the split tube, coupling the first strip to a second strip, the second strip defining a lumen extending longitudinally through the second strip.

Aspect 7.4 The method of claim Aspect 7.3, wherein the lumen is one of a plurality of lumens extending longitudinally through the second strip.

Aspect 7.5 The method of claim Aspect 7.3, wherein the lumen is a working lumen.

Aspect 7.6 The method of claim Aspect 7.3, wherein the lumen is a fluid transportation lumen.

Aspect 7.7 The method of claim Aspect 7.1, wherein the strip is a first strip, the method further including, prior to forming the split tube, coupling the first strip to a second strip, the second strip defining a channel extending longitudinally along the second strip such that, when the first strip is coupled to the second strip, a lumen is formed, the lumen being defined by the channel and the first strip.

Aspect 7.8 The method of claim Aspect 7.1, wherein the strip is a first strip, the method further includes, prior to forming the split tube, coupling the first strip to a second strip, and the split tube is formed such that the first strip is disposed inwardly of the second strip.

Aspect 7.9 The method of claim Aspect 7.1, wherein the strip is a first strip, the method further includes, prior to forming the split tube, coupling the first strip to a second strip, and

the split tube is formed such that the first strip is disposed outwardly of the second strip.

Aspect 7.10 The method of claim Aspect 7.1, wherein the strip reinforcement is a laterally extending rib and the laterally extending rib protrudes from a surface of the strip.

Aspect 7.11 The method of claim Aspect 7.1, wherein the strip reinforcement is a laterally extending rib and the laterally extending rib is flush with a surface of the strip.

Aspect 7.12 The method of claim Aspect 7.1, wherein the strip reinforcement is a laterally extending rib, and the strip defines a reinforcement recess within which the laterally extending rib is disposed.

Aspect 7.13 The method of claim Aspect 7.1, wherein the strip reinforcement is a laterally extending rib, and the laterally extending rib is formed from a plurality of laterally extending reinforcement members.

Aspect 7.14 The method of claim Aspect 7.1, wherein the strip reinforcement is a first portion of the strip having a greater thickness than a second portion of the strip.

Aspect 7.15 The method of claim Aspect 7.1, wherein the strip includes a braid and the strip reinforcement is a first portion of the strip where the braid has a first weave and the strip includes a second portion where the braid has a second weave different than the first weave.

Aspect 7.16 The method of claim Aspect 7.1, further including forming a sheet, wherein forming the strip includes cutting the sheet into a plurality of strips including the strip.

Aspect 7.17 The method of claim Aspect 7.16, wherein forming the sheet includes forming a sheet reinforcement, and a portion of the sheet reinforcement forms the reinforcement of the strip when the sheet is cut to form the strip.

Aspect 8.1 A method of manufacturing a split overtube including disposing a first overtube layer on a mandrel; subsequently disposing a second overtube layer on the mandrel over the first overtube layer; inducing reflow to form an overtube from the first overtube layer and the second overtube layer; removing the overtube from the mandrel; and forming a longitudinal split along the length of the overtube.

Aspect 8.2 The method of claim Aspect 8.1, further including, prior to disposing the first overtube layer on the mandrel, disposing a low friction liner on the mandrel.

Aspect 8.3 The method of claim Aspect 8.2, wherein the low friction liner is formed of polytetrafluoroethylene.

Aspect 8.4 The method of claim Aspect 8.1, wherein the first overtube layer includes a braid.

Aspect 8.5 The method of claim Aspect 8.1, wherein the second overtube layer is an elastomeric layer.

Aspect 8.6 The method of claim Aspect 8.1, wherein the elastomeric layer is formed from Pebax®.

Aspect 8.7 The method of claim Aspect 8.1, wherein the first overtube layer includes a braid and the second overtube layer is an elastomeric layer.

Aspect 8.8 The method of claim Aspect 8.1, further including, subsequent to forming the longitudinal split, sealing an inner edge of the split.

Aspect 8.9 The method of claim Aspect 8.1, wherein the first overtube layer includes a braid and does not extend fully about the mandrel.

Aspect 8.10 The method of claim Aspect 8.9, further including, subsequent to disposing the first overtube layer on the mandrel, disposing a retainer onto the mandrel to retain the first overtube layer on the mandrel.

Aspect 8.11 The method of claim Aspect 8.10, wherein the retainer is a split ring including a split, and forming the longitudinal split includes forming the longitudinal split to be aligned with the split.

Aspect 8.12 The method of Aspect 8.10, wherein the retainer is a ring, and forming the longitudinal split includes forming a split in the ring.

Aspect 8.13 The method of claim Aspect 8.10, wherein the retainer is radiopaque.

Aspect 8.14 The method of claim Aspect 8.1, further including, prior to disposing the second overtube layer onto the mandrel, disposing a radiopaque marker onto the mandrel such that, the overtube is formed with the radiopaque marker disposed between the first overtube layer and the second overtube layer.

Aspect 8.15 The method of claim Aspect 8.1, wherein the first overtube layer forms a primary lumen of the overtube, the method further including prior to disposing the second overtube layer onto the mandrel, disposing a secondary lumen adjacent the first overtube layer such that the second overtube layer further extends over the secondary lumen.

Aspect 8.16 The method of claim Aspect 8.1, wherein the longitudinal split includes a first portion having a first width and a second portion having a second width different than the first width.

Aspect 8.17 The method of claim Aspect 8.1, further including disposing a reinforcing member onto the mandrel such that the overtube is formed with the reinforcing member disposed radially inward of the first overtube layer.

Aspect 8.18 The method of claim Aspect 8.1, further including disposing a reinforcing member onto the mandrel such that the overtube is formed with the reinforcing member disposed between the first overtube layer and the second overtube layer.

Aspect 8.19 The method of claim Aspect 8.1, further including disposing a reinforcing member onto the mandrel such that the overtube is formed with the reinforcing member disposed outward of the second overtube layer.

Aspect 8.20 The method of claim Aspect 8.1, further including subsequent to forming the longitudinal split, coupling an inflatable balloon to a distal end of the overtube such that a longitudinally extending split of the inflatable balloon is aligned with the longitudinal split.

Aspect 8.21 The method of claim Aspect 8.1, further including, subsequent to forming the longitudinal split, coupling each of a first inflatable balloon and a second inflatable balloon to a distal end of the overtube such that a gap is defined between the first inflatable balloon and the second inflatable balloon and the gap is aligned with the longitudinal split.

Aspect 8.22 The method of claim Aspect 8.1, further including, subsequent to forming the longitudinal split, coupling a handle to a proximal end of the overtube such that a longitudinally extending slot of the handle is aligned with the longitudinal split.

Aspect 8.23 The method of claim Aspect 8.1, wherein the first overtube layer forms a primary lumen of the overtube, the method further including prior to disposing the second overtube layer onto the mandrel, disposing a secondary lumen adjacent the first overtube layer such that the second overtube layer further extends over the secondary lumen; coupling a handle to a proximal end of the overtube, the handle including a primary port and a secondary port separate from the primary port, wherein coupling the handle to the proximal end of the overtube includes aligning the primary port to be in communication with the primary lumen and the secondary port to be aligned with the secondary lumen.

Aspect 9.1 An overtube assembly for use with an elongate medical device, the overtube assembly including a flexible tubular body having a proximal end and a distal end, the flexible tubular body including a tube split extending longitudinally from the proximal end to the distal end, wherein the flexible tubular body defines each of a primary tool lumen extending from the proximal end to the distal end and accessible through the tube split and a secondary lumen separate from the primary tool lumen, and wherein the secondary lumen is collapsible.

Aspect 9.2 The overtube assembly of Aspect 9.1, wherein the secondary lumen expands outwardly from the flexible tubular body.

Aspect 9.3 The overtube assembly of Aspect 9.1, wherein the secondary lumen expands inwardly into the primary lumen.

Aspect 9.4 The overtube assembly of Aspect 9.1, wherein the secondary lumen is biased into a collapsed state.

Aspect 9.5 The overtube assembly of Aspect 9.1, wherein the secondary lumen is bistable between a collapsed state and an open state.

What is claimed is:

1. An overtube assembly for use with an elongate medical device, the overtube assembly comprising:
a flexible tubular body having a proximal end and a distal end, the flexible tubular body including a tube split extending longitudinally from the proximal end to the distal end,
wherein the flexible tubular body defines each of a primary lumen extending from the proximal end to the distal end and accessible through the tube split and a secondary lumen separate from the primary lumen.

2. The overtube assembly of claim 1, wherein the flexible tubular body comprises a primary tubular portion defining the primary lumen and a lobe portion coupled to the primary tubular portion defining the secondary lumen.

3. The overtube assembly of claim 1, further comprising a reinforcing rib extending about the flexible tubular body, wherein the reinforcing rib defines a rib split aligned with the tube split.

4. The overtube assembly of claim 1, further comprising a reinforcing rib extending about the flexible tubular body, wherein the reinforcing rib defines a rib split aligned with the tube split, and the reinforcing rib extends about each of the primary lumen and the secondary lumen.

5. The overtube assembly of claim 1, wherein:
the flexible tubular body comprises a wall defining the primary lumen, and
the wall defines the secondary lumen.

6. The overtube assembly of claim 1, further comprising a handle disposed on the proximal end of the flexible tubular body, wherein the secondary lumen includes a proximal opening disposed distal at least a portion of the handle.

7. The overtube assembly of claim 5, further comprising a handle disposed on the proximal end of the flexible tubular body, wherein the secondary lumen is at least partially defined by the handle.

8. The overtube assembly of claim 1, further comprising an inflatable balloon coupled to a distal portion of the flexible tubular body.

9. The overtube assembly of claim 1, further comprising an inflatable balloon coupled to a distal portion of the flexible tubular body, wherein the secondary lumen includes a distal opening and the distal opening is proximal the inflatable balloon.

10. The overtube assembly of claim 1, wherein the secondary lumen is one of a plurality of secondary lumens defined by the flexible tubular body.

11. The overtube assembly of claim 1, wherein the primary lumen defines a longitudinal axis and wherein the secondary lumen includes a distal opening that is non-perpendicular relative to the longitudinal axis.

12. The overtube assembly of claim 1, wherein the secondary lumen is collapsible.

13. The overtube assembly of claim 1, wherein the secondary lumen is bistable between an open configuration and a closed configuration.

14. The overtube assembly of claim 1 further comprising a tubule disposed along a length of the secondary lumen.

15. An overtube assembly comprising:
a tubular body having a proximal end and a distal end, the tubular body including a tube split extending longitudinally from the proximal end to the distal end, wherein the flexible tubular body defines each of (i) a primary lumen accessible through the tube split and extending from the proximal end to the distal end; (ii) a secondary lumen separate from the primary lumen; and (iii) a fluid supply lumen separate from each of the primary lumen and the secondary lumen; and
an inflatable balloon disposed on a distal portion of the tubular body an in communication with the fluid supply lumen such that inflation of the inflatable balloon is controllable by selectively providing or removing fluid via the fluid supply lumen.

16. The overtube assembly of claim 15, wherein the secondary lumen includes a distal opening disposed proximal the inflatable balloon.

17. The overtube assembly of claim 15, wherein the secondary lumen includes a distal opening disposed distal the inflatable balloon.

18. The overtube assembly of claim 15 further comprising an electronic component, wherein the electronic component is at least one of disposed within and routed through the secondary lumen.

19. A method, comprising:
disposing an overtube assembly onto an elongate tool, wherein:
the overtube assembly includes a flexible tubular body having a proximal end and a distal end, the flexible tubular body including a tube split extending longitudinally from the proximal end to the distal end,
the flexible tubular body defines a primary lumen accessible through the tube split and a secondary lumen separate from the primary lumen, and
disposing the overtube assembly onto the elongate tool includes inserting the elongate tool through the tube split;
locating the overtube assembly within a patient; and
subsequent to locating the overtube assembly within the patient, inserting a secondary tool into the secondary lumen.

20. The method of claim 19, wherein the overtube assembly further includes an inflatable balloon disposed on a distal portion of the flexible tubular body, and the method further comprises anchoring the overtube assembly subsequent to locating the overtube assembly within the patient by inflating the balloon.
